SEQUENCE LISTING (3 sequences)

SEQ ID NO: 1 - protein, 14 aa, Artificial Sequence
  polypeptide
  MOD_RES (1)..(1): 5FAM
  MOD_RES (13)..(13): (5TAMRA)
TEGEARGSVILLKK

SEQ ID NO: 2 - protein, 473 aa, Homo sapiens
FASLSRFVETLVVADDKMAAFHGAGLKRYLLTVMAAAAKAFKHPSIRNPVSLVVTRLVILGSGEEGPQVGPSAAQTLRSFCAWQRGLNTPEDSDPDHFDTAILFTRQDLCGVSTCDTLGMADVGTVCDPARSCAIVEDDGLQSAFTAAHELGHVFNMLHDNSKPCISLNGPLSTSRHVMAPVMAHVDPEEPWSPCSARFITDFLDNGYGHCLLDKPEAPLHLPVTFPGKDYDADRQCQLTFGPDSRHCPQLPPPCAALWCSGHLNGHAMCQTKHSPWADGTPCGPAQACMGGRCLHMDQLQDFNIPQAGGWGPWGPWGDCSRTCGGGVQFSSRDCTRPVPRNGGKYCEGRRTRFRSCNTEDCPTGSALTFREEQCAAYNHRTDLFKSFPGPMDWVPRYTGVAPQDQCKLTCQAQALGYYYVLEPRVVDGTPCSPDSSSVCVQGRCIHAGCDRIIGSKKKFDKCMVCGGDGSGC

SEQ ID NO: 3 - protein, 361 aa, Homo sapiens
SISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCP

(19) United States
(12) Patent Application Publication
(10) Pub. No.: US 2024/0299360 A1
(43) Pub. Date: Sep. 12, 2024

(54) ADAMTS INHIBITORS, PREPARATION METHODS AND MEDICINAL USES THEREOF

(71) Applicant: Jiangsu Hengrui Pharmaceuticals Co., Ltd., Lianyungang, Jiangsu (CN)

(72) Inventors: Dong Liu, Basking Ridge, NJ (US); Peng Zhao, Jamesburg, NJ (US); Jian Liu, Edison, NJ (US); Linghang Zhuang, Chalfont, PA (US); Fengqi Zhang, Edison, NJ (US); Xinzhu Zhang, Frnklin Park, NJ (US); Chunying Song, Newtown, PA (US); Suxing Liu, Edison, NJ (US); Jing Li, Skillman, NJ (US)

(21) Appl. No.: 17/760,169

(22) PCT Filed: Feb. 3, 2021
(86) PCT No.: PCT/US21/16364

§ 371 Date: Aug. 4, 2022

Related U.S. Application Data
(60) Provisional application No. 62/969,992, filed on Feb. 4, 2020.
(60) Provisional application No. 63/066,148, filed on Aug. 14, 2020.
(60) Provisional application No. 63/087,656, filed on Oct. 5, 2020.

(51) Int. Cl. A61K 31/4178; A61K 31/422; A61K 31/427; A61K 31/437; A61K 31/4439; A61K 31/4725; A61K 31/497; A61K 31/506; A61K 31/55; C07D 401/06; C07D 401/14; C07D 403/06; C07D 403/14; C07D 413/14; C07D 417/14; C07D 471/04

(52) U.S. Cl. CPC A61K 31/4178; A61K 31/422; A61K 31/427; A61K 31/437; A61K 31/4439; A61K 31/4725; A61K 31/497; A61K 31/506; A61K 31/55; C07D 401/06; C07D 401/14; C07D 403/06; C07D 403/14; C07D 413/14; C07D 417/14; C07D 471/04

(57) ABSTRACT

Compounds of formula (I) useful as inhibitors of ADAMTS-5 and/or ADAMTS-4, pharmaceutical compositions thereof, and use of them as therapeutic agents for the treatment of diseases involving degradation of cartilage or disruption of cartilage homeostasis, in particular osteoarthrosis and/or rheumatoid arthritis, are disclosed.

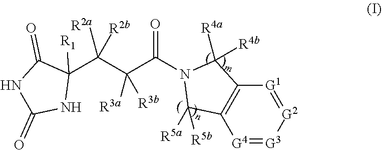

### CROSS-REFERENCE TO RELATED APPLICATIONS

This application claims priority under 35 U.S.C. § 119(e) to U.S. Provisional Patent Application No. 62/969,992, filed on Feb. 4, 2020; No. 63/046,267, filed on Jun. 30, 2020; No. 63/066,148, filed on Aug. 14, 2020; and No. 63/087,656, filed on Oct. 5, 2020, the disclosures of all of which are incorporated herein by reference in their entireties.

### FIELD OF THE DISCLOSURE

The present disclosure relates to compounds and methods in inhibiting the function of ADAMTS-5 and/or ADAMTS-4 and their application in the treatment of diseases involving degradation of cartilage or disruption of cartilage homeostasis, such as osteoarthritis and/or rheumatoid arthritis.

### BACKGROUND OF THE DISCLOSURE

Cartilage is the highly specialized connective tissue of diarthrodial joints. Its principal function is to provide the joints the capability of load bearing and compression resistance. Chondrocyte is the cellular component of articular cartilage, taking about only 5% of the tissue volume. The main component of cartilage is extracellular matrix comprising aggrecan and collagen. Under physiological conditions, cartilage homeostasis is maintained by a balance between production (anabolism) and degradation (catabolism) of aggrecan and collagen. However, the balance is shifted to catabolism in diseases such as osteoarthritis.

Osteoarthritis is the most common chronic joint disease and a leading cause of pain and disability in developed countries. It can happen to the joints of the hips, knees, spines, hands and others. It was estimated that worldwide 250 million people are currently being affected by osteoarthritis, and the prevalence is progressively rising (Hunter et al., Lancet. 2019, 393: 1745-1759). Pain and loss of functional capacity are accompanied by an increased risk of additional disease conditions such as diabetes, cancer or cardiovascular disease (Valdes AM and Stocks J. Osteoarthritis and ageing. Eur Med J. 2018, 3:116-123). Osteoarthritis is a whole joint disease, the structural changes of which are found to be degradation of articular cartilage, synovitis and alterations in subchondral bone and other periarticular tissues (Goldring MB and Otero M. Inflammation in osteoarthritis. Curr Opin Rheumatol. 2011, 23: 471-478). The pathogenesis of osteoarthritis is not very clear, with mechanical damage, inflammation, aging, and metabolism factors being involved. Osteoarthritis is not a passive degenerative disease, but an active dynamic alteration arising from an imbalance between the repair and destruction of joint tissues (Hunter et al., Lancet. 2019, 393: 1745-1759). Currently, the pharmacological treatments available for osteoarthritis are limited to symptomatic relief of pain and inflammation. Disease-modifying drugs that arrest or slow down disease progression are not available.

Progressive loss of articular cartilage is currently viewed as an early event in osteoarthritis. Aggrecan may have a role protecting loss of collagen (Pratta et al., J Biol Chem. 2003, 278: 45539-45545). These studies suggest the critical role of aggrecan in osteoarthritis and other joint diseases. Aggrecan is a proteoglycan, possessing a core protein with covalently attached sulfated glycosaminoglycan (GAG) chains. Its core protein has three globular domains, G1 and G2 domains in the N-terminus, and G3 in the C-terminus. The extensive region between the G2 and G3 domains is heavily modified by GAG keratan sulfate (KS) and chondroitin sulfate (CS). Based on the difference in the amino acid sequence, the CS domain is further divided into two subdomains, CS1 and CS2. The GAG chains provide aggrecan with its high anionic charge. Multiple aggrecan monomers bind to hyaluronan (HA) through G1 domains, which is stabilized by a link protein, forming large supramolecular aggregates. The large aggrecan aggregates absorb water and provide the resilient properties for the cartilage (Roughley et al., The Journal of Experimental Orthopaedics. 2014, 1: 8). A high concentration of aggrecan, a high degree of sulfation and the ability to form large aggregation is required for the normal function of cartilage.

The extended structure of aggrecan can be cleaved by proteolytic enzymes, leading to impaired normal function of cartilage. ADAMTS (a disintegrin and metalloproteinase with thrombospondin motifs) is a family of zinc ion-dependent metalloproteases. ADAMTS-4 and -5, also termed “aggrecanases”, degrade aggrecan at several specific locations in the IGD and the CS2 domain. It was demonstrated that ADAMTS-5 deficiency protects against aggrecan loss and cartilage damage in mouse osteoarthritis disease model induced by surgeries (Glasson et al., Nature. 2005, 434: 644-648; Stanton et al., Nature. 2005, 434:648-652), implicating ADAMTS-5 in driving cartilage loss and osteoarthritis disease severity. However, some studies in human cartilage explant culture suggested that not only ADAMTS-5, but also ADAMTS-4 are important for human osteoarthritis (Verma et al., Journal of Cellular Biochemistry. 2011, 112: 3507-3514). These studies strongly suggest that inhibiting the enzymatic function of ADAMTS-5 and ADAMT-4 might provide a protecting role in osteoarthritis.

In sum, the role of ADAMTS-5 and/or ADAMTS-4 in cartilage degradation has been well-established. Therefore, compounds that can inhibit ADAMTS-5 and/or ADAMTS-4 may be of therapeutic value in the treatement of arthritis.

### SUMMARY OF THE DISCLOSURE

The compounds of this disclosure inhibit the function of ADAMTS-5 and/or ADAMTS-4 and accordingly may serve as therapeutic agents for the treatment of diseases involving degradation of cartilage or disruption of cartilage homeostasis, in particular, osteoarthritis and/or rheumatoid arthritis.

The present disclosure, in one aspect, provides a compound of formula (I), or a tautomer, mesomer, racemate, enantiomer, diastereomer thereof, or mixture thereof, or a pharmaceutically acceptable salt, solvate or prodrug thereof:

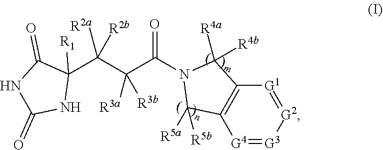

wherein:
G¹, G², G³ and G⁴ are each independently N or CR⁶, provided that no more than two of them are N;
R¹ is selected from the group consisting of alkyl, haloalkyl, hydroxyalkyl, cycloalkyl, heterocyclyl and heteroaryl, wherein the alkyl, cycloalkyl, heterocyclyl and heteroaryl are each optionally substituted with one or more, preferably one to five, and sometimes more preferably one to three, groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NH C(═O)R^{11a}, NH C(═O)OR^{11a}, cycloalkyl, heterocyclyl, aryl and heteroaryl;
R^{2a}, R^{2b}, R^{3a} and R^{3b} are each independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, OR^{12a}, cycloalkyl, heterocyclyl, aryl and heteroaryl;
or alternatively, two of R^{2a}, R^{2b}, R^{3a} and R^{3b} together with the carbon atom to which they are attached form cycloalkyl or heterocyclyl;
R^{4a}, R^{4b}, R^{5a} and R^{5b} are each independently selected from the group consisting of hydrogen, deuterium, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, alkyl, alkoxy, hydroxy, hydroxyalkyl, cyano, amino, nitro, cycloalkyl, heterocyclyl, aryl and heteroaryl;
or alternatively, two of R^{4a}, R^{4b}, R^{5a} and R^{5b} together with the carbon atom they are attached form cycloalkyl or heterocyclyl;
R⁶ at each occurrence is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl and heteroaryl are each optionally substituted with one or more, preferably one to five, and sometimes more preferably one to three, groups independently selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
each of R^{11a}, R^{12a}, R^{13a} and R^{14a} is independently selected from the group consisting of hydrogen, alkyl, cycloalkyl, heterocyclyl, aryl and heteroaryl, wherein the alkyl, cycloalkyl, heterocyclyl, aryl and heteroaryl are each optionally substituted with halogen, hydroxy, alkoxy, alkyl, aryl and cycloalkyl;
each of R^{11a}, R^{12b}, R^{13b}, and R^{14b} is independently selected from the group consisting of hydrogen and alkyl, wherein alkyl is optionally substituted with halogen, hydroxyl and alkoxy;
n is 1 or 2; and
m is 1 or 2.

In another aspect, this disclosure provides a preparation process of a compound of formula (I), or a tautomer, mesomer, racemate, enantiomer, diastereomer, or mixture thereof, or a pharmaceutically acceptable salt, solvate or prodrug thereof, the preparation process comprising the steps of:

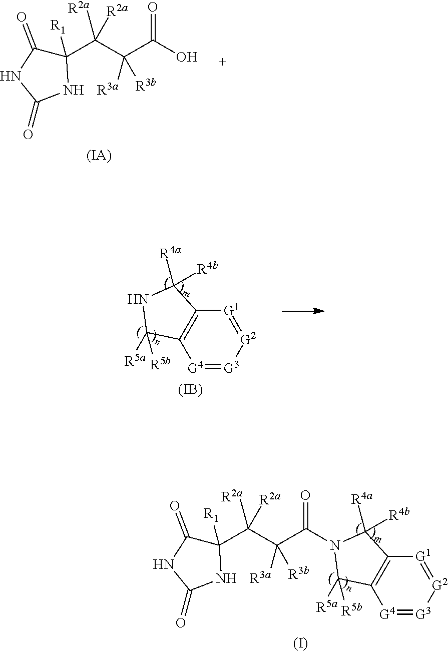

reacting a compound of formula (IA) with a compound of formula (IB), or a pharmaceutically acceptable salt thereof, to obtain the compound of formula (I), wherein:
the pharmaceutically acceptable salt of the compound (IB) preferably is hydrochloride; and
G¹, G², G³, G⁴, R¹, R^{2a} to R^{5a}, R^{2b} to R^{5b}, n and m are each as defined in formula (I).

In another aspect, the present disclosure provides a pharmaceutical composition comprising a compound of formula (I), or an isomer, pharmaceutically acceptable salt, solvate or prodrug thereof, and a pharmaceutically acceptable carrier. In another aspect, the present disclosure provides a method of preventing and/or treating inflammatory conditions, and/or diseases involving degradation of cartilage, and/or disruption of cartilage homeostasis, comprising a step of administering to a subject in need thereof a therapeutically effective amount of a compound of formula (I), or an isomer, pharmaceutically acceptable salt, solvate, or prodrug, or a pharmaceutical composition containing the compound.

In another aspect, the present disclosure also relates to use of a compound of formula (I), or an isomer, pharmaceutically acceptable salt, solvate or prodrug thereof, or a pharmaceutical composition containing the compound, in the manufacture of a medicament for treatment of an inflammatory condition, and/or a disease involving degradation of cartilage, and/or disruption of cartilage homeostasis.

The disease or condition includes arthritis, preferably, rheumatoid arthritis, psoriatic arthritis, osteoarthrosis and hypertropic arthritis, which are further preferably related to the activity of ADAMTS-5 and/or ADAMTS-4.

Other aspects or advantages of the disclosure will be better appreciated in view of the following detailed description, examples, and claims.

### DETAILED DESCRIPTION OF THE DISCLOSURE

In one aspect, the present disclosure provides a compound of formula (I), or an isomer, pharmaceutically acceptable salt, solvate or prodrug thereof:

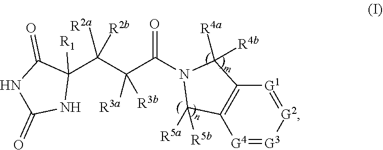

wherein:
G¹, G², G³ and G⁴ are each identical or different, and each is N or CR⁶, provided that no more than two of them are N;
R¹ is selected from the group consisting of alkyl, haloalkyl, hydroxyalkyl, cycloalkyl, heterocyclyl and heteroaryl, wherein the alkyl, cycloalkyl, heterocyclyl or heteroaryl is optionally substituted with one or more groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NHC(═O)R^{11a}, NHC(═O)OR^{11a}, cycloalkyl, heterocyclyl, aryl and heteroaryl;
R^{2a}, R^{2b}, R^{3a} and R^{3b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, OR^{12a}, cycloalkyl, heterocyclyl, aryl and heteroaryl; or two of R^{2a}, R^{2b}, R^{3a} and R^{3b} together with the carbon atom to which they are attached form cycloalkyl or heterocyclyl;
R^{4a}, R^{4b}, R^{5a} and R^{5b} are each identical or different, and each is independently selected from the group consisting of hydrogen, deuterium, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, cycloalkyl, heterocyclyl, aryl and heteroaryl;
or two of R^{4a}, R^{4b}, R^{5a} and R^{5b} together with the carbon atom to which they are attached form cycloalkyl or heterocyclyl;
each R⁶ is identical or different, and each is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
R^{11a}, R^{12a}, R^{13a}, and R^{14a} are each independently selected from the group consisting of hydrogen, alkyl, cycloalkyl, heterocyclyl, aryl and heteroaryl, wherein the alkyl, cycloalkyl, heterocyclyl, aryl or heteroaryl is optionally substituted with one or more groups selected from the group consisting of halogen, hydroxy, alkoxy, alkyl, aryl and cycloalkyl;
R^{11c}, R^{12b}, R^{13b}, R^{14b} are each independently selected from the group consisting of hydrogen and alkyl, wherein alkyl is optionally substituted with one or more groups selected from the group consisting of halogen, hydroxyl and alkoxy;
n is 1 or 2; and
m is 1 or 2.

In some embodiments of the disclosure, in the compound of formula (I), or a tautomer, mesomer, racemate, enantiomer, diastereomer, or mixture thereof, or a pharmaceutically acceptable salt, solvate or prodrug thereof, G¹, G², G³ and G⁴ are each identical or different, and each is N or CR⁶, provided that no more than two of them are N;
R¹ is selected from the group consisting of alkyl, haloalkyl, hydroxyalkyl, cycloalkyl, heterocyclyl and heteroaryl, wherein the alkyl, cycloalkyl, heterocyclyl or heteroaryl is optionally substituted with one or more groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NHC(═O)R^{11a}, NHC(═O)OR^{11a}, cycloalkyl, heterocyclyl, aryl and heteroaryl;
R^{2a}, R^{2b}, R^{3a} and R^{3b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;
or two of R^{2a}, R^{2b}, R^{3a} and R^{3b} together with the carbon atom to which they are attached form cycloalkyl or heterocyclyl;
R^{4a}, R^{4b}, R^{5a} and R^{5b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, cycloalkyl, heterocyclyl, aryl and heteroaryl;
or two of R^{4a}, R^{4b}, R^{5a} and R^{5b} together with the carbon atom to which they are attached form cycloalkyl or heterocyclyl;
each R⁶ is identical or different, and each is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
R^{11a}, R^{12a}, R^{13a}, and R^{14a} are each independently selected from the group consisting of hydrogen, alkyl, cycloalkyl, heterocyclyl, aryl and heteroaryl, wherein the alkyl, cycloalkyl, heterocyclyl, aryl or heteroaryl is optionally substituted with one or more groups selected from the group consisting of halogen, hydroxy, alkoxy, alkyl and cycloalkyl;
R^{11b}, R^{12b}, R^{13b}, R^{14b} are each independently selected from the group consisting of hydrogen and alkyl, wherein alkyl is optionally substituted with one or more groups selected from the group consisting of halogen, hydroxyl and alkoxy;
n is 1 or 2; and
m is 1 or 2.

In some embodiments of the disclosure, the compound of formula (I), or a tautomer, mesomer, racemate, enantiomer, diastereomer, or mixture thereof, or a pharmaceutically acceptable salt, solvate or prodrug thereof, is a compound of formula (II), or a tautomer, mesomer, racemate, enantiomer, diastereomer, or mixture thereof, or a pharmaceutically acceptable salt, solvate or prodrug thereof:

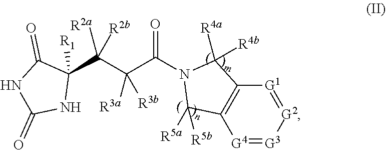

wherein G¹, G², G³, G⁴, R¹, R^{2a} to R^{5a}, R^{2b} to R^{5b}, n and m are each as defined in formula (I).

In some embodiments of the disclosure, in the compound of formula (I) or formula (II), or a tautomer, mesomer, racemate, enantiomer, diastereomer, or mixture thereof, or a pharmaceutically acceptable salt, solvate or prodrug thereof, G¹ and G² are each independently N or CR⁶; G³ and G⁴ are each CR⁶; and R⁶ is as defined in formula (I).

In some embodiments of the disclosure, the compound of formula (I), or a tautomer, mesomer, racemate, enantiomer, diastereomer, or mixture thereof, or a pharmaceutically acceptable salt, solvate or prodrug thereof, is a compound of formula (III) or (IIIa), or a tautomer, mesomer, racemate, enantiomer, diastereomer, or mixture thereof, or a pharmaceutically acceptable salt, solvate or prodrug thereof:

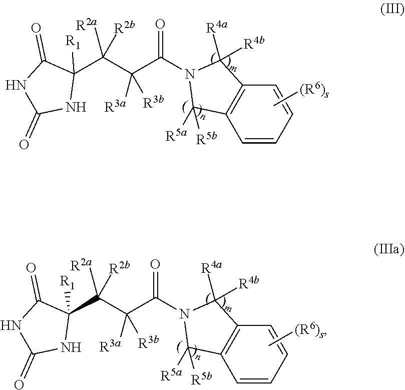

wherein:
s is 0, 1 or 2; and
R¹, R^{2a} to R^{5a}, R^{2b} to R^{5b}, R⁶, n and m are each as defined in formula (I) above.

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R¹ is selected from the group consisting of alkyl, cycloalkyl and heteroaryl, wherein the alkyl, cycloalkyl and heteroaryl are optionally substituted with one or more groups selected from the group consisting of alkyl and alkoxy; preferably R¹ is selected from the group consisting of C₁₋₆ alkyl, 3 to 8-member cycloalkyl and 5 to 10-member heteroaryl, wherein the C₁₋₆ alkyl, 3 to 8-member cycloalkyl and 5 to 10-member heteroaryl are optionally substituted with one or more, some times preferably one to three, groups independently selected from the group consisting of C₁₋₆ alkyl and C₁₋₆ alkoxy; more preferably, R¹ is selected from the group consisting of

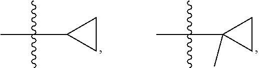

—CH₂OCH₃,

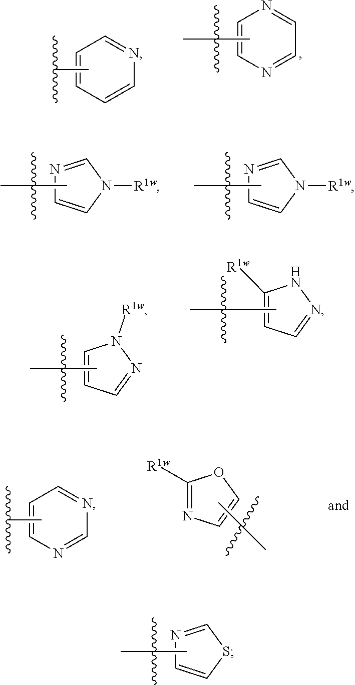

and R^{1w} is C₁₋₆ alkyl.

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R¹ is alkyl, cycloalkyl or heteroaryl, each optionally substituted by an alkyl or alkoxy; preferably R¹ is cycloalkyl, sometimes more preferably cyclopropyl.

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R¹ is cycloalkyl or heteroaryl, each optionally substituted by alkyl; preferably R¹ is cycloalkyl, sometimes more preferably cyclopropyl.

In some embodiments of the disclosure, in the compound of formula (I), formula (I), formula (II), formula (III) or formula (IIIa), or a tautomer, mesomer, racemate, enantiomer, diastereomer, or mixture thereof, or a pharmaceutically acceptable salt, solvate or prodrug thereof, R¹ is heteroaryl, optionally substituted by alkyl; preferably R¹ is pyrazolyl, thiazolyl, imidazolyl, pyridyl or pyrimidyl, each optionally substituted by C₁₋₆ alkyl; more preferably R¹ is imidazolyl, optionally substituted by C₁₋₆ alkyl.

In some embodiments of the disclosure, in the compound of formula (I), formula (I), formula (II), formula (III) or formula (IIIa), or a tautomer, mesomer, racemate, enantiomer, diastereomer, or mixture thereof, or a pharmaceutically acceptable salt, solvate or prodrug thereof, R^{2a} and R^{2b} are each identical or different, and each is independently selected from the group consisting of hydrogen, halogen, alkyl, alkoxy, hydroxy, haloalkyl, haloalkoxy, hydroxyalkyl and cyano; preferably R^{2a} and R^{2b} are each hydrogen.

In some embodiments of the disclosure, the compound of formula (I) or formula (III), or a tautomer, mesomer, racemate, enantiomer, diastereomer, or mixture thereof, or a pharmaceutically acceptable salt, solvate or prodrug thereof, is a compound of formula (IV) or (IVa), or a tautomer, mesomer, racemate, enantiomer, diastereomer, or mixture thereof, or a pharmaceutically acceptable salt, solvate or prodrug thereof:

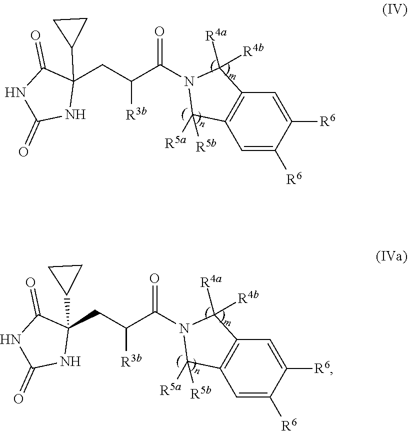

wherein:
R^{4a}, R^{5a}, R^{3b} to R^{5b}, R⁶, n and m are each as defined in formula (I).

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R^{3a} and R^{3b} are each independently selected from the group consisting of hydrogen, halogen, alkyl, alkoxy and hydroxyalkyl, wherein the alkyl is optionally substituted with one or more, sometimes preferably one to three, groups independently selected from the group consisting of halogen, OR^{12a} and alkoxy.

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R^{3a} and R^{3b} are each independently selected from the group consisting of hydrogen, halogen, alkyl, alkoxy and hydroxyalkyl, wherein the alkyl is optionally substituted with one or more, sometimes preferably one to three, groups independently selected from the group consisting of halogen and alkoxy.

In some embodiments of the disclosure, in the compound of formula (I), formula (II), formula (III) or formula (IIIa), or a tautomer, mesomer, racemate, enantiomer, diastereomer, or mixture thereof, or a pharmaceutically acceptable salt, solvate or prodrug thereof, R^{3a} and R^{3b} are identical or different, and each is selected from the group consisting of hydrogen, halogen, C₁₋₆ alkyl, C₁₋₆ alkoxy and C₁₋₆ hydroxyalkyl, wherein the C₁₋₆ alkyl is optionally substituted with one or more, sometimes preferably one to three, groups independently selected from the group consisting of halogen and C₁₋₆ alkoxy.

In some embodiments of the disclosure, in the compound of formula (I), formula (II), formula (III), formula (IIIa), formula (IV) or formula (IVa), or a tautomer, mesomer, racemate, enantiomer, diastereomer, or mixture thereof, or a pharmaceutically acceptable salt, solvate or prodrug thereof, R^{4a}, R^{4b}, R^{5a} and R^{5b} are each identical or different, and each is independently selected from the group consisting of hydrogen, deuterium, halogen and C₁₋₆ alkyl; or R^{5a} and R^{5b} together with the carbon atom to which they are attached form 3 to 8-member cycloalkyl, R^{4a} and R^{4b} are each identical or different, and each is independently selected from the group consisting of hydrogen, deuterium, halogen and C₁₋₆ alkyl; or R^{4a} and R^{4b} together with the carbon atom to which they are attached form 3 to 8-member cycloalkyl, R^{5a} and R^{5b} are identical or different, and each is independently selected from the group consisting of hydrogen, deuterium, halogen and C₁₋₆ alkyl.

In some embodiments of the disclosure, in the compound of formula (I), formula (II), formula (III), formula (IIIa), formula (IV) or formula (IVa), or a tautomer, mesomer, racemate, enantiomer, diastereomer, or mixture thereof, or a pharmaceutically acceptable salt, solvate or prodrug thereof, R^{4a}, R^{4b}, R^{5a} and R^{5b} are each identical or different, and each is independently selected from the group consisting of hydrogen, deuterium, halogen, alkyl, alkoxy, hydroxy, haloalkyl, haloalkoxy, hydroxyalkyl and cyano; preferably R^{4a}, R^{4b}, R^{5a} and R^{5b} are each independently selected from the group consisting of hydrogen, deuterium, halogen and alkyl; and sometimes more preferably R^{4a}, R^{4b}, R^{5a} and R^{5b} are each independently selected from the group consisting of hydrogen, halogen and alkyl.

In some embodiments of the disclosure, in the compound of formula (I) or formula (II), or a tautomer, mesomer, racemate, enantiomer, diastereomer, or mixture thereof, or a pharmaceutically acceptable salt, solvate or prodrug thereof,

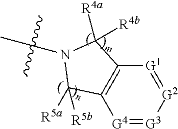

is selected from the group consisting of

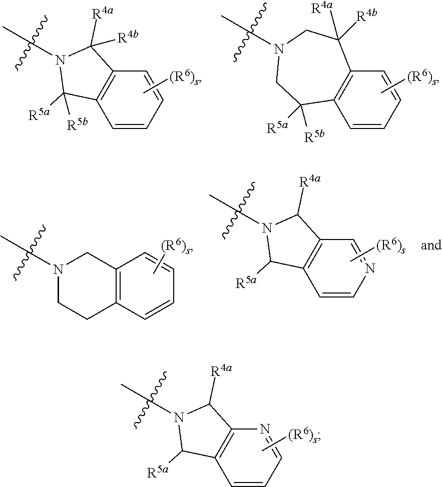

sometimes preferably

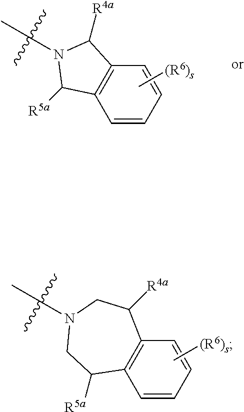

s is 0, 1 or 2; and
R^{4a}, R^{4b}, R^{5a}, R^{5b}, R⁶ and s are as defined in formula (I).

In some embodiments of the disclosure, in the compound of formula (I) or formula (II), or a tautomer, mesomer, racemate, enantiomer, diastereomer, or mixture thereof, or a pharmaceutically acceptable salt, solvate or prodrug thereof,

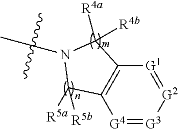

is selected from the group consisting of

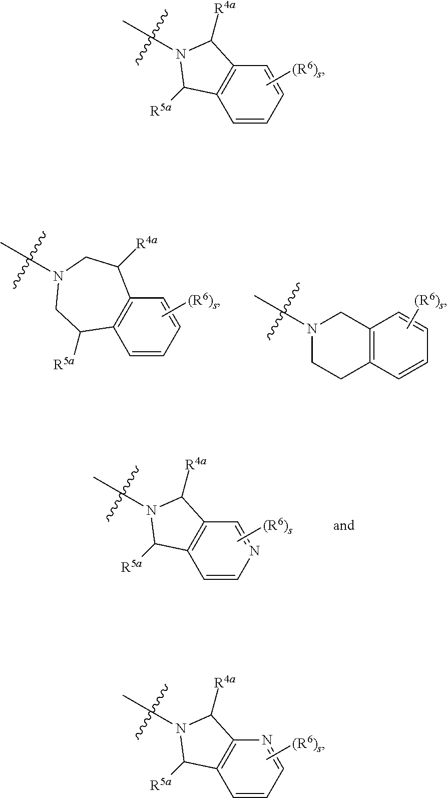

and preferably

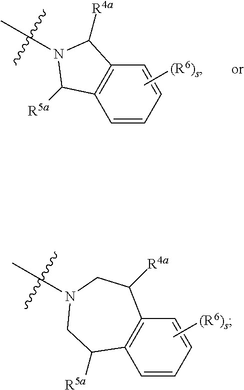

s is 0, 1 or 2; and
R^{4a}, R^{5a} and R⁶ are as defined in formula (I) above.

In some embodiments of the disclosure, in the compound of formula (I) or formula (II), or a tautomer, mesomer, racemate, enantiomer, diastereomer, or mixture thereof, or a pharmaceutically acceptable salt, solvate or prodrug thereof,

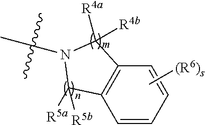

is selected from the group consisting of

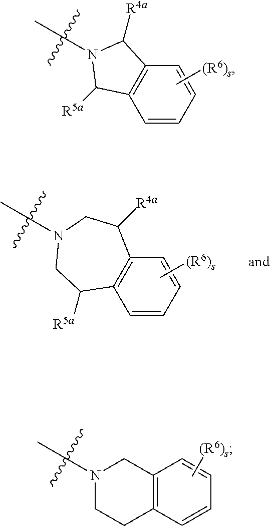

s is 0, 1 or 2; and
R^{4a}, R^{5a} and R⁶ are as defined in formula (I), above.

In some embodiments of the disclosure, in the compound of formula (I) or formula (II), or a tautomer, mesomer, racemate, enantiomer, diastereomer, or mixture thereof, or a pharmaceutically acceptable salt, solvate or prodrug thereof,

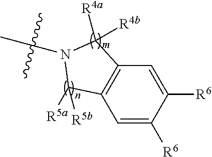

is selected from the group consisting of

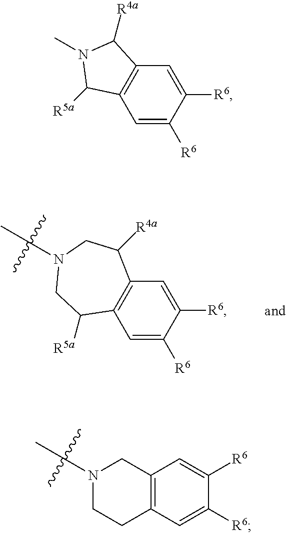

s is 0, 1 or 2; and
R^{4a}, R^{5a} and R⁶ are as defined in formula (I) above.

In some embodiments of the disclosure, the compound of formula (I), formula (III), formula (IV), or a tautomer, mesomer, racemate, enantiomer, diastereomer, or mixture thereof, or a pharmaceutically acceptable salt, solvate or prodrug thereof, is a compound of formula (V), (Va) or (Vb), or a tautomer, mesomer, racemate, enantiomer, diastereomer, or mixture thereof, or a pharmaceutically acceptable salt, solvate or prodrug thereof:

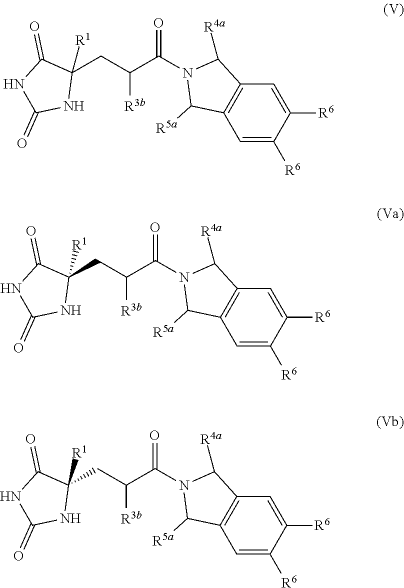

wherein:
R¹ is selected from the group consisting of cycloalkyl and heteroaryl, wherein the cycloalkyl or heteroaryl is optionally substituted with one or more groups independently selected from the group consisting of halogen, hydroxy, cyano, alkyl, alkoxy and hydroxyalkyl; and
R^{3b}, R^{4a}, R^{5a} and R⁶ are each as defined in formula (I) above.

In some embodiments of the disclosure, in the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt thereof, solvate or prodrug thereof, R⁶ is identical or different, and at each occurrence is independently selected from the group consisting of hydrogen, halogen, alkyl, alkoxy, haloalkyl, haloalkoxy, cyano and C(═O)OR^{13a}; R^{13a} is as defined in formula (I); preferably, R⁶ is identical or different, and at each occurrence is independently selected from the group consisting of hydrogen, halogen, C₁₋₆ alkyl, C₁₋₆ alkoxy, C₁₋₆ haloalkyl, C₁₋₆ haloalkoxy, cyano and C(═O)OR^{13a}; and R^{13a} is hydrogen or C₁₋₆ alkyl.

In some embodiments of the disclosure, in the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt thereof, solvate or prodrug thereof, R⁶ at each occurrence is independently selected from the group consisting of hydrogen, halogen, alkyl, alkoxy, haloalkyl, hydroxyalkyl, amino, nitro, C(═O)OR^{13a}, NR^{14a}R^{14b}, haloalkoxy and cyano; sometimes preferably R⁶ at each occurrence is independently selected from the group consisting of hydrogen, halogen, alkyl, alkoxy, haloalkyl, haloalkoxy and cyano.

In some embodiments of the disclosure, in the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt thereof, solvate or prodrug thereof, R^{11al}, R^{12a}, R^{13a}, and R^{14a} are each independently selected from the group consisting of hydrogen and alkyl, wherein the alkyl is optionally substituted with one or more, sometimes preferably one to three, groups independently selected from the group consisting of halogen, hydroxy, alkoxy and aryl.

In some embodiments of the disclosure, in the compound of formula (I), or a tautomer, mesomer, racemate, enantiomer, diastereomer, or mixture thereof, or a pharmaceutically acceptable salt thereof, solvate or prodrug thereof, R^{11b}, R^{12b}, R^{11b}, R^{14b} are each independently selected from the group consisting of hydrogen and alkyl.

In some embodiments of the disclosure, in the compound of formula (I), formula (II), formula (III), formula (IIa), formula (IV) or formula (IVa), or a tautomer, mesomer, racemate, enantiomer, diastereomer, or mixture thereof, or a pharmaceutically acceptable salt thereof, solvate or prodrug thereof, n is 1.

In some embodiments of the disclosure, in the compound of formula (I), formula (II), formula (III), formula (IIIa), formula (IV) or formula (IVa), or a tautomer, mesomer, racemate, enantiomer, diastereomer, or mixture thereof, or a pharmaceutically acceptable salt thereof, solvate or prodrug thereof, m is 1.

Exemplified compounds of the disclosure include, but are not limited to:

| Example | |
|---|---|
| No. | Compound structure and name |
| 1 | 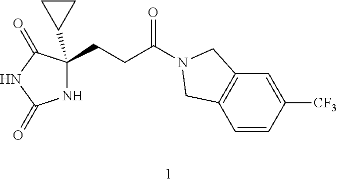 [SMILES: O=C1NC(=O)[C@](CCC(=O)N2Cc3ccc(C(F)(F)F)cc3C2)(C2CC2)N1] |
| | (S)-5-cyclopropyl-5-(3-oxo-3-(5-(trifluoromethyl)isoindolin-2- |
| | yl)propyl)imidazolidine-2,4-dione |
| 2 | 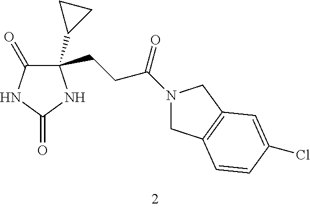 [SMILES: O=C1NC(=O)[C@](CCC(=O)N2Cc3ccc(Cl)cc3C2)(C2CC2)N1] |
| | (S)-5-(3-(5-chloroisoindolin-2-yl)-3-oxopropyl)-5-cyclopropylimidazolidine- |
| | 2,4-dione |
| 3 | 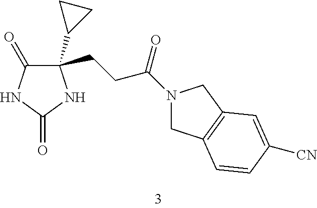 [SMILES: N#Cc1ccc2c(c1)CN(C(=O)CC[C@@]1(C3CC3)NC(=O)NC1=O)C2] |
| | (S)-2-(3-(4-cyclopropyl-2,5-dioxoimidazolidin-4-yl)propanoyl)isoindoline-5- |
| | carbonitrile |
| 4 | 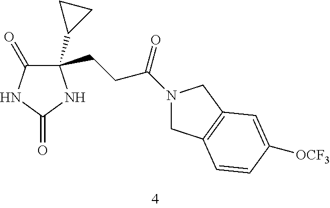 [SMILES: O=C1NC(=O)[C@](CCC(=O)N2Cc3ccc(OC(F)(F)F)cc3C2)(C2CC2)N1] |
| | (S)-5-cyclopropyl-5-(3-oxo-3-(5-(trifluoromethoxy)isoindolin-2- |
| | yl)propyl)imidazolidine-2,4-dione |
| 5 | 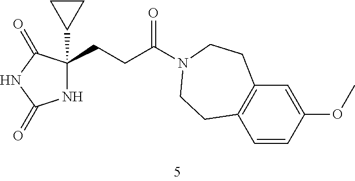 [SMILES: COc1ccc2c(c1)CCN(C(=O)CC[C@@]1(C3CC3)NC(=O)NC1=O)CC2] |
| | (S)-5-cyclopropyl-5-(3-(7-methoxy-4,5-dihydro-1H-benzo[d]azepin-3(2H)- |
| | y1)-3-oxopropyl)imidazolidine-2,4-dione |
| 6 | 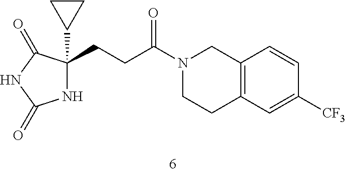 [SMILES: O=C1NC(=O)[C@](CCC(=O)N2CCc3cc(C(F)(F)F)ccc3C2)(C2CC2)N1] |
| | (S)-5-cyclopropyl-5-(3-oxo-3-(6-(trifluoromethyl)-3,4-dihydroisoquinolin- |
| | 2(1H)-yl)propyl)imidazolidine-2,4-dione |
| 7 | 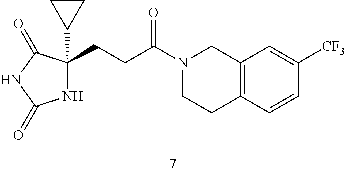 [SMILES: O=C1NC(=O)[C@](CCC(=O)N2CCc3ccc(C(F)(F)F)cc3C2)(C2CC2)N1] |
| | (S)-5-cyclopropyl-5-(3-oxo-3-(7-(trifluoromethyl)-3,4-dihydroisoquinolin- |
| | 2(1H)-yl)propyl)imidazolidine-2,4-dione |
| 8 | 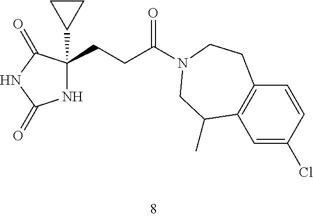 [SMILES: CC1CN(C(=O)CC[C@@]2(C3CC3)NC(=O)NC2=O)CCc2ccc(Cl)cc21] |
| | (5S)-5-(3-(8-chloro-1-methyl-4,5-dihydro-1H-benzo[d]azepin-3(2H)-yl)-3- |
| | oxopropyl)-5-cyclopropylimidazolidine-2,4-dione |
| 9 | 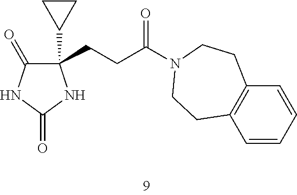 [SMILES: O=C1NC(=O)[C@](CCC(=O)N2CCc3ccccc3CC2)(C2CC2)N1] |
| | (S)-5-cyclopropyl-5-(3-(4,5-dihydro-1H-benzo[d]azepin-3(2H)-yl)-3- |
| | oxopropyl)imidazolidine-2,4-dione |
| 10 | 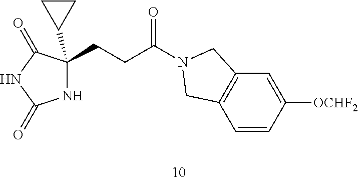 [SMILES: O=C1NC(=O)[C@](CCC(=O)N2Cc3ccc(OC(F)F)cc3C2)(C2CC2)N1] |
| | (S)-5-cyclopropyl-5-(3-(5-(difluoromethoxy)isoindolin-2-yl)-3- |
| | oxopropyl)imidazolidine-2,4-dione |
| 11 | 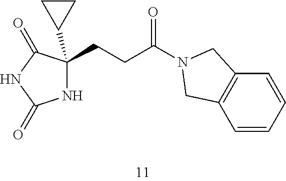 [SMILES: O=C1NC(=O)[C@](CCC(=O)N2Cc3ccccc3C2)(C2CC2)N1] |
| | (S)-5-cyclopropyl-5-(3-(isoindolin-2-yl)-3-oxopropyl)imidazolidine-2,4-dione |
| 12 | 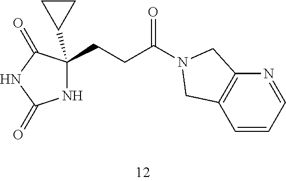 [SMILES: O=C1NC(=O)[C@](CCC(=O)N2Cc3cccnc3C2)(C2CC2)N1] |
| | (S)-5-cyclopropyl-5-(3-oxo-3-(5H-pyrrolo[3,4-b]pyridin-6(7H)- |
| | yl)propyl)imidazolidine-2,4-dione |
| 13 | 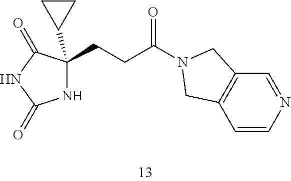 [SMILES: O=C1NC(=O)[C@](CCC(=O)N2Cc3ccncc3C2)(C2CC2)N1] |
| | (S)-5-cyclopropyl-5-(3-oxo-3-(1H-pyrrolo[3,4-c]pyridin-2(3H)- |
| | yl)propyl)imidazolidine-2,4-dione |
| 14 | 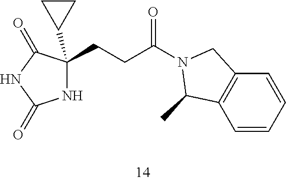 [SMILES: C[C@@H]1c2ccccc2CN1C(=O)CC[C@@]1(C2CC2)NC(=O)NC1=O] |
| | (S)-5-cyclopropyl-5-(3-((R)-1-methylisoindolin-2-yl)-3- |
| | oxopropyl)imidazolidine-2,4-dione |
| 15 | 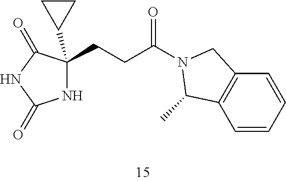 [SMILES: C[C@H]1c2ccccc2CN1C(=O)CC[C@@]1(C2CC2)NC(=O)NC1=O] |
| | (S)-5-cyclopropyl-5-(3-((S)-1-methylisoindolin-2-yl)-3- |
| | oxopropyl)imidazolidine-2,4-dione |
| 16 | 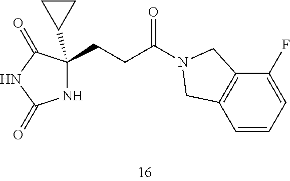 [SMILES: O=C1NC(=O)[C@](CCC(=O)N2Cc3cccc(F)c3C2)(C2CC2)N1] |
| | (S)-5-cyclopropyl-5-(3-(4-fluoroisoindolin-2-yl)-3-oxopropyl)imidazolidine- |
| | 2,4-dione |
| 17 | 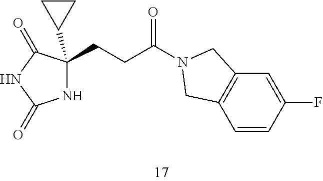 [SMILES: O=C1NC(=O)[C@](CCC(=O)N2Cc3ccc(F)cc3C2)(C2CC2)N1] |
| | (S)-5-cyclopropyl-5-(3-(5-fluoroisoindolin-2-yl)-3-oxopropyl)imidazolidine- |
| | 2,4-dione |
| 18 | 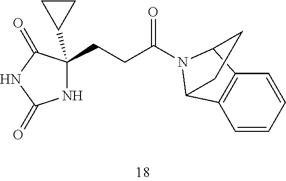 [SMILES: O=C1NC(=O)[C@](CCC(=O)N2C3CCC2c2ccccc23)(C2CC2)N1] |
| | (5S)-5-cyclopropyl-5-(3-oxo-3-(1,2,3,4-tetrahydro-1,4-epiminonaphthalen-9- |
| | yl)propyl)imidazolidine-2,4-dione |
| 19 | 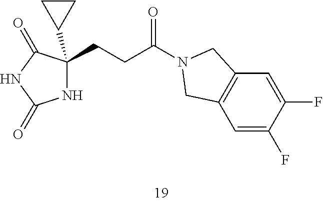 [SMILES: O=C1NC(=O)[C@](CCC(=O)N2Cc3cc(F)c(F)cc3C2)(C2CC2)N1] |
| | (S)-5-cyclopropyl-5-(3-(5,6-difluoroisoindolin-2-yl)-3- |
| | oxopropyl)imidazolidine-2,4-dione |
| 20 | 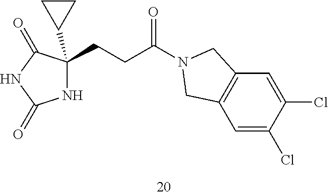 [SMILES: O=C1NC(=O)[C@](CCC(=O)N2Cc3cc(Cl)c(Cl)cc3C2)(C2CC2)N1] |
| | (S)-5-cyclopropyl-5-(3-(5,6-dichloroisoindolin-2-yl)-3- |
| | oxopropyl)imidazolidine-2,4-dione |
| 21 | 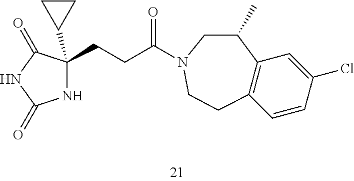 [SMILES: C[C@H]1CN(C(=O)CC[C@@]2(C3CC3)NC(=O)NC2=O)CCc2ccc(Cl)cc21] |
| | (S)-5-(3-((R)-8-chloro-1-methyl-4,5-dihydro-1H-benzo[d]azepin-3(2H)-yl)-3- |
| | oxopropyl)-5-cyclopropylimidazolidine-2,4-dione |
| 22 | 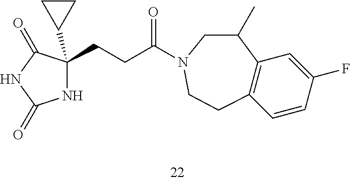 [SMILES: CC1CN(C(=O)CC[C@@]2(C3CC3)NC(=O)NC2=O)CCc2ccc(F)cc21] |
| | (5S)-5-cyclopropyl-5-(3-(8-fluoro-1-methyl-4,5-dihydro-1H-benzo[d]azepin- |
| | 3(2H)-yl)-3-oxopropyl)imidazolidine-2,4-dione (Mixture of two |
| | diastereomers) |
| 23 | 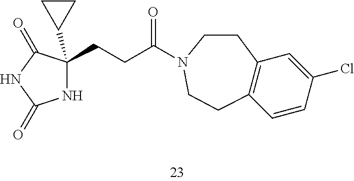 [SMILES: O=C1NC(=O)[C@](CCC(=O)N2CCc3ccc(Cl)cc3CC2)(C2CC2)N1] |
| | (S)-5-(3-(7-chloro-4,5-dihydro-1H-benzo[d]azepin-3(2H)-yl)-3-oxopropyl)-5- |
| | cyclopropylimidazolidine-2,4-dione |
| 24 | 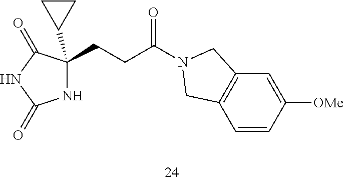 [SMILES: COc1ccc2c(c1)CN(C(=O)CC[C@@]1(C3CC3)NC(=O)NC1=O)C2] |
| | (S)-5-cyclopropyl-5-(3-(5-methoxyisoindolin-2-yl)-3- |
| | oxopropyl)imidazolidine-2,4-dione |
| 25 | 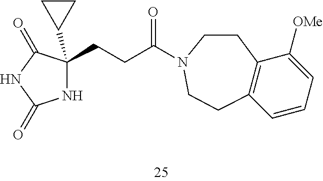 [SMILES: COc1cccc2c1CCN(C(=O)CC[C@@]1(C3CC3)NC(=O)NC1=O)CC2] |
| | (S)-5-cyclopropyl-5-(3-(6-methoxy-4,5-dihydro-1H-benzo[d]azepin-3(2H)- |
| | yl)-3-oxopropyl)imidazolidine-2,4-dione |
| 26 | 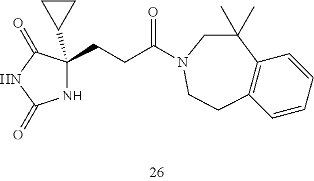 [SMILES: CC1(C)CN(C(=O)CC[C@@]2(C3CC3)NC(=O)NC2=O)CCc2ccccc21] |
| | (S)-5-cyclopropyl-5-(3-(1,1-dimethyl-4,5-dihydro-1H-benzo[d]azepin-3(2H)- |
| | yl)-3-oxopropyl)imidazolidine-2,4-dione |
| 27 | 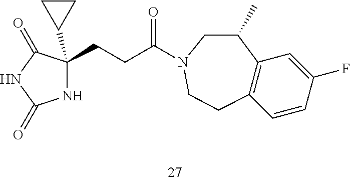 [SMILES: C[C@H]1CN(C(=O)CC[C@@]2(C3CC3)NC(=O)NC2=O)CCc2ccc(F)cc21] |
| | (S)-5-cyclopropyl-5-(3-((R)-8-fluoro-1-methyl-1,2,4,5-tetrahydro-3H- |
| | benzo[d]azepin-3-yl)-3-oxopropyl)imidazolidine-2,4-dione |
| 28 | 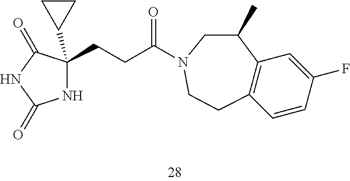 [SMILES: C[C@@H]1CN(C(=O)CC[C@@]2(C3CC3)NC(=O)NC2=O)CCc2ccc(F)cc21] |
| | (S)-5-cyclopropyl-5-(3-((S)-8-fluoro-1-methyl-1,2,4,5-tetrahydro-3H- |
| | benzo[d]azepin-3-yl)-3-oxopropyl)imidazolidine-2,4-dione |
| 29 | 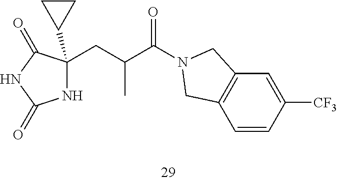 [SMILES: CC(C[C@@]1(C2CC2)NC(=O)NC1=O)C(=O)N1Cc2ccc(C(F)(F)F)cc2C1] |
| | (5S)-5-cyclopropyl-5-(2-methyl-3-oxo-3-(5-(trifluoromethyl)isoindolin-2- |
| | yl)propyl)imidazolidine-2,4-dione |
| 29-1 | 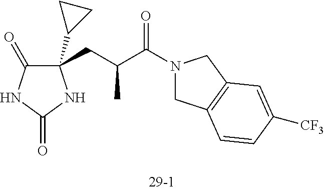 [SMILES: C[C@@H](C[C@@]1(C2CC2)NC(=O)NC1=O)C(=O)N1Cc2ccc(C(F)(F)F)cc2C1] |
| | (S)-5-cyclopropyl-5-((S)-2-methyl-3-oxo-3-(5-(trifluoromethyl)isoindolin-2- |
| | yl)propyl)imidazolidine-2,4-dione |
| 29-2 | 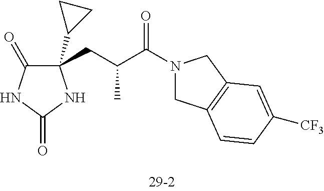 [SMILES: C[C@H](C[C@@]1(C2CC2)NC(=O)NC1=O)C(=O)N1Cc2ccc(C(F)(F)F)cc2C1] |
| | (S)-5-cyclopropyl-5-((R)-2-methyl-3-oxo-3-(5-(trifluoromethyl)isoindolin-2- |
| | yl)propyl)imidazolidine-2,4-dione |
| 30 | 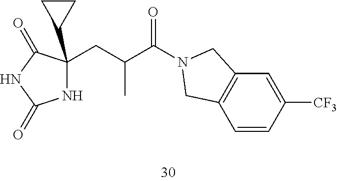 [SMILES: CC(C[C@]1(C2CC2)NC(=O)NC1=O)C(=O)N1Cc2ccc(C(F)(F)F)cc2C1] |
| | (5R)-5-cyclopropyl-5-(2-methyl-3-oxo-3-(5-(trifluoromethyl)isoindolin-2- |
| | yl)propyl)imidazolidine-2,4-dione |
| 30-1 | 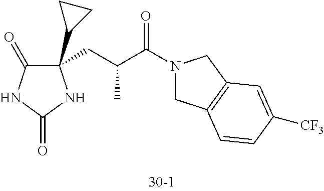 [SMILES: C[C@H](C[C@]1(C2CC2)NC(=O)NC1=O)C(=O)N1Cc2ccc(C(F)(F)F)cc2C1] |
| | (R)-5-cyclopropyl-5-((R)-2-methyl-3-oxo-3-(5-(trifluoromethyl)isoindolin-2- |
| | yl)propyl)imidazolidine-2,4-dione |
| 30-2 | 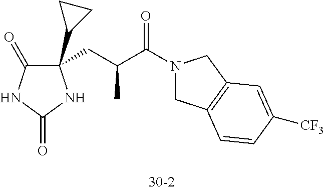 [SMILES: C[C@@H](C[C@]1(C2CC2)NC(=O)NC1=O)C(=O)N1Cc2ccc(C(F)(F)F)cc2C1] |
| | (R)-5-cyclopropyl-5-((S)-2-methyl-3-oxo-3-(5-(trifluoromethyl)isoindolin-2- |
| | yl)propyl)imidazolidine-2,4-dione |
| 31 | 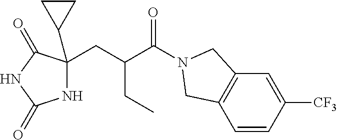 [SMILES: CCC(CC1(C2CC2)NC(=O)NC1=O)C(=O)N1Cc2ccc(C(F)(F)F)cc2C1] |
| | 5-cyclopropyl-5-(2-(5-(trifluoromethyl)isoindoline-2- |
| | carbonyl)butyl)imidazolidine-2,4-dione |
| 31-1 | 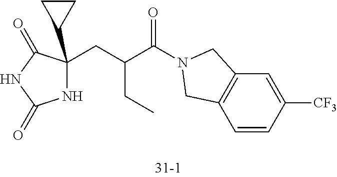 [SMILES: CCC(C[C@]1(C2CC2)NC(=O)NC1=O)C(=O)N1Cc2ccc(C(F)(F)F)cc2C1] |
| | (5R)-5-cyclopropyl-5-(2-(5-(trifluoromethyl)isoindoline-2- |
| | carbonyl)butyl)imidazolidine-2,4-dione |
| 31-2 | 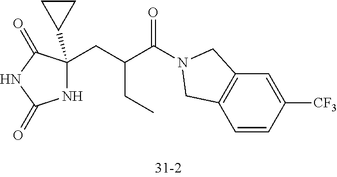 [SMILES: CCC(C[C@@]1(C2CC2)NC(=O)NC1=O)C(=O)N1Cc2ccc(C(F)(F)F)cc2C1] |
| | (5S)-5-cyclopropyl-5-(2-(5-(trifluoromethyl)isoindoline-2- |
| | carbonyl)butyl)imidazolidine-2,4-dione |
| 32 | 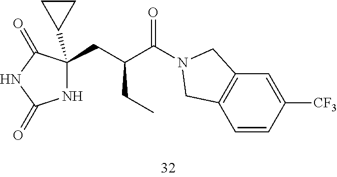 [SMILES: CC[C@@H](C[C@@]1(C2CC2)NC(=O)NC1=O)C(=O)N1Cc2ccc(C(F)(F)F)cc2C1] |
| | (S)-5-cyclopropyl-5-((S)-2-(5-(trifluoromethyl)isoindoline-2- |
| | carbonyl)butyl)imidazolidine-2,4-dione |
| 33 | 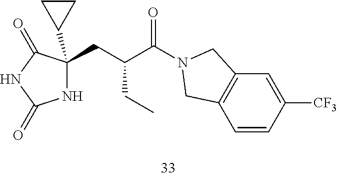 [SMILES: CC[C@H](C[C@@]1(C2CC2)NC(=O)NC1=O)C(=O)N1Cc2ccc(C(F)(F)F)cc2C1] |
| | (S)-5-cyclopropyl-5-((R)-2-(5-(trifluoromethyl)isoindoline-2- |
| | carbonyl)butyl)imidazolidine-2,4-dione |
| 34 | 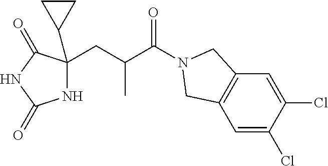 [SMILES: CC(CC1(C2CC2)NC(=O)NC1=O)C(=O)N1Cc2cc(Cl)c(Cl)cc2C1] |
| | 5-cyclopropyl-5-(3-(5,6-dichloroisoindolin-2-yl)-2-methyl-3- |
| | oxopropyl)imidazolidine-2,4-dione |
| 34-1 | 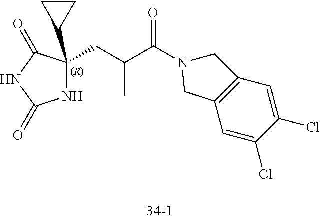 |
| | (5R)-5-cyclopropyl-5-(3-(5,6-dichloroisoindolin-2-yl)-2-methyl-3- |
| | oxopropyl)imidazolidine-2,4-dione |
| 34-2 | 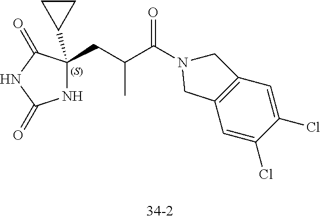 |
| | (5S)-5-cyclopropyl-5-(3-(5,6-dichloroisoindolin-2-yl)-2-methyl-3- |
| | oxopropyl)imidazolidine-2,4-dione |
| 34-2-A | 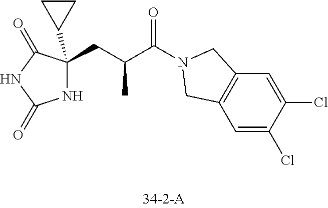 [SMILES: C[C@@H](C[C@@]1(C2CC2)NC(=O)NC1=O)C(=O)N1Cc2cc(Cl)c(Cl)cc2C1] |
| | (5S)-5-cyclopropyl-5-((S)-(3-(5,6-dichloroisoindolin-2-yl)-2-methyl-3- |
| | oxopropyl))imidazolidine-2,4-dione |
| 34-2-B | 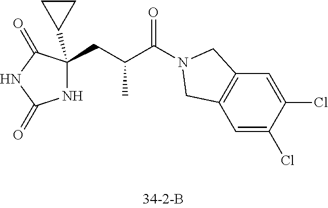 [SMILES: C[C@H](C[C@@]1(C2CC2)NC(=O)NC1=O)C(=O)N1Cc2cc(Cl)c(Cl)cc2C1] |
| | (5S)-5-cyclopropyl-5-((R)-(3-(5,6-dichloroisoindolin-2-yl)-2-methyl-3- |
| | oxopropyl))imidazolidine-2,4-dione |
| 35 | 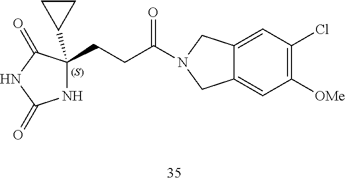 |
| | (S)-5-(3-(5-chloro-6-methoxyisoindolin-2-yl)-3-oxopropyl)-5- |
| | cyclopropylimidazolidine-2,4-dione |
| 36 | 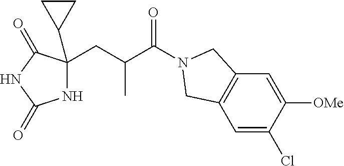 [SMILES: COc1cc2c(cc1Cl)CN(C(=O)C(C)CC1(C3CC3)NC(=O)NC1=O)C2] |
| | 5-(3-(5-chloro-6-methoxyisoindolin-2-yl)-2-methyl-3-oxopropyl)-5- |
| | cyclopropylimidazolidine-2,4-dione |
| 36-1 | 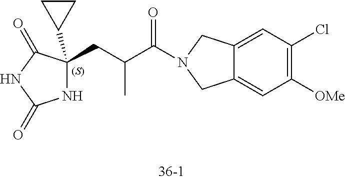 |
| | (5S)-5-(3-(5-chloro-6-methoxyisoindolin-2-yl)-2-methyl-3-oxopropyl)-5- |
| | cyclopropylimidazolidine-2,4-dione |
| 37 | 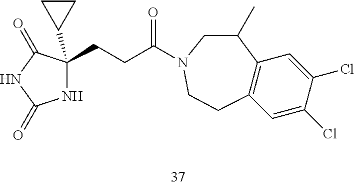 [SMILES: CC1CN(C(=O)CC[C@@]2(C3CC3)NC(=O)NC2=O)CCc2cc(Cl)c(Cl)cc21] |
| | (S)-5-cyclopropyl-5-(3-(7,8-dichloro-1-methyl-1,2,4,5-tetrahydro-3H- |
| | benzo[d]azepin-3-yl)-3-oxopropyl)imidazolidine-2,4-dione |
| 37-1 | 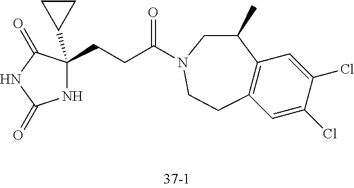 [SMILES: C[C@@H]1CN(C(=O)CC[C@@]2(C3CC3)NC(=O)NC2=O)CCc2cc(Cl)c(Cl)cc21] |
| | (S)-5-cyclopropyl-5-(3-((S)-7,8-dichloro-1-methyl-1,2,4,5-tetrahydro-3H- |
| | benzo[d]azepin-3-yl)-3-oxopropyl)imidazolidine-2,4-dione |
| 37-2 | 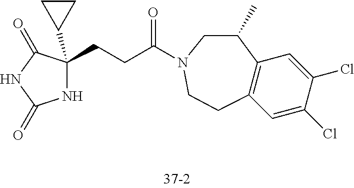 [SMILES: C[C@H]1CN(C(=O)CC[C@@]2(C3CC3)NC(=O)NC2=O)CCc2cc(Cl)c(Cl)cc21] |
| | (S)-5-cyclopropyl-5-(3-((R)-7,8-dichloro-1-methyl-1,2,4,5-tetrahydro-3H- |
| | benzo[d]azepin-3-yl)-3-oxopropyl)imidazolidine-2,4-dione |
| 38 | 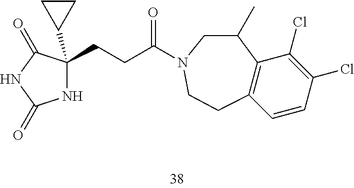 [SMILES: CC1CN(C(=O)CC[C@@]2(C3CC3)NC(=O)NC2=O)CCc2ccc(Cl)c(Cl)c21] |
| | (S)-5-cyclopropyl-5-(3-(8,9-dichloro-1-methyl-1,2,4,5-tetrahydro-3H- |
| | benzo[d]azepin-3-yl)-3-oxopropyl)imidazolidine-2,4-dione |
| 38-1 | 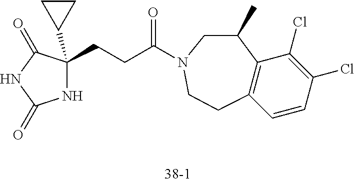 [SMILES: C[C@@H]1CN(C(=O)CC[C@@]2(C3CC3)NC(=O)NC2=O)CCc2ccc(Cl)c(Cl)c21] |
| | (S)-5-cyclopropyl-5-(3-((S)-8,9-dichloro-1-methyl-1,2,4,5-tetrahydro-3H- |
| | benzo[d]azepin-3-yl)-3-oxopropyl)imidazolidine-2,4-dione |
| 38-2 | 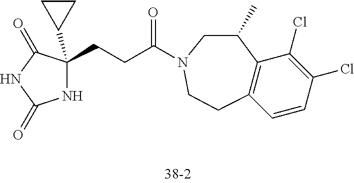 [SMILES: C[C@H]1CN(C(=O)CC[C@@]2(C3CC3)NC(=O)NC2=O)CCc2ccc(Cl)c(Cl)c21] |
| | (S)-5-cyclopropyl-5-(3-((R)-8,9-dichloro-1-methyl-1,2,4,5-tetrahydro-3H- |
| | benzo[d]azepin-3-yl)-3-oxopropyl)imidazolidine-2,4-dione |
| 39 | 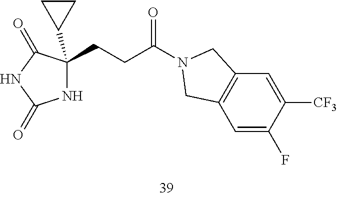 [SMILES: O=C1NC(=O)[C@](CCC(=O)N2Cc3cc(F)c(C(F)(F)F)cc3C2)(C2CC2)N1] |
| | (S)-5-cyclopropyl-5-(3-(5-fluoro-6-(trifluoromethyl)isoindolin-2-yl)-3- |
| | oxopropyl)imidazolidine-2,4-dione |
| 40 | 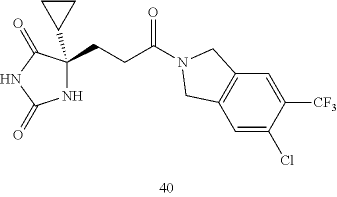 [SMILES: O=C1NC(=O)[C@](CCC(=O)N2Cc3cc(Cl)c(C(F)(F)F)cc3C2)(C2CC2)N1] |
| | (S)-5-(3-(5-chloro-6-(trifluoromethyl)isoindolin-2-yl)-3-oxopropyl)-5- |
| | cyclopropylimidazolidine-2,4-dione |
| 41 | 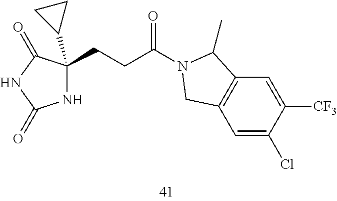 [SMILES: CC1c2cc(C(F)(F)F)c(Cl)cc2CN1C(=O)CC[C@@]1(C2CC2)NC(=O)NC1=O] |
| | (5S)-5-(3-(5-chloro-1-methyl-6-(trifluoromethyl)isoindolin-2-yl)-3- |
| | oxopropyl)-5-cyclopropylimidazolidine-2,4-dione |
| 41-1 | 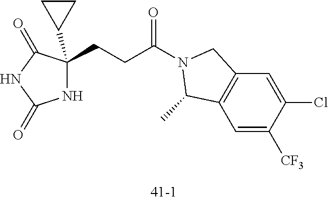 [SMILES: C[C@H]1c2cc(C(F)(F)F)c(Cl)cc2CN1C(=O)CC[C@@]1(C2CC2)NC(=O)NC1=O] |
| | (S)-5-(3-((S)-5-chloro-1-methyl-6-(trifluoromethyl)isoindolin-2-yl)-3- |
| | oxopropyl)-5-cyclopropylimidazolidine-2,4-dione |
| 41-2 | 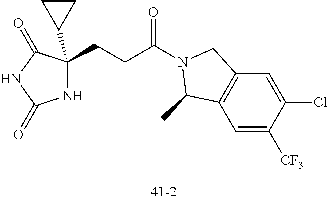 [SMILES: C[C@@H]1c2cc(C(F)(F)F)c(Cl)cc2CN1C(=O)CC[C@@]1(C2CC2)NC(=O)NC1=O] |
| | (S)-5-(3-((R)-5-chloro-1-methyl-6-(trifluoromethyl)isoindolin-2-yl)-3- |
| | oxopropyl)-5-cyclopropylimidazolidine-2,4-dione |
| 42 | 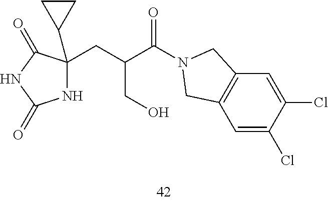 [SMILES: O=C1NC(=O)C(CC(CO)C(=O)N2Cc3cc(Cl)c(Cl)cc3C2)(C2CC2)N1] |
| | 5-cyclopropyl-5-(3-(5,6-dichloroisoindolin-2-yl)-2-(hydroxymethyl)-3- |
| | oxopropyl)imidazolidine-2,4-dione |
| 43 | 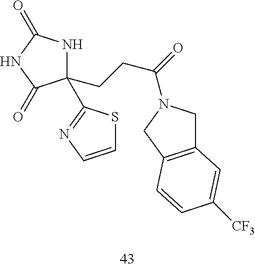 [SMILES: O=C1NC(=O)C(CCC(=O)N2Cc3ccc(C(F)(F)F)cc3C2)(c2nccs2)N1] |
| | 5-(3-oxo-3-(5-(trifluoromethyl)isoindolin-2-yl)propyl)-5-(thiazol-2- |
| | yl)imidazolidine-2,4-dione |
| 44 | 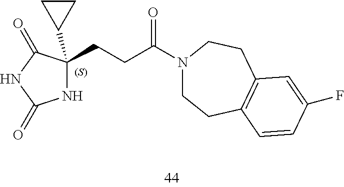 |
| | (S)-5-cyclopropyl-5-(3-(7-fluoro-4,5-dihydro-1H-benzo[d]azepin-3(2H)-yl)-3- |
| | oxopropyl)imidazolidine-2,4-dione |
| 45 | 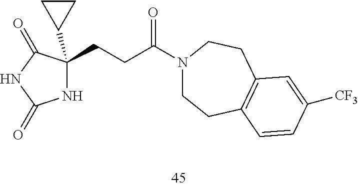 [SMILES: O=C1NC(=O)[C@](CCC(=O)N2CCc3ccc(C(F)(F)F)cc3CC2)(C2CC2)N1] |
| | (S)-5-cyclopropyl-5-(3-oxo-3-(7-(trifluoromethyl)-4,5-dihydro-1H- |
| | benzo[d]azepin-3(2H)-yl)propyl)imidazolidine-2,4-dione |
| 46 | 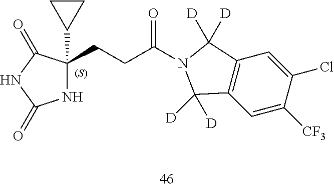 |
| | (S)-5-(3-(5-chloro-6-(trifluoromethyl)isoindolin-2-yl-1,1,3,3-d4)-3- |
| | oxopropyl)-5-cyclopropylimidazolidine-2,4-dione |
| 47 | 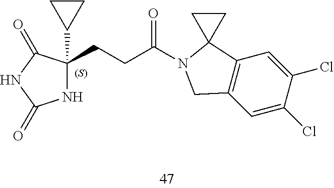 |
| | (S)-5-cyclopropyl-5-(3-(5',6'-dichlorospiro[cyclopropane-1,1'-isoindolin]-2'- |
| | yl)-3-oxopropyl)imidazolidine-2,4-dione |
| 48 | 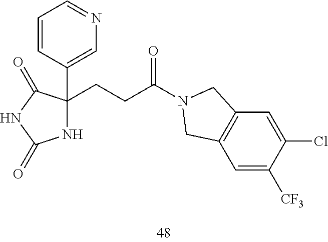 [SMILES: O=C1NC(=O)C(CCC(=O)N2Cc3cc(Cl)c(C(F)(F)F)cc3C2)(c2cccnc2)N1] |
| | 5-(3-(5-chloro-6-(trifluoromethyl)isoindolin-2-yl)-3-oxopropyl)-5-(pyridin-3- |
| | yl)imidazolidine-2,4-dione |
| 49 | 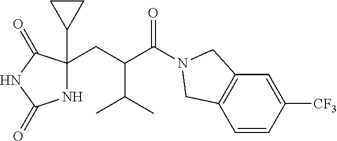 [SMILES: CC(C)C(CC1(C2CC2)NC(=O)NC1=O)C(=O)N1Cc2ccc(C(F)(F)F)cc2C1] |
| | 5-cyclopropyl-5-(3-methyl-2-(5-(trifluoromethyl)isoindoline-2- |
| | carbonyl)butyl)imidazolidine-2,4-dione |
| 49-1 | 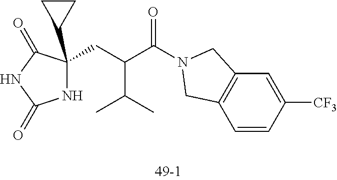 [SMILES: CC(C)C(C[C@]1(C2CC2)NC(=O)NC1=O)C(=O)N1Cc2ccc(C(F)(F)F)cc2C1] |
| | (5R)-5-cyclopropyl-5-(3-methyl-2-(5-(trifluoromethyl)isoindoline-2- |
| | carbonyl)butyl)imidazolidine-2,4-dione |
| 49-2 | 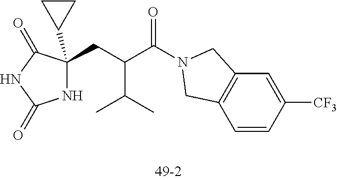 [SMILES: CC(C)C(C[C@@]1(C2CC2)NC(=O)NC1=O)C(=O)N1Cc2ccc(C(F)(F)F)cc2C1] |
| | (5S)-5-cyclopropyl-5-(3-methyl-2-(5-(trifluoromethyl)isoindoline-2- |
| | carbonyl)butyl)imidazolidine-2,4-dione |
| 49-2a | 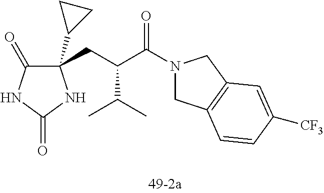 [SMILES: CC(C)[C@H](C[C@@]1(C2CC2)NC(=O)NC1=O)C(=O)N1Cc2ccc(C(F)(F)F)cc2C1] |
| | (5S)-5-cyclopropyl-5-[(2R)-3-methyl-2-[5-(trifluoromethyl)-1, 3, 3a, 7a- |
| | tetrahydroisoindole-2-carbonyl] butyl] imidazolidine-2, 4-dione |
| 49-2b | 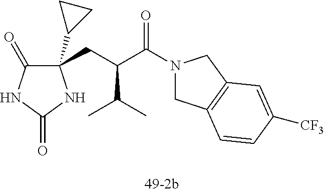 [SMILES: CC(C)[C@@H](C[C@@]1(C2CC2)NC(=O)NC1=O)C(=O)N1Cc2ccc(C(F)(F)F)cc2C1] |
| | (5R)-5-cyclopropyl-5-[(2S)-3-methyl-2-[5-(trifluoromethyl)-1, 3, 3a, 7a- |
| | tetrahydroisoindole-2-carbonyl] butyl] imidazolidine-2, 4-dione |
| 50 | 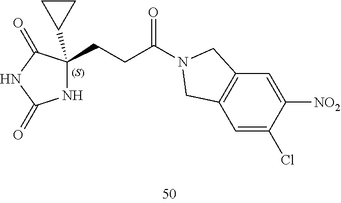 |
| | (S)-5-(3-(5-chloro-6-nitroisoindolin-2-yl)-3-oxopropyl)-5- |
| | cyclopropylimidazolidine-2,4-dione |
| 51 | 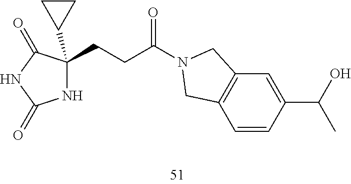 [SMILES: CC(O)c1ccc2c(c1)CN(C(=O)CC[C@@]1(C3CC3)NC(=O)NC1=O)C2] |
| | (5S)-5-cyclopropyl-5-(3-(5-(1-hydroxyethyl)isoindolin-2-yl)-3- |
| | oxopropyl)imidazolidine-2,4-dione |
| 52 | 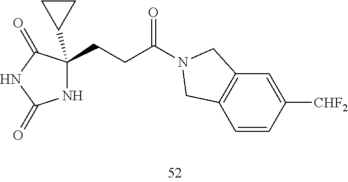 [SMILES: O=C1NC(=O)[C@](CCC(=O)N2Cc3ccc(C(F)F)cc3C2)(C2CC2)N1] |
| | (S)-5-cyclopropyl-5-(3-(5-(difluoromethyl)isoindolin-2-yl)-3- |
| | oxopropyl)imidazolidine-2,4-dione |
| 53 | 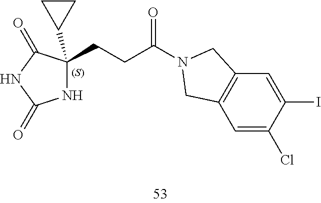 |
| | (S)-5-(3-(5-chloro-6-iodoisoindolin-2-yl)-3-oxopropyl)-5- |
| | cyclopropylimidazolidine-2,4-dione |
| 54 | 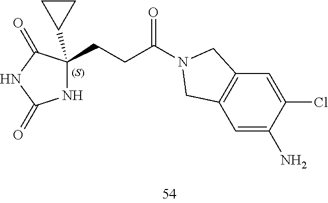 |
| | (S)-5-(3-(5-amino-6-chloroisoindolin-2-yl)-3-oxopropyl)-5- |
| | cyclopropylimidazolidine-2,4-dione |
| 55 | 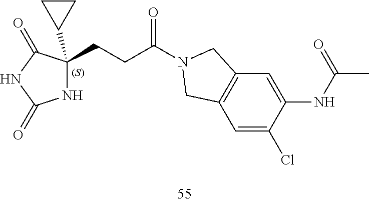 |
| | (S)-N-(6-chloro-2-(3-(4-cyclopropyl-2,5-dioxoimidazolidin-4- |
| | yl)propanoyl)isoindolin-5-yl)acetamide |
| 56 | 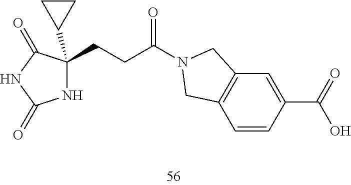 [SMILES: O=C1NC(=O)[C@](CCC(=O)N2Cc3ccc(C(=O)O)cc3C2)(C2CC2)N1] |
| | (S)-2-(3-(4-cyclopropyl-2,5-dioxoimidazolidin-4-yl)propanoyl)isoindoline-5- |
| | carboxylic acid |
| 57 | 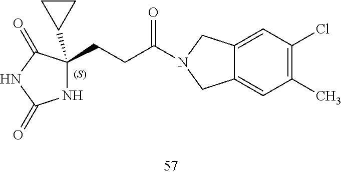 |
| | (S)-5-(3-(5-chloro-6-methylisoindolin-2-yl)-3-oxopropyl)-5- |
| | cyclopropylimidazolidine-2,4-dione |
| 58 | 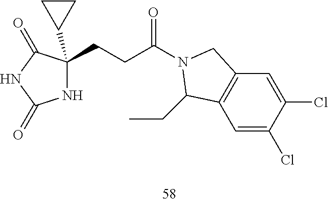 [SMILES: CCC1c2cc(Cl)c(Cl)cc2CN1C(=O)CC[C@@]1(C2CC2)NC(=O)NC1=O] |
| | (5S)-5-cyclopropyl-5-(3-(5,6-dichloro-1-ethylisoindolin-2-yl)-3- |
| | oxopropyl)imidazolidine-2,4-dione |
| 59 | 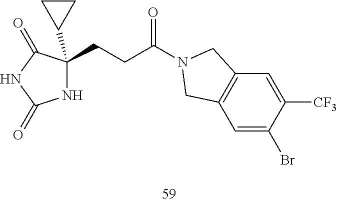 [SMILES: O=C1NC(=O)[C@](CCC(=O)N2Cc3cc(Br)c(C(F)(F)F)cc3C2)(C2CC2)N1] |
| | (S)-5-(3-(5-bromo-6-(trifluoromethyl)isoindolin-2-yl)-3-oxopropyl)-5- |
| | cyclopropylimidazolidine-2,4-dione |
| 60 | 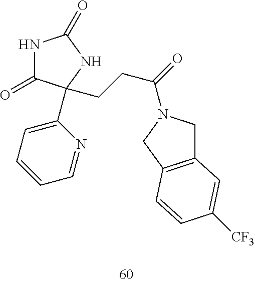 [SMILES: O=C1NC(=O)C(CCC(=O)N2Cc3ccc(C(F)(F)F)cc3C2)(c2ccccn2)N1] |
| | 5-(3-oxo-3-(5-(trifluoromethyl)isoindolin-2-yl)propyl)-5-(pyridin-2- |
| | yl)imidazolidine-2,4-dione |
| 61 | 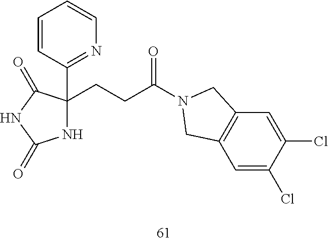 [SMILES: O=C1NC(=O)C(CCC(=O)N2Cc3cc(Cl)c(Cl)cc3C2)(c2ccccn2)N1] |
| | 5-(3-(5,6-dichloroisoindolin-2-yl)-3-oxopropyl)-5-(pyridin-2- |
| | yl)imidazolidine-2,4-dione |
| 62 | 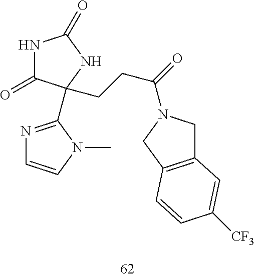 [SMILES: Cn1ccnc1C1(CCC(=O)N2Cc3ccc(C(F)(F)F)cc3C2)NC(=O)NC1=O] |
| | 5-(1-methyl-1 H-imidazol-2-yl)-5-(3-oxo-3-(5-(trifluoromethyl)isoindolin-2- |
| | yl)propyl)imidazolidine-2,4-dione |
| 62-1 | 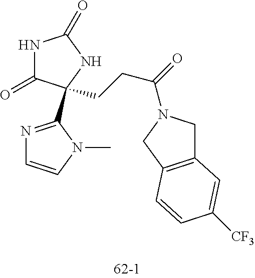 [SMILES: Cn1ccnc1[C@]1(CCC(=O)N2Cc3ccc(C(F)(F)F)cc3C2)NC(=O)NC1=O] |
| | (S)-5-(1-methyl-1H-imidazol-2-yl)-5-(3-oxo-3-(5-(trifluoromethyl)isoindolin- |
| | 2-yl)propyl)imidazolidine-2,4-dione |
| 62-2 | 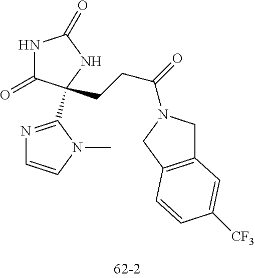 [SMILES: Cn1ccnc1[C@@]1(CCC(=O)N2Cc3ccc(C(F)(F)F)cc3C2)NC(=O)NC1=O] |
| | (R)-5-(1-methyl-1H-imidazol-2-yl)-5-(3-oxo-3-(5-(trifluoromethyl)isoindolin- |
| | 2-yl)propyl)imidazolidine-2,4-dione |
| 63 | 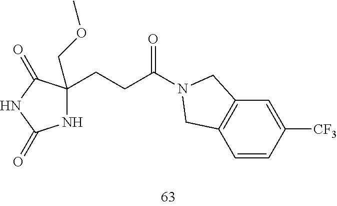 [SMILES: COCC1(CCC(=O)N2Cc3ccc(C(F)(F)F)cc3C2)NC(=O)NC1=O] |
| | 5-(methoxymethyl)-5-(3-oxo-3-(5-(trifluoromethyl)isoindolin-2- |
| | yl)propyl)imidazolidine-2,4-dione |
| 63-1 | 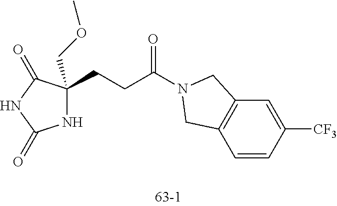 [SMILES: COC[C@]1(CCC(=O)N2Cc3ccc(C(F)(F)F)cc3C2)NC(=O)NC1=O] |
| | (S)-5-(methoxymethyl)-5-(3-oxo-3-(5-(trifluoromethyl)isoindolin-2- |
| | yl)propyl)imidazolidine-2,4-dione |
| 63-2 | 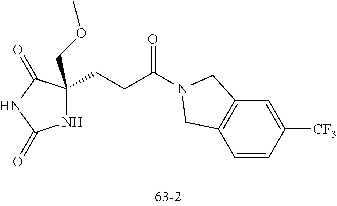 [SMILES: COC[C@@]1(CCC(=O)N2Cc3ccc(C(F)(F)F)cc3C2)NC(=O)NC1=O] |
| | (R)-5-(methoxymethyl)-5-(3-oxo-3-(5-(trifluoromethyl)isoindolin-2- |
| | yl)propyl)imidazolidine-2,4-dione |
| 64 | 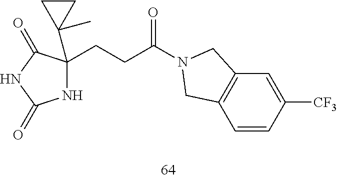 [SMILES: CC1(C2(CCC(=O)N3Cc4ccc(C(F)(F)F)cc4C3)NC(=O)NC2=O)CC1] |
| | 5-(1-methylcyclopropyl)-5-(3-oxo-3-(5-(trifluoromethyl)isoindolin-2- |
| | yl)propyl)imidazolidine-2,4-dione |
| 64-1 | 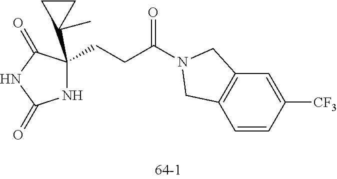 [SMILES: CC1([C@@]2(CCC(=O)N3Cc4ccc(C(F)(F)F)cc4C3)NC(=O)NC2=O)CC1] |
| | (R)-5-(1-methylcyclopropyl)-5-(3-oxo-3-(5-(trifluoromethyl)isoindolin-2- |
| | yl)propyl)imidazolidine-2,4-dione |
| 64-2 | 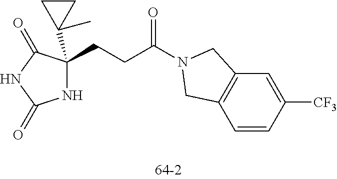 [SMILES: CC1([C@]2(CCC(=O)N3Cc4ccc(C(F)(F)F)cc4C3)NC(=O)NC2=O)CC1] |
| | (S)-5-(1-methylcyclopropyl)-5-(3-oxo-3-(5-(trifluoromethyl)isoindolin-2- |
| | yl)propyl)imidazolidine-2,4-dione |
| 65 | 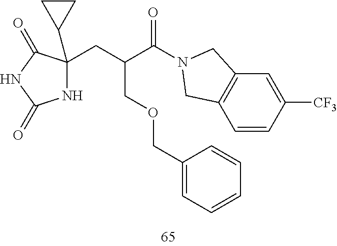 [SMILES: O=C1NC(=O)C(CC(COCc2ccccc2)C(=O)N2Cc3ccc(C(F)(F)F)cc3C2)(C2CC2)N1] |
| | 5-(2-((benzyloxy)methyl)-3-oxo-3-(5-(trifluoromethyl)isoindolin-2-yl)propyl)- |
| | 5-cyclopropylimidazolidine-2,4-dione |
| 66 | 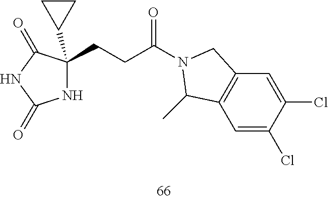 [SMILES: CC1c2cc(Cl)c(Cl)cc2CN1C(=O)CC[C@@]1(C2CC2)NC(=O)NC1=O] |
| | (5S)-5-cyclopropyl-5-(3-(5,6-dichloro-1-methylisoindolin-2-yl)-3- |
| | oxopropyl)imidazolidine-2,4-dione |
| 66-1 | 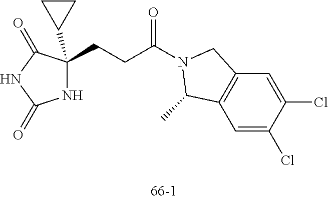 [SMILES: C[C@H]1c2cc(Cl)c(Cl)cc2CN1C(=O)CC[C@@]1(C2CC2)NC(=O)NC1=O] |
| | (S)-5-cyclopropyl-5-(3-((S)-5,6-dichloro-1-methylisoindolin-2-yl)-3- |
| | oxopropyl)imidazolidine-2,4-dione |
| 66-2 | 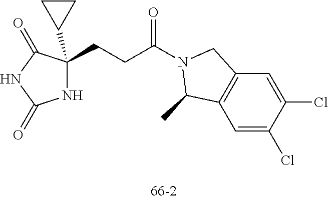 [SMILES: C[C@@H]1c2cc(Cl)c(Cl)cc2CN1C(=O)CC[C@@]1(C2CC2)NC(=O)NC1=O] |
| | (S)-5-cyclopropyl-5-(3-((R)-5,6-dichloro-1-methylisoindolin-2-yl)-3- |
| | oxopropyl)imidazolidine-2,4-dione |
| 67 | 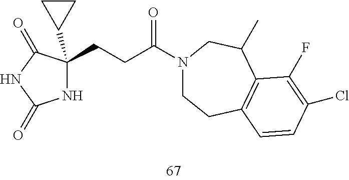 [SMILES: CC1CN(C(=O)CC[C@@]2(C3CC3)NC(=O)NC2=O)CCc2ccc(Cl)c(F)c21] |
| | (5S)-5-(3-(8-chloro-9-fluoro-1-methyl-1,2,4,5-tetrahydro-3H-benzo[d]azepin- |
| | 3-yl)-3-oxopropyl)-5-cyclopropylimidazolidine-2,4-dione |
| 67-1 | 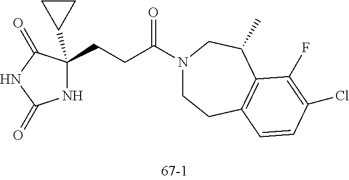 [SMILES: C[C@H]1CN(C(=O)CC[C@@]2(C3CC3)NC(=O)NC2=O)CCc2ccc(Cl)c(F)c21] |
| | (S)-5-(3-((R)-8-chloro-9-fluoro-1-methyl-1,2,4,5-tetrahydro-3H- |
| | benzo[d]azepin-3-yl)-3-oxopropyl)-5-cyclopropylimidazolidine-2,4-dione |
| 67-2 | 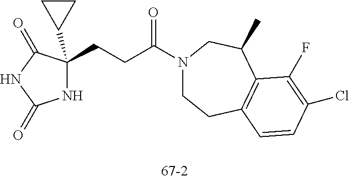 [SMILES: C[C@@H]1CN(C(=O)CC[C@@]2(C3CC3)NC(=O)NC2=O)CCc2ccc(Cl)c(F)c21] |
| | (S)-5-(3-((S)-8-chloro-9-fluoro-1-methyl-1,2,4,5-tetrahydro-3H- |
| | benzo[d]azepin-3-yl)-3-oxopropyl)-5-cyclopropylimidazolidine-2,4-dione |
| 68 | 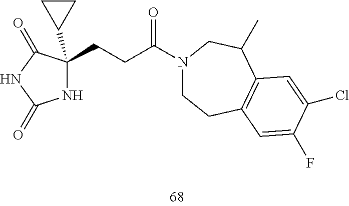 [SMILES: CC1CN(C(=O)CC[C@@]2(C3CC3)NC(=O)NC2=O)CCc2cc(F)c(Cl)cc21] |
| | (5S)-5-(3-(8-chloro-7-fluoro-1-methyl-1,2,4,5-tetrahydro-3H-benzo[d]azepin- |
| | 3-yl)-3-oxopropyl)-5-cyclopropylimidazolidine-2,4-dione |
| 68-1 | 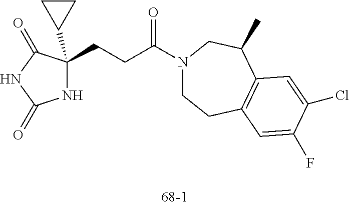 [SMILES: C[C@@H]1CN(C(=O)CC[C@@]2(C3CC3)NC(=O)NC2=O)CCc2cc(F)c(Cl)cc21] |
| | (S)-5-(3-((S)-8-chloro-7-fluoro-1-methyl-1,2,4,5-tetrahydro-3H- |
| | benzo[d]azepin-3-yl)-3-oxopropyl)-5-cyclopropylimidazolidine-2,4-dione |
| 68-2 | 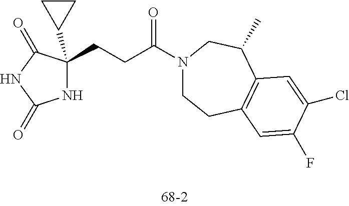 [SMILES: C[C@H]1CN(C(=O)CC[C@@]2(C3CC3)NC(=O)NC2=O)CCc2cc(F)c(Cl)cc21] |
| | (S)-5-(3-((R)-8-chloro-7-fluoro-1-methyl-1,2,4,5-tetrahydro-3H- |
| | benzo[d]azepin-3-yl)-3-oxopropyl)-5-cyclopropylimidazolidine-2,4-dione |
| 69 | 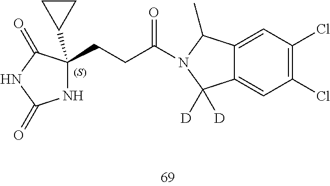 |
| | (5S)-5-cyclopropyl-5-(3-(5,6-dichloro-1-methylisoindolin-2-yl-3,3-d2)-3- |
| | oxopropyl)imidazolidine-2,4-dione |
| 69-1 | 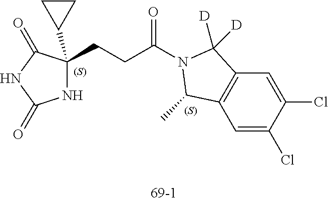 |
| | (S)-5-cyclopropyl-5-(3-((S)-5,6-dichloro-1-methylisoindolin-2-yl-3,3-d2)-3- |
| | oxopropyl)imidazolidine-2,4-dione |
| 69-2 | 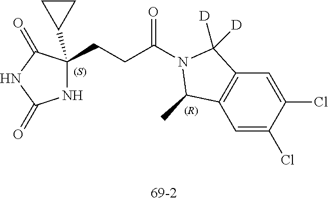 |
| | (S)-5-cyclopropyl-5-(3-((R)-5,6-dichloro-1-methylisoindolin-2-yl-3,3- d2)-3- |
| | oxopropyl)imidazolidine-2,4-dione |
| 70 | 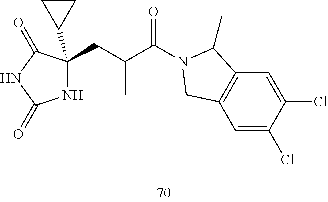 [SMILES: CC(C[C@@]1(C2CC2)NC(=O)NC1=O)C(=O)N1Cc2cc(Cl)c(Cl)cc2C1C] |
| | (5S)-5-cyclopropyl-5-(3-(5,6-dichloro-1-methylisoindolin-2-yl)-2-methyl-3- |
| | oxopropyl)imidazolidine-2,4-dione |
| 71 | 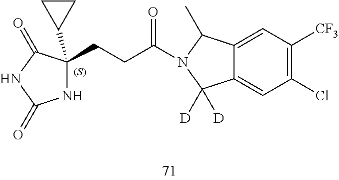 |
| | (5S)-5-(3-(5-chloro-1-methyl-6-(trifluoromethyl)isoindolin-2-yl-3,3-d2)-3- |
| | oxopropyl)-5-cyclopropylimidazolidine-2,4-dione |
| 71-1 | 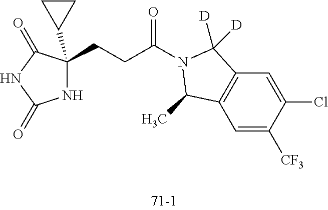 [SMILES: [2H]C1([2H])c2cc(Cl)c(C(F)(F)F)cc2[C@@H](C)N1C(=O)CC[C@@]1(C2CC2)NC(=O)NC1=O] |
| | (S)-5-(3-((R)-5-chloro-1-methyl-6-(trifluoromethyl)isoindolin-2-yl-3,3-d2)-3- |
| | oxopropyl)-5-cyclopropylimidazolidine-2,4-dione |
| 71-2 | 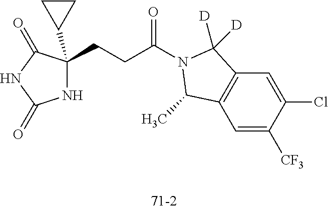 [SMILES: [2H]C1([2H])c2cc(Cl)c(C(F)(F)F)cc2[C@H](C)N1C(=O)CC[C@@]1(C2CC2)NC(=O)NC1=O] |
| | (S)-5-(3-((S)-5-chloro-1-methyl-6-(trifluoromethyl)isoindolin-2-yl-3,3-d2)-3- |
| | oxopropyl)-5-cyclopropylimidazolidine-2,4-dione |
| 72 | 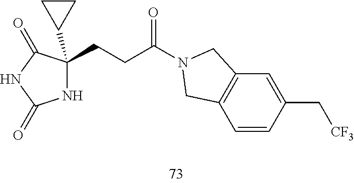 [SMILES: O=C1NC(=O)[C@](CCC(=O)N2Cc3ccc(CC(F)(F)F)cc3C2)(C2CC2)N1] |
| | (S)-5-cyclopropyl-5-(3-oxo-3-(5-(2,2,2-trifluoroethyl)isoindolin-2- |
| | yl)propyl)imidazolidine-2,4-dione |
| 73 | 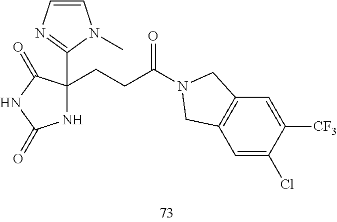 [SMILES: Cn1ccnc1C1(CCC(=O)N2Cc3cc(Cl)c(C(F)(F)F)cc3C2)NC(=O)NC1=O] |
| | 5-(3-(5-chloro-6-(trifluoromethyl)isoindolin-2-yl)-3-oxopropyl)-5-(1-methyl- |
| | 1H-imidazol-2-yl)imidazolidine-2,4-dione |
| 73-1 | 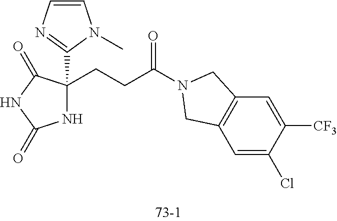 [SMILES: Cn1ccnc1[C@]1(CCC(=O)N2Cc3cc(Cl)c(C(F)(F)F)cc3C2)NC(=O)NC1=O] |
| | (S)-5-(3-(5-chloro-6-(trifluoromethyl)isoindolin-2-yl)-3-oxopropyl)-5-(1- |
| | methyl-1H-imidazol-2-yl)imidazolidine-2,4-dione |
| 73-2 | 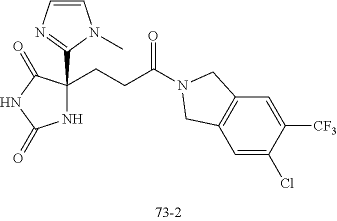 [SMILES: Cn1ccnc1[C@@]1(CCC(=O)N2Cc3cc(Cl)c(C(F)(F)F)cc3C2)NC(=O)NC1=O] |
| | (R)-5-(3-(5-chloro-6-(trifluoromethyl)isoindolin-2-yl)-3-oxopropyl)-5-(1- |
| | methyl-1H-imidazol-2-yl)imidazolidine-2,4-dione |
| 74 | 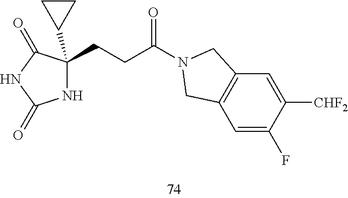 [SMILES: O=C1NC(=O)[C@](CCC(=O)N2Cc3cc(F)c(C(F)F)cc3C2)(C2CC2)N1] |
| | (S)-5-cyclopropyl-5-(3-(5-(difluoromethyl)-6-fluoroisoindolin-2-yl)-3- |
| | oxopropyl)imidazolidine-2,4-dione |
| 75 | 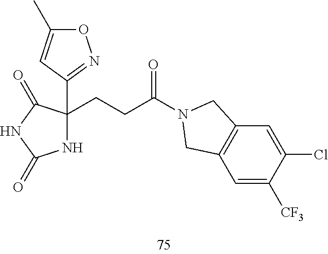 [SMILES: Cc1cc(C2(CCC(=O)N3Cc4cc(Cl)c(C(F)(F)F)cc4C3)NC(=O)NC2=O)no1] |
| | 5-(3-(5-chloro-6-(trifluoromethyl)isoindolin-2-yl)-3-oxopropyl)-5-(5- |
| | methylisoxazol-3-yl)imidazolidine-2,4-dione |
| 76 | 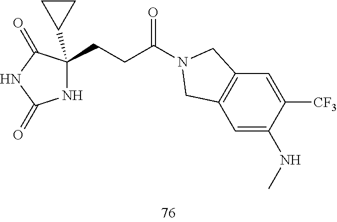 [SMILES: CNc1cc2c(cc1C(F)(F)F)CN(C(=O)CC[C@@]1(C3CC3)NC(=O)NC1=O)C2] |
| | (S)-5-cyclopropyl-5-(3-(5-(methylamino)-6-(trifluoromethyl)isoindolin-2-yl)- |
| | 3-oxopropyl)imidazolidine-2,4-dione |
| 77 | 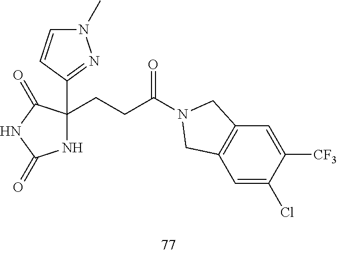 [SMILES: Cn1ccc(C2(CCC(=O)N3Cc4cc(Cl)c(C(F)(F)F)cc4C3)NC(=O)NC2=O)n1] |
| | 5-(3-(5-chloro-6-(trifluoromethyl)isoindolin-2-yl)-3-oxopropyl)-5-(1-methyl- |
| | 1 H-pyrazol-3-yl)imidazolidine-2,4-dione |
| 78 | 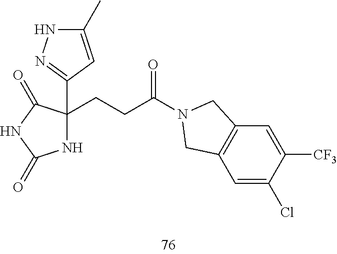 [SMILES: Cc1cc(C2(CCC(=O)N3Cc4cc(Cl)c(C(F)(F)F)cc4C3)NC(=O)NC2=O)n[nH]1] |
| | 5-(3-(5-chloro-6-(trifluoromethyl)isoindolin-2-yl)-3-oxopropyl)-5-(5-methyl- |
| | 1H-pyrazol-3-yl)imidazolidine-2,4-dione |
| 79 | 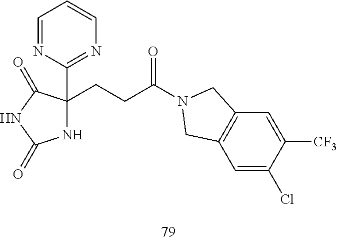 [SMILES: O=C1NC(=O)C(CCC(=O)N2Cc3cc(Cl)c(C(F)(F)F)cc3C2)(c2ncccn2)N1] |
| | 5-(3-(5-chloro-6-(trifluoromethyl)isoindolin-2-yl)-3-oxopropyl)-5-(pyrimidin- |
| | 2-yl)imidazolidine-2,4-dione |
| 80 | 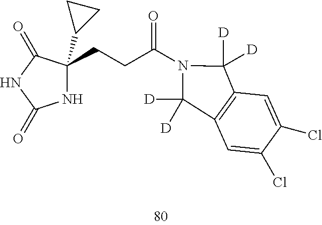 [SMILES: [2H]C1([2H])c2cc(Cl)c(Cl)cc2C([2H])([2H])N1C(=O)CC[C@@]1(C2CC2)NC(=O)NC1=O] |
| | (S)-5-cyclopropyl-5-(3-(5,6-dichloroisoindolin-2-yl-1,1,3,3-d4)-3- |
| | oxopropyl)imidazolidine-2,4-dione |
| 81 | 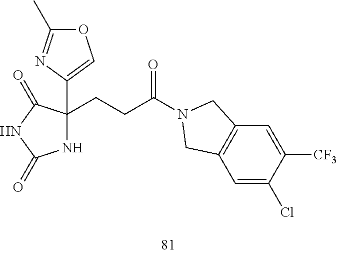 [SMILES: Cc1nc(C2(CCC(=O)N3Cc4cc(Cl)c(C(F)(F)F)cc4C3)NC(=O)NC2=O)co1] |
| | 5-(3-(5-chloro-6-(trifluoromethyl)isoindolin-2-yl)-3-oxopropyl)-5-(2- |
| | methyloxazol-4-yl)imidazolidine-2,4-dione |
| 82 | 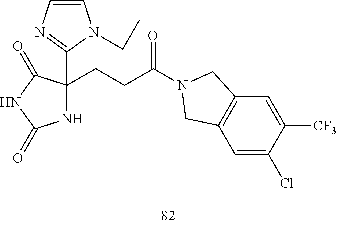 [SMILES: CCn1ccnc1C1(CCC(=O)N2Cc3cc(Cl)c(C(F)(F)F)cc3C2)NC(=O)NC1=O] |
| | 5-(3-(5-chloro-6-(trifluoromethyl)isoindolin-2-yl)-3-oxopropyl)-5-(1-ethyl- |
| | 1H-imidazol-2-yl)imidazolidine-2,4-dione |
| 83 | 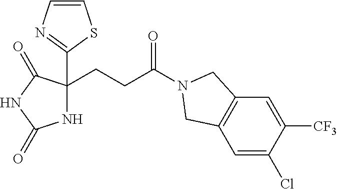 [SMILES: O=C1NC(=O)C(CCC(=O)N2Cc3cc(Cl)c(C(F)(F)F)cc3C2)(c2nccs2)N1] |
| | 5-(3-(5-chloro-6-(trifluoromethyl)isoindolin-2-yl)-3-oxopropyl)-5-(thiazol-2- |
| | yl)imidazolidine-2,4-dione |
| 83-1 | 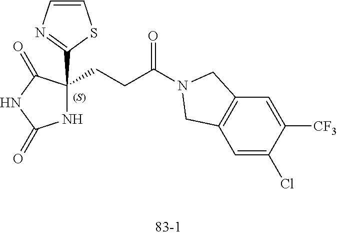 |
| | (S)-5-(3-(5-chloro-6-(trifluoromethyl)isoindolin-2-yl)-3-oxopropyl)-5- |
| | (thiazol-2-yl)imidazolidine-2,4-dione |
| 83-2 | 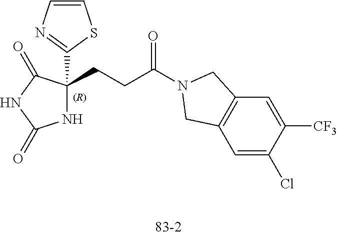 |
| | (R)-5-(3-(5-chloro-6-(trifluoromethyl)isoindolin-2-yl)-3-oxopropyl)-5- |
| | (thiazol-2-yl)imidazolidine-2,4-dione |
| 84 | 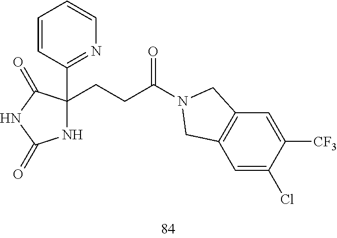 [SMILES: O=C1NC(=O)C(CCC(=O)N2Cc3cc(Cl)c(C(F)(F)F)cc3C2)(c2ccccn2)N1] |
| | 5-(3-(5-chloro-6-(trifluoromethyl)isoindolin-2-yl)-3-oxopropyl)-5-(pyridin-2- |
| | yl)imidazolidine-2,4-dione |
| 84-1 | 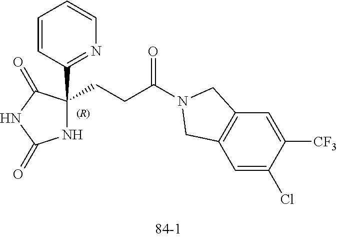 |
| | (R)-5-(3-(5-chloro-6-(trifluoromethyl)isoindolin-2-yl)-3-oxopropyl)-5- |
| | (pyridin-2-yl)imidazolidine-2,4-dione |
| 84-2 | 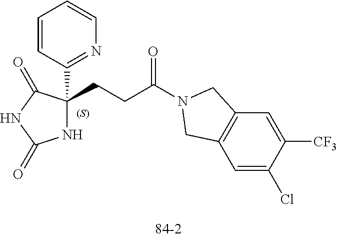 |
| | (S)-5-(3-(5-chloro-6-(trifluoromethyl)isoindolin-2-yl)-3-oxopropyl)-5- |
| | (pyridin-2-yl)imidazolidine-2,4-dione |
| 85 | 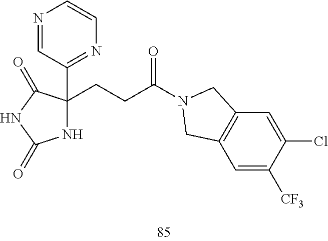 [SMILES: O=C1NC(=O)C(CCC(=O)N2Cc3cc(Cl)c(C(F)(F)F)cc3C2)(c2cnccn2)N1] |
| | 5-(3-(5-chloro-6-(trifluoromethyl)isoindolin-2-yl)-3-oxopropyl)-5-(pyrazin-2- |
| | yl)imidazolidine-2,4-dione |
| 85-1 | 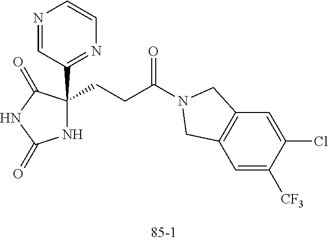 [SMILES: O=C1NC(=O)[C@@](CCC(=O)N2Cc3cc(Cl)c(C(F)(F)F)cc3C2)(c2cnccn2)N1] |
| | (R)-5-(3-(5-chloro-6-(trifluoromethyl)isoindolin-2-yl)-3-oxopropyl)-5- |
| | (pyrazin-2-yl)imidazolidine-2,4-dione |
| 85-2 | 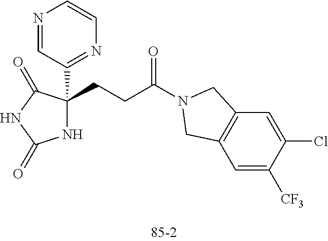 [SMILES: O=C1NC(=O)[C@](CCC(=O)N2Cc3cc(Cl)c(C(F)(F)F)cc3C2)(c2cnccn2)N1] |
| | (S)-5-(3-(5-chloro-6-(trifluoromethyl)isoindolin-2-yl)-3-oxopropyl)-5- |
| | (pyrazin-2-yl)imidazolidine-2,4-dione |
| 86-1 | 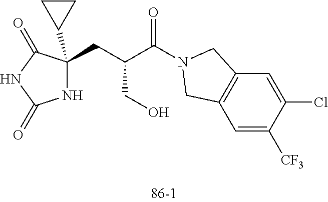 [SMILES: O=C1NC(=O)[C@](C[C@@H](CO)C(=O)N2Cc3cc(Cl)c(C(F)(F)F)cc3C2)(C2CC2)N1] |
| | (S)-5-((S)-3-(5-chloro-6-(trifluoromethyl)isoindolin-2-yl)-2-(hydroxymethyl)- |
| | 3-oxopropyl)-5-cyclopropylimidazolidine-2,4-dione |
| 86-2 | 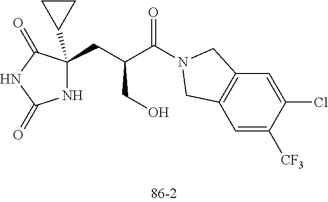 [SMILES: O=C1NC(=O)[C@](C[C@H](CO)C(=O)N2Cc3cc(Cl)c(C(F)(F)F)cc3C2)(C2CC2)N1] |
| | (S)-5-((R)-3-(5-chloro-6-(trifluoromethyl)isoindolin-2-yl)-2-(hydroxymethyl)- |
| | 3-oxopropyl)-5-cyclopropylimidazolidine-2,4-dione |
| 86-3 | 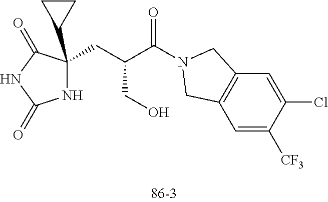 [SMILES: O=C1NC(=O)[C@@](C[C@@H](CO)C(=O)N2Cc3cc(Cl)c(C(F)(F)F)cc3C2)(C2CC2)N1] |
| | (R)-5-((S)-3-(5-chloro-6-(trifluoromethyl)isoindolin-2-yl)-2- |
| | (hydroxymethyl)-3-oxopropyl)-5-cyclopropylimidazolidine-2,4-dione |
| 86-4 | 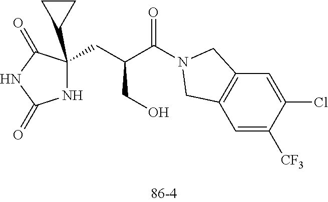 [SMILES: O=C1NC(=O)[C@@](C[C@H](CO)C(=O)N2Cc3cc(Cl)c(C(F)(F)F)cc3C2)(C2CC2)N1] |
| | (R)-5-((R)-3-(5-chloro-6-(trifluoromethyl)isoindolin-2-yl)-2-(hydroxymethyl)- |
| | 3-oxopropyl)-5-cyclopropylimidazolidine-2,4-dione |
| | 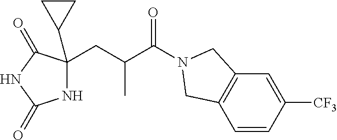 [SMILES: CC(CC1(C2CC2)NC(=O)NC1=O)C(=O)N1Cc2ccc(C(F)(F)F)cc2C1] |
| | 5-cyclopropyl-5-(2-methyl-3-oxo-3-(5-(trifluoromethyl)isoindolin-2- |
| | yl)propyl)imidazolidine-2,4-dione |
| | 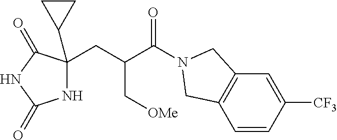 [SMILES: COCC(CC1(C2CC2)NC(=O)NC1=O)C(=O)N1Cc2ccc(C(F)(F)F)cc2C1] |
| | 5-cyclopropyl-5-(2-(methoxymethyl)-3-oxo-3-(5-(trifluoromethyl)isoindolin- |
| | 2-yl)propyl)imidazolidine-2,4-dione |
| | 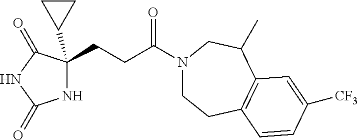 [SMILES: CC1CN(C(=O)CC[C@@]2(C3CC3)NC(=O)NC2=O)CCc2ccc(C(F)(F)F)cc21] |
| | (5S)-5-cyclopropyl-5-(3-(1-methyl-8-(trifluoromethyl)-4,5-dihydro-1H- |
| | benzo[d]azepin-3(2H)-yl)-3-oxopropyl)imidazolidine-2,4-dione |
| | 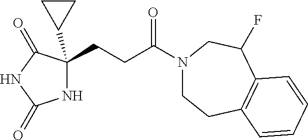 [SMILES: O=C1NC(=O)[C@](CCC(=O)N2CCc3ccccc3C(F)C2)(C2CC2)N1] |
| | (5S)-5-cyclopropyl-5-(3-(1-fluoro-4,5-dihydro-1H-benzo[d]azepin-3(2H)-yl)- |
| | 3-oxopropyl)imidazolidine-2,4-dione |
| | 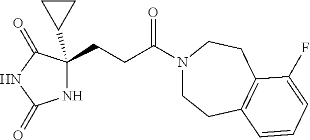 [SMILES: O=C1NC(=O)[C@](CCC(=O)N2CCc3cccc(F)c3CC2)(C2CC2)N1] |
| | (S)-5-cyclopropyl-5-(3-(6-fluoro-4,5-dihydro-1H-benzo[d]azepin-3(2H)-yl)-3- |
| | oxopropyl)imidazolidine-2,4-dione |
| | 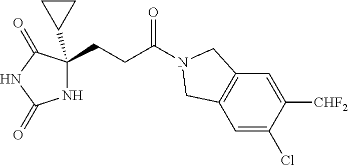 [SMILES: O=C1NC(=O)[C@](CCC(=O)N2Cc3cc(Cl)c(C(F)F)cc3C2)(C2CC2)N1] |
| | (S)-5-(3-(5-chloro-6-(difluoromethyl)isoindolin-2-yl)-3-oxopropyl)-5- |
| | cyclopropylimidazolidine-2,4-dione |
| | 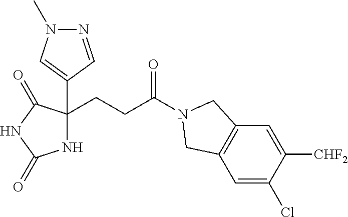 [SMILES: Cn1cc(C2(CCC(=O)N3Cc4cc(Cl)c(C(F)F)cc4C3)NC(=O)NC2=O)cn1] |
| | 5-(3-(5-chloro-6-(trifluoromethyl)isoindolin-2-yl)-3-oxopropyl)-5-(1-methyl- |
| | 1H-pyrazol-4-yl)imidazolidine-2,4-dione |

In another aspect, this disclosure provides a process for preparation of a compound of formula (I), or a tautomer, mesomer, racemate, enantiomer, diastereomer, or mixture thereof, or a pharmaceutically acceptable salt, solvate or prodrug thereof, the preparation process comprising the steps of:

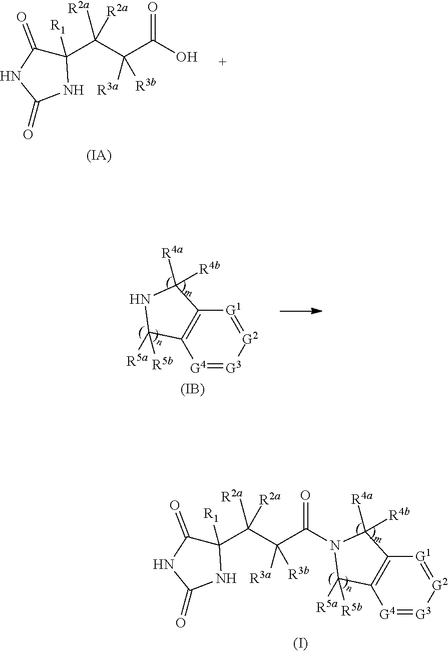

reacting a compound of formula (IA) or a tautomer, mesomer, racemate, enantiomer, diastereomer, or mixture thereof, or a salt thereof, with a compound of formula (IB), or a salt thereof, to obtain the compound of formula (I) or a tautomer, mesomer, racemate, enantiomer, diastereomer, or mixture thereof, or a pharmaceutically acceptable salt, solvate or prodrug thereof,
wherein:
the salt of the compound (IB) preferably is hydrochloride; and
G¹, G², G³, G⁴, R¹, R^{2a} to R^{5a}, R^{2b} to R^{5b}, n and m are each as defined in formula (I).

In another aspect, this disclosure provides a process for preparation of a compound of formula (II), or a tautomer, mesomer, racemate, enantiomer, diastereomer, or mixture thereof, or a pharmaceutically acceptable salt, solvate or prodrug thereof, the preparation process comprising the steps of:

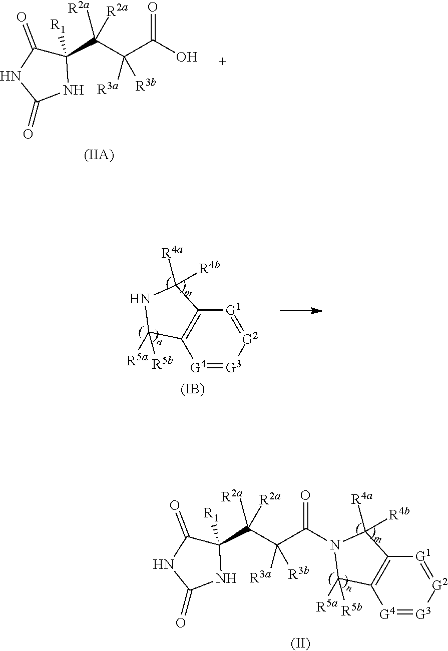

reacting a compound of formula (IIA) or a tautomer, mesomer, racemate, enantiomer, diastereomer, or mixture thereof, or a salt thereof, with a compound of formula (IB), or a salt thereof, to obtain the compound of formula (II) or a tautomer, mesomer, racemate, enantiomer, diastereomer, or mixture thereof, or a pharmaceutically acceptable salt, solvate or prodrug thereof;
wherein:
the salt of the compound (IB) preferably is hydrochloride; and
G¹, G², G³, G⁴, R¹, R^{2a} to R^{5a}, R^{2b} to R^{5b}, n and m are each as defined in formula (II).

In another aspect, this disclosure provides a process for preparation of a compound of formula (III), or a tautomer, mesomer, racemate, enantiomer, diastereomer, or mixture thereof, or a pharmaceutically acceptable salt, solvate or prodrug thereof, the preparation process comprising the steps of:

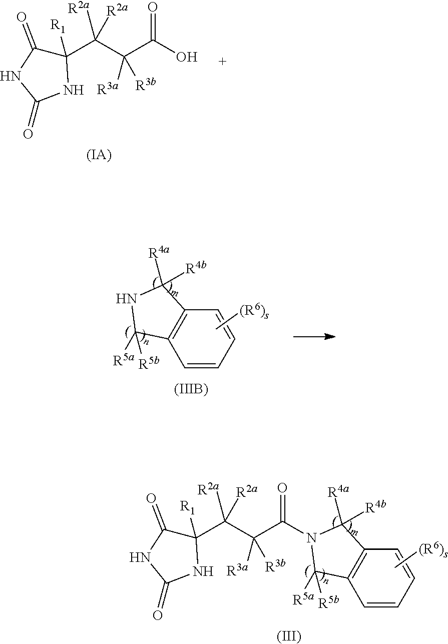

reacting a compound of formula (IA) or a tautomer, mesomer, racemate, enantiomer, diastereomer, or mixture thereof, or a salt thereof, with a compound of formula (IIIB), or a salt thereof, to obtain the compound of formula (III) or a tautomer, mesomer, racemate, enantiomer, diastereomer, or mixture thereof, or a pharmaceutically acceptable salt, solvate or prodrug thereof;
wherein:
the salt of the compound (IIIB) preferably is hydrochloride; and
R¹, R^{2a} to R^{5a}, R^{2b} to R^{5b}, R⁶, n, m and s are each as defined in formula (III).

In another aspect, this disclosure provides a process for preparation of a compound of formula (IIIa), or a tautomer, mesomer, racemate, enantiomer, diastereomer, or mixture thereof, or a pharmaceutically acceptable salt, solvate or prodrug thereof, the preparation process comprising the steps of:

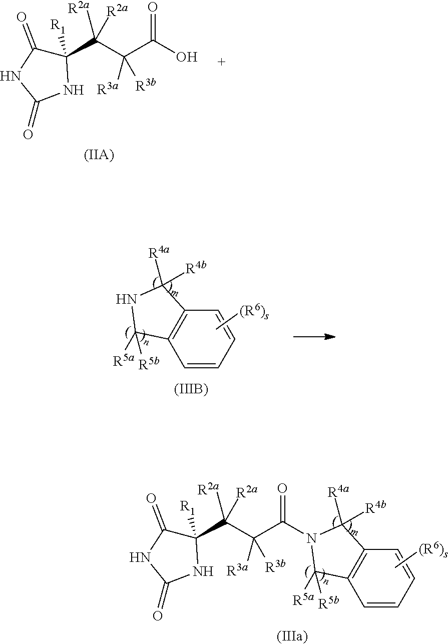

reacting a compound of formula (IIA) or a tautomer, mesomer, racemate, enantiomer, diastereomer, or mixture thereof, or a salt thereof, with a compound of formula (IIIB), or a salt thereof, to obtain the compound of formula (IIIa) or a tautomer, mesomer, racemate, enantiomer, diastereomer, or mixture thereof, or a pharmaceutically acceptable salt, solvate or prodrug thereof;
wherein:
the salt of the compound (IIIB) preferably is hydrochloride; and
R¹, R^{2a} to R^{5a}, R^{2b} to R^{5b}, R⁶, n, m and s are each as defined in formula (IIIa).

In another aspect, this disclosure provides a process for preparation of a compound of formula (IV), or a tautomer, mesomer, racemate, enantiomer, diastereomer, or mixture thereof, or a pharmaceutically acceptable salt, solvate or prodrug thereof, the preparation process comprising the steps of:

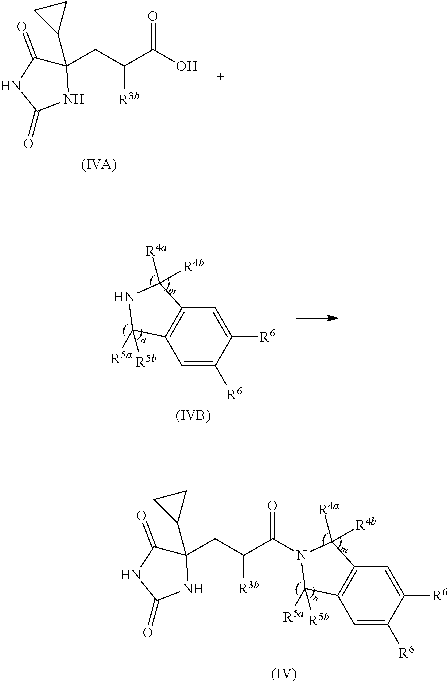

reacting a compound of formula (IVA) or a tautomer, mesomer, racemate, enantiomer, diastereomer, or mixture thereof, or a salt thereof, with a compound of formula (IVB), or a salt thereof, to obtain the compound of formula (IV) or a tautomer, mesomer, racemate, enantiomer, diastereomer, or mixture thereof, or a pharmaceutically acceptable salt, solvate or prodrug thereof;
wherein:
the salt of the compound (IV) preferably is hydrochloride; and
R^{4a}, R^{5a}, R^{3b} to R^{5b}, R⁶, n and m are each as defined in formula (IV).

In another aspect, this disclosure provides a process for preparation of a compound of formula (IVa), or a tautomer, mesomer, racemate, enantiomer, diastereomer, or mixture thereof, or a pharmaceutically acceptable salt, solvate or prodrug thereof, the preparation process comprising the steps of:

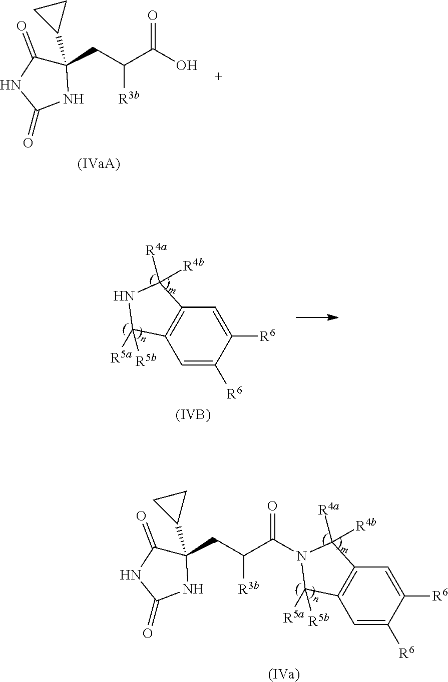

reacting a compound of formula (IVaA) or a tautomer, mesomer, racemate, enantiomer, diastereomer, or mixture thereof, or a salt thereof, with a compound of formula (IVB), or a salt thereof, to obtain the compound of formula (IVa) or a tautomer, mesomer, racemate, enantiomer, diastereomer, or mixture thereof, or a pharmaceutically acceptable salt, solvate or prodrug thereof;
wherein:
the salt of the compound (IVB) preferably is hydrochloride; and
R^{4a}, R^{5a}, R^{3b} to R^{5b}, R⁶, n and m are each as defined in formula (IVa).

In another aspect, this disclosure provides a process for preparation of a compound of formula (V), or a tautomer, mesomer, racemate, enantiomer, diastereomer, or mixture thereof, or a pharmaceutically acceptable salt, solvate or prodrug thereof, the preparation process comprising the steps of:

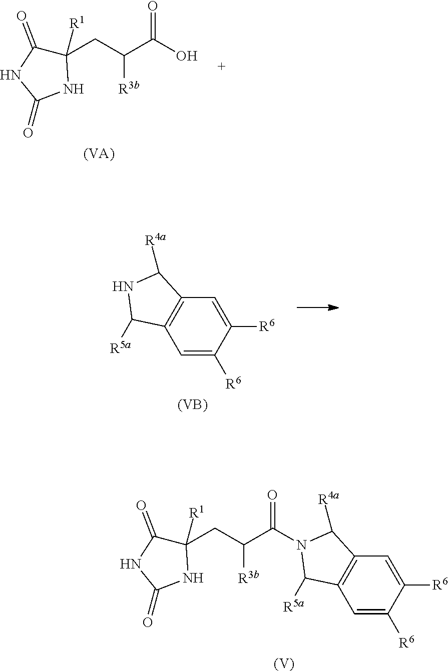

reacting a compound of formula (VA) or a tautomer, mesomer, racemate, enantiomer, diastereomer, or mixture thereof, or a salt thereof, with a compound of formula (VB), or a salt thereof, to obtain the compound of formula (V) or a tautomer, mesomer, racemate, enantiomer, diastereomer, or mixture thereof, or a pharmaceutically acceptable salt, solvate or prodrug thereof;
wherein:
the salt of the compound (VB) preferably is hydrochloride; and
R^{4a}, R^{5a}, R^{3b}, R¹ and R⁶ are each as defined in formula (V).

In another aspect, this disclosure provides a process for preparation of a compound of formula (Va) or (Vb), or a tautomer, mesomer, racemate, enantiomer, diastereomer, or mixture thereof, or a pharmaceutically acceptable salt, solvate or prodrug thereof, the preparation process comprising the steps of:

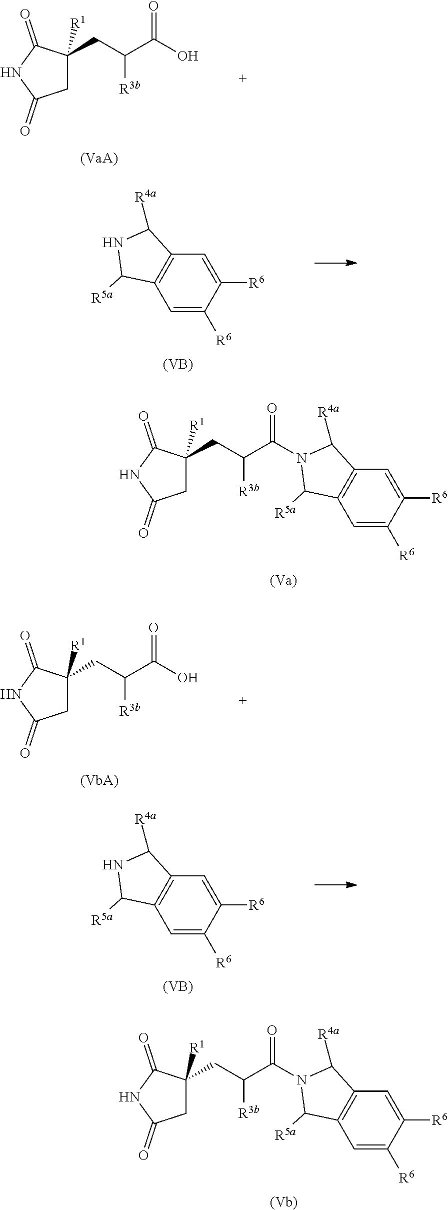

reacting a compound of formula (VaA) or a tautomer, mesomer, racemate, enantiomer, diastereomer, or mixture thereof, or a salt thereof, with a compound of formula (VB), or a salt thereof, to obtain the compound of formula (Va) or a tautomer, mesomer, racemate, enantiomer, diastereomer, or mixture thereof, or a pharmaceutically acceptable salt, solvate or prodrug thereof;
reacting a compound of formula (VbA) or a tautomer, mesomer, racemate, enantiomer, diastereomer, or mixture thereof, or a salt thereof, with a compound of formula (VB), or a salt thereof, to obtain the compound of formula (Vb) or a tautomer, mesomer, racemate, enantiomer, diastereomer, or mixture thereof, or a pharmaceutically acceptable salt, solvate or prodrug thereof;
wherein:
the salt of the compound (VB) preferably is hydrochloride; and
R^{4a}, R^{5a}, R^{3b}, R¹ and R⁶ are each as defined in formula (V).

In another aspect, the present disclosure also provides a pharmaceutical composition, comprising a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, and one or more pharmaceutically acceptable carriers, diluents and/or other excipients.

In another aspect, the present disclosure provides a method of inhibiting ADAMTS-5 and/or ADAMTS-4, comprising a step of administering to a subject in need thereof a therapeutically effective amount of a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound.

In another aspect, the present disclosure provides a method of preventing and/or treating inflammatory conditions, and/or diseases involving degradation of cartilage, and/or disruption of cartilage homeostasis, comprising a step of administering to a subject in need thereof a therapeutically effective amount of a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound.

In another aspect, the present disclosure provides a method of preventing and/or treating arthritis, comprising a step of administering to a subject in need thereof a therapeutically effective amount of the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound; preferably, wherein arthritis is selected from the group consisting of rheumatoid arthritis, psoriatic arthritis, osteoarthrosis and hypertropic arthritis.

In another aspect, the present disclosure also relates to use of a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for the inhibition of ADAMTS-5 and/or ADAMTS-4.

In another aspect, the present disclosure also relates to use of a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for preventing and/or treating inflammatory conditions, and/or diseases involving degradation of cartilage, and/or disruption of cartilage homeostasis.

In another aspect, the present disclosure also relates to use of a compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, in the manufacture of a medicament for preventing and/or treating arthritis; preferably, rheumatoid arthritis, psoriatic arthritis, osteoarthrosis and hypertropic arthritis.

The present disclosure further relates to the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the same, for use as a medicament.

The present disclosure also relates to the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, for use in inhibiting ADAMTS-5 and/or ADAMTS-4.

The present disclosure also relates to the combination of the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, for use in preventing and/or treating inflammatory conditions, and/or diseases involving degradation of cartilage, and/or disruption of cartilage homeostasis.

The present disclosure also relates to the combination of the compound of formula (I), formula (II), formula (III), formula (IIIa), formula (IV), formula (IVa), formula (V), formula (Va) or formula (Vb), or a tautomer, mesomer, racemate, enantiomer, diastereomer, or mixture thereof, or a pharmaceutically acceptable salt, solvate or prodrug thereof, or a pharmaceutical composition containing the compound, for use in preventing and/or treating arthritis; preferably, rheumatoid arthritis, psoriatic arthritis, osteoarthrosis and hypertropic arthritis.

Further, the present disclosure encompasses all compounds that can exist based on any chemically plausible combinations of the embodiments disclosed or substituents on the exemplified compounds, as would be understood by a person of skill in the art.

The term “inflammatory conditions” refers to the group of conditions including rheumatoid arthritis, osteoarthritis, juvenile idiopathic arthritis, psoriasis, psoriatic arthritis, allergic airway disease (e.g. asthma, rhinitis), chronic obstructive pulmonary disease (COPD), inflammatory bowel diseases (e.g. Crohn's disease, ulcerative colitis), endotoxin-driven disease states (e.g.complications after bypass surgery or chronic endotoxin states contributing to e.g. chronic cardiacfailure), and related diseases involving cartilage, such as that of the joints. Particularly refers to rheumatoid arthritis, osteoarthritis, allergic airway disease (e.g. asthma), chronic obstructive pulmonary disease (COPD) and inflammatory bowel diseases. More particularly refers to rheumatoid arthritis, and osteoarthritis (OA). Most particularly refers to osteoarthritis (OA).

The term “diseases involving degradation of cartilage and/or disruption of cartilage homeostasis” includes conditions such as osteoarthritis, psoriatic arthritis, juvenile rheumatoid arthritis, gouty arthritis, septic or infectious arthritis, reactive arthritis, reflex sympathetic dystrophy, algodystrophy, achondroplasia, Paget's disease, Tietze syndrome or costal chondritis, fibromyalgia, osteochondritis, neurogenic or neuropathic arthritis, arthropathy, sarcoidosis, amylosis, hydarthrosis, periodical disease, rheumatoid spondylitis, endemic forms of arthritis like osteoarthritis deformans endemic, Mseleni disease and Handigodu disease; degeneration resulting from fibromyalgia, systemic lupus erythematosus, scleroderma and ankylosing spondylitis; and particularly, refers to osteoarthritis (OA).

The compositions of this disclosure can be formulated by conventional methods using one or more pharmaceutically acceptable carriers. Thus, the active compounds of this disclosure can be formulated as various dosage forms for oral, buccal, intranasal, parenteral (e.g., intravenous, intramuscular or subcutaneous), rectal administration, inhalation or insufflation administration. The compounds of this disclosure can also be formulated as sustained release dosage forms.

Suitable dosage forms include, but are not limited to, a tablet, troche, lozenge, aqueous or oily suspension, dispersible powder or granule, emulsion, hard or soft capsule, or syrup or elixir. Oral compositions can be prepared according to any known method in the art for the preparation of pharmaceutical compositions. Such compositions can contain one or more additives selected from the group consisting of sweeteners, flavoring agents, colorants and preservatives, in order to provide a pleasing and palatable pharmaceutical preparation. Tablets contain the active ingredient and nontoxic pharmaceutically acceptable excipients suitable for the manufacture of tablets. These excipients can be inert excipients, granulating agents, disintegrating agents, and lubricants. The tablet can be uncoated or coated by means of a known technique to mask the taste of the drug or delay the disintegration and absorption of the drug in the gastrointestinal tract, thereby providing sustained release over an extended period. For example, water soluble taste masking materials can be used.

Oral formulations can also be provided as soft gelatin capsules in which the active ingredient is mixed with an inert solid diluent, or the active ingredient is mixed with a water soluble carrier.

An aqueous suspension contains the active ingredient in admixture with excipients suitable for the manufacture of an aqueous suspension. Such excipients are suspending agents, dispersants or humectants, and can be naturally occurring phospholipids. The aqueous suspension can also contain one or more preservatives, one or more colorants, one or more flavoring agents, and one or more sweeteners.

An oil suspension can be formulated by suspending the active ingredient in a vegetable oil, or in a mineral oil. The oil suspension can contain a thickener. The aforementioned sweeteners and flavoring agents can be added to provide a palatable preparation. These compositions can be preserved by adding an antioxidant.

The present pharmaceutical composition can also be in the form of an oil-in-water emulsion. The oil phase can be a vegetable oil, or a mineral oil, or mixture thereof. Suitable emulsifying agents can be naturally occurring phospholipids. Sweeteners can be used. Such formulations can also contain moderators, preservatives, colorants and antioxidants.

The pharmaceutical composition can be in the form of a sterile injectable aqueous solution. The acceptable vehicles and solvents that can be employed are water, Ringer's solution and isotonic sodium chloride solution. The sterile injectable preparation can also be a sterile injectable oil-in-water microemulsion in which the active ingredient is dissolved in the oil phase. The injectable solution or microemulsion can be introduced into an individual's bloodstream by local bolus injection. Alternatively, it can be advantageous to administer the solution or microemulsion in such a way as to maintain a constant circulating concentration of the present compound. In order to maintain such a constant concentration, a continuous intravenous delivery device can be utilized. An example of such a device is Deltec CADD-PLUS. TM. 5400 intravenous injection pump.

The pharmaceutical composition can be in the form of a sterile injectable aqueous or oily suspension for intramuscular and subcutaneous administration. Such a suspension can be formulated with suitable dispersants or wetting agents and suspending agents as described above according to known techniques. The sterile injectable preparation can also be a sterile injectable solution or suspension prepared in a nontoxic parenterally acceptable diluent or solvent. Moreover, sterile fixed oils can easily be used as a solvent or suspending medium, and fatty acids can also be used to prepare injections.

The present compound can be administered in the form of a suppository for rectal administration. These pharmaceutical compositions can be prepared by mixing the drug with a suitable non-irritating excipient that is solid at ordinary temperatures, but liquid in the rectum, thereby melting in the rectum to release the drug.

For buccal administration, the compositions can be formulated as tablets or lozenges by conventional means.

For intranasal administration or administration by inhalation, the active compounds of the present disclosure are conveniently delivered in the form of a solution or suspension released from a pump spray container that is squeezed or pumped by the patient, or as an aerosol spray released from a pressurized container or nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. The pressurized container or nebulizer can contain a solution or suspension of the active compound. Capsules or cartridges (for example, made from gelatin) for use in an inhaler or insufflator can be formulated containing a powder mix of the present disclosure and a suitable powder base such as lactose or starch.

It is well known to those skilled in the art that the dosage of a drug depends on a variety of factors, including but not limited to, the following factors: activity of the specific compound, age, weight, general health, behavior, diet of the patient, administration time, administration route, excretion rate, drug combination and the like. In addition, the best treatment, such as treatment mode, daily dose of the compound of formula (I) or the type of pharmaceutically acceptable salt thereof can be verified by traditional therapeutic regimens.

Unless otherwise stated, the terms used in the specification and claims take ordinary meanings as understood by those in the ordinary skill in the relevant art. Certain terms have the meanings described below.

“Alkyl” refers to a saturated aliphatic hydrocarbon group including C₁-C₁₂ (for example, including 1, 2, 3, 4, 5, 6, 7, 8, 9, 10, 11 and 12 carbons) straight chain and branched chain groups. Preferably an alkyl group is an alkyl having 1 to 8 carbon atoms, sometimes more preferably 1 to 6 carbon atoms, and sometime more preferably 1 to 4 carbon atoms. Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. The alkyl group can be substituted or unsubstituted. When substituted, the substituent group(s) can be substituted at any available connection point, preferably the substituent group(s) is one or more, preferably one to five, and more preferably one to three, groups independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc. preferably C₂₋₁₂ (for example, including 2, 3, 4, 5, 6, 7, 8, 9, 10, 11 and 12 carbons) alkenyl, more preferably C₂₋₈ alkenyl, sometimes more preferably C₂₋₆ alkenyl, and sometimes more preferable C₂₄ alkenyl. The alkenyl group can be substituted or unsubstituted. When substituted, the substituent group(s) is preferably one or more, preferably one to five, and more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₁₂ (for example, including 2, 3, 4, 5, 6, 7, 8, 9, 10, 11 and 12 carbons) alkynyl, more preferably C₂₋₆ alkynyl, some times more preferably C₂₋₆ alkynyl, and sometimes more preferable C₂₄ alkynyl. The alkynyl group can be substituted or unsubstituted. When substituted, the substituent group(s) is preferably one or more, preferably one to five, and more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched divalent aliphatic hydrocarbon group, derived by removing two hydrogen atoms from the same carbon atom or two different carbon atoms of the parent alkane. The straight or branched chain group containing 1 to 12 (for example, including 1, 2, 3, 4, 5, 6, 7, 8, 9, 10, 11 and 12 carbons) carbon atoms, preferably has 1 to 8 carbon atoms, more preferably 1 to 6 carbon atoms, and sometimes more preferably 1 to 4 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted.

When substituted, the substituent group(s) is preferably one or more, sometimes preferably 1 to 5, and sometimes more preferably 1 to 3, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkenylene” refers to an alkylene defined as above that has at least two carbon atoms and at least one carbon-carbon double bond, preferably C₂₋₁₂ (for example, including 2, 3, 4, 5, 6, 7, 8, 9, 10, 11 and 12 carbons) alkenylene, more preferably C₂₋₈ alkenylene, sometimes more preferably C₂₋₆ alkenylene, and sometimes even more preferably C₂₄ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably 1 to 5, and sometimes more preferably 1 to 3, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 (for example, including 3, 4, 5, 6, 7, 8, 9, 10, 11 and 12 carbons) carbon atoms, more preferably 3 to 10 carbon atoms, sometimes more preferably 3 to 8 carbon atoms, and sometimes even more preferably 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds. Preferably a spiro cycloalkyl is 6 to 14 membered (for example, including 6, 7, 8, 9, 10, 11, 12, 13 and 14 carbons), and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following groups:

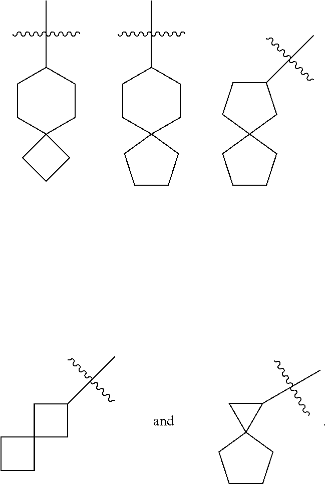

“Fused Cycloalkyl” refers to a 5 to 20 membered polycyclic hydrocarbon group, wherein each ring in the system shares an adjacent pair of carbon atoms with another ring, wherein one or more rings can contain one or more double bonds. Preferably, a fused cycloalkyl group is 6 to 14 membered (for example, including 6, 7, 8, 9, 10, 11, 12, 13 and 14 carbons), more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following groups:

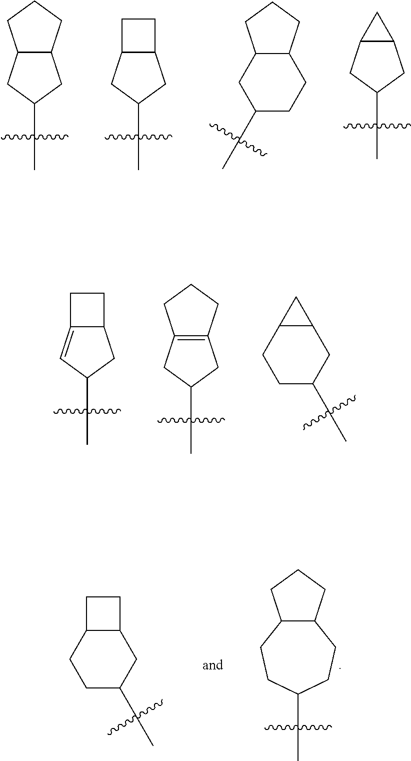

“Bridged Cycloalkyl” refers to a 5 to 20 membered polycyclic hydrocarbon group, wherein every two rings in the system share two disconnected carbon atoms. The rings can have one or more double bonds. Preferably, a bridged cycloalkyl is 6 to 14 membered (for example, including 6, 7, 8, 9, 10, 11, 12, 13 and 14 carbons), and more preferably 7 to 10 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following groups:

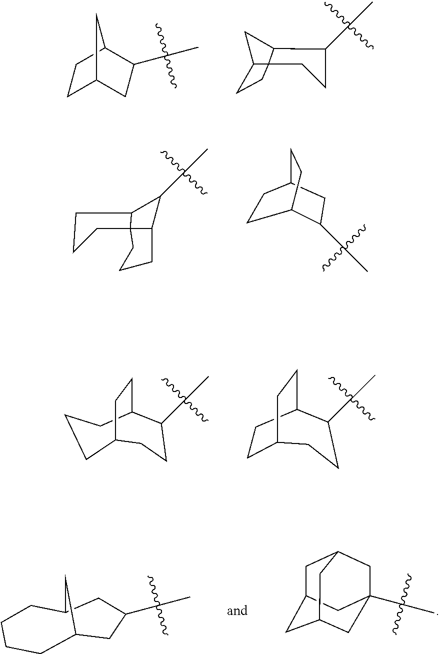

The cycloalkyl includes the cycloalkyl said above fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on. The cycloalkyl is optionally substituted or unsubstituted. When substituted, the substituent group(s) is preferably one or more, sometimes preferably 1 to 5, and sometimes more preferably 1 to 3, groups independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more heteroatoms selected from the group consisting of N, O, S, S(O) and S(O)₂ as ring atoms, but excluding —O—O—, —O—S— or —S—S— in the ring, the remaining ring atoms being C. Preferably, heterocyclyl is a 3 to 12 membered having 1 to 4 heteroatoms (for example, including 1, 2, 3 or 4 heteroatoms.); more preferably a 3 to 10 membered (for example, including 3, 4, 5, 6, 7, 8, 9 or 10 ring atoms) having 1 to 3 heteroatoms (for example, including 1, 2 or 3 heteroatoms.); most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more heteroatoms selected from the group consisting of N, O, S, S(O) and S(O)₂ as ring atoms, the remaining ring atoms being C, wherein one or more rings can contain one or more double bonds. Preferably a spiro heterocyclyl is 6 to 14 membered (for example, including 6, 7, 8, 9, 10, 11, 12, 13 or 14 ring atoms), and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following groups:

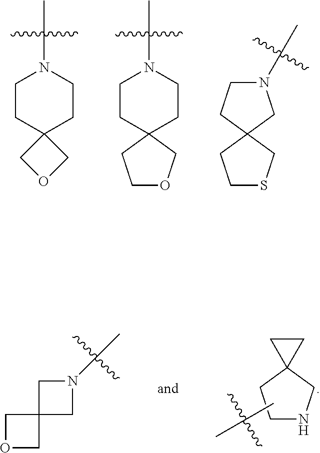

“Fused heterocyclyl” refers to a 5 to 20 membered polycyclic heterocyclyl group, wherein each ring in the system shares an adjacent pair of carbon atoms with the other ring, wherein one or more rings can contain one or more double bonds, and wherein said rings have one or more heteroatoms selected from the group consisting of N, O, S, S(O) and S(O)₂ as ring atoms, the remaining ring atoms being C. Preferably a fused heterocyclyl is 6 to 14 membered (for example, including 6, 7, 8, 9, 10, 11, 12, 13 or 14 ring atoms), and more preferably 7 to membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following groups:

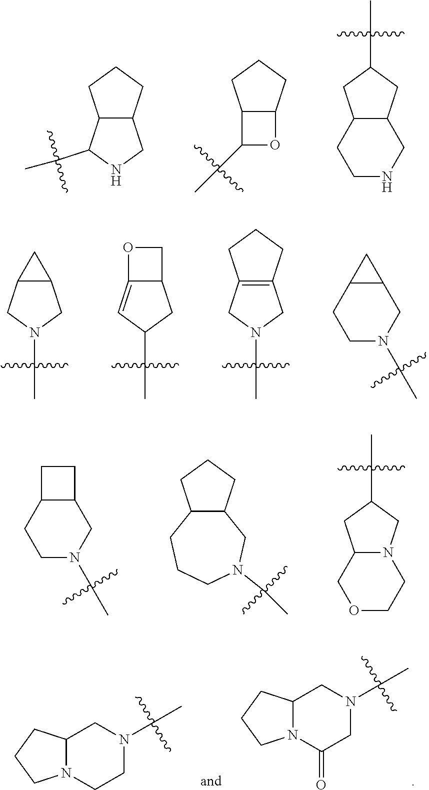

“Bridged heterocyclyl” refers to a 5 to 14 membered polycyclic heterocyclic alkyl group, wherein every two rings in the system share two disconnected atoms, the rings can have one or more double bonds, and the rings have one or more heteroatoms selected from the group consisting of N, O, S, S(O) and S(O)₂ as ring atoms, the remaining ring atoms being C. Preferably a bridged heterocyclyl is 6 to 14 membered (for example, including 6, 7, 8, 9, 10, 11, 12, 13 or 14 ring atoms), and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following groups:

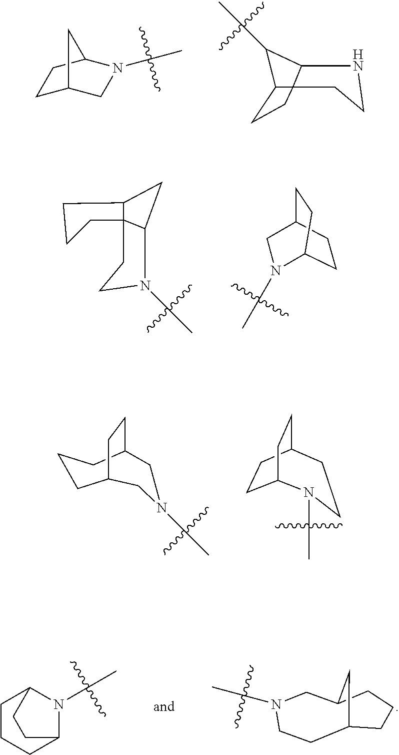

The ring of said heterocyclyl include the heterocyclyl said above which fused to the ring of an aryl, heteroaryl or cycloalkyl, wherein the ring bound to the parent structure is heterocyclyl. Representative examples include, but are not limited to the following groups:

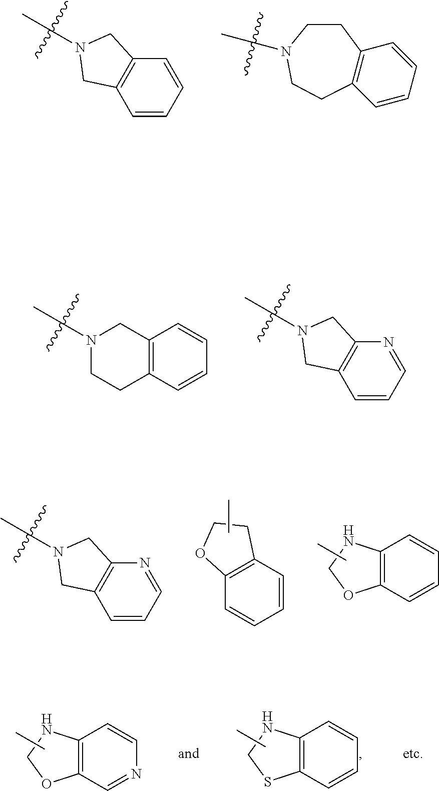

The heterocyclyl is optionally substituted or unsubstituted. When substituted, the substituent group(s) is preferably one or more, sometimes preferably 1 to 5, and sometimes more preferably 1 to 3, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl include the aryl said above which fused to the ring of heteroaryl, heterocyclyl or cycloalkyl, wherein the ring bound to parent structure is aryl. Representative examples include, but are not limited to, the following groups:

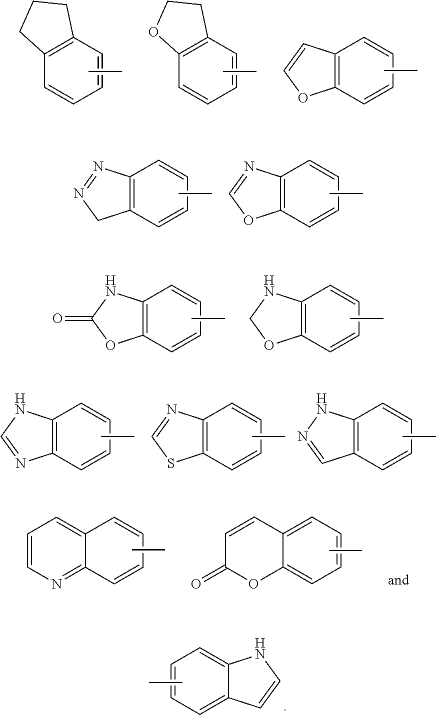

The aryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Heteroaryl” refers to an aryl system having 1 to 4 heteroatoms (for example, including 1, 2, 3 or 4 heteroatoms) selected from the group consisting of O, S and N as ring atoms and having 5 to 14 annular atoms (for example, including 5, 6, 7, 8, 9, 10, 11, 12, 13 or 14 annular atoms). Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl include the heteroaryl said above which fused with the ring of an aryl, heterocyclyl or cycloalkyl, wherein the ring bound to parent structure is heteroaryl. Representative examples include, but are not limited to, the following groups:

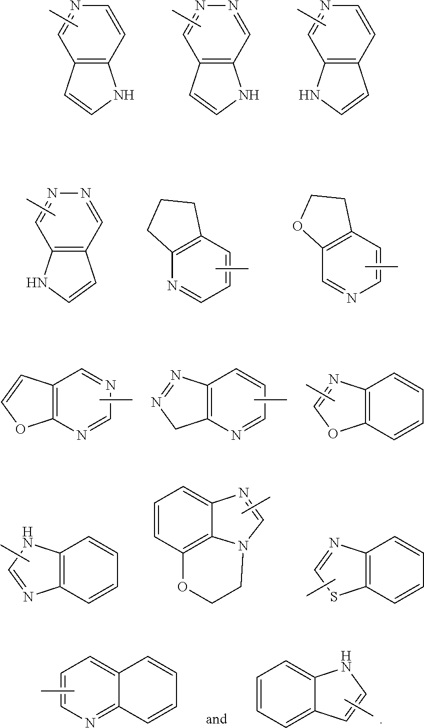

The heteroaryl group can be substituted or unsubstituted. When substituted, the substituent group(s) is preferably one or more, sometimes preferably 1 to 5, and sometimes more preferably 1 to 3, groups independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkoxy” refers to an —O-(alkyl) group, wherein the alkyl is defined as above. Representative examples include, but are not limited to, methoxy, ethoxy, propoxy, butoxy, and the like. The alkoxyl can be substituted or unsubstituted. When substituted, the substituent is preferably one or more, sometimes preferably 1 to 5, and sometimes more preferably 1 to 3, groups independently selected from the group consisting of alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Bond” refers to a covalent bond using a sign of “-”.

“Hydroxyalkyl” refers to an alkyl group substituted by one or more hydroxy group(s), wherein alkyl is as defined above.

“Hydroxy” refers to an —OH group.

“Halogen” refers to fluoro, chloro, bromo or iodo atoms.

“Amino” refers to a —NH₂ group.

“Cyano” refers to a —CN group.

“Nitro” refers to a —NO₂ group.

“Oxo group” refers to a ═O group.

“Carboxyl” refers to a —C(═O)OH group.

“Alkoxycarbonyl” refers to a —C(═O)O(alkyl) or —C(═O)O (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen atoms in the group, preferably up to 5, more preferably 1 to 3 hydrogen atoms, independently substituted with a corresponding number of substituents. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present disclosure or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds of the disclosure, such salts being safe and effective when used in a mammal and have corresponding biological activity. The salts can be prepared during the final isolation and purification of the compounds or separately by reacting a suitable nitrogen atom with a suitable acid. Acids commonly employed to form pharmaceutically acceptable salts include inorganic acids such as hydrochloric acid, hydrobromic acid, hydroiodic acid, sulfuric acid, phosphoric acid, hydrogen bisulfide as well as organic acids, such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, acetic acid acid, and related inorganic and organic acids.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include, but are not limited to, lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, and N-methylmorpholine.

As a person skilled in the art would understand, the compounds of formula (I) or pharmaceutically acceptable salts thereof disclosed herein may exist in prodrug or solvate forms, which are all encompassed by the present disclosure.

The term “solvate,” as used herein, means a physical association of a compound of this disclosure with one or more, preferably one to three, solvent molecules, whether organic or inorganic. This physical association includes hydrogen bonding. In certain instances the solvate will be capable of isolation, for example, when one or more, preferably one to three, solvent molecules are incorporated in the crystal lattice of the crystalline solid. Exemplary solvates include, but are not limited to, hydrates, ethanolates, methanolates, and isopropanolates. Methods of solvation are generally known in the art.

“Prodrug” refers to compounds that can be transformed in vivo to yield the active parent compound under physiological conditions, such as through hydrolysis in blood. Common examples include, but are not limited to, ester and amide forms of a compound having an active form bearing a carboxylic acid moiety. Amides and esters of the compounds of the present disclosure may be prepared according to conventional methods. In particular, in the present disclosure, a prodrug may also be formed by acylation of an amino group or a nitrogen atom in a heterocyclyl ring structure, which acyl group can be hydrolyzed in vivo. Such acyl group includes, but is not limited to, a C₁-C₆ acyl, preferably C₁-C₄ acyl, and more preferably C₁-C₂ (formyl or acetyl) group, or benzoyl.

The term “pharmaceutically acceptable,” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The term “therapeutically effective amount,” as used herein, refers to the total amount of each active component that is sufficient to show a meaningful patient benefit, e.g., a sustained reduction in viral load. When applied to an individual active ingredient, administered alone, the term refers to that ingredient alone. When applied to a combination, the term refers to combined amounts of the active ingredients that result in the therapeutic effect, whether administered in combination, serially, or simultaneously.

The term “treat”, “treating”, “treatment”, or the like, refers to: (i) inhibiting the disease, disorder, or condition, i.e., arresting its development; and (ii) relieving the disease, disorder, or condition, i.e., causing regression of the disease, disorder, and/or condition. In addition, the compounds of present disclosure may be used for their prophylactic effects in preventing a disease, disorder or condition from occurring in a subject that may be predisposed to the disease, disorder, and/or condition but has not yet been diagnosed as having it.

As used herein, the singular forms “a”, “an”, and “the” include plural reference, and vice versa, unless the context clearly dictates otherwise.

When the term “about” is applied to a parameter, such as pH, concentration, temperature, or the like, it indicates that the parameter can vary by ±10%, and some times more preferably within ±5%. As would be understood by a person skilled in the art, when a parameter is not critical, a number is often given only for illustration purpose, instead of being limiting.

The compound of the present disclosure, any atom not specifically designated as a specific isotope means any stable isotope of that atom. Unless otherwise stated, when a position is specifically designated as “H” or “hydrogen”, the position should be understood as having hydrogen according to its natural abundance isotopic composition. Likewise, unless otherwise specified, when a position is specifically designated as “D” or “deuterium”, the position should be understood as deuterium having an abundance of at least 3000 times greater than the natural abundance of deuterium (which is 0.015%) (That is, at least 45% of deuterium is incorporated).

### Synthesis Methods

In order to complete the purpose of the disclosure, the present disclosure applies, but is not limited to, the following technical solution:

### Scheme 1

A preparation process of a compound of formula (I), or a tautomer, mesomer, racemate, enantiomer, diastereomer, or mixture thereof, or a pharmaceutically acceptable salt, solvate or prodrug thereof, comprising a step of:

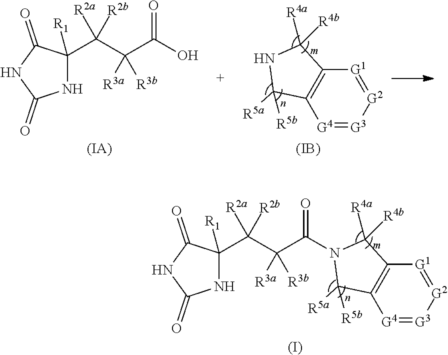

reacting a compound of formula (IA) or a tautomer, mesomer, racemate, enantiomer, diastereomer, or mixture thereof, or a salt thereof with a compound of formula (IB) or a salt thereof under alkaline conditions and in the presence of a condensing agent to obtain the compound of formula (I) or a tautomer, mesomer, racemate, enantiomer, diastereomer, or mixture thereof, or a pharmaceutically acceptable salt, solvate or prodrug thereof;
wherein:
the salt of the compound (IB) preferably is hydrochloride salt; and
G¹, G², G³, G⁴, R¹, R^{2a} to R^{5a}, R^{2b} to R^{5b}, n and m are each as defined in formula (I).

### Scheme 2

A preparation process of a compound of formula (II), or a tautomer, mesomer, racemate, enantiomer, diastereomer, or mixture thereof, or a pharmaceutically acceptable salt, solvate or prodrug thereof, comprising a step of:

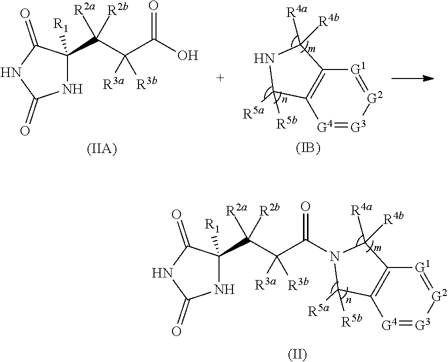

reacting a compound of formula (IIA) or a tautomer, mesomer, racemate, enantiomer, diastereomer, or mixture thereof, or a salt thereof with a compound of formula (IB) or a salt thereof under alkaline conditions and in the presence of a condensing agent to obtain the compound of formula (II) or a tautomer, mesomer, racemate, enantiomer, diastereomer, or mixture thereof, or a pharmaceutically acceptable salt, solvate or prodrug thereof;
wherein:
the salt of the compound (IB) preferably is hydrochloride salt; and
G¹, G², G³, G⁴, R¹, R^{2a} to R^{5a}, R^{2b} to R^{5b}, n and m are each as defined in formula (II).

### Scheme 3

A preparation process of a compound of formula (III), or a tautomer, mesomer, racemate, enantiomer, diastereomer, or mixture thereof, or a pharmaceutically acceptable salt, solvate or prodrug thereof, comprising a step of:

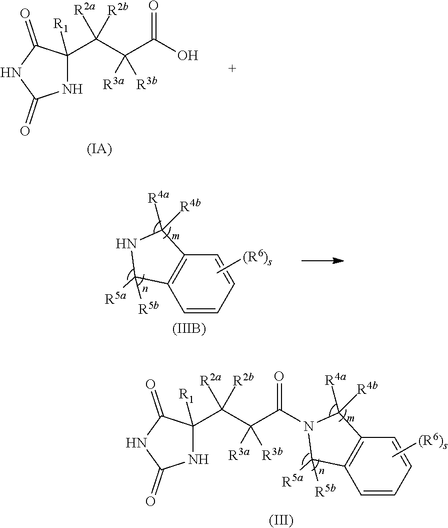

reacting a compound of formula (IA) or a tautomer, mesomer, racemate, enantiomer, diastereomer, or mixture thereof, or a salt thereof with a compound of formula (IIIB) or a salt thereof under alkaline conditions and in the presence of a condensing agent to obtain the compound of formula (III) or a tautomer, mesomer, racemate, enantiomer, diastereomer, or mixture thereof, or a pharmaceutically acceptable salt, solvate or prodrug thereof;
wherein:
the salt of the compound (IIIB) preferably is hydrochloride salt; and
R¹, R^{2a} to R^{5a}, R^{2b} to R^{5b}, R⁶, n, m and s are each as defined in formula (III).

### Scheme 4

A preparation process of a compound of formula (IIIa), or a tautomer, mesomer, racemate, enantiomer, diastereomer, or mixture thereof, or a pharmaceutically acceptable salt, solvate or prodrug thereof, comprising a step of:

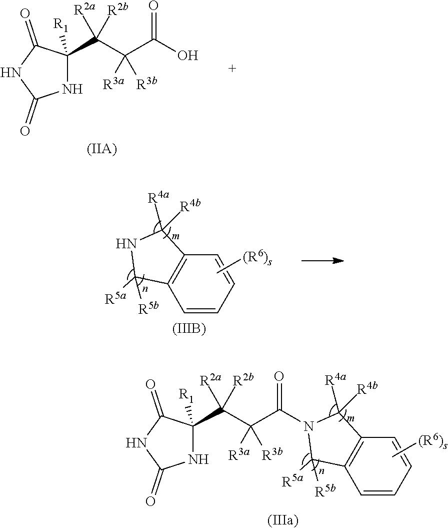

reacting a compound of formula (IIA) or a tautomer, mesomer, racemate, enantiomer, diastereomer, or mixture thereof, or a salt thereof with a compound of formula (IIIB) or a salt thereof under alkaline conditions and in the presence of a condensing agent to obtain the compound of formula (IIIa) or a tautomer, mesomer, racemate, enantiomer, diastereomer, or mixture thereof, or a pharmaceutically acceptable salt, solvate or prodrug thereof;
wherein:
the salt of the compound (IIIB) preferably is hydrochloride salt; and
R¹, R^{2a} to R^{5a}, R^{2b} to R^{5b}, R⁶, n, m and s are each as defined in formula (IIIa).

### Scheme 5

A preparation process of a compound of formula (IV), or a tautomer, mesomer, racemate, enantiomer, diastereomer, or mixture thereof, or a pharmaceutically acceptable salt, solvate or prodrug thereof, the preparation process comprising the steps of:

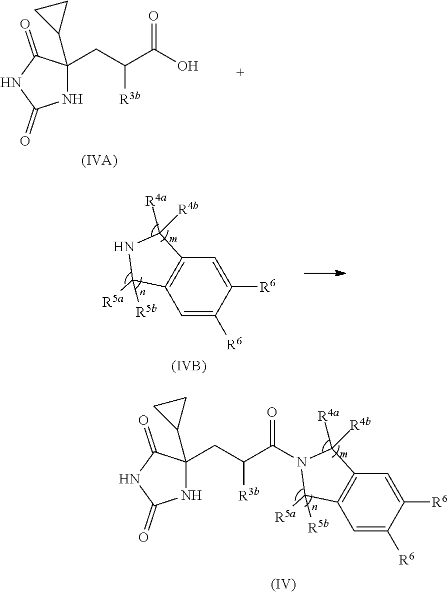

reacting a compound of formula (IVA) or a tautomer, mesomer, racemate, enantiomer, diastereomer, or mixture thereof, or a salt thereof with a compound of formula (IVB) or a salt thereof under alkaline conditions and in the presence of a condensing agent to obtain the compound of formula (IV) or a tautomer, mesomer, racemate, enantiomer, diastereomer, or mixture thereof, or a pharmaceutically acceptable salt, solvate or prodrug thereof;
wherein:
the salt of the compound (IVB) preferably is hydrochloride salt; and
R^{4a}, R^{5a}, R^{3b} to R^{5b}, R⁶, n and m are each as defined in formula (IV).

### Scheme 6

A preparation process of a compound of formula (IVa), or a tautomer, mesomer, racemate, enantiomer, diastereomer, or mixture thereof, or a pharmaceutically acceptable salt, solvate or prodrug thereof, the preparation process comprising the steps of:

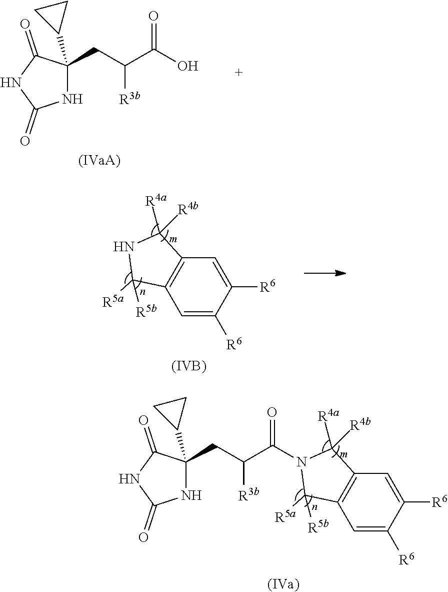

reacting a compound of formula (IVaA) or a tautomer, mesomer, racemate, enantiomer, diastereomer, or mixture thereof, or a salt thereof with a compound of formula (IVB) or a salt thereof under alkaline conditions and in the presence of a condensing agent to obtain the compound of formula (IVa) or a tautomer, mesomer, racemate, enantiomer, diastereomer, or mixture thereof, or a pharmaceutically acceptable salt, solvate or prodrug thereof;
wherein:
the salt of the compound (IVB) preferably is hydrochloride salt; and
R^{4a}, R^{5a}, R^{3b} to R^{5b}, R⁶, n and m are each as defined in formula (IVa).

### Scheme 7

A preparation process of a compound of formula (V), or a tautomer, mesomer, racemate, enantiomer, diastereomer, or mixture thereof, or a pharmaceutically acceptable salt, solvate or prodrug thereof, the preparation process comprising the steps of:

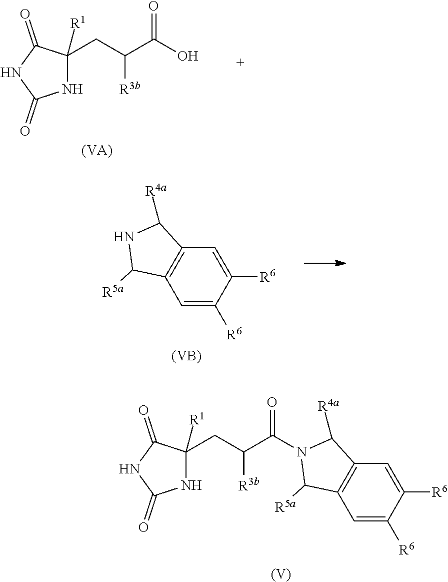

reacting a compound of formula (VA) or a tautomer, mesomer, racemate, enantiomer, diastereomer, or mixture thereof, or a salt thereof with a compound of formula (VB) or a salt thereof under alkaline conditions and in the presence of a condensing agent to obtain the compound of formula (V) or a tautomer, mesomer, racemate, enantiomer, diastereomer, or mixture thereof, or a pharmaceutically acceptable salt, solvate or prodrug thereof;
wherein:
the salt of the compound (VB) preferably is hydrochloride salt; and
R^{4a}, R^{5a}, R^{3b}, R¹ and R⁶ are each as defined in formula (V).

### Scheme 8

A preparation process of a compound of formula (Va) or (Vb), or a tautomer, mesomer, racemate, enantiomer, diastereomer, or mixture thereof, or a pharmaceutically acceptable salt, solvate or prodrug thereof, the preparation process comprising the steps of:

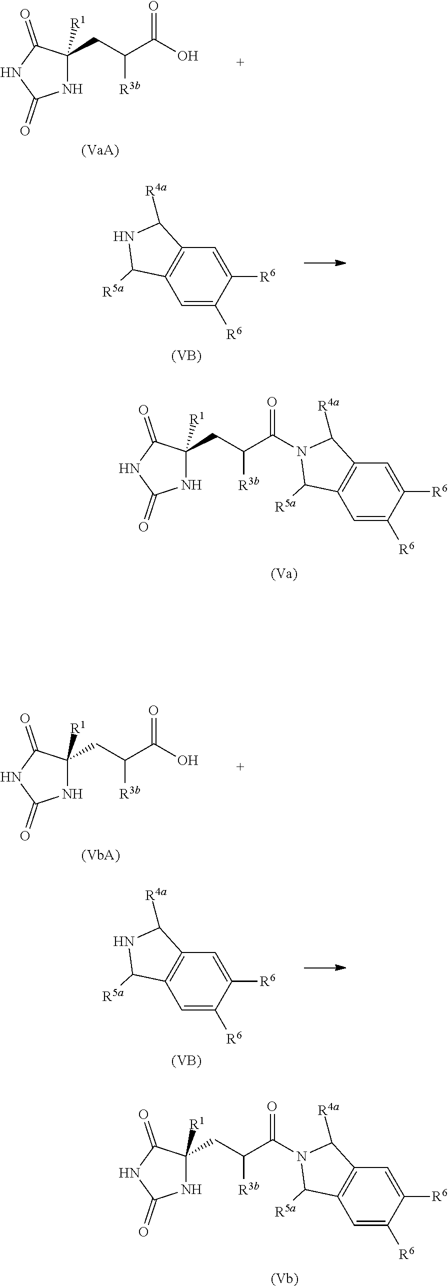

reacting a compound of formula (VaA) or a tautomer, mesomer, racemate, enantiomer, diastereomer, or mixture thereof, or a salt thereof with a compound of formula (VB) or a salt thereof under alkaline conditions and in the presence of a condensing agent to obtain the compound of formula (Va) or a tautomer, mesomer, racemate, enantiomer, diastereomer, or mixture thereof, or a pharmaceutically acceptable salt, solvate or prodrug thereof;
reacting a compound of formula (VbA) or a tautomer, mesomer, racemate, enantiomer, diastereomer, or mixture thereof, or a salt thereof with a compound of formula (VB) or a salt thereof under alkaline conditions and in the presence of a condensing agent to obtain the compound of formula (Vb) or a tautomer, mesomer, racemate, enantiomer, diastereomer, or mixture thereof, or a pharmaceutically acceptable salt, solvate or prodrug thereof;
wherein:
the salt of the compound (VB) preferably is hydrochloride salt; and
R^{4a}, R^{5a}, R^{3b}, R¹ and R⁶ are each as defined in formula (V).

### Scheme 9

A preparation process of a compound of formula (II) or (IIb), or a tautomer, mesomer, racemate, enantiomer, diastereomer, or mixture thereof, or a pharmaceutically acceptable salt, solvate or prodrug thereof, the preparation process comprising the steps of:

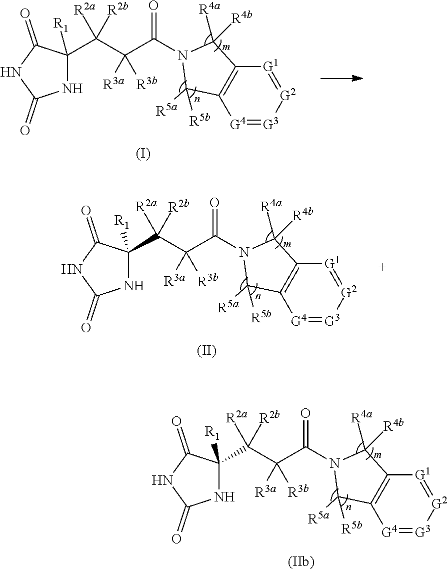

Formula (I) or a tautomer, mesomer, racemate, enantiomer, diastereomer, or mixture thereof, or a pharmaceutically acceptable salt, solvate or prodrug thereof, was chiral separated to give Formula (II) and (IIb) or a tautomer, mesomer, racemate, enantiomer, diastereomer, or mixture thereof, or a pharmaceutically acceptable salt, solvate or prodrug thereof;
wherein:
G¹, G², G³, G⁴, R¹, R^{2a} to R^{5a}, R^{2b} to R^{5b}, n and m are each as defined in formula (II).

### Scheme 10

A preparation process of a compound of formula (IIIa) or (IIIb), or a tautomer, mesomer, racemate, enantiomer, diastereomer, or mixture thereof, or a pharmaceutically acceptable salt, solvate or prodrug thereof, the preparation process comprising the steps of:

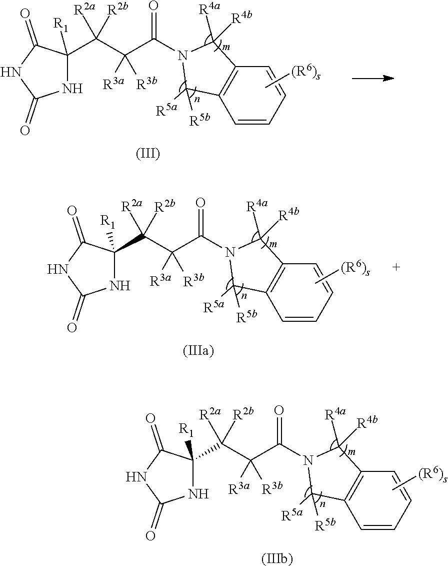

Formula (III) or a tautomer, mesomer, racemate, enantiomer, diastereomer, or mixture thereof, or a pharmaceutically acceptable salt, solvate or prodrug thereof, was chiral separated to give Formula (IIIa) and (IIIb) or a tautomer, mesomer, racemate, enantiomer, diastereomer, or mixture thereof, or a pharmaceutically acceptable salt, solvate or prodrug thereof;
wherein:
R¹, R^{2a} to R^{5a}, R^{2b} to R^{5b}, R⁶, s, n and m are each as defined in formula (III).

### Scheme 11

A preparation process of a compound of formula (IVa) or (IVb), or a tautomer, mesomer, racemate, enantiomer, diastereomer, or mixture thereof, or a pharmaceutically acceptable salt, solvate or prodrug thereof, the preparation process comprising the steps of:

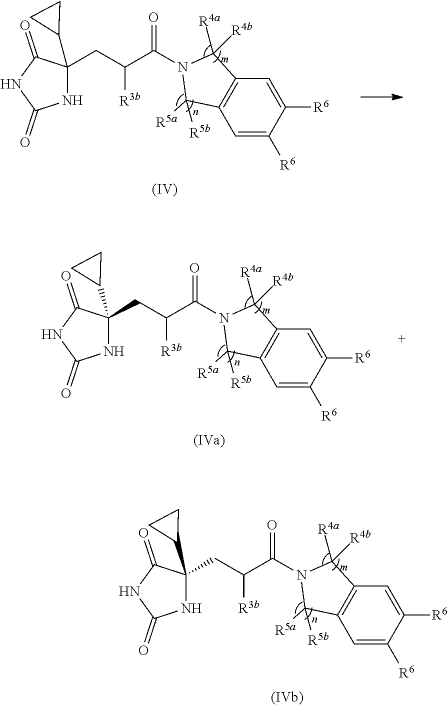

Formula (IV) or a tautomer, mesomer, racemate, enantiomer, diastereomer, or mixture thereof, or a pharmaceutically acceptable salt, solvate or prodrug thereof, was chiral separated to give Formula (IVa) and (IVb) or a tautomer, mesomer, racemate, enantiomer, diastereomer, or mixture thereof, or a pharmaceutically acceptable salt, solvate or prodrug thereof;
wherein:
R^{4a} to R^{5a}, R^{3b} to R^{5b}, R⁶, n and m are each as defined in formula (IV).

### Scheme 12

A preparation process of a compound of formula (Va) or (Vb), or a tautomer, mesomer, racemate, enantiomer, diastereomer, or mixture thereof, or a pharmaceutically acceptable salt, solvate or prodrug thereof, the preparation process comprising the steps of:

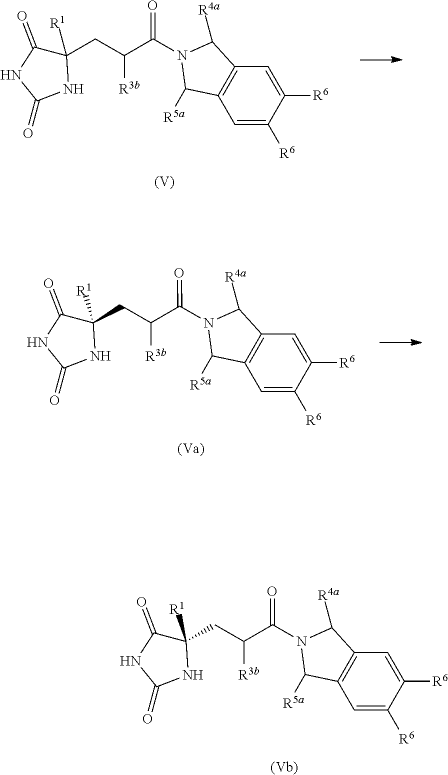

Formula (V) or a tautomer, mesomer, racemate, enantiomer, diastereomer, or mixture thereof, or a pharmaceutically acceptable salt, solvate or prodrug thereof, was chiral separated to give Formula (Va) and (Vb) or a tautomer, mesomer, racemate, enantiomer, diastereomer, or mixture thereof, or a pharmaceutically acceptable salt, solvate or prodrug thereof;
wherein:
R¹, R^{4a} to R^{5a}, R^{3b}, and R⁶ are each as defined in formula (V).

### Scheme 13

A preparation process of a compound of formula (I), or a tautomer, mesomer, racemate, enantiomer, diastereomer, or mixture thereof, or a pharmaceutically acceptable salt, solvate or prodrug thereof, comprising a step of:

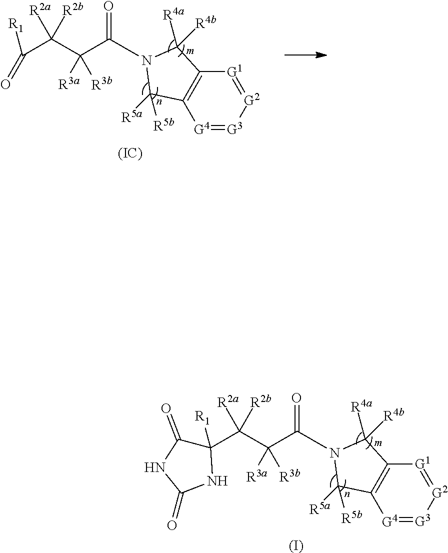

reacting a compound of formula (IC) or a tautomer, mesomer, racemate, enantiomer, diastereomer, or mixture thereof, or a salt thereof with ammonium carbonate (or ammonium bicarbonate) and sodium cyanide (or potassium cyanide) to obtain the compound of formula (I) or a tautomer, mesomer, racemate, enantiomer, diastereomer, or mixture thereof, or a pharmaceutically acceptable salt, solvate or prodrug thereof;
wherein:
G¹, G², G³, G⁴, R¹, R^{2a} to R^{5a}, R^{2b} to R^{5b}, n and m are each as defined in formula (I).

### Scheme 14

A preparation process of a compound of formula (III), or a tautomer, mesomer, racemate, enantiomer, diastereomer, or mixture thereof, or a pharmaceutically acceptable salt, solvate or prodrug thereof, comprising a step of:

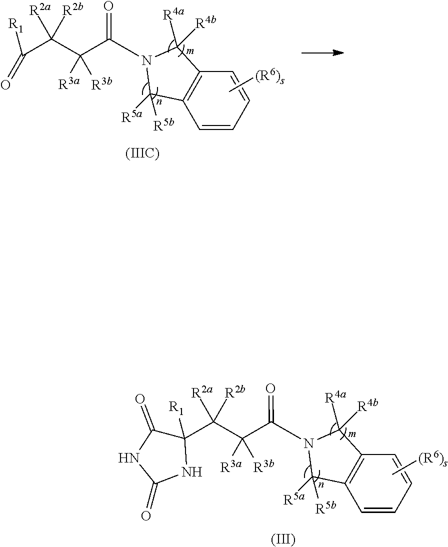

reacting a compound of formula (IIIC) or a tautomer, mesomer, racemate, enantiomer, diastereomer, or mixture thereof, or a salt thereof with ammonium carbonate (or ammonium bicarbonate) and sodium cyanide (or potassium cyanide) to obtain the compound of formula (III) or a tautomer, mesomer, racemate, enantiomer, diastereomer, or mixture thereof, or a pharmaceutically acceptable salt, solvate or prodrug thereof;
wherein:
R¹, R^{2a} to R^{5a}, R^{2b} to R^{5b}, R⁶, n, m and s are each as defined in formula (III).

### Scheme 15

A preparation process of a compound of formula (IV), or a tautomer, mesomer, racemate, enantiomer, diastereomer, or mixture thereof, or a pharmaceutically acceptable salt, solvate or prodrug thereof, comprising a step of:

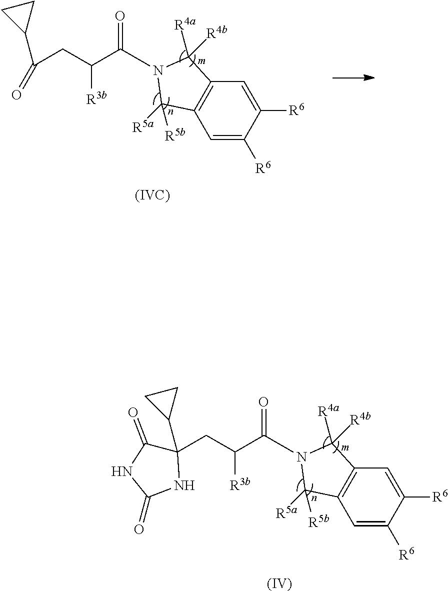

reacting a compound of formula (IVC) or a tautomer, mesomer, racemate, enantiomer, diastereomer, or mixture thereof, or a salt thereof with ammonium carbonate (or ammonium bicarbonate) and sodium cyanide (or potassium cyanide) to obtain the compound of formula (IV) or a tautomer, mesomer, racemate, enantiomer, diastereomer, or mixture thereof, or a pharmaceutically acceptable salt, solvate or prodrug thereof;
wherein:
R^{4a}, R^{5a}, R^{3b} to R^{5b}, R⁶, n and m are each as defined in formula (IV).

### Scheme 16

A preparation process of a compound of formula (V), or a tautomer, mesomer, racemate, enantiomer, diastereomer, or mixture thereof, or a pharmaceutically acceptable salt, solvate or prodrug thereof, comprising a step of:

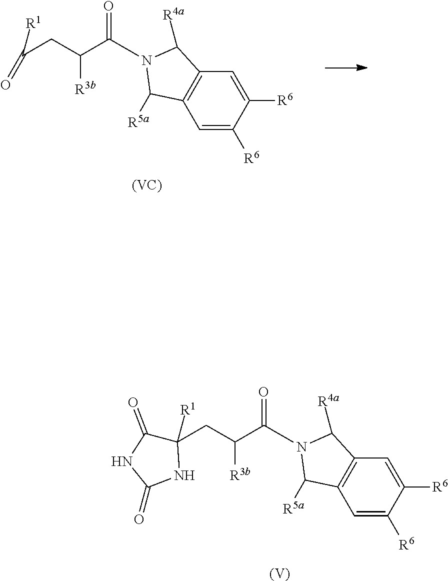

reacting a compound of formula (VC) or a tautomer, mesomer, racemate, enantiomer, diastereomer, or mixture thereof, or a salt thereof with ammonium carbonate (or ammonium bicarbonate) and sodium cyanide (or potassium cyanide) to obtain the compound of formula (V) or a tautomer, mesomer, racemate, enantiomer, diastereomer, or mixture thereof, or a pharmaceutically acceptable salt, solvate or prodrug thereof;
wherein:
R^{4a}, R^{5a}, R^{3b}, R¹ and R⁶ are each as defined in formula (V).

The agent which provides the alkaline condition includes organic bases and inorganic bases, wherein the organic base includes, but is not limited to, triethylamine, N,N-disopropylethylamine, n-butyllithium, lithium diisopropylamide, potassium acetate, sodium tert-butoxide and potassium tert-butoxide, and wherein the inorganic base includes, but is not limited to, magnesium chloride, sodium hydride, potassium phosphate, sodium carbonate, potassium carbonate, cesium carbonate and N-(3-Dimethylaminopropyl)-N′-ethylcarbodiimide hydrochloride (EDCI).

The condensing agent includes, but is not limited to, 1-(3-dimethylaminopropyl)-3-ethylcarbodiimide hydrochloride, N,N′-dicyclohexylcarbodiimide, N,N′-diisopropylcarbodiimide, O-benzotriazole-N,N,N′,N′-tetramethyluronium tetrafluoroborate, 1-hydroxybenzotriazole, 1-hydroxy-7-azobenzotriazole, 0-benzotriazole-N,N,N′,N′-tetramethyluronium hexafluoro phosphate, 2-(7-oxobenzotriazole)-N,N,N′,N′-tetramethyluronium hexafluorophosphate, 2-(7-azobenzotriazole)-N,N,N′,N′-tetramethyluronium hexafluorophosphate, benzotriazol-1-yloxytris(dimethylamino)phosphonium hexafluorophosphate and benzotriazol-1-yl-oxytripyrrolidinylphosphonium phosphate.

The reaction is preferably conducted in a solvent, wherein solvent used herein includes, but is not limited to, acetic acid, methanol, ethanol, toluene, tetrahydrofuran, dichloromethane, dimethylsulfoxide, 1,4-dioxane, water, N, N-dimethylformamide, and the mixtures thereof.

The following examples serve to illustrate the disclosure, but the examples should not be considered as limiting the scope of the disclosure. If specific conditions for the experimental method are not specified in the examples of the present disclosure, they are generally in accordance with conventional conditions or recommended conditions of the raw materials and the product manufacturer. The reagents without a specific source indicated are commercially available, conventional reagents.

The structures of the compounds were identified by nuclear magnetic resonance (NMR) and/or mass spectrometry (MS). NMR was determined by a Bruker AVANCE II (or III)-400 MHz. The solvents are deuterated-dimethyl sulfoxide (DMSO-d₆), deuterated-chloroform (CDCl₃) and deuterated-methanol (CD₃OD) with tetramethylsilane (TMS) as an internal standard. NMR chemical shifts (6) are given in 10⁻⁶ (ppm).

LC/MS (ESI) analyses were performed on a Shimadzu LCMS2020 equipped with a Sunfire C18 (5 um 50×4.6 mm) column, Waters UPLC-QDa equipped with an ACQUITY UPLC® BEH (2.1*50 mm 1.7 um) column, Agilent Agilent6120 equipped with a Xbridge C18 (5 um 50×4.6 mm) column.

HPLC analyses were performed on an Agilent 1200DAD equipped with a Sunfire C18 (5 um 150×4.6 mm) column and Shimadzu UFLC equipped with an Xbridge C18 (5 um 150×4.6 mm) column.

Chiral HPLC analyses were performed on a Waters-UPC² instrument.

The known raw materials of the present disclosure were prepared by the conventional synthesis methods in the art, or purchased from Aldrich Chemical Company, Fisher Scientific or Combi-Blocks, etc.

Unless otherwise stated, the reactions were carried out under nitrogen atmosphere.

Unless otherwise stated, the reaction temperature in the reactions refers to room temperature, and the range of the temperature was 20° C. to 30° C.

The reaction process was monitored by LC-MS or thin layer chromatography (TLC), and the developing solvent system includes: A: dichloromethane and methanol, B: hexane and ethyl acetate. The ratio of the volume of the solvent was adjusted according to the polarity of the compounds. The elution system for purification of the compounds by column chromatography, thin layer chromatography and CombiFlash flash rapid preparation instrument includes: A: dichloromethane and methanol, B: hexane and ethyl acetate. The ratio of the volume of the solvent was adjusted according to the polarity of the compounds, and sometimes a small amount of basic reagent such as ammonia or acidic reagent such as acetic acid was added.

Prep-HPLC was performed on Shimadzu (LC-20AD, SPD20A) Preparative HPLC (Phenomenex Gemini-NX 5 uM C18 21.2×100 mm column), Waters 2767 equipped with a Sunfire Pre C18 (10 um 19×250 mm) column and Waters 2767-QDa equipped with an Xbridge Pre C18 (10 um 19×250 mm) column instrument.

Pre-SFC was performed on a Waters-SFC80 equipped with Daciel AD/OD/OJ/IC/IA/ID (10 um 20×250 mm) column instrument.

CombiFlash was performed on systems from Teledyne ISCO or Agela Technologies.

The following abbreviations are used:
AIBN is 2,2′-Azobis(2-methylpropionitrile),
DAST is (Diethylamino)sulfur trifluoride,
DIPEA (or DIEA) is N,N-diisopropylethylamine,
EDCI is N-(3-Dimethylaminopropyl)-N′-ethylcarbodiimide hydrochloride,
HOBt is 1-Hydroxybenzotriazole hydrate,
HATU is O-(7-Azabenzotriazol-1-yl)-N,N,N′,N′-te-tramethyluronium hexafluorophosphate,
HBTU is O-(Benzotriazol-1-yl)-N,N,N′,N′-tetramethyluronium hexafluorophosphate,
HCl is hydrogen chloride,
LDA is Lithium diisopropylamide,
LiHMDS is Lithium bis(trimethylsilyl)amide,
n-BuLi is n-Butyllithium,
NBS is N-Bromosuccinimide,
NCS is N-Chlorosuccinimide,
Pd(dppf)C₂ is [1,1′-Bis(diphenylphosphino)ferrocene]dichloropalladium(II),
TEA is triethylamine,
DCE is 1,2-dichloroethane,
DCM is dichloromathane,
DMF is N,N-dimethylformamide,
EtOAc (or EA) is ethyl acetate,
EtOH is ethanol,
MeCN or ACN is acetonitrile,
MeOH is methanol,
n-BuOH is n-Butanol,
PE is petroleum ether,
THF is Tetrahydrofuran,
NMR is nuclear magnetic resonance,
MS is mass spectroscopy with (+) referring to the positive mode which generally gives a M+1 (or M+H) absorption where M=the molecular mass.
Prep HPLC is Preparative High performance liquid chromatography.
SFC is Supercritical fluid chromatography.

### Intermediate 1 (Int-1)

### (S)-3-(4-cyclopropyl-2,5-dioxoimidazolidin-4-yl)propanoic acid Intermediate 1 (Int-1)

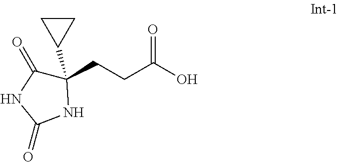 [SMILES: O=C(O)CC[C@@]1(C2CC2)NC(=O)NC1=O]

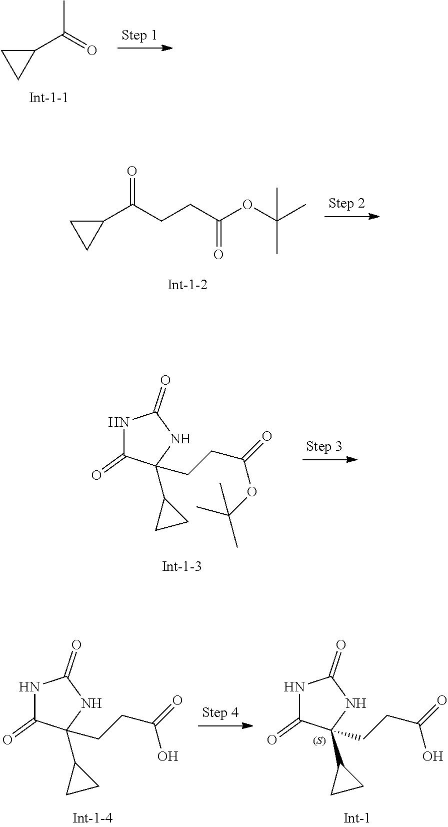

### Step 1

### Tert-butyl 4-cyclopropyl-4-oxobutanoate Int-1-2

The solution of LDA (15.28 g, 142.66 mmol, 71.43 mL) in THF (50 mL) was cooled to −78° C. before the solution of cyclopropyl methyl ketone Int-1-1 (10 g, 118.88 mmol) in THF (10 mL) was added dropwise. The resulting solution was warmed to 20° C. and stirred for 30 min. The reaction mixture was then re-cooled to −78° C. and tert-butyl 2-bromoacetate (23.19 g, 118.88 mmol) in THF (10 mL) was added slowly. The reaction was stirred at room temperature overnight. After the reaction completed, the reaction was quenched with saturated NH₄Cl (50 mL, aq.), the mixture was extracted with EtOAc (50 mL×3), the organic phase was washed with brine (100 mL), dried over Na₂SO₄ and concentrated to give the crude tittle compound Int-1-2 (22 g, 110.97 mmol, 93.34% yield).

¹H NMR (400 MHz, CDCl₃): δ 2.83 (t, 2H), 2.50 (t, 2H), 1.97-1.92 (m, 1H), 1.45 (s, 9H), 1.06-1.01 (m, 2H), 0.91-0.86 (m, 2H).

### Step 2

### Tert-butyl 3-(4-cyclopropyl-2,5-dioxoimidazolidin-4-yl)propanoate Int-1-3

The mixture of Int-1-2 (8.2 g, 41.36 mmol), ammonium carbonate (33.78 g, 351.56 mmol), sodium cyanide (5.07 g, 103.40 mmol), EtOH (50 mL) and water (50 mL) was sealed and heated to 80° C. for 18 h. The reaction mixture was cooled and poured into a mixture of EtOAc (100 mL) and water (100 mL), the layers were separated, and the aq. layer was extracted with EtOAc (100 mL×3). The organic solution was combined and washed with brine, dried over Na₂SO₄ and concentrated. The residue was purified by silica gel chromatagraphy (EtOAc/hexane=1/2) to give the tittle compound Int-1-3 (5.7 g, 21.24 mmol, 51.36% yield).

¹H NMR (400 MHz, DMSO-d6): δ 10.61 (s, 1H), 7.66 (s, 1H), 2.29-2.08 (m, 2H), 1.93-1.88 (m, 2H), 1.29 (s, 9H), 1.09-1.02 (m, 1H), 0.47-0.26 (m, 3H), 0.11-0.04 (m, 1H).

### Steps 3 and 4

### (S)-3-(4-cyclopropyl-2,5-dioxoimidazolidin-4-yl)propanoic acid Int-1

The solution of Int-1-3 (7.2 g, 26.83 mmol) in HCl/Dioxane (4M, 50 mL) was stirred at room temperature for 4 h and concentrated. The resulting solid was triturated in MeCN (30 mL) for 1 h and filtrated to give pure racemic target as a white solid. The solid was chiral separated by SFC (using a chiral column CHIRALPAK AD-H 10 um 2.5*25 cm; Flow Rate/detection: 70 g/min; Detector Wavelength: 214 nm; Mobile phase A: Supercritical CO₂; Mobile phase B: methanol) to give the tittle compound Int-1 (2 g, 9.42 mmol, 35.12% yield).

¹H NMR (400 MHz, DMSO-d6): δ 12.20 (s, 1H), 10.63 (s, 1H), 7.71 (s, 1H), 2.32-2.09 (m, 2H), 1.99-1.87 (m, 2H), 1.11-1.03 (m, 1H), 0.48-0.27 (m, 3H), 0.12-0.05 (m, 1H).

Chiral HPLC: 98.04% ee, Rt: 2.918 min.

LCMS: MS m/z (ESI): 213.1 [M+1]

### Intermediate 2 (Int-2)

### 3-(4-cyclopropyl-2,5-dioxoimidazolidin-4-yl)-2-methylpropanoic acid Int-2

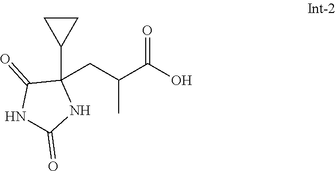 [SMILES: CC(CC1(C2CC2)NC(=O)NC1=O)C(=O)O]

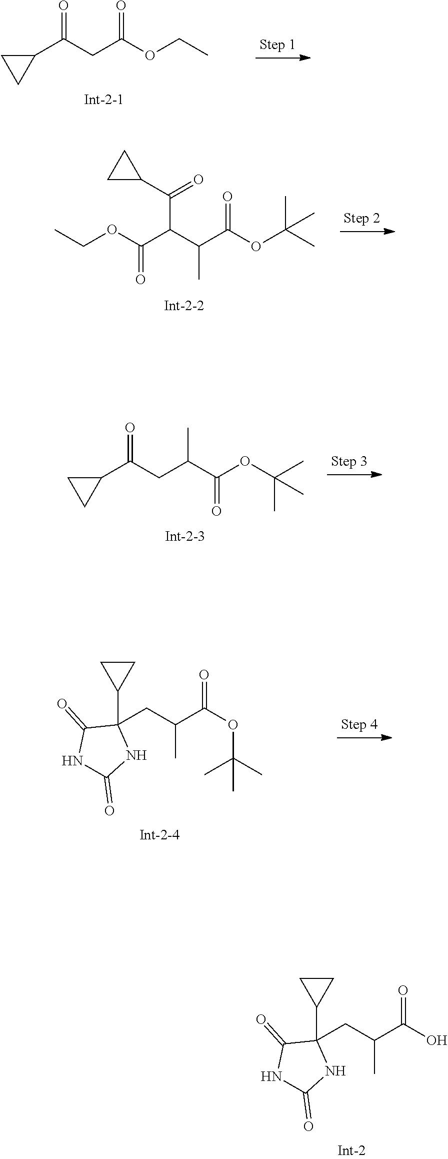 [SMILES: CCOC(=O)CC(=O)C1CC1 ; CCOC(=O)C(C(=O)C1CC1)C(C)C(=O)OC(C)(C)C ; CC(CC(=O)C1CC1)C(=O)OC(C)(C)C ; CC(CC1(C2CC2)NC(=O)NC1=O)C(=O)OC(C)(C)C ; CC(CC1(C2CC2)NC(=O)NC1=O)C(=O)O]

### Step 1

### 1-(tert-butyl) 4-ethyl 3-(cyclopropanecarbonyl)-2-methylsuccinate Int-2-2

To a mixture of ethyl 3-cyclopropyl-3-oxopropanoate Int-2-1 (32 g, 204.89 mmol) in butan-2-one (400 mL) was added tert-butyl 2-bromopropanoate (44.54 g, 213.04 mmol), K₂CO₃ (57 g, 409.79 mmol) and NaI (3.07 g, 20.49 mmol). The reaction mixture was heated at 100° C. for 16 h and cooled to RT. Water was added and the reaction mixture acidified to pH 8. The the mixture was extracted with EtOAc. The combined organic layers were washed with water and brine, dried over anhydrous Na₂SO₄, filtered and concentrated in vacuum. The residue was purified by silica gel chromatagraphy (hexane) to give Int-2-2 (11.7 g, 41.15 mmol, 20.08% yield).

¹HNMR (400 MHz, CDCl₃): δ 4.25-4.21 (m, 2H), 3.92 (dd, 1H), 3.19-3.09 (m, 1H), 2.17-2.07 (m, 1H), 1.43 (d, 9H), 1.30-1.24 (m, 3H), 1.19-1.13 (m, 3H), 1.11-1.06 (m, 2H), 0.98-0.91 (m, 2H).

### Step 2

### tert-butyl 4-cyclopropyl-2-methyl-4-oxo-butanoate Int-2-3

To a solution of the Int-2-2 (4 g, 14.07 mmol) in THF (50 mL) was added LiOH (1.68 g, 70.34 mmol) and H₂O (50 mL). The reaction mixture was stirred at room temperature overnight. To the mixture was added aq. Citric acid solution to adjust to pH<5 and then stirred at 40-50° C. for 10 min. After the reaction was completed, water was added and the reaction mixture was extracted with EtOAc. The combined organic layers were washed with water and brine, dried over anhydrous Na₂SO₄, filtered and concentrated in vacuo. The residue was purified by silica gel chromatagraphy to give Int-2-3 (1.81 g, 8.55 mmol, 60.74% yield). ¹HNMR (400 MHz, CDCl₃): δ 2.97 (dd, 1H), 2.89-2.79 (m, 1H), 2.56 (dd, 1H), 1.96-1.88 (m, 1H), 1.43 (s, 9H), 1.15 (d, 3H), 1.05-1.00 (m, 2H), 0.90-0.84 (m, 2H).

### Step 3

### Tert-butyl 3-[4-cyclopropyl-2,5-dioxo-imidazolidin-4-yl]-2-methyl-propanoate Int-2-4

To a solution of the Int-2-3 (1.89 g, 8.91 mmol) in EtOH (60 mL) was added (NH₄)₂CO₃ (6.86 g, 71.36 mmol), H₂O (60 mL) and NaCN (1.09 g, 22.30 mmol). The reaction mixture was stirred at 80° C. in a seal tube overnight and cooled to rt. Water was added and reaction mixture extracted with EtOAc. The combined organic layers were washed with water and brine, dried over anhydrous Na₂SO₄, filtered and concentrated in vacuo. The residue was triturated with EtOAc/hexane (1/5), filtered and dried to give Int-2-4 (1.23 g, 4.34 mmol, 48.71% yield). ¹HNMR (400 MHz, DMSO-d₆): δ 10.54 (brs, 0.5H) 10.52 (brs, 0.5H), 7.64 (brs, 0.5H), 7.52 (brs, 0.5H), 2.41-2.06 (m, 2H), 1.70 (dd, 0.5H), 1.56 (dd, 0.5H), 1.37 (d, 9H), 1.07 (d, 1.5H), 1.08-1.01 (m, 1H), 1.02 (d, 1.5H), 0.48-0.29 (m, 3H), 0.14-0.05 (m, 1H).

### Step 4

### 3-(4-cyclopropyl-2,5-dioxoimidazolidin-4-yl)-2-methylpropanoic acid Int-2

The solution of Int-2-4 (100 mg, 354.19 umol) in HCl/Dioxane (2N, 4 mL) was stirred at room temperature overnight. The mixture was concentrated in vacuum to give Int-2 (100 mg). The product was used directly in the next step without further purification.

### Intermediate 2A (Int-2A) and Intermediate 2B (Int-2B)

### 3-((S)-4-cyclopropyl-2,5-dioxoimidazolidin-4-yl)-2-methylpropanoic acid Int-2A

### 3-((R)-4-cyclopropyl-2,5-dioxoimidazolidin-4-yl)-2-methylpropanoic acid Int-2B

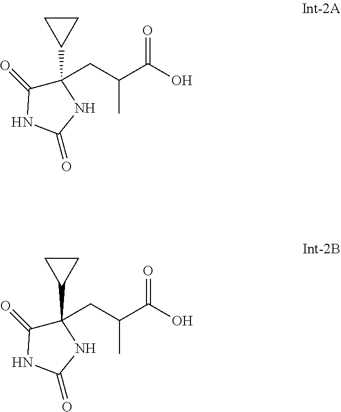 [SMILES: CC(C[C@@]1(C2CC2)NC(=O)NC1=O)C(=O)O ; CC(C[C@]1(C2CC2)NC(=O)NC1=O)C(=O)O]

Int-2-4 (1.22 g) was separated by silica gel chromagraphy (hexane:EtOAc=20:1) to give diastereoisomers 1 (360 mg) and diastereoisomers 2 (350 mg). Diastereoisomers 1 were converted to Int-2A using similar procdure as Step 4 for Int-2. Diastereoisomers 2 were converted to Int-2B using similar procdure as Step 4 for Int-2.

### Int-2A

¹HNMR (400 MHz, DMSO-d₆): δ 12.15 (brs, 1H), 10.62 (s, 1H), 7.64 (s, 1H), 2.33-2.27 (m, 1H), 2.19-2.13 (m, 1H), 1.77-1.71 (m, 1H), 1.05 (d, 4H), 0.45-0.26 (m, 3H), 0.09-0.04 (m, 1H).

LCMS: MS m/z (ESI): 227.2 [M+H]⁺.

### Int-2B

¹HNMR (400 MHz, DMSO-d₆): δ 12.16 (brs, 1H), 10.57 (s, 1H), 7.68 (s, 1H), 2.40-2.35 (m, 1H), 2.30-2.24 (m, 1H), 1.60 (dd, 1H), 1.12-1.05 (m, 1H), 1.11 (d, 3H), 0.43-0.30 (m, 3H), 0.13-0.07 (m, 1H).

LCMS: MS m/z (ESI): 227.2 [M+H]⁺.

### EXAMPLES

### Example 1

### (S)-5-cyclopropyl-5-(3-oxo-3-(5-(trifluoromethyl)isoindolin-2-yl)propyl)imidazolidine-2,4-dione 1

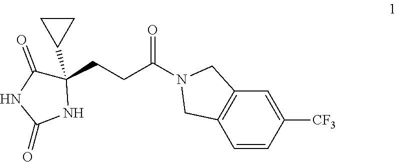 [SMILES: O=C1NC(=O)[C@](CCC(=O)N2Cc3ccc(C(F)(F)F)cc3C2)(C2CC2)N1]

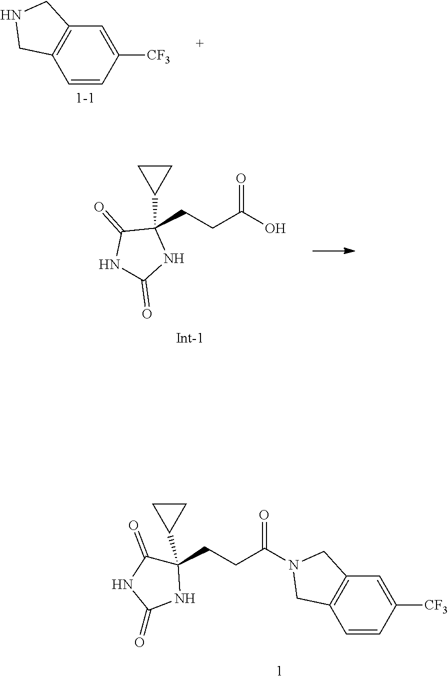 [SMILES: FC(F)(F)c1ccc2c(c1)CNC2 ; O=C(O)CC[C@@]1(C2CC2)NC(=O)NC1=O ; O=C1NC(=O)[C@](CCC(=O)N2Cc3ccc(C(F)(F)F)cc3C2)(C2CC2)N1]

To a mixture of 5-(trifluoromethyl)isoindoline 1-1 (150 mg, 801.45 umol) in DMF (10 mL) was added triethylamine (324 mg, 3.21 mmol), Int-1 (170 mg, 801.45 umol) and HATU (365 mg, 961.74 umol). The reaction was stirred at r.t for 18 h. Water (20 mL) was added and the mixture was extracted with EtOAc (20 mL×2). The combined layer was washed with brine, dried over Na₂SO₄ and concentrated. The residue was purified with Prep-HPLC to give title compound example 1 (80 mg, 209.78 umol, 26.18% yield).

¹H NMR (400 MHz, DMSO-d6): δ 10.64 (s, 1H), 7.77-7.74 (m, 2H), 7.66 (d, 1H), 7.58 (d, 1H), 4.89-4.69 (m, 4H), 2.48-2.38 (m, 1H), 2.33-2.24 (m, 1H), 2.04-1.99 (m, 2H), 1.16-1.08 (m, 1H), 0.50-0.46 (m, 1H), 0.43-0.29 (m, 2H), 0.15-0.08 (m, 1H).

LCMS: MS m/z (ESI): 382.1 [M+H]⁺.

### Example 2

### (S)-5-(3-(5-chloroisoindolin-2-yl)-3-oxopropyl)-5-cyclopropylimidazolidine-2,4-dione 2

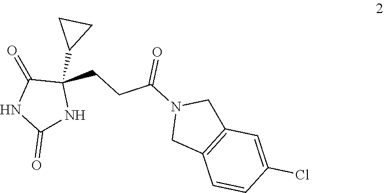 [SMILES: O=C1NC(=O)[C@](CCC(=O)N2Cc3ccc(Cl)cc3C2)(C2CC2)N1]

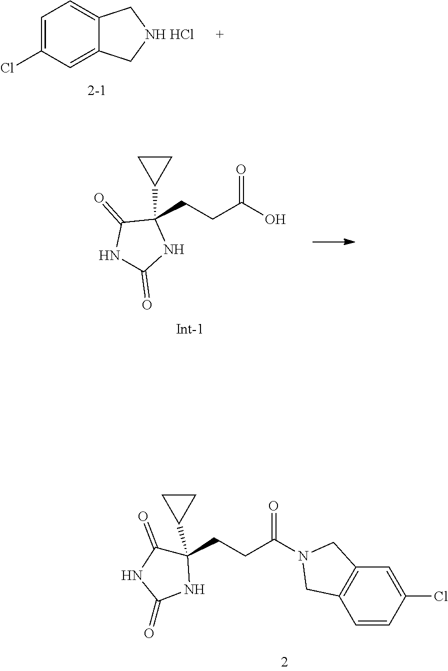

To a solution of 5-chloroisoindoline hydrochloride 2-1 (100 mg, 526.12 umol) in DMF (3 mL) was added TEA (0.22 mL), Int-1 (134 mg, 631.35 umol) and HATU (240 mg, 631.35 umol). The mixture was stirred at room temperature overnight. LCMS showed starting material was completely reacted. Water (20 mL) was added and the mixture extracted with EtOAc (15 mL×2). The combined organic layers are washed with water (30 mL) and brine (30 mL×2), dried over anhydrous Na₂SO₄, filtered and concentrated in vacuum. The crude was purified by prep-HPLC to give title compound 2 (70 mg, 38.03%).

¹HNMR (400 MHz, DMSO-d6): δ 10.62 (s, 1H), 7.73 (s, 1H), 7.45 (s, 1H), 7.39-7.33 (m, 2H), 4.77 (d, 2H), 4.61 (d, 2H), 2.43-2.33 (m, 1H), 2.31-2.22 (m, 1H), 2.00 (t, 2H), 1.15-1.07 (m, 1H), 0.49-0.45 (m, 1H), 0.40-0.29 (m, 2H), 0.13-0.08 (m, 1H).

LCMS: MS m/z (ESI): 348.1 [M+H]⁺.

### Example 3

### (S)-2-(3-(4-cyclopropyl-2,5-dioxoimidazolidin-4-yl)propanoyl)isoindoline-5-carbonitrile 3

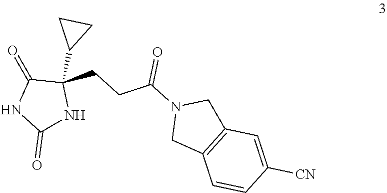 [SMILES: N#Cc1ccc2c(c1)CN(C(=O)CC[C@@]1(C3CC3)NC(=O)NC1=O)C2]

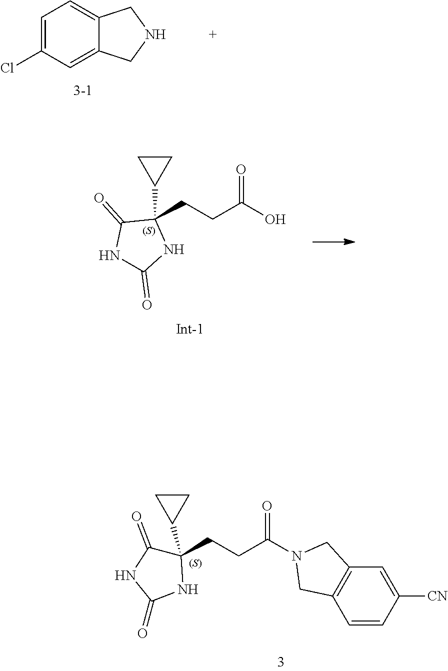

To a mixture of isoindoline-5-carbonitrile 3-1 (50 mg, 346.81 umol) in DMF (10 mL) was added Triethylamine (140 mg, 1.39 mmol), Int-1 (88 mg, 416.17 umol) and HATU (158 mg, 416.17 umol). The reaction was stirred at r.t for 18 h. Water (20 mL) was added and the mixture was extracted with EtOAc (20 mL×2). The combined layer was washed with brine, dried over Na₂SO₄ and concentrated. The residue was purified with prep-HPLC to give title compound 3 (25 mg, 73.89 umol, 21.30% yield).

¹H NMR (400 MHz, CDCl₃): δ 7.97-7.92 (m, 1H), 7.65-7.57 (m, 2H), 7.45-7.38 (m, 1H), 6.15 (brs, 1H), 4.84 (brs, 4H), 2.58-2.30 (m, 4H), 1.25-1.18 (m, 1H), 0.64-0.57 (m, 1H), 0.50-0.44 (m, 1H), 0.42-0.32 (m, 2H).

LCMS: MS m/z (ESI): 339.1 [M+H]⁺.

### Example 4

### (S)-5-cyclopropyl-5-(3-oxo-3-(5-(trifluoromethoxy)isoindolin-2-yl)propyl)imidazolidine-2,4-dione 4

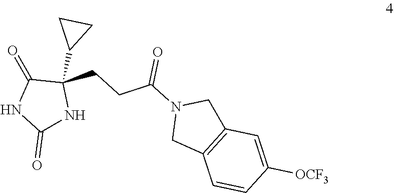 [SMILES: O=C1NC(=O)[C@](CCC(=O)N2Cc3ccc(OC(F)(F)F)cc3C2)(C2CC2)N1]

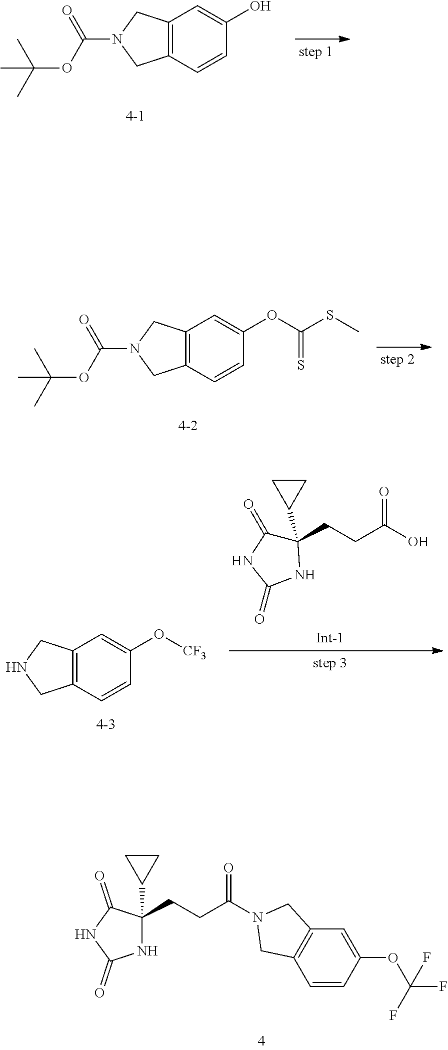 [SMILES: CC(C)(C)OC(=O)N1Cc2ccc(O)cc2C1 ; CSC(=S)Oc1ccc2c(c1)CN(C(=O)OC(C)(C)C)C2 ; O=C(O)CC[C@@]1(C2CC2)NC(=O)NC1=O ; FC(F)(F)Oc1ccc2c(c1)CNC2 ; O=C1NC(=O)[C@](CCC(=O)N2Cc3ccc(OC(F)(F)F)cc3C2)(C2CC2)N1]

### Step 1

### tert-butyl 5-(((methylthio)carbonothioyl)oxy)isoindoline-2-carboxylate 4-2

To a solution of tert-butyl 5-hydroxyisoindoline-2-carboxylate 4-1 (1.5 g, 6.38 mmol) in DMF (20 ml) was added sodium hydride (0.4 g, 9.57 mmol), at 0° C. After stirring for 30 minutes, carbon disulfide (0.5 ml) was added. It was stirred for one hour, and methyl iodide (0.6 ml, 8.30 mmol) was added. The reaction mixture was stirred at ambient temperature for 14 hours. It was quenched with ice water and extracted with ethyl acetate. The organic layer was washed with water, dried, and concentrated to get 4-2 as a pale solid (1.95 g, 93.8% yield).

¹H NMR (400 MHz, CDCl₃): 7.24 (d, 1H), 7.18 (s, 1H), 6.95 9d, 1H), 4.61 (dd, 4H), 2.60 (s, 3H), 1.44 (s, 9H).

LCMS: MS m/z (ESI): 326 [M+H]⁺.

### Step 2

### 5-(trifluoromethoxy)isoindoline 4-3

To a solution of 4-2 in pyridine-hydrogen fluoride complex (15 ml) was added 1,3-dibromo-5,5-dimethylimidazolidine-2,4-dione (5.5 g, 19.2 mmol), at 0° C. The reaction mixture was stirred at ambient temperature for 14 hours. It was quenched with ice water and extracted with ethyl acetate. The organic layer was washed with water, dried, and concentrated. The residue was purified on a silica gel column, eluting with ethyl acetate in hexanes, to get 4-3, as a white solid (910 mg).

LCMS: MS m/z (ESI): 204 [M+H]⁺.

### Step 3

### (S)-5-cyclopropyl-5-(3-oxo-3-(5-(trifluoromethoxy)isoindolin-2-yl)propyl)imidazolidine-2,4-dione 4

A solution of Int-1 (10 mg. 0.047 mmol), EDCI (14 mg, 0.071 mmol) and HATU (27 mg, 0.071 mmol) in DMF (1.5 ml) was stirred at ambient temperature for 20 minutes, before 4-3 (10 mg, 0.047 mmol) was added. The reaction mixture was stirred at ambient temperature for 2 hours. It was directly loaded onto and purified on a reverse phase HPLC. The appropriate fraction was lyophilized to afford example 4.

¹H NMR (400 MHz, CD₃OD): 8.00-7.78 (m, 3H) 4.87 (m, 2H), 4.68 (m, 2H), 3.70 (m, 1H), 3.10 (m, 1H), 2.26 (m, 2H), 1.14 (m, 1H), 0.49 (m, 1H), 0.32 (m, 2H), 0.25 (m, 1H).

LCMS: MS m/z (ESI): 398 [M+H]⁺.

### Example 5

### (S)-5-cyclopropyl-5-(3-(7-methoxy-4,5-dihydro-1H-benzo [d]azepin-3 (2H)-yl)-3-oxopropyl)imidazolidine-2,4-dione 5

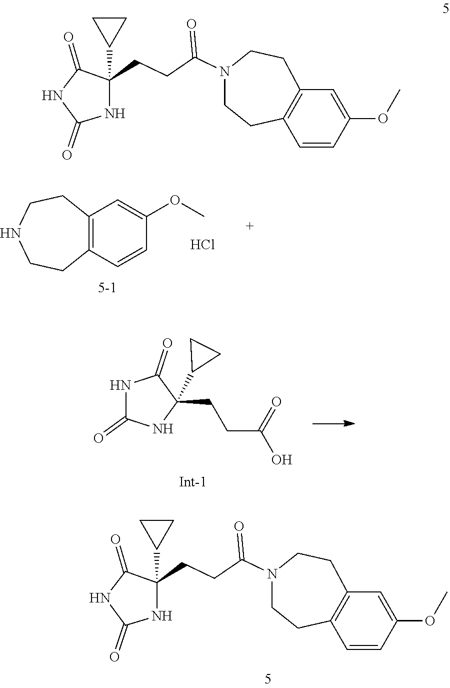

A solution of Int-1 (10 mg. 0.047 mmol), EDCI (14 mg, 0.071 mmol) and HATU (27 mg, 0.071 mmol) in DMF (1.5 ml) was stirred at ambient temperature for 20 minutes, before 7-methoxy-2,3,4,5-tetrahydro-1H-benzo[d]azepine hydrochloride 5-1 (10 mg, 0.047 mmol) was added. The reaction mixture was stirred at ambient temperature for 2 hours. It was directly loaded onto and purified on a reverse phase HPLC. The appropriate fraction was lyophilized to afford 5.

¹H NMR (400 MHz, CD₃OD): 7.06 (dd, 1H), 6.74 (m, 1H), 6.70 (dt, 1H), 3.77 (d, 3H), 3.72-3.59 (m, 4H), 2.96-2.84 (m, 4H), 2.54 (m, 1H), 2.42 (m, 1H), 2.11 (m, 2H), 1.21 (m, 1H), 0.56 (m, 1H), 0.44-0.30 (m, 3H).

LCMS: MS m/z (ESI): 372 [M+H]⁺.

### Example 6

### (S)-5-cyclopropyl-5-(3-oxo-3-(6-(trifluoromethyl)-3,4-dihydroisoquinolin-2 (1H)-yl)propyl)imidazolidine-2,4-dione 6

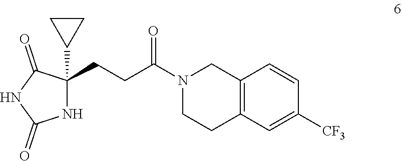 [SMILES: O=C1NC(=O)[C@](CCC(=O)N2CCc3cc(C(F)(F)F)ccc3C2)(C2CC2)N1]

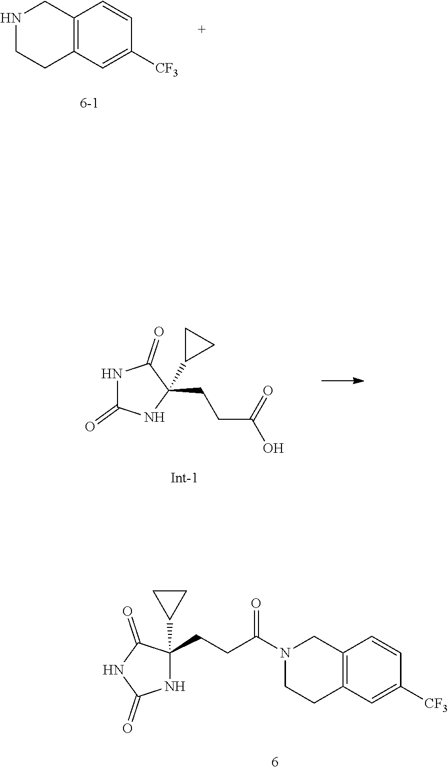 [SMILES: FC(F)(F)c1ccc2c(c1)CCNC2 ; O=C(O)CC[C@@]1(C2CC2)NC(=O)NC1=O ; O=C1NC(=O)[C@](CCC(=O)N2CCc3cc(C(F)(F)F)ccc3C2)(C2CC2)N1]

A solution of Int-1 (10 mg. 0.047 mmol), EDCI (14 mg, 0.071 mmol) and HATU (27 mg, 0.071 mmol) in DMF (1.5 ml) was stirred at ambient temperature for 20 minutes, before 6-(trifluoromethyl)-1,2,3,4-tetrahydroisoquinoline 6-1 (10 mg, 0.047 mmol) was added. The reaction mixture was stirred at ambient temperature for 2 hours. It was directly loaded onto and purified on a reverse phase HPLC. The appropriate fraction was lyophilized to afford 6.

¹H NMR (400 MHz, CD₃OD): 8.15-7.50 (m, 3H), 4.10 (m, 1H), 3.55-3.85 (m, 4H), 3.00 (m, 2H), 2.50-2.10 (m, 3H), 1.21 (m, 1H), 0.60 (m, 1H), 0.49-0.30 (m, 3H).

LCMS: MS m/z (ESI): 396 [M+H]⁺.

### Example 7

### (S)-5-cyclopropyl-5-(3-oxo-3-(7-(trifluoromethyl)-3,4-dihydroisoquinolin-2 (1H)-yl)propyl)imidazolidine-2,4-dione 7

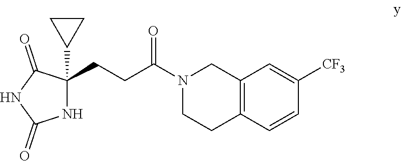 [SMILES: O=C1NC(=O)[C@](CCC(=O)N2CCc3ccc(C(F)(F)F)cc3C2)(C2CC2)N1]

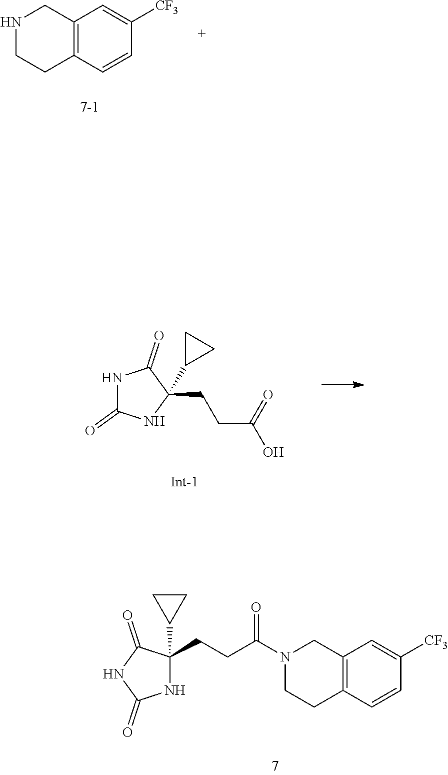 [SMILES: FC(F)(F)c1ccc2c(c1)CNCC2 ; O=C(O)CC[C@@]1(C2CC2)NC(=O)NC1=O ; O=C1NC(=O)[C@](CCC(=O)N2CCc3ccc(C(F)(F)F)cc3C2)(C2CC2)N1]

A solution of Int-1 (10 mg. 0.047 mmol), EDCI (14 mg, 0.071 mmol) and HATU (27 mg, 0.071 mmol) in DMF (1.5 ml) was stirred at ambient temperature for 20 minutes, before 7-(trifluoromethyl)-1,2,3,4-tetrahydroisoquinoline 7-1 (10 mg, 0.047 mmol) was added. The reaction mixture was stirred at ambient temperature for 2 hours. It was directly loaded onto and purified on a reverse phase HPLC. The appropriate fraction was lyophilized to afford 7, as a white solid.

¹H NMR (400 MHz, CD₃OD): 8.10-7.60 (m, 3H), 4.12 (m, 1H), 3.55-3.85 (m, 4H), 3.00 (m, 2H), 2.50-2.10 (m, 3H), 1.21 (m, 1H), 0.60 (m, 1H), 0.49-0.30 (m, 3H).

LCMS: MS m/z (ESI): 396 [M+H]⁺.

### Example 8

### (5S)-5-(3-(8-chloro-1-methyl-4,5-dihydro-1H-benzo[d]azepin-3 (2H)-yl)-3-oxopropyl)-5-cyclopropylimidazolidine-2,4-dione 8

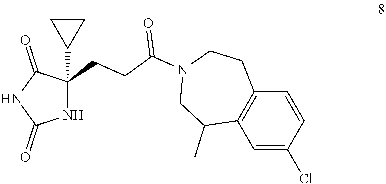 [SMILES: CC1CN(C(=O)CC[C@@]2(C3CC3)NC(=O)NC2=O)CCc2ccc(Cl)cc21]

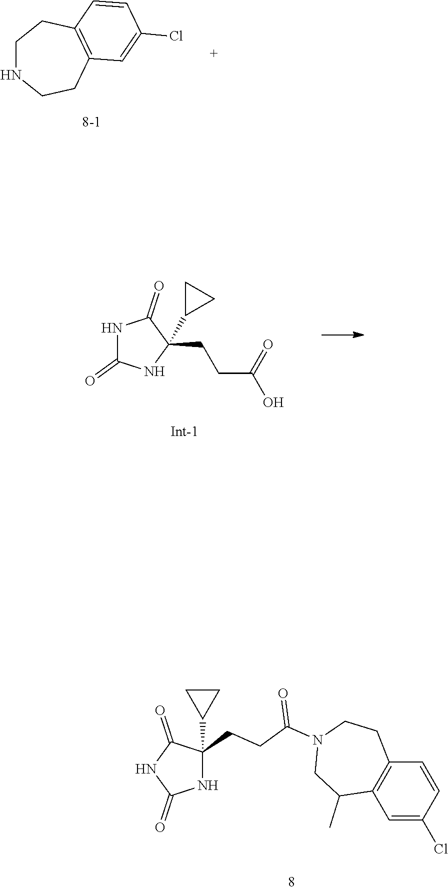 [SMILES: Clc1ccc2c(c1)CCNCC2 ; O=C(O)CC[C@@]1(C2CC2)NC(=O)NC1=O ; CC1CN(C(=O)CC[C@@]2(C3CC3)NC(=O)NC2=O)CCc2ccc(Cl)cc21]

A solution of Int-1 (10 mg. 0.047 mmol), EDCI (14 mg, 0.071 mmol) and HATU (27 mg, 0.071 mmol) in DMF (1.5 ml) was stirred at ambient temperature for 20 minutes, before 8-chloro-1-methyl-2,3,4,5-tetrahydro-1H-benzo[d]azepine 8-1 (12 mg, 0.047 mmol) was added. The reaction mixture was stirred at ambient temperature for 2 hours. It was directly loaded onto and purified on a reverse phase HPLC. The appropriate fraction was lyophilized to afford 8 (mixture of two diastereomers).

LCMS: MS m/z (ESI): 390 [M+H]⁺.

### Example 9

### (S)-5-cyclopropyl-5-(3-(4,5-dihydro-1H-benzo[d]azepin-3 (2H)-yl)-3-oxopropyl)imidazolidine-2,4-dione 9

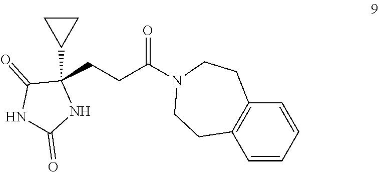 [SMILES: O=C1NC(=O)[C@](CCC(=O)N2CCc3ccccc3CC2)(C2CC2)N1]

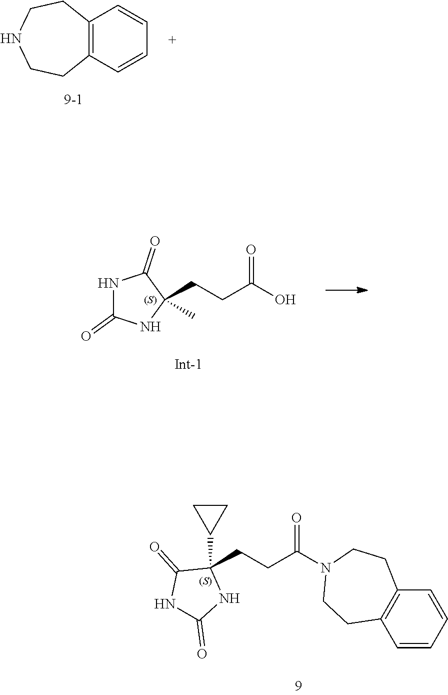

To a solution of 2,3,4,5-tetrahydro-1H-benzo[d]azepine 9-1 (100 mg, 679.27 umol) in DMF (4 mL) was added Triethylamine (275 mg, 2.72 mmol, 377.67 uL), Int-1 (173 mg, 815.13 umol) and HATU (310 mg, 815.13 umol). The mixture was stirred at room temperature overnight. LCMS showed starting material was completely reacted. Water (25 mL) was added and the reaction mixture extracted with EtOAc (20 mL×2). The combined organic layers are washed with water (40 mL) and brine (40 mL×2), dried over anhydrous Na₂SO₄, filtered and concentrated in vacuum. The crude was purified by prep-HPLC to give title compound 9 (11 mg, 4.74%).

¹H NMR (400 MHz, DMSO-d₆): δ 10.62 (s, 1H), 7.72 (s, 1H), 7.15-7.13 (m, 4H), 3.58-3.51 (m, 4H), 2.91-2.90 (m, 2H), 2.82-2.81 (m, 2H), 2.45-2.35 (m, 1H), 2.30-2.25 (m, 1H), 61.94 (m, 2H), 1.12-1.08 (m, 1H), 0.48-0.46 (m, 1H), 0.40-0.29 (m, 2H), 0.10-0.08 (m, 1H).

LCMS: MS m/z (ESI): 342.2 [M+H]⁺.

### Example 20

### (S)-5-cyclopropyl-5-(3-(5,6-dichloroisoindolin-2-yl)-3-oxopropyl)imidazolidine-2,4-dione

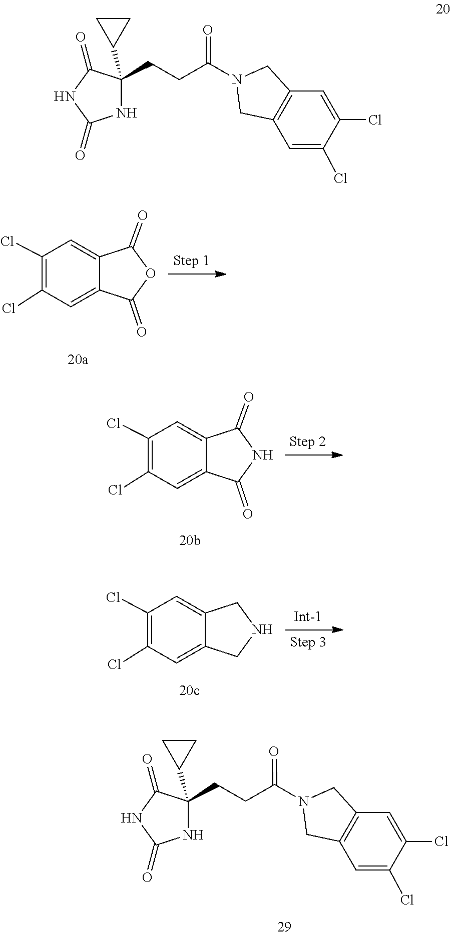 [SMILES: O=C1NC(=O)[C@](CCC(=O)N2Cc3cc(Cl)c(Cl)cc3C2)(C2CC2)N1 ; O=C1OC(=O)c2cc(Cl)c(Cl)cc21 ; O=C1NC(=O)c2cc(Cl)c(Cl)cc21 ; Clc1cc2c(cc1Cl)CNC2 ; O=C1NC(=O)[C@](CCC(=O)N2Cc3cc(Cl)c(Cl)cc3C2)(C2CC2)N1]

### Step 1

### 5,6-dichloroisoindoline-1,3-dione 20b

The mixture of 5,6-dichloroisobenzofuran-1,3-dione 20a (5 g, 23.04 mmol) in formamide (1.04 g, 23.04 mmol) was stirred at 200° C. for 2 h. The mixture was poured into ice water (100 mL), the mixture was filtered and the solid was dried to afford the tittle compound 20b (4.5 g, 20.83 mmol, 90.41% yield).

¹H NMR (400 MHz, DMSO-d₆): δ 11.62 (s, 1H), 8.12 (s, 2H).

### Step 2

### 5,6-dichloroisoindoline 20c

To a solution of 20b (1 g, 4.63 mmol) in THF (5 mL) was added BH₃ (1 M in THF, 46.29 mL). The resulting mixture was stirred at 80° C. for 16 h. Con. HCl (10 mL) was added to quench the reaction. Then aq NaOH was added to adjust the mixture to pH-13, the aqueous phase was extracted with EtOAc (80 mL×3), the combined organic phases were washed with brine (80 mL), dried over Na₂SO₄, filtered and concentrated under reduced pressure. The residue was purified by silica gel chromatography (eluting with DCM/MeOH=10/1) to afford the tittle compound 20c (150 mg, 797.64 umol, 17.23% yield).

¹H NMR (400 MHz, DMSO-d₆): δ 7.55 (s, 2H), 4.06 (s, 4H).

### Step 3

### (S)-5-cyclopropyl-5-(3-(5,6-dichloroisoindolin-2-yl)-3-oxopropyl)imidazolidine-2,4-dione 20

To a solution of Int-1 (140 mg, 659.75 umol) in DMF (7 mL) was added DIEA (111 mg, 857.67 umol). The resulting mixture was followed by the addition of 20c (149 mg, 791.70 umol) and EDCI (116 mg, 857.67 umol). The resulting mixture was stirred at room temperature for 2 h. The mixture was purified by prep-HPLC to afford the tittle compound 20 (109 mg, 283.66 umol, 43.00% yield).

¹H NMR (400 MHz, DMSO-d₆): δ 10.63 (s, 1H), 7.74 (s, 1H), 7.67 (s, 2H), 4.77 (brs, 2H), 4.60 (brs, 2H), 2.50-2.37 (m, 1H), 2.29-2.24 (m, 1H), 2.02-1.97 (m, 2H), 1.12-1.10 (m, 1H), 0.46-0.33 (m, 3H), 0.13-0.11 (m, 1H).

LCMS: MS m/z (ESI): 382.0[M+H]⁺.

### Examples 29 and 30

### (5S)-5-cyclopropyl-5-(2-methyl-3-oxo-3-(5-(trifluoromethyl)isoindolin-2-yl)propyl)imidazolidine-2,4-dione 29

### (5R)-5-cyclopropyl-5-(2-methyl-3-oxo-3-(5-(trifluoromethyl)isoindolin-2-yl)propyl)imidazolidine-2,4-dione 30

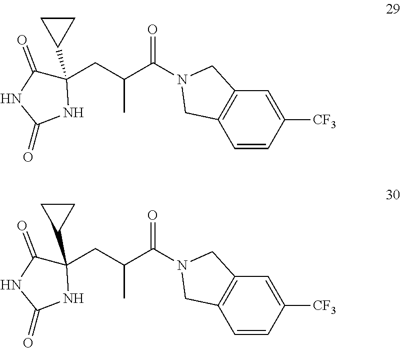 [SMILES: CC(C[C@@]1(C2CC2)NC(=O)NC1=O)C(=O)N1Cc2ccc(C(F)(F)F)cc2C1 ; CC(C[C@]1(C2CC2)NC(=O)NC1=O)C(=O)N1Cc2ccc(C(F)(F)F)cc2C1]

To a solution of 5-(trifluoromethyl)isoindoline 1-1 (1.0 g, 4.47 mmol, HCl salt) in DMF (30 mL) was added TEA (3.5 mL), Int-2 (1.21 g, 5.37 mmol) and HATU (2.04 g, 5.37 mmol). The mixture was stirred at room temperature overnight. LCMS monitoring, the starting material was completely reacted. Water was added and the mixture was extracted with EtOAc. The combined organic layer was washed with water and brine, dried over anhydrous Na₂SO₄, filtered and concentrated. The residue was purified by prep-HPLC to give two sets of diastereomers 29 (960 mg, 2.43 mmol, 56.56% yield) and 30 (300 mg, 0.76 mmol, 17.68% yield).

### Examples 29-1 and 29-2

### (S)-5-cyclopropyl-5-((S)-2-methyl-3-oxo-3-(5-(trifluoromethyl)isoindolin-2-yl)propyl)imidazolidine-2,4-dione 29-1

### (S)-5-cyclopropyl-5-((R)-2-methyl-3-oxo-3-(5-(trifluoromethyl)isoindolin-2-yl)propyl)imidazolidine-2,4-dione 29-2

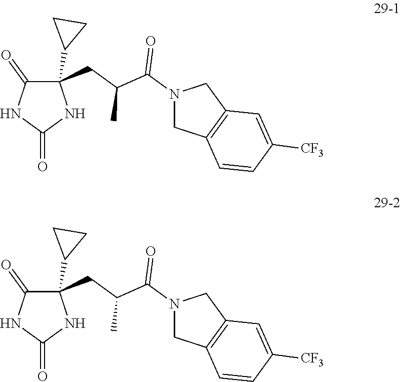 [SMILES: C[C@@H](C[C@@]1(C2CC2)NC(=O)NC1=O)C(=O)N1Cc2ccc(C(F)(F)F)cc2C1 ; C[C@H](C[C@@]1(C2CC2)NC(=O)NC1=O)C(=O)N1Cc2ccc(C(F)(F)F)cc2C1]

29 (200 mg) was separated by SFC (Daicel CHIRALPAK AS) to give two single enantiomers (67 mg and 75 mg).

### Enantiomer (Shorter Retention Time):

¹HNMR (400 MHz, DMSO-d₆): δ 10.64 (s, 1H), 7.75 (d, 1H), 7.69-7.65 (m, 2H), 7.58 (t, 1H), 4.92 (d, 2H), 4.75-4.64 (m, 2H), 2.66 (br, 1H), 2.39-2.32 (m, 1H), 1.74-1.70 (m, 1H), 1.06 (d, 3H), 1.06-1.00 (m, 1H), 0.43-0.38 (m, 1H), 0.31-0.21 (m, 2H), 0.03-0.01 (m, 1H).

LCMS: MS m/z (ESI): 396.1 [M+H]⁺.

Chiral HPLC (CO₂/MeOH/DEA 5%-40% 1.5 ml/min AS,3 um,3*100(Daicel)): ee: 100%, Rt: 1.370 min.

### Enantiomer (Longer Retention Time):

¹HNMR (400 MHz, DMSO-d₆): δ 10.64 (s, 1H), 7.75 (d, 1H), 7.69-7.66 (m, 2H), 7.59 (t, 1H), 4.93 (d, 2H), 4.75-4.64 (m, 2H), 2.65 (br, 1H), 2.39-2.32 (m, 1H), 1.74-1.70 (m, 1H), 1.07 (d, 3H), 1.07-1.05 (m, 1H), 0.38 (br, 1H), 0.29-0.25 (m, 2H), 0.03-0.00 (m, 1H).

LCMS: MS m/z (ESI): 396.1 [M+H]⁺.

Chiral HPLC (CO₂/MeOH/DEA 5%-40% 1.5 ml/min AS,3 um,3*100(Daicel)): ee: 96.62%, Rt: 1.972 min.

### Examples 30-1 and 30-2

### (R)-5-cyclopropyl-5-((R)-2-methyl-3-oxo-3-(5-(trifluoromethyl)isoindolin-2-yl)propyl)imidazolidine-2,4-dione 30-1

### (R)-5-cyclopropyl-5-((S)-2-methyl-3-oxo-3-(5-(trifluoromethyl)isoindolin-2-yl)propyl)imidazolidine-2,4-dione 30-2

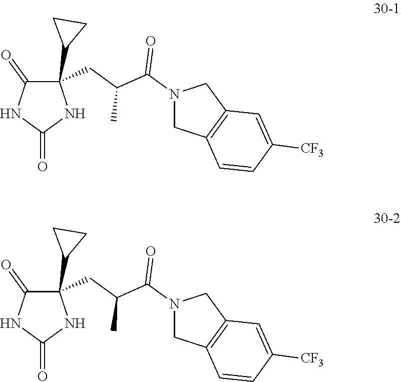 [SMILES: C[C@H](C[C@]1(C2CC2)NC(=O)NC1=O)C(=O)N1Cc2ccc(C(F)(F)F)cc2C1 ; C[C@@H](C[C@]1(C2CC2)NC(=O)NC1=O)C(=O)N1Cc2ccc(C(F)(F)F)cc2C1]

(300 mg) was separated by SFC (Daicel CHIRALPAK IA) to give two single enantiomers (70.6 mg and 71.6 mg).

### Enantiomer (Shorter Retention Time):

¹HNMR (400 MHz, DMSO-d₆): δ 10.50 (s, 1H), 7.75 (d, 1H), 7.70-7.65 (m, 2H), 7.59 (t, 1H), 4.98-4.86 (m, 2H), 4.72-4.59 (m, 2H), 2.81 (br, 1H), 2.41-2.34 (m, 1H), 1.69-1.64 (m, 1H), 1.11 (d, 3H), 1.11-1.06 (m, 1H), 0.46-0.40 (m, 1H), 0.35-0.26 (m, 2H), 0.11-0.06 (m, 1H).

¹⁹FNMR (376.5 MHz, DMSO-d₆): δ −60.72

LCMS: MS m/z (ESI): 396.0 [M+H]⁺.

Chiral HPLC (CO₂/MeOH/DEA 5%-40% 1.5 ml/min IA, 3 um 3.0*100(Daicel)): ee: 99.48%, Rt: 3.580 min.

### Enantiomer (Longer Retention Time):

¹HNMR (400 MHz, DMSO-d₆): δ 10.50 (s, 1H), 7.75 (d, 1H), 7.70-7.65 (m, 2H), 7.60-7.56 (m, 1H), 4.98-4.86 (m, 2H), 4.72-4.59 (m, 2H), 2.84-2.78 (m, 1H), 2.41-2.34 (m, 1H), 1.69-1.64 (m, 1H), 1.11 (d, 3H), 1.11-1.04 (m, 1H), 0.45-0.40 (m, 1H), 0.36-0.27 (m, 2H), 0.12-0.09 (m, 1H).

¹⁹FNMR (376.5 MHz, DMSO-d₆): δ-60.57

LCMS: MS m/z (ESI): 396.1 [M+H]⁺.

Chiral HPLC (CO₂/MeOH/DEA 5%-40% 1.5 ml/min IA, 3 um 3.0*100(Daicel)): ee: 96.26%, Rt: 4.368 min.

### Examples 31-1 and 31-2

### (5R)-5-cyclopropyl-5-(2-(5-(trifluoromethyl)isoindoline-2-carbonyl)butyl)imidazolidine-2,4-dione 31-1

### (5S)-5-cyclopropyl-5-(2-(5-(trifluoromethyl)isoindoline-2-carbonyl)butyl)imidazolidine-2,4-dione 31-2

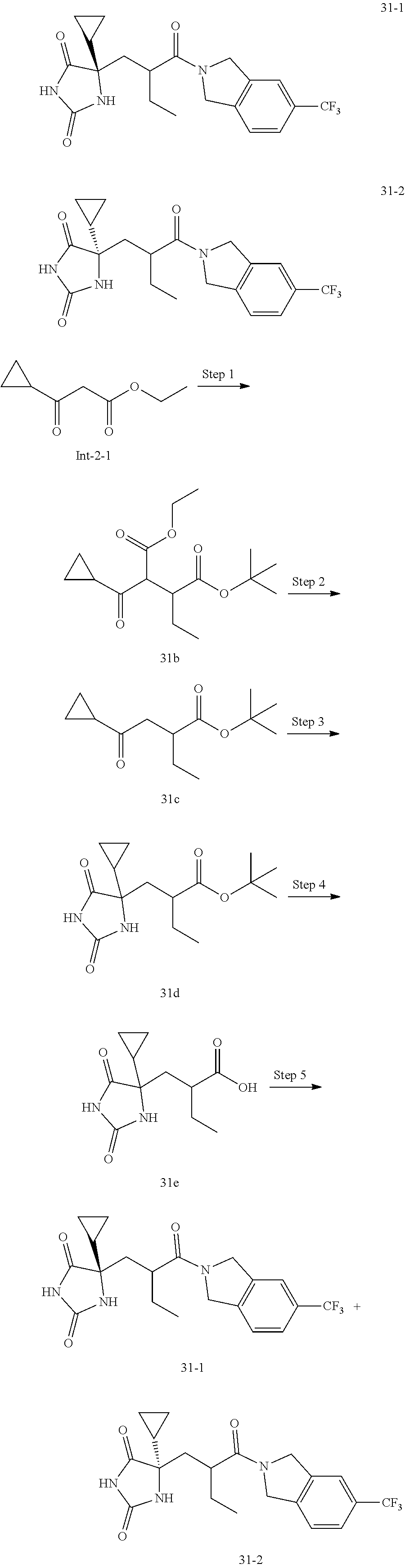

### Step 1

### 1-(tert-butyl) 4-ethyl 3-(cyclopropanecarbonyl)-2-ethylsuccinate 31b

To a mixture of Int-2-1 (30 g, 192.09 mmol) and tert-butyl 2-bromobutanoate (45.00 g, 201.69 mmol) in 2-butanone (400 mL) was added potassium carbonate (53.10 g, 384.18 mmol) and Sodium iodide (2.88 g, 19.21 mmol). The reaction was stirred at 100° C. for 72 h. The mixture was filtered and the filtrate was diluted with water (500 mL); the mixture was extracted with EtOAc (200 mL×3), the combined organic solution was washed with brine, dried over Na₂SO₄ and concentrated. The residue was purified by silica gel chromatography to give 31b (55.6 g, 186.34 mmol, 97.01% yield).

### Step 2

### Tert-butyl 4-cyclopropyl-2-ethyl-4-oxobutanoate 31c

To a mixture of 31b (7.5 g, 25.14 mmol) in THF (80 mL) was added a solution of LiOH·H₂O (3.17 g, 75.41 mmol) in water (80 mL). The reaction was stirred at r.t for 18 h. The reaction mixture was diluted with Citric acid to pH=6 and then stirred at 50° C. for 10 min. The mixture was diluted with water (100 mL) and the mixture was extracted with EtOAc (100 mL×3). The combined organic layers were washed with brine, dried over Na₂SO₄ and concentrated. The residue was purified by silica gel chromatography (hexane:EtOAc=20:1) to give 31c (5.1 g, 22.54 mmol, 89.65% yield).

### Step 3

### Tert-butyl 2-((4-cyclopropyl-2,5-dioxoimidazolidin-4-yl)methyl)butanoate 31d

To a mixture of 31c (5.1 g, 22.54 mmol) in EtOH (80 mL) was added to a solution of ammonium carbonate (17.32 g, 180.28 mmol) in water (80 mL). Then sodium cyanide (2.76 g, 56.34 mmol) was added. The reaction was stirred at 80° C. for 18 h in the sealed tube. The reaction was cooled to r.t and the mixture was poured into a mixture of water and EtOAc, the obtained mixture was extracted with EtOAc (100 mL×2). The organic layers were washed with brine, dried over Na₂SO₄ and concentrated. The residue was triturated with hexane (50 mL) for 1 h and filtered; the solid was dried to give 31d (mixture, 1.5 g, 5.06 mmol, 22.46% yield).

### Step 4

### 2-((4-cyclopropyl-2,5-dioxoimidazolidin-4-yl)methyl)butanoic acid 31e

The solution of 31d (1.5 g, 5.06 mmol) in HCl/Dioxane (4N, 20 mL) was stirred at r.t for 18 h. The mixture was concentrated, the residue was triturated with hexane (50 mL) and filtered, the solid was dried to give 31e (1.4 g, 5.06 mmol, 99.96% yield).

¹HNMR (400 MHz, DMSO-d₆): δ 12.13 (brs, 1H), 10.58, 10.53 (s, 1H), 7.64, 7.60 (s, 1H), 2.27-2.06 (m, 2H), 1.81-1.77 (m, 1H), 1.64-1.36 (m, 2H), 1.21-1.00 (m, 1H), 0.84-0.79 (m, 3H), 0.47-0.25 (m, 3H), 0.14-0.02 (m, 1H).

### Step 5

### (5R)-5-cyclopropyl-5-(2-(5-(trifluoromethyl)isoindoline-2-carbonyl)butyl)imidazolidine-2,4-dione (diastereoisomers 1, Rt: 6.567 min) 31-1

### (5S)-5-cyclopropyl-5-(2-(5-(trifluoromethyl)isoindoline-2-carbonyl)butyl)imidazolidine-2,4-dione (diastereoisomers 2, Rt: 7.063 min) 31-2

To a solution of 31e (400 mg, 1.66 mmol) in DMF (15 mL) was added 5-(trifluoromethyl)isoindoline 1-1 (372.31 mg, 1.66 mmol, HCl salt) and Triethylamine (673.88 mg, 6.66 mmol), then HATU (696.35 mg, 1.83 mmol) was added. The reaction was stirred at r.t for 4 h. Water (100 mL) was added and the mixture was extracted with EtOAc (50 mL×2), the combined organic layers were washed with brine, dried over Na₂SO₄ and concentrated. The residue was purified by prep-HPLC to give 31-1 (12.5 mg) and 31-2 (55 mg).

31-1:

¹HNMR (400 MHz, DMSO-d₆): δ 10.48 (s, 1H), 7.78-7.56 (m, 4H), 4.93-4.90 (m, 1H), 4.81-4.50 (m, 3H), 2.69-2.67 (m, 1H), 2.40-2.32 (m, 1H), 1.78-1.65 (m, 1H), 1.59-1.39 (m, 3H), 1.08-1.02 (m, 1H), 0.89-0.79 (m, 3H), 0.49-0.25 (m, 3H), 0.12-0.05 (m, 1H).

HPLC (SunFire C18 5 um4.6*150 mm 1.0 ml/min 16 min, 0.03% CH₃CN/H₂O): Rt: 6.567 min.

LCMS: MS m/z (ESI): 410.2 [M+H]⁺.

31-2:

¹HNMR (400 MHz, DMSO-d₆): δ 10.62 (s, 1H), 7.78-7.55 (m, 4H), 4.92-4.90 (m, 1H), 4.78-4.53 (m, 3H), 2.48-2.42 (m, 1H), 2.33-2.24 (m, 1H), 1.84-1.76 (m, 1H), 1.58-1.37 (m, 2H), 1.08-1.00 (m, 1H), 0.89-0.83 (m, 3H), 0.45-0.22 (m, 3H), 0.05-0.00 (m, 1H).

HPLC (SunFire C18 5 um 4.6*150 mm 1.0 ml/min 16 min, 0.03% CH₃CN/H₂O): Rt: 7.063 min.

LCMS: MS m/z (ESI): 410.1 [M+H]⁺.

### Examples 32 and 33

### (S)-5-cyclopropyl-5-((S)-2-(5-(trifluoromethyl)isoindoline-2-carbonyl)butyl)imidazolidine-2,4-dione 32

### (S)-5-cyclopropyl-5-((R)-2-(5-(trifluoromethyl)isoindoline-2-carbonyl)butyl)imidazolidine-2,4-dione 33

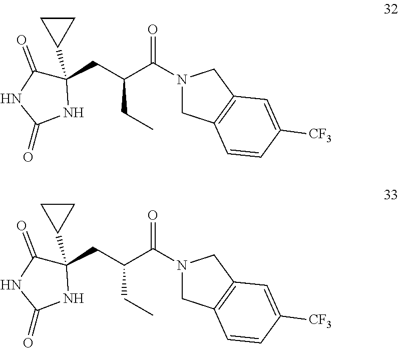 [SMILES: CC[C@@H](C[C@@]1(C2CC2)NC(=O)NC1=O)C(=O)N1Cc2ccc(C(F)(F)F)cc2C1 ; CC[C@H](C[C@@]1(C2CC2)NC(=O)NC1=O)C(=O)N1Cc2ccc(C(F)(F)F)cc2C1]

31-2 (350 mg) was separated by SFC (Daicel CHIRALPAK IG, 250*25 mm 10% m) to give single enantiomers (120 mg and 100 mg).

### Enantiomer (Shorter Retention Time):

¹HNMR (400 MHz, DMSO-d₆): δ 10.64 (s, 1H), 7.75 (d, 1H), 7.68 (d, 1H), 7.63-7.55 (m, 2H), 4.96-4.85 (m, 2H), 4.79-4.66 (m, 2H), 2.48-2.43 (m, 1H), 2.32-2.25 (m, 1H), 1.83-1.78 (m, 1H), 1.56-1.37 (m, 2H), 1.08-1.00 (m, 1H), 0.86 (t, 3H), 0.44-0.40 (m, 1H), 0.32-0.22 (m, 2H), 0.03-0.00 (m, 1H).

LCMS: MS m/z (ESI): 410.1 [M+H]⁺.

Chiral HPLC (CO₂/EtOH/DEA 5%-40% 1.5 ml/min IG,3 um,3*100(Daicel)): ee: 100%, Rt: 3.222 min.

### Enantiomer (Longer Retention Time):

¹HNMR (400 MHz, DMSO-d₆): δ10.64 (s, 1H), 7.75 (d, 1H), 7.68 (d, 1H), 7.63-7.55 (m, 2H), 4.96-4.85 (m, 2H), 4.79-4.66 (m, 2H), 2.48-2.43 (m, 1H), 2.33-2.25 (m, 1H), 1.84-1.78 (m, 1H), 1.57-1.38 (m, 2H), 1.08-1.00 (m, 1H), 0.86 (t, 3H), 0.44-0.39 (m, 1H), 0.29-0.22 (m, 1H), 0.04-0.00 (m, 1H).

LCMS: MS m/z (ESI): 410.1 [M+H]⁺.

Chiral HPLC (CO₂/EtOH/DEA 5%-40% 1.5 ml/min IG,3 um,3*100(Daicel)): ee: 99.40%, Rt: 3.854 min.

### Example 34-1

### (5R)-5-cyclopropyl-5-(3-(5,6-dichloroisoindolin-2-yl)-2-methyl-3-oxopropyl)imidazolidine-2,4-dione

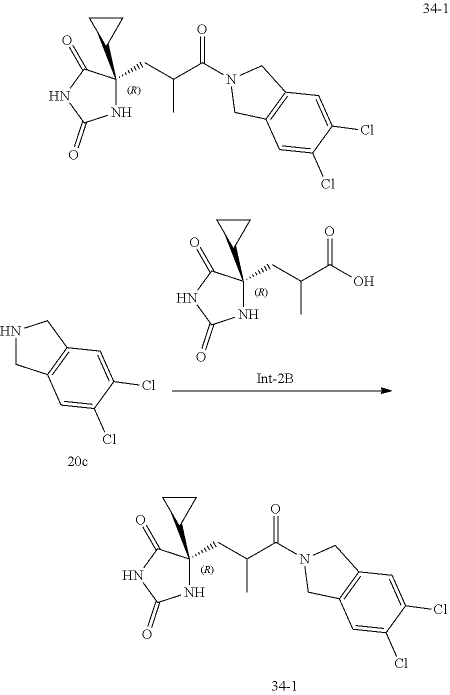

To a mixture of 5,6-dichloroisoindoline 20c (9 mg, 0.047 mmol) in DMF (2 mL) was added Triethylamine (17.22 mg, 0.132 mmol), Int-2B (10 mg, 0.044 mmol) and HATU (21.8 mg, 0.0574 mmol). The reaction was stirred at r.t for 18 h. Water (3 mL) added and the mixture was extracted with EtOAc (20 mL×2). The combined layer was washed with brine, dried over Na₂SO₄ and concentrated. The residue was purified with prep-HPLC to give title compound 34-1 (5 mg, 0.0125 mmol, 26% yield).

¹H NMR (400 MHz, Methanol-d₄): δ 7.53 (d, 2H), 4.70 (d, 4H), 2.58 (dd, 1H), 1.86 (dd, 2H), 1.25-1.14 (m, 3H), 0.62-0.50 (m, 1H), 0.48-0.36 (m, 2H), 0.40-0.25 (m, 2H).

LCMS: MS m/z (ESI): 397 [M+H]⁺.

### Example 34-2

### (5S)-5-cyclopropyl-5-(3-(5,6-dichloroisoindolin-2-yl)-2-methyl-3-oxopropyl)imidazolidine-2,4-dione

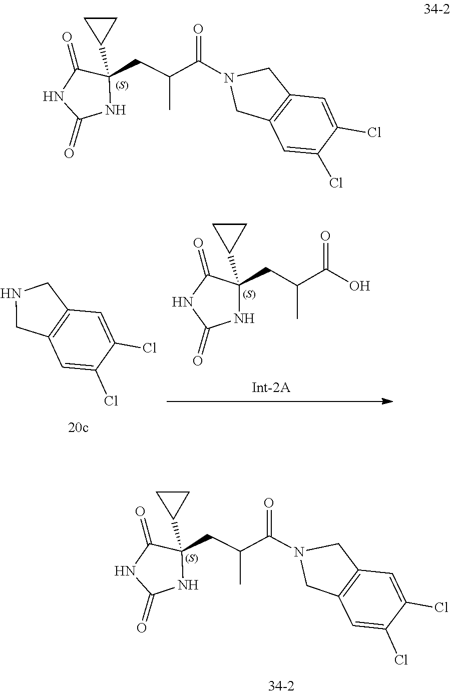

To a mixture of 5,6-dichloroisoindoline 20c (72 mg, 0.38 mmol) in DMF (4 mL) was added Triethylamine (144 mg, 1.11 mmol), Int-2A (80 mg, 0.37 mmol) and HATU (171.8 mg, 0.45 mmol). The reaction was stirred at r.t for 18 h. Water (5 mL) was added and the mixture was extracted with EtOAc (40 mL×3). The combined organic layer was washed with brine, dried over Na₂SO₄ and concentrated. The residue was purified with prep-HPLC to give title compound 34-2 (55 mg, 0.138 mmol, 37% yield).

¹H NMR (400 MHz, Methanol-d₄): δ 7.53 (d, 2H), 4.73 (s, 4H), 2.72 (m, 1H), 2.08-1.94 (m, 2H), 1.31-1.16 (m, 3H), 0.53 (td, 1H), 0.49-0.34 (m, 2H), 0.34-0.24 (m, 2H).

LCMS: MS m/z (ESI): 396 [M+H]⁺.

### Example 34-2-A and Example 34-2-B

### (5S)-5-cyclopropyl-5-((S)-(3-(5,6-dichloroisoindolin-2-yl)-2-methyl-3-oxopropyl))imidazolidine-2,4-dione 34-2-A

### (5S)-5-cyclopropyl-5-((R)-(3-(5,6-dichloroisoindolin-2-yl)-2-methyl-3-oxopropyl))imidazolidine-2,4-dione 34-2-B

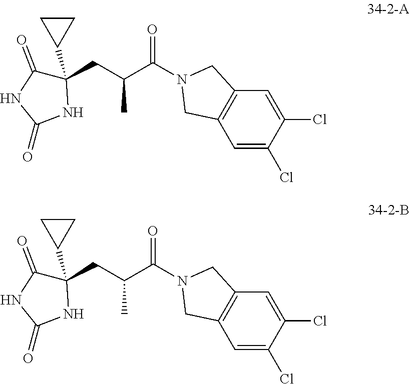 [SMILES: C[C@@H](C[C@@]1(C2CC2)NC(=O)NC1=O)C(=O)N1Cc2cc(Cl)c(Cl)cc2C1 ; C[C@H](C[C@@]1(C2CC2)NC(=O)NC1=O)C(=O)N1Cc2cc(Cl)c(Cl)cc2C1]

34-2 (27 mg) was separated by chiral HPLC (column: CHIRALPAK IG-3) to give title compounds (9 mg and 10 mg).

### Enantiomer (Shorter Retention Time):

### Chiral HPLC: (CHIRALPAK IG-3, 100% MeOH): Rt=4.119 min.

LCMS: m/z (ESI): 394.2 [M−H]⁻.

### Enantiomer (Longer Retention Time):

### Chiral HPLC: (CHIRALPAK IG-3, 100% MeOH): Rt=5.239 min.

LCMS: m/z (ESI): 394.1 [M−H]⁻.

### Example 35

### (S)-5-(3-(5-chloro-6-methoxyisoindolin-2-yl)-3-oxopropyl)-5-cyclopropylimidazolidine-2,4-dione

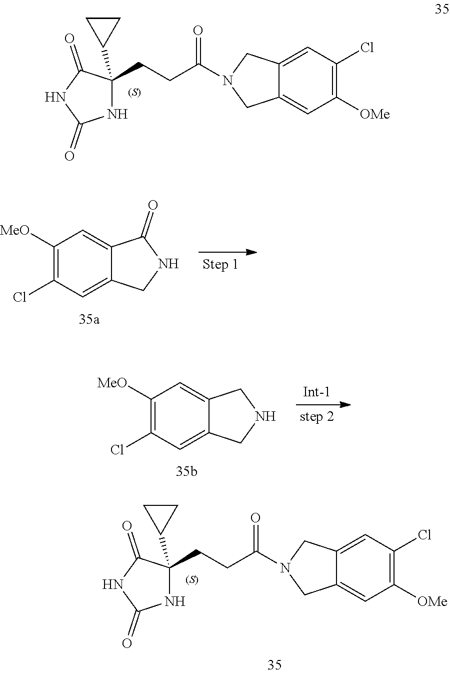

### Step 1

### 5-chloro-6-methoxyisoindoline 35b

To a solution of 5-chloro-6-methoxyisoindolin-1-one 35a (1 g, 5 mmol) in THF (10 mL) was added borane-tetrahydrofuran (1M, 30 mL) dropwise under N₂. The resulting mixture was stirred at 60° C. for 24 h. The reaction mixture was cooled to ambient temperature and quenched with MeOH (5 mL) until the bubbling ceased. Then 4N HCl in water (5 mL) was added and the mixture was heated at 80° C. for 3 h. After cooled down to RT, 5N KOH was added to adjust pH to 7. The mixture was concentrated under reduced pressure and the residue was purified by silica-gel column (DCM: MeOH(2% NH₄₀H)=10:1) to afford 5-chloro-6-methoxyisoindoline 35b (300 mg, 60% yield).

### Step 2

### (S)-5-(3-(5-chloro-6-methoxyisoindolin-2-yl)-3-oxopropyl)-5-cyclopropylimidazolidine-2,4-dione 35

To a mixture of 5-chloro-6-methoxyisoindoline 35b (10 mg, 0.0514 mmol) in DMF (2 mL) was added triethylamine (18 mg, 0.141 mmol), Int-1 (10 mg, 0.047 mmol) and HATU (22 mg, 0.0567 mmol). The reaction was stirred at r.t for 18 h. Water (4 mL) added and the mixture was extracted with EtOAc (20 mL×2). The combined organic layers were washed with brine, dried over Na₂SO₄ and concentrated. The residue was purified with prep-HPLC to give title compound 35 (5 mg, 132.2 umol, 28% yield).

¹H NMR (400 MHz, Methanol-d₄): δ 7.52 (s, 2H), 4.66 (t, 4H), 3.69 (d, 3H), 1.25 (m, 4H), 0.60 (m, 1H), 0.46 (m, 2H), 0.42-0.30 (m, 2H).

LCMS: MS m/z (ESI): 378 [M+H]⁺.

### Example 36-1

### (5S)-5-(3-(5-chloro-6-methoxyisoindolin-2-yl)-2-methyl-3-oxopropyl)-5-cyclopropylimidazolidine-2,4-dione

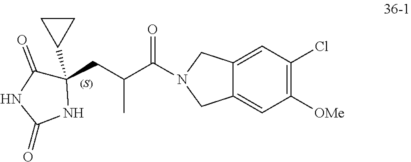

To a mixture of 5-chloro-6-methoxyisoindoline 35b (10 mg, 546.44 umol) in DMF (2 mL) was added triethylamine (18 mg, 0.141 mmol), Int-2A (10 mg, 442.4 umol) and HATU (21 mg, 564 umol). The reaction was stirred at r.t for 18 h. Water (4 mL) was added and the mixture was extracted with EtOAc (20 mL×2). The combined organic layers were washed with brine, dried over Na₂SO₄ and concentrated. The residue was purified with prep-HPLC to give the title compound 36-1 (5 mg, 127 umol, 28% yield).

¹H NMR (400 MHz, Methanol-d4): δ 7.52 (s, 2H), 4.66 (t, 4H), 3.69 (d, 3H), 2.25 (m, 1H), 1.95 (m, 2H), 1.25 (m, 1H), 0.60 (m, 4H), 0.46 (m, 2H), 0.42-0.30 (m, 2H).

LCMS: MS m/z (ESI): 392 [M+H]⁺.

### Example 37

### (S)-5-cyclopropyl-5-(3-(7,8-dichloro-1-methyl-1,2,4,5-tetrahydro-3H-benzo [d]azepin-3-yl)-3-oxopropyl)imidazolidine-2,4-dione 37

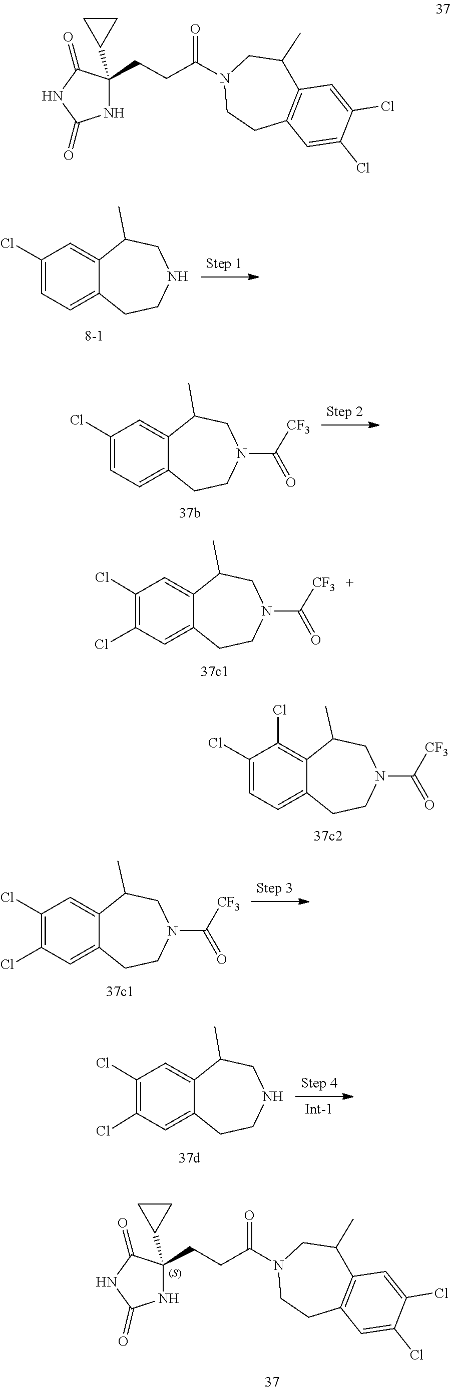

### Step 1

### 1-(8-chloro-1-methyl-1,2,4,5-tetrahydro-3H-benzo [d]azepin-3-yl)-2,2,2-trifluoroethan-1-one 37b

The solution of 8-chloro-1-methyl-2,3,4,5-tetrahydro-1H-benzo[d]azepine 8-1 (5 g, 21.54 mmol, HCl salt) in DCM (100 mL) was cooled to 0° C. before pyridine (3.41 g, 43.08 mmol) was added, then trifluoroacetic anhydride (5.43 g, 25.85 mmol) was added. The reaction was stirred at r.t for 1 h. The reaction solution was washed with HCl (2N, aq. 150 mL), NaHCO₃ (aq., 150 mL) and brine, dried over Na₂SO₄ and concentrated to give crude 37b (6.2 g, 21.25 mmol, 98.69% yield).

¹HNMR (400 MHz, CDCl₃): δ 7.18-7.12 (m, 2H), 7.08-7.02 (m, 1H), 4.09-3.39 (m, 2H), 3.70-3.25 (m, 2H), 3.20-2.89 (m, 3H), 1.38-1.31 (m, 3H).

### Step 2

### 1-(7,8-dichloro-1-methyl-1,2,4,5-tetrahydro-3H-benzo [d]azepin-3-yl)-2,2,2-trifluoroethan-1-one 37c1

### 1-(8,9-dichloro-1-methyl-1,2,4,5-tetrahydro-3H-benzo [d]azepin-3-yl)-2,2,2-trifluoroethan-1-one 37c2

To a solution of 37b (1 g, 3.43 mmol) in DCE (20 mL) was added NCS (595.10 mg, 4.46 mmol) and trifluoromethanesulfonic Acid (5.15 g, 34.28 mmol). The reaction was stirred at 90° C. for 72 h. Then the mixture was diluted with DCM (100 mL) and washed with brine, the organic solution was dried over Na₂SO₄ and concentrated. The residue was purified with prep-HPLC to give 37c1 (270 mg) and 37c2 (210 mg). 37c1

¹HNMR (400 MHz, DMSO-d₆): δ 7.48-7.42 (m, 2H), 3.81-3.74 (m, 2H), 3.59-3.41 (m, 2H), 3.33-3.30 (m, 1H), 3.12-3.01 (m, 2H), 1.27-1.20 (m, 3H). 37c2

¹HNMR (400 MHz, DMSO-d₆): δ 7.46 (d, 1H), 7.22-7.19 (m, 1H), 4.05-3.47 (m, 5H), 3.30-3.21 (m, 1H), 3.03-2.95 (m, 1H), 1.21-1.17 (m, 3H).

### Step 3

### 7,8-dichloro-1-methyl-2,3,4,5-tetrahydro-1H-benzo[d]azepine 37d

To a solution of 37c1 (100.00 mg, 306.62 umol) in MeOH (5 mL) was added a solution of NaOH (61.32 mg, 1.53 mmol) in water (5 mL). The reaction was stirred at r.t for 2 h. Water (20 mL) was added and the mixture was extracted with EA (20 mL×3), the combined organic layers were washed with brine, dried over Na₂SO₄ and concentrated to give crude 37d (70 mg, 304.17 umol, 99.20% yield).

LCMS: MS m/z (ESI): 230.1 [M+H]⁺.

### Step 4

### (S)-5-cyclopropyl-5-(3-(7,8-dichloro-1-methyl-1,2,4,5-tetrahydro-3H-benzo [d]azepin-3-yl)-3-oxopropyl)imidazolidine-2,4-dione 37

To a solution of 37d (70.00 mg, 304.17 umol) and Int-1 (71.00 mg, 334.59 umol) in DMF (10 mL) was added triethylamine (92.34 mg, 912.52 umol) and HATU (127.22 mg, 334.59 umol). The reaction was stirred at r.t for 2 h. The reaction mixture was diluted with water (50 mL) and the mixture was extracted with EA (50 mL×2). The combined layers were washed with brine, dried over Na₂SO₄ and concentrated. The residue was purified by prep-HPLC to give 37 (40 mg, 94.27 umol, 30.99% yield).

¹HNMR (400 MHz, DMSO-d₆): δ 10.62, 10.61 (s, 1H), 7.76-7.70 (m, 1H), 7.46-7.39 (m, 2H), 3.66-3.32 (m, 4H), 3.24-2.81 (m, 3H), 2.45-2.19 (m, 2H), 1.91-1.76 (m, 2H), 1.24-1.15 (m, 3H), 1.09-1.01 (m, 1H), 0.46-0.27 (m, 3H), 0.13-0.05 (m, 1H).

LCMS: MS m/z (ESI): 424.1 [M+H]⁺.

### Examples 37-1 and 37-2

### (S)-5-cyclopropyl-5-(3-((S)-7,8-dichloro-1-methyl-1,2,4,5-tetrahydro-3H-benzo [d]azepin-3-yl)-3-oxopropyl)imidazolidine-2,4-dione 37-1

### (S)-5-cyclopropyl-5-(3-((R)-7,8-dichloro-1-methyl-1,2,4,5-tetrahydro-3H-benzo [d]azepin-3-yl)-3-oxopropyl)imidazolidine-2,4-dione 37-2

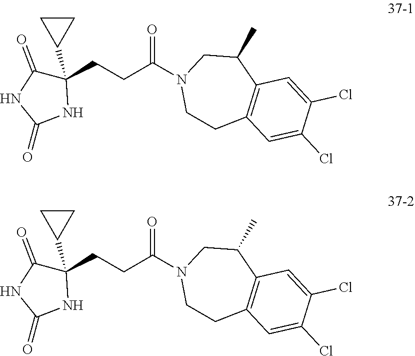 [SMILES: C[C@@H]1CN(C(=O)CC[C@@]2(C3CC3)NC(=O)NC2=O)CCc2cc(Cl)c(Cl)cc21 ; C[C@H]1CN(C(=O)CC[C@@]2(C3CC3)NC(=O)NC2=O)CCc2cc(Cl)c(Cl)cc21]

37 (55 mg) was chiral separated by SFC to give title compounds (15 mg and 17 mg).

### Enantiomer (Shorter Retention Time):

¹H NMR (400 MHz, DMSO-d₆): δ 10.60 (s, 1H), 7.71 (d, 1H), 7.44 (d, 1H), 7.40 (d, 1H), 3.65-3.38 (m, 4H), 3.23-2.81 (m, 3H), 2.41-2.21 (m, 2H), 1.93-1.77 (m, 2H), 1.20 (dd, 3H), 1.10-1.01 (m, 1H), 0.47-0.26 (m, 3H), 0.13-0.04 (m, 1H).

LCMS: m/z (ESI): 424.1 [M+H]⁺.

ChirHPLC (CO₂/EtOH/DEA 60/40/0.04 1.8 ml/min IA, 3 um 3*100(Daicel)): Rt:1.446 min, ee:100%.

### Enantiomer (Longer Retention Time):

¹H NMR (400 MHz, DMSO-d₆): δ 10.61 (br, 1H), 7.72 (d, 1H), 7.44 (d, 1H), 7.40 (d, 1H), 3.66-3.36 (m, 4H), 3.26-2.80 (m, 3H), 2.44-2.17 (m, 2H), 1.93-1.77 (m, 2H), 1.19 (dd, 3H), 1.09-1.02 (m, 1H), 0.48-0.27 (m, 3H), 0.12-0.05 (m, 1H).

LCMS: m/z (ESI): 424.1 [M+H]⁺

ChirHPLC (CO₂/EtOH/DEA 60/40/0.04 1.8 ml/min IA, 3 um 3*100(Daicel)): Rt:2.547 min, ee:99.38%.

### Example 38

### (S)-5-cyclopropyl-5-(3-(8,9-dichloro-1-methyl-1,2,4,5-tetrahydro-3H-benzo [d]azepin-3-yl)-3-oxopropyl)imidazolidine-2,4-dione 38

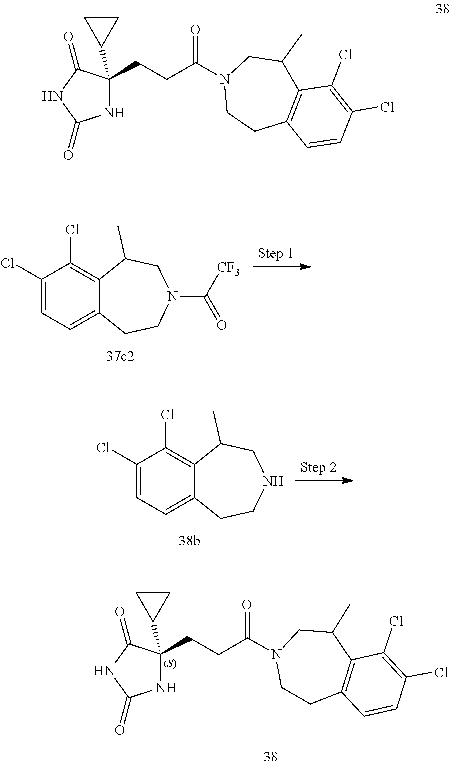

### Step 1

### 8,9-dichloro-1-methyl-2,3,4,5-tetrahydro-1H-benzo[d]azepine 38b

To a solution of 37c2 (100 mg, 306.62 umol) in MeOH (5 mL) was added a solution of NaOH (61.32 mg, 1.53 mmol) in water (5 mL). The reaction was stirred at r.t for 2 h. Water (20 mL) was added and the mixture was extracted with EtOAc (20 mL×3), the combined organic layers were washed with brine, dried over Na₂SO₄ and concentrated to give crude 38b (70 mg, 304.17 umol, 99.20% yield).

LCMS: MS m/z (ESI): 230.1 [M+H]⁺.

### Step 2

### (S)-5-cyclopropyl-5-(3-(8,9-dichloro-1-methyl-1,2,4,5-tetrahydro-3H-benzo [d]azepin-3-yl)-3-oxopropyl)imidazolidine-2,4-dione 38

To a solution of 38b (70 mg, 304.17 umol) and Int-1 (71.00 mg, 334.59 umol) in DMF (10 mL) was added triethylamine (92.34 mg, 912.52 umol) and HATU (127.22 mg, 334.59 umol). The reaction was stirred at r.t for 2 h. The reaction was diluted with water (50 mL) and the mixture was extracted with EtOAc (50 mL×2). The combined organic layers were washed with brine, dried over Na₂SO₄ and concentrated. The residue was purified by prep-HPLC to give 38 (35 mg, 82.48 umol, 27.12% yield).

¹HNMR (400 MHz, DMSO-d₆): δ 10.61, 10.58 (s, 1H), 7.74, 7.65 (s, 1H), 7.42 (d, 1H), 7.16 (t, 1H), 3.90-3.35 (m, 5H), 3.25-3.10 (m, 1H), 2.92-2.81 (m, 1H), 2.45-2.07 (m, 2H), 1.85-1.68 (m, 2H), 1.19-1.13 (m, 3H), 1.07-0.95 (m, 1H), 0.45-0.25 (m, 3H), 0.11-0.02 (m, 1H).

LCMS: MS m/z (ESI): 424.1 [M+H]⁺.

### Examples 38-1 and 38-2

### (S)-5-cyclopropyl-5-(3-((S)-8,9-dichloro-1-methyl-1,2,4,5-tetrahydro-3H-benzo[d]azepin-3-yl)-3-oxopropyl)imidazolidine-2,4-dione 38-1

### (S)-5-cyclopropyl-5-(3-((R)-8,9-dichloro-1-methyl-1,2,4,5-tetrahydro-3H-benzo [d]azepin-3-yl)-3-oxopropyl)imidazolidine-2,4-dione 38-2

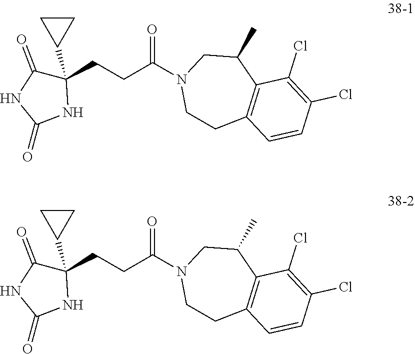 [SMILES: C[C@@H]1CN(C(=O)CC[C@@]2(C3CC3)NC(=O)NC2=O)CCc2ccc(Cl)c(Cl)c21 ; C[C@H]1CN(C(=O)CC[C@@]2(C3CC3)NC(=O)NC2=O)CCc2ccc(Cl)c(Cl)c21]

38 (53 mg) was chiral separated by SFC to give title compounds (14 mg and 13 mg).

### Enantiomer (Shorter Retention Time):

¹H NMR (400 MHz, DMSO-d₆): δ 10.59 (d, 1H), 7.68 (d, 1H), 7.41 (d, 1H), 7.17 (t, 1H), 3.90-3.47 (m, 5H), 3.30-3.10 (m, 1H), 2.92-2.81 (m, 1H), 2.38-2.09 (m, 2H), 1.91-1.66 (m, 2H), 1.16 (dd, 3H), 1.09-0.95 (m, 1H), 0.45-0.24 (m, 3H), 0.11-0.03 (m, 1H).

LCMS: m/z (ESI): 424.1 [M+H]⁺.

ChirHPLC (CO₂/EtOH/DEA 60/40/0.04 1.8 ml/min IA, 3 um 3*100(Daicel)): Rt:1.076 min, ee:100%.

### Enantiomer (Longer Retention Time):

¹H NMR (400 MHz, DMSO-d₆): δ 10.58 (brs, 1H), 7.68 (d, 1H), 7.41 (d, 1H), 7.16 (t, 1H), 3.90-3.37 (m, 5H), 3.27-3.10 (m, 1H), 2.91-2.80 (m, 1H), 2.42-2.07 (m, 2H), 1.99-1.69 (m, 2H), 1.19-1.13 (dd, 3H), 1.09-0.98 (m, 1H), 0.46-0.24 (m, 3H), 0.12-0.02 (m, 1H).

LCMS: m/z (ESI): 424.1 [M+H]⁺.

ChirHPLC (CO₂/EtOH/DEA 60/40/0.04 1.8 ml/min IA, 3 um 3*100(Daicel)): Rt:2.237 min, ee:99.70%.

### Example 40

### (S)-5-(3-(5-chloro-6-(trifluoromethyl)isoindolin-2-yl)-3-oxopropyl)-5-cyclopropylimidazolidine-2,4-dione 40

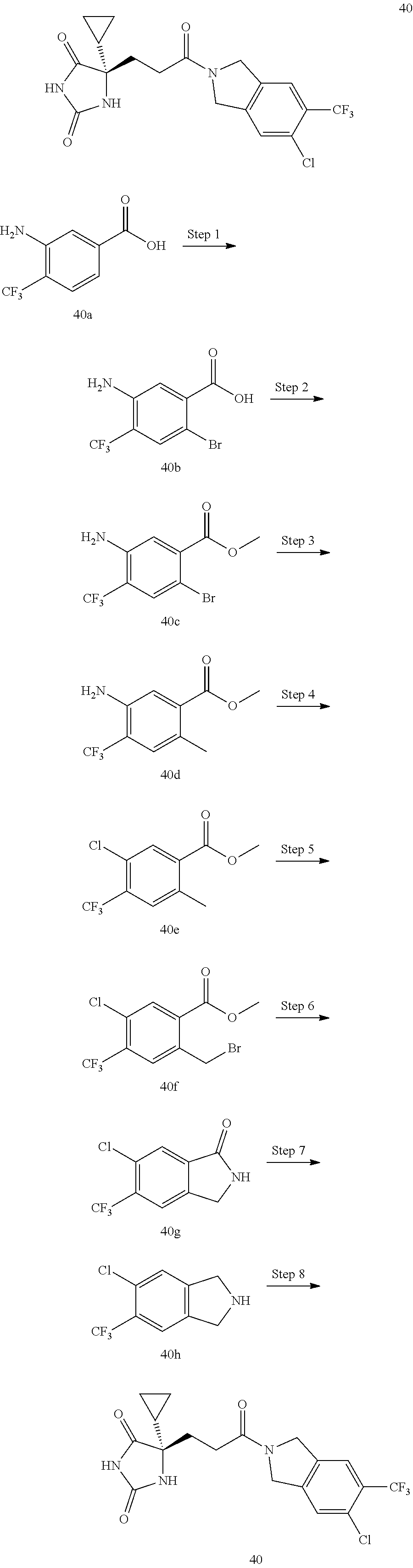

### Step 1

### 5-amino-2-bromo-4-(trifluoromethyl)benzoic acid 40b

To a solution of 3-amino-4-(trifluoromethyl)benzoic acid 40a (1 g, 4.87 mmol) in DMF (20 mL) was added NBS (870 mg, 4.89 mmol). The mixture was stirred at room temperature for 2 hours, the resulting mixture was poured into ice water (20 mL) and the mixture was extracted with EtOAc (20 mL×2). The combined organic phase was washed with water (20 mL), brine (20 mL), dried over Na₂SO₄(s) and filtered. The filtrate was concentrated to afford crude 40b (1 g, 3.52 mmol, 72.22% yield).

### Step 2

### Methyl 5-amino-2-bromo-4-(trifluoromethyl)benzoate 40c

To a solution of 40b (1 g, 3.52 mmol) in MeOH (10 mL) was added H₂SO₄ (18 M, 0.7 mL) dropwise. After the mixture was stirred at 75° C. overnight, the mixture was cooled down to room temperature and poured into ice water (20 mL), the mixture was extracted with EtOAc (50 mL). The organic fraction was dried over Na₂SO₄(s) and filtered. The filtrate was concentrated to afford crude 40c (1 g, 3.36 mmol, 95.29% yield).

¹H NMR (400 MHz, DMSO-d₆): δ 7.57 (s, 1H), 7.21 (s, 1H), 6.11 (brs, 2H), 3.85 (s, 3H).

### Step 3

### Methyl 5-amino-2-methyl-4-(trifluoromethyl)benzoate 40d

To a solution of 40c (1 g, 3.36 mmol) in DMF (10 mL) was added Pd(PPh₃)₄ (430 mg, 372.11 umol), K₃PO₄ (2.2 g, 10.36 mmol) and methylboronic acid (1 g, 16.71 mmol). After the mixture was stirred at 130° C. under N₂ atmosphere overnight, the mixture was cooled down to room temperature and filtered. The filtrate was concentrated, and the residue was purified by silica gel chromatography column to afford 40d (500 mg, 2.14 mmol, 63.91% yield).

LCMS: MS m/z (ESI): 234.1 [M+H]⁺.

### Step 4

### Methyl 5-chloro-2-methyl-4-(trifluoromethyl)benzoate 40e

Concentrated HCl (2 mL) was added to a solution of 40d (2.0 g, 8.58 mmol) in acetone (20 mL), and the mixture was stirred at room temperature for 20 min. The mixture was cooled to −5-0° C., a solution of NaNO₂ (600 mg, 8.70 mmol) in H₂O (2.5 mL) was added dropwise, and the mixture was stirred at an ambient temperature for 30 min. CuCl (849.11 mg, 8.58 mmol) was added portion-wise at 0° C., and the mixture was stirred at room temperature for 2 h. After completion of the reaction, the mixture was poured into 1N HCl (50 mL) and the mixture was extracted with EtOAc. The combined organic layer was washed with water and brine, dried over anhydrous Na₂SO₄, filtered and concentrated in vacuo. The residue was purified by column chromatography to afford 40e (1.3 g, 5.15 mmol, 60.00% yield).

### Step 5

### Methyl 2-bromo-5-chloro-4-(trifluoromethyl)benzoate 40f

To a solution of 40e (1.3 g, 5.15 mmol) in CCl₄ (20 mL) was added NBS (1.10 g, 6.18 mmol) and AIBN (25.35 mg, 154.38 umol), the mixture was heated to 70° C. and stirred overnight. The mixture was colded to room temperature and filtered, the cake was washed with CCl₄, the filtrate was concentrated in vacuo to give crude 40f (1.9 g, 5.73 mmol, 111.37% yield).

### Step 6

### 6-chloro-5-(trifluoromethyl)isoindolin-1-one 40 g

To a solution of 40f (1.9 g, 5.73 mmol) in MeOH (10 mL) was added NH₃/MeOH (20 mL) and the mixture was stirred at room temperature overnight. The reaction mixture was concentrated in vacuo. The residue was purified by column chromatography (hexane: EtOAc=1:1) to afford 40 g (920 mg, 3.91 mmol, 68.14% yield).

LCMS: MS m/z (ESI): 236.0 [M+H]⁺.

### Step 7

### 5-chloro-6-(trifluoromethyl)isoindoline 40 h

To a solution of 40 g (570 mg, 2.42 mmol) in THF (5 mL) was added BH₃/THF (167.36 mg, 12.10 mmol, 15 mL) and the mixture was stirred at 60° C. overnight. The reaction was colded to room temperature and quenched with methanol. The mixture was adjusted to pHI-2 with 1M HCl. Then the mixture was heated to 45° C. and stirred for 30 min. After cooled to rt, the mixture was adjusted to pH 7-8 with 1M NaOH. Water was added and the mixture was extracted with EtOAc. The combined organic layer was washed with water and brine, dried over anhydrous Na₂SO₄, filtered and concentrated in vacuo. The residue was purified prep-TLC (DCM: MeOH=10:1) to give 40 h (10 mg, 45.13 umol, 1.87% yield).

¹HNMR (400 MHz, DMSO-d6): δ 7.78 (s, 1H), 7.65 (s, 1H), 4.16 (br, 2H), 4.14 (br, 2H).

LCMS: MS m/z (ESI): 222.1 [M+H]⁺.

### Step 8

### (S)-5-(3-(5-chloro-6-(trifluoromethyl)isoindolin-2-yl)-3-oxopropyl)-5-cyclopropylimidazolidine-2,4-dione 40

To a solution of 40 h (10 mg, 45.12 umol) in DMF (2 mL) was added TEA (50 uL), Int-1 (10 mg, 47.12 umol), and HATU (17.16 mg, 45.12 umol). The reaction mixture was stirred at room temperature for 3 h. Water was added, the mixture extracted with EtOAc. The combined organic layers were washed with water and brine, dried over anhydrous Na₂SO₄, filtered and concentrated in vacuo. The crude was purified by prep-HPLC to give 40 (5 mg, 12.03 umol, 26.65% yield).

¹H NMR (400 MHz, DMSO-d₆): δ 10.63 (s, 1H), 7.90 (s, 1H), 7.76 (s, 1H), 7.75 (s, 1H), 4.85 (d, 2H), 4.67 (d, 2H), 2.46-2.22 (m, 2H), 2.03-1.98 (m, 2H), 1.15-1.08 (m, 1H), 0.49-0.31 (m, 3H), 0.15-0.08 (m, 1H).

¹⁹F NMR (376.5 MHz, DMSO-d₆): δ-60.86.

LCMS: MS m/z (ESI): 416.4 [M+H]⁺.

### Example 41

### (5S)-5-(3-(5-chloro-1-methyl-6-(trifluoromethyl)isoindolin-2-yl)-3-oxopropyl)-5-cyclopropylimidazolidine-2,4-dione 41

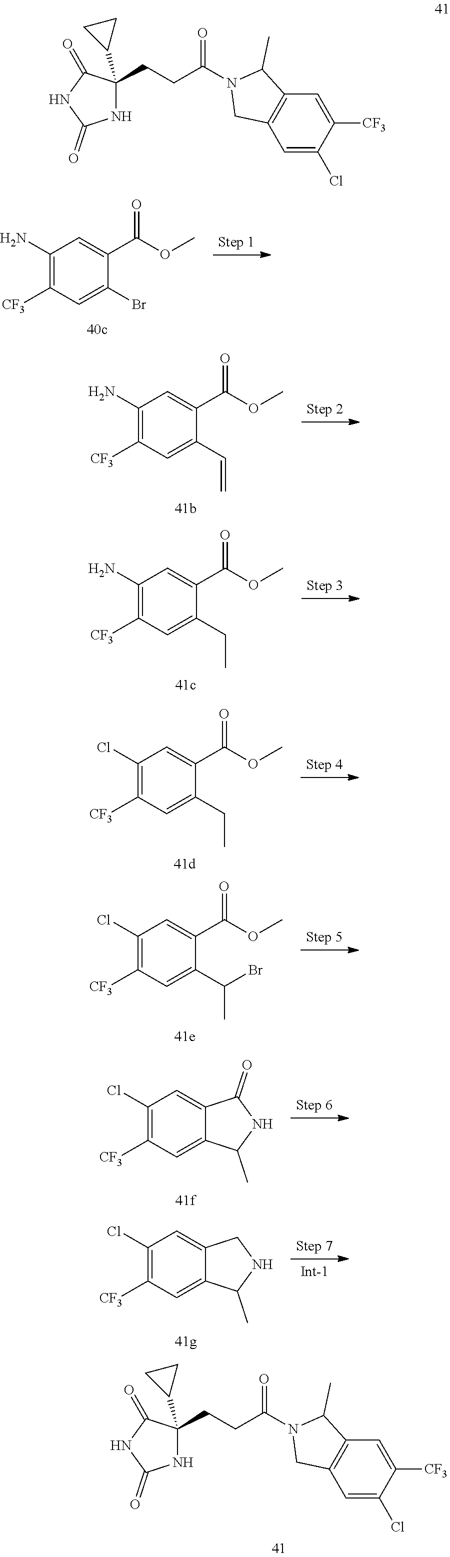

### Step 1

### Methyl 5-amino-4-(trifluoromethyl)-2-vinyl-benzoate 41b

To a solution of 40c (5.45 g, 18.29 mmol) and potassium vinyltrifluoroborate (2.45 g, 18.29 mmol) in dioxane (50 mL) and water (10 mL) was added Pd(dppf)Cl₂ (1.34 g, 1.83 mmol) and K₂CO₃ (6.35 g, 45.71 mmol). The resulting mixture was evacuated and refilled with N₂ for 3 times. The resulting mixture was stirred at 80° C. for 16 h. The mixture was diluted with EtOAc (100 mL), the combined organic phase was washed with brine (100 mL), dried over Na₂SO₄, filtered and concentrated under reduced pressure. The residue was purified by silica gel column chromatography to afford the tittle compound 41b (3.56 g, 14.52 mmol, 79.40% yield).

LCMS: MS m/z (ESI): 246.1 [M+H]⁺.

### Step 2

### Methyl 5-amino-2-ethyl-4-(trifluoromethyl) benzoate 41c

To a solution of 41b (3.56 g, 14.52 mmol) in MeOH (20 mL) was added Pd/C (1.55 g, 1.45 mmol, 285.48 uL, 10% purity). The resulting mixture was evacuated and refilled with H₂. The resulting mixture was stirred at room temperature for 16 h and the LCMS indicated the reaction was finished. The mixture was filtered and the cake was washed with MeOH, the filtrate was concentrated under reduced pressure to afford the tittle compound 41c (3.45 g, 13.96 mmol, 96.12% yield).

LCMS: MS m/z (ESI): 248.1 [M+H]⁺.

### Step 3

### Methyl 5-chloro-2-ethyl-4-(trifluoromethyl) benzoate 41d

To a solution of 41c (3.36 g, 13.59 mmol) in acetone (34 mL) was added HCl (3.36 mL). The resulting mixture was stirred at room temperature for 20 min. After the mixture was cooled to 0° C., a solution of NaNO₂ (1.88 g, 27.18 mmol) in water (5 mL) was added. Then CuCl (1.48 g, 14.95 mmol) was added in small portions at 0° C. The resulting mixture was stirred at room temperature for 1 h. The mixture was poured into 1M HCl (60 mL), the aqueous phase was extracted with EtOAc (100 mL×3), the combined organic phases were washed with brine (100 mL), dried over Na₂SO₄, filtered and concentrated under reduced pressure. The residue was purified by silica gel column chromatography (eluting with hexane/EtOAc=50/1) to afford the title compound 41d (2.23 g, 8.36 mmol, 61.53% yield).

¹H NMR (400 MHz, DMSO-d₆): δ 7.99 (s, 1H), 7.87 (s, 1H), 3.88 (s, 3H), 2.92 (q, 2H), 1.17 (t, 3H).

### Step 4

### Methyl 2-(1-bromoethyl)-5-chloro-4-(trifluoromethyl) benzoate 41e

To a solution of 41d (2.23 g, 8.36 mmol) in CCl₄ (35 mL) was added AIBN (412.00 mg, 2.51 mmol) and NBS (1.64 g, 9.20 mmol). The resulting mixture was stirred at 80° C. for 16 h. The mixture was filtered. The solid was washed with DCM and the filtrate was concentrated in vacuo to afford the crude tittle compound 41e (2.5 g, 7.24 mmol, 86.51% yield).

¹H NMR (400 MHz, DMSO-d₆): δ 8.17 (s, 1H), 8.04 (s, 1H), 6.08 (q, 1H), 3.92 (s, 3H), 2.05 (d, 3H).

### Step 5

### 6-chloro-3-methyl-5-(trifluoromethyl) isoindolin-1-one 41f

To a solution of 41e (2.5 g, 7.24 mmol) in MeOH (10 mL) was added NH₃/MeOH (7 M, 30 mL). The resulting mixture was stirred at room temperature for 16 h. The mixture was purified by prep-HPLC to afford the tittle compound 41f (1.18 g, 4.73 mmol, 65.34% yield).

¹H NMR (400 MHz, DMSO-d₆): δ 9.11 (brs, 1H), 8.20 (s, 1H), 7.91 (s, 1H), 4.71 (q, 1H), 1.42 (d, 3H).

¹⁹F NMR (376.5 MHz, DMSO-d₆): δ-60.99.

LCMS: MS m/z (ESI): 250.0 [M+H]⁺.

### Step 6

### 5-chloro-1-methyl-6-(trifluoromethyl) isoindoline 41 g

To a solution of 41f (730 mg, 2.92 mmol) in THF (5 mL) was added BH₃/THF (2 M, 30.93 mL). The resulting mixture was stirred at 60° C. for 16 h. The mixture was quenched with MeOH (5 mL) and HCl (4 M, 5 mL), the mixture was stirred at 60° C. for 3 h. The mixture was basified with aq. NaOH and extracted with EtOAc (50 mL×3), the combined organic phase was washed with brine (50 mL), dried over Na₂SO₄, filtered and concentrated under reduced pressure. The residue was purified by silica gel column chromatography (eluting with DCM/MeOH=20/1) to afford the tittle compound 41g (300 mg, 1.27 mmol, 43.53% yield).

LCMS: MS m/z (ESI): 236.1 [M+H]⁺.

### Step 7

### (5S)-5-(3-(5-chloro-1-methyl-6-(trifluoromethyl)isoindolin-2-yl)-3-oxopropyl)-5-cyclopropylimidazolidine-2,4-dione 41

To a solution of Int-1 (54.03 mg, 254.63 umol) in DMF (4 mL) was added TEA (53.68 mg, 530.49 umol), and then 41 g (50 mg, 212.19 umol) and HATU (96.82 mg, 254.63 umol). The resulting mixture was stirred at room temperature for 2 h. The mixture was purified by prep-HPLC to afford the tittle compound 41 (25 mg, 57.89 umol, 27.28% yield).

¹H NMR (400 MHz, DMSO-d₆): δ 10.63 (s, 1H), 7.88 (s, 1H), 7.79-7.70 (m, 2H), 5.22-5.18 (m, 1H), 4.91-4.49 (m, 2H), 2.44-2.19 (m, 2H), 2.02-1.97 (m, 2H), 1.42 (d, 3H), 1.14-1.08 (m, 1H), 0.46-0.31 (m, 3H), 0.14-0.08 (m, 1H).

¹⁹F NMR (376.5 MHz, DMSO-d₆): δ-60.74.

LCMS: MS m/z (ESI): 430.0 [M+H]⁺.

### Examples 41-1 and 41-2

### (S)-5-(3-((S)-5-chloro-1-methyl-6-(trifluoromethyl)isoindolin-2-yl)-3-oxopropyl)-5-cyclopropylimidazolidine-2,4-dione 41-1

### (S)-5-(3-((R)-5-chloro-1-methyl-6-(trifluoromethyl)isoindolin-2-yl)-3-oxopropyl)-5-cyclopropylimidazolidine-2,4-dione 41-2

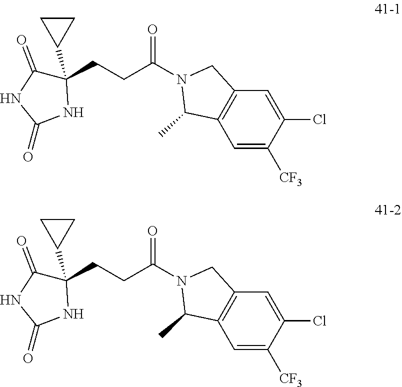 [SMILES: C[C@H]1c2cc(C(F)(F)F)c(Cl)cc2CN1C(=O)CC[C@@]1(C2CC2)NC(=O)NC1=O ; C[C@@H]1c2cc(C(F)(F)F)c(Cl)cc2CN1C(=O)CC[C@@]1(C2CC2)NC(=O)NC1=O]

The two enantiomers of 41 (60 mg) was separated by chiral SFC (DAICELCHIRALCEL IG, 250*25 mm 10 m) to give title compound as single enantiomers (20 mg and 18 mg).

### Enantiomer (Shorter Retention Time):

¹HNMR (400 MHz, DMSO-d₆): δ 10.63 (s, 1H), 7.92-7.88 (m, 1H), 7.80-7.71 (m, 2H), 5.25-5.18 (m, 1H), 4.90-4.54 (m, 2H), 2.37-2.28 (m, 2H), 2.04-1.97 (m, 2H), 1.44 (dd, 3H), 1.15-1.08 (m, 1H), 0.46-0.32 (m, 3H), 0.14-0.09 (m, 1H).

¹⁹F NMR (376.5 MHz, DMSO-d₆): δ-60.75.

LCMS: MS m/z (ESI): 430.1 [M+H]⁺.

Chiral HPLC (CO₂/MeOH/DEA 60/40/0.04 1.8 ml/min IG,3 um,3*100(Daicel)): Rt: 1.171 min, ee: 100%.

### Enantiomer (Longer Retention Time):

¹HNMR (400 MHz, DMSO-d₆): δ 10.64 (s, 1H), 7.92-7.88 (m, 1H), 7.80-7.73 (m, 2H), 5.21-5.19 (m, 1H), 4.89-4.80 (m, 2H), 2.46-2.24 (m, 2H), 2.01-1.97 (m, 2H), 1.44 (dd, 3H), 1.13-1.08 (m, 1H), 0.45-0.32 (m, 3H), 0.13-0.11 (m, 1H).

¹⁹FNMR (376.5 MHz, DMSO-d₆): δ-60.74.

LCMS: MS m/z (ESI): 430.1 [M+H]⁺.

Chiral HPLC (CO₂/MeOH/DEA 60/40/0.04 1.8 ml/min IG,3 um,3*100(Daicel)): Rt: 1.982 min, ee: 100%.

### Example 42

### 5-cyclopropyl-5-(3-(5,6-dichloroisoindolin-2-yl)-2-(hydroxymethyl)-3-oxopropyl)imidazolidine-2,4-dione 42

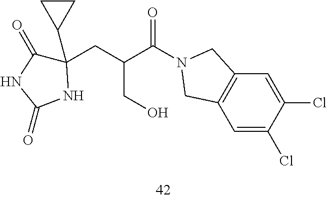 [SMILES: O=C1NC(=O)C(CC(CO)C(=O)N2Cc3cc(Cl)c(Cl)cc3C2)(C2CC2)N1]

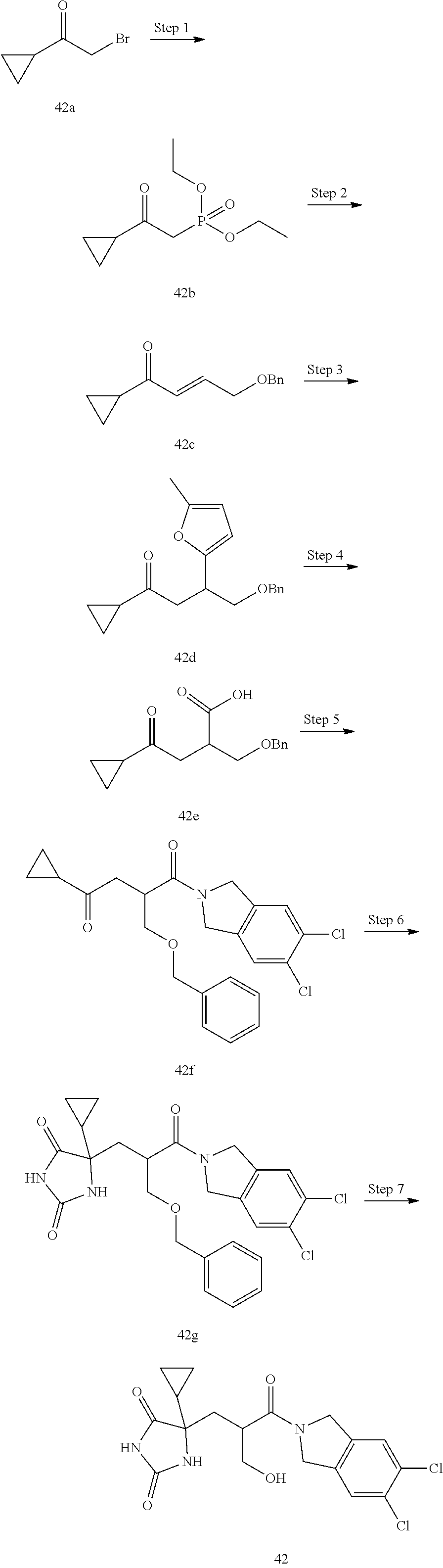

### Step 1

### Diethyl (2-cyclopropyl-2-oxoethyl)phosphonate 42b

To a mixture of triethyl phosphite (5 g, 30.09 mmol) in acetonitrile (150 mL) was added sodium iodide (460 mg, 3.07 mmol). Then 2-bromo-1-cyclopropylethanone 42a (25 g, 153.36 mmol) was added. The reaction was stirred at r.t for 18 h. Water (100 mL) was added and the mixture was extracted with EtOAc (100 mL×2), the organic solution was washed with brine, dried over Na₂SO₄ and concentrated. The residue was purified with silica gel chromatography (hexane: EtOAc=1:1) to give 42b (5.4 g, 79.95% yield).

¹HNMR (400 MHz, CDCl₃): δ 4.18-4.13 (m, 4H), 3.24 (s, 1H), 3.18 (s, 1H), 2.23-2.16 (m, 1H), 1.34 (t, 6H), 1.13-1.10 (m, 2H), 0.99-0.96 (m, 2H).

### Step 2

### (E)-4-(benzyloxy)-1-cyclopropylbut-2-en-1-one 42c

To a mixture of 42b (5.4 g, 24.52 mmol) in acetonitrile (90 mL) was added LiCl (987.55 mg, 23.30 mmol). Then 2-benzyloxyacetaldehyde (3.13 g, 20.84 mmol) was added. The reaction was stirred at r.t for 18 h. Water (100 mL) was added and the mixture was extracted with EtOAc (100 mL×2), the organic solution was washed with brine, dried over Na₂SO₄ and concentrted. The residue was purified with silica gel chromatography (hexane:EtOAc=1:1) to give 42c (3.4 g, 64.11% yield).

¹HNMR (400 MHz, CDCl₃): δ 7.37-7.28 (m, 5H), 6.92-6.86 (dt, 1H), 6.53-6.48 (dt, 1H), 4.58 (s, 2H), 4.22 (dd, 2H), 2.17-2.10 (m, 1H), 1.12-1.07 (m, 2H), 0.95-0.90 (m, 2H).

### Step 3

### 4-(benzyloxy)-1-cyclopropyl-3-(5-methylfuran-2-yl)butan-1-one 42d

To a solution of 42c (3.4 g, 15.72 mmol) in MeOH (75 mL) was added 2-methylfuran (2.58 g, 31.44 mmol) and palladium chloride (279 mg, 1.57 mmol). The reaction was stirred at r.t for 18 h. The solution was filtered, the filtrate was concentrated. The residue was purified by silica gel chromatography (hexane: EtOAc=10:1) to give 42d (2.6 g, 55.43% yield).

¹HNMR (400 MHz, CDCl₃): δ 7.33-7.25 (m, 5H), 5.93 (d, 1H), 5.85-5.83 (m, 1H), 4.49 (s, 2H), 3.69-3.55 (m, 3H), 3.02-2.85 (m, 2H), 2.23 (d, 3H), 1.94-1.87 (m 1H), 1.00-0.95 (m, 2H), 0.84-0.79 (m, 2H).

### Step 4

### 2-((benzyloxy)methyl)-4-cyclopropyl-4-oxobutanoic acid 42e

To a solution of 42d (1 g, 3.35 mmol) in hexane (6 mL), ethyl acetate (18 mL) and water (24 mL) was added, followed by sodium periodate (5.02 g, 23.46 mmol). The reaction was stirred at room temperature for 10 min, ruthenium(III) chloride (695 mg, 3.35 mmol) was added. The reaction was stirred at room temperature for 1 h. The mixture was filtered, the filtrate was concentrated. The residue was purified by silica gel chromatography (DCM:MeOH=20:1) to give 42e (810 mg, 92.14% yield).

LCMS: m/z (ESI): 261.1 [M−1]⁻.

### Step 5

### 2-((benzyloxy)methyl)-4-cyclopropyl-1-(5,6-dichloroisoindolin-2-yl)butane-1,4-dione 42f

To a solution of 42e (200 mg, 762.48 umol), 20c (174 mg, 914.98 umol) and HATU (435 mg, 1.14 mmol) in DMF (3 mL) was added DIEA (295 mg, 2.29 mmol), the mixture was stirred at room temperature for 2 h. The mixture was diluted with water and extracted with EtOAc, the organic solution was concentrated and the residue was purified by silica gel chromatagraphy (hexane:EtOAc=1:1) to give the target 42f (180 mg, 414.41 umol, 54.35% yield).

LCMS: MS m/z (ESI): 432.4 [M+H]⁺.

### Step 6

### 5-(2-((benzyloxy)methyl)-3-(5,6-dichloroisoindolin-2-yl)-3-oxopropyl)-5-cyclopropylimidazolidine-2,4-dione 42 g

The mixture of 42f (120 mg, 276.27 umol), (NH₄)₂CO₃ (27 mg, 276.27 umol) and NaCN (15 mg, 276.27 umol) in MeOH (5 mL) and H₂O (1 mL) was sealed and stirred at 90° C. under Ar for 16 h. The mixture was concentrated and the residue was purified by prep-HPLC to give the target 42 g (20 mg, 39.65 umol, 14.35% yield).

¹H NMR (400 MHz, Methanol-d₄): δ 7.51 (s, 1H), 7.44 (s, 1H), 7.20 (brs, 5H), 5.00-4.96 (m, 1H), 4.82-4.73 (m, 2H), 4.64-4.59 (m, 1H), 4.50-4.42 (m, 2H), 3.65-3.59 (m, 2H), 3.30-3.20 (m, 1H), 2.47 (dd, 1H), 1.83 (dd, 1H), 1.18-1.14 (m, 1H), 0.54-0.51 (m, 1H), 0.42-0.38 (m, 1H), 0.33-0.29 (m, 2H).

LCMS: MS m/z (ESI): 502.5 [M+H]⁺.

### Step 7

### 5-cyclopropyl-5-(3-(5,6-dichloroisoindolin-2-yl)-2-(hydroxymethyl)-3-oxopropyl)imidazolidine-2,4-dione 42

To a solution of 42 g (15 mg, 29.74 umol) in EtOAc (3 mL) was added Pd/C (2 mg, 11.30 umol), then the mixture was stirred at room temperature for 15 min under H₂. The mixture was filtered, and the filtrate was concentrated, the residue was purified by prep-HPLC to give the target 42 (8.0 mg, 19.31 umol, 64.94% yield).

¹H NMR (400 MHz, Methanol-d₄): δ 7.51 (s, 1H), 7.48 (s, 1H), 5.04-4.99 (m, 1H), 4.86-4.71 (m, 2H), 4.67-4.57 (m, 1H), 3.67-3.57 (m, 2H), 2.87-2.83 (m, 1H), 2.30 (dd, 1H), 2.03-1.97 (m, 1H), 1.19-1.16 (m, 1H), 0.52-0.50 (m, 1H), 0.42-0.35 (m, 2H), 0.29-0.26 (m, 1H).

LCMS: MS m/z (ESI): 412.0 [M+H]⁺.

### Example 43

### 5-(3-oxo-3-(5-(trifluoromethyl)isoindolin-2-yl)propyl)-5-(thiazol-2-yl)imidazolidine-2,4-dione 43

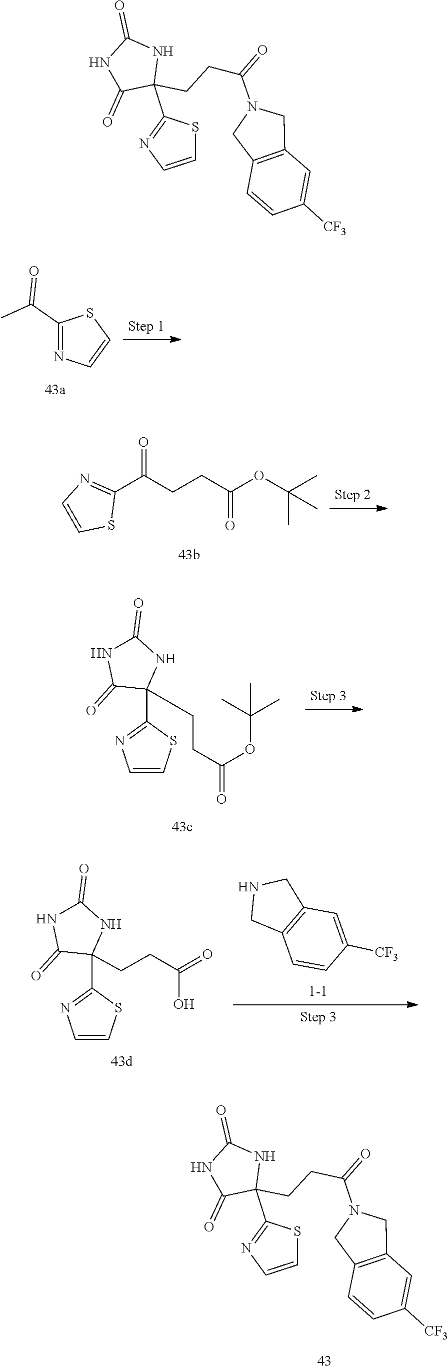

### Step 1

### Tert-butyl 4-oxo-4-(thiazol-2-yl)butanoate 43b

To a solution of 1-(thiazol-2-yl)ethanone 43a (21.88 g, 172.06 mmol) in THF (175 mL) was added LiHMDS (34.55 g, 206.48 mmol) and 1,3-dimethyltetrahydropyrimidin-2 (1H)-one (44 mL) at −78° C., then tert-butyl 2-bromoacetate (40.27 g, 206.47 mmol) was added at −78° C.

The resulting mixture was stirred at −78° C. for 16 h, and then the mixture was diluted with EtOAc (200 mL) and H₂O (100 mL), the organic phase was washed with water (100 mL×2), brine (100 mL), dried over Na₂SO₄, filtered and concentrated under reduced pressure. The residue was purified by silica gel column chromatography to afford 43b (16 g, 66.31 mmol, 38.54% yield).

LCMS: MS m/z (ESI): 242.1 [M+H]⁺.

### Step 2

### tert-butyl 3-(2,5-dioxo-4-(thiazol-2-yl)imidazolidin-4-yl)propanoate 43c

A mixture of 43b (5.0 g, 20.72 mmol), (NH₄)₂CO₃ (16.91 g, 176.12 mmol), NaCN (2.75 g, 51.80 mmol), EtOH (25 mL) and H₂O (25 mL) was heated in an autoclave at 85° C. for 18 hours.

The resulting mixture was diluted with water (60 mL), and extracted with EtOAc (200 mL×5). The organic layers were combined, washed with brine (200 mL), dried over Na₂SO₄ and filtered. The filtrate was concentrated under reduced pressure. The residue was purified by column chromatography to afford 43c (650 mg, 2.09 mmol, 10.08% yield).

LCMS: MS m/z (ESI): 312.1 [M+H]⁺.

### Step 3

### 3-(2,5-dioxo-4-(thiazol-2-yl)imidazolidin-4-yl)propanoic acid 43d

To a solution of 43c (650 mg, 2.09 mmol) in DCM (5 mL) was added HCl/dioxane (10 mL) drop-wise. The reaction mixture was stirred at room temperature for 18 hours. Then the reaction mixture was concentrated under reduced pressure to afford crude 43d (600 mg, 2.35 mmol, 112.60% yield).

LCMS: MS m/z (ESI): 256.0[M+H]⁺.

### Step 4

### 5-(3-oxo-3-(5-(trifluoromethyl)isoindolin-2-yl)propyl)-5-(thiazol-2-yl)imidazolidine-2,4-dione 43

A mixture of 43d (100 mg, 391.77 umol), 1-1 hydrochloride (87.61 mg, 391.77 umol), Et₃N (118.71 mg, 1.18 mmol), HATU (148.96 mg, 391.77 umol) and DMF (10 mL) was stirred at room temperature for 18 hours. Then the reaction mixture was purified by prep-HPLC to afford 43 (20 mg, 47.13 umol, 12.03% yield).

¹H NMR (400 MHz, DMSO-d₆): δ 11.10 (br, 1H), 8.47-8.44 (m, 1H), 7.82 (d, 1H), 7.76-7.73 (m, 2H), 7.66 (d, 1H), 7.59-7.55 (m, 1H), 4.83 (d, 1H), 4.69 (brs, 2H), 2.46-2.38 (m, 4H).

LCMS: MS m/z (ESI): 425.2 [M+H]⁺.

### Example 44

### (S)-5-cyclopropyl-5-(3-(7-fluoro-1,2,4,5-tetrahydro-3H-benzo[d]azepin-3-yl)-3-oxopropyl)imidazolidine-2,4-dione

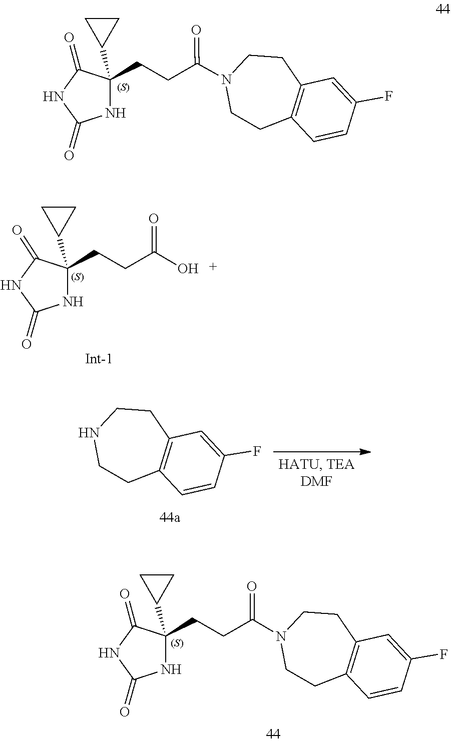

To a mixture of 7-fluoro-2,3,4,5-tetrahydro-1H-benzo[d]azepine 44a (23 mg, 0.139 mmol, purchased from Acme Bioscience) in DMF (5 mL) was added Triethylamine (70 mg, 0.51 mmol), Int-1 (30 mg, 0.141 mmol) and HATU (57 mg, 0.151 mmol). The reaction was stirred at room temperature for 18 h. Water (3 mL) was added and the mixture was extracted with EtOAc (20 mL×2). The combined layer was washed with brine, dried over Na₂SO₄ and concentrated. The residue was purified with prep-HPLC to give title compound 44 (25 mg, 69.5 umol, 48% yield).

¹H NMR (400 MHz, Methanol-d₄): δ 7.16 (dt, 1H), 6.98-6.81 (m, 2H), 3.80-3.55 (m, 4H), 3.37 (s, 1H), 3.03-2.90 (m, 4H), 2.19-2.03 (m, 4H), 1.22 (m, 1H), 0.59 (m, 1H), 0.40 (m, 3H).

LCMS: MS m/z (ESI): 360 [M+H]⁺.

### Example 45

### (S)-5-cyclopropyl-5-(3-oxo-3-(7-(trifluoromethyl)-1,2,4,5-tetrahydro-3H-benzo[d]azepin-3-yl)propyl)imidazolidine-2,4-dione

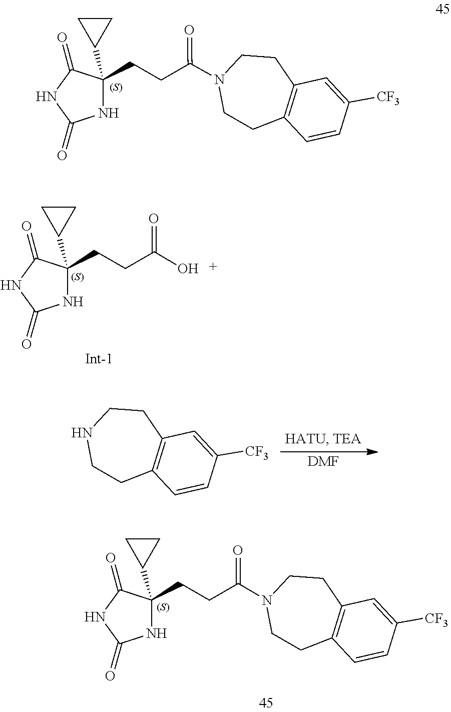

To a mixture of 7-(trifluoromethyl)-2,3,4,5-tetrahydro-1H-benzo[d]azepine (23 mg, 0.139 mmol) in DMF (5 mL) was added Triethylamine (70 mg, 0.51 mmol), Int-1 (30 mg, 0.141 mmol) and HATU (57 mg, 0.151 mmol). The reaction was stirred at r.t for 18 h. Water (3 mL) added and the mixture was extracted with EtOAc (20 mL×2). The combined organic layers were washed with brine, dried over Na₂SO₄ and concentrated. The residue was purified with prep-HPLC to give the title compound (18 mg, 31% yield).

¹H NMR (400 MHz, CDCl₃): δ 7.31 (q, 2H), 7.15 (t, 1H), 3.65 (m, 2H), 3.59-3.45 (m, 2H), 2.94-2.79 (m, 4H), 2.45 (hept, 2H), 2.23 (dt, 1H), 2.14 (dt, 7.4 Hz, 1H), 1.12 (td, 1H), 0.40-0.26 (m, 4H).

LCMS: MS m/z (ESI): 410 [M+H]⁺.

### Example 48

### 5-(3-(5-chloro-6-(trifluoromethyl)isoindolin-2-yl)-3-oxopropyl)-5-(pyridin-3-yl)imidazolidine-2,4-dione 48

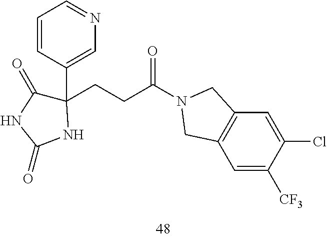 [SMILES: O=C1NC(=O)C(CCC(=O)N2Cc3cc(Cl)c(C(F)(F)F)cc3C2)(c2cccnc2)N1]

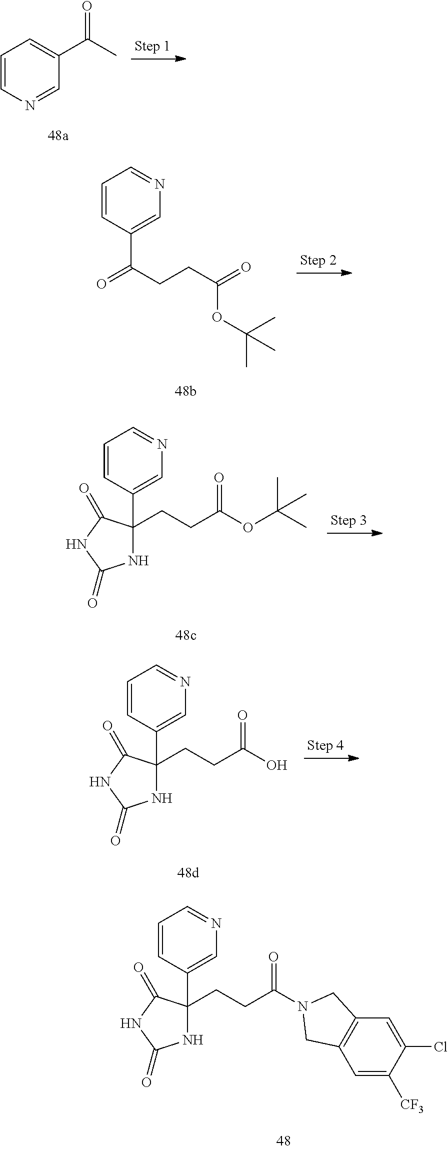

### Step 1

### Tert-butyl 4-oxo-4-(pyridin-3-yl)butanoate 48b

To a solution of 1-(pyridin-3-yl)ethan-1-one 48a (4.97 g, 41.01 mmol) in THF (50 mL) was added NaHMDS (7.52 g, 41.01 mmol) dropwise at −70° C. The resulting mxiture was stirred at this temperature for 30 min before tert-butyl 2-bromoacetate (8.0 g, 41.01 mmol) was added dropwsie. After addition, the reaction mixture was stirred at −20° C. for 1.0 hour, then warmed to room temperature and stirred for 18 hours. The resulting mixture was cooled to 0° C., quenched with aq. NaHCO₃ (20 mL). The whole solution was extracted with EtOAc (30 mL×4). The organic layers were combined, dried over Na₂SO₄ and filtered. The filtrate was concentrated under reduced pressure. The residue was purified by silica gel chromatography (EtOAc/hexane=1/20 to 1/4) to afford 48b (800 mg, 3.40 mmol, 8.29% yield).

LCMS: MS m/z (ESI): 236.1 [M+H]⁺.

### Step 2

### Tert-butyl 3-(2,5-dioxo-4-(pyridin-3-yl)imidazolidin-4-yl)propanoate 48c

To the solution of 48b (500 mg, 2.13 mmol) in H₂O (8 mL) and EtOH (10 mL) was added NaCN (282 mg, 5.31 mmol), (NH₄)₂CO₃ (1.63 g, 17.00 mmol). The reaction was stirred at 85° C. overnight. LCMS showed product produced. Then the reaction mixture was diluted with water and extracted with EtOAc (10 mL×2). The organic solution was concentrated, and the residue was purified by silica gel chromatography (EtOAc/hexane=1/2 to 1/1) to give 48c (250 mg, 818.79 umol, 38.53% yield).

¹H NMR (400 MHz, DMSO-d₆): δ 11.21 (s, 1H), 9.07 (s, 1H), 8.92 (s, 1H), 8.90 (s, 1H), 8.45 (d, 1H), 7.90 (s, 1H), 2.57-2.30 (m, 3H), 2.19-2.15 (m, 1H), 1.41 (s, 9H).

LCMS: MS m/z (ESI): 306.1 [M+H]⁺.

### Step 3

### 3-(2,5-dioxo-4-(pyridin-3-yl)imidazolidin-4-yl)propanoic acid 48d

The mixture of 48c (250 mg, 818.79 umol) in HCl/1,4-dioxane (2 mL, 2N) was stirred at room temperature for 1 h. LCMS showed that the product produced. The mixture was concentrated to give 48d (220 mg, 882.75 umol, 107.81% yield).

LCMS: MS m/z (ESI): 250.1 [M+H]⁺.

### Step 4

### 5-(3-(5-chloro-6-(trifluoromethyl)isoindolin-2-yl)-3-oxopropyl)-5-(pyridin-3-yl)imidazolidine-2,4-dione 48

To the solution of 48d (100 mg, 401 umol) in THF (5 mL) was added HATU (232 mg, 609.19 umol), DIEA (63 mg, 487.35 umol) and 40 h (90 mg, 406.13 umol). The reaction was stirred at room temperature overnight. LCMS showed that the product produced. The mixture was purified by prep-HPLC to give title compound 48 (5.55 mg, 12.26 umol, 3.02% yield).

¹H NMR (400 MHz, DMSO-d₆): δ 11.01 (brs, 1H), 8.87 (d, 1.6 Hz, 1H), 8.72 (d, 1H), 8.56 (dd, 1H), 7.94-7.88 (m, 2H), 7.55 (d, 1H), 7.46 (dd, 1H), 4.81-4.77 (m, 2H), 4.67-4.63 (m, 2H), 2.42-2.23 (m, 4H).

LCMS: MS m/z (ESI): 453.4 [M+H]⁺.

### Examples 49-1 and 49-2

### (5R)-5-cyclopropyl-5-(3-methyl-2-(5-(trifluoromethyl)isoindoline-2-carbonyl)butyl)imidazolidine-2,4-dione 49-1

### (5S)-5-cyclopropyl-5-(3-methyl-2-(5-(trifluoromethyl)isoindoline-2-carbonyl)butyl)imidazolidine-2,4-dione 49-2

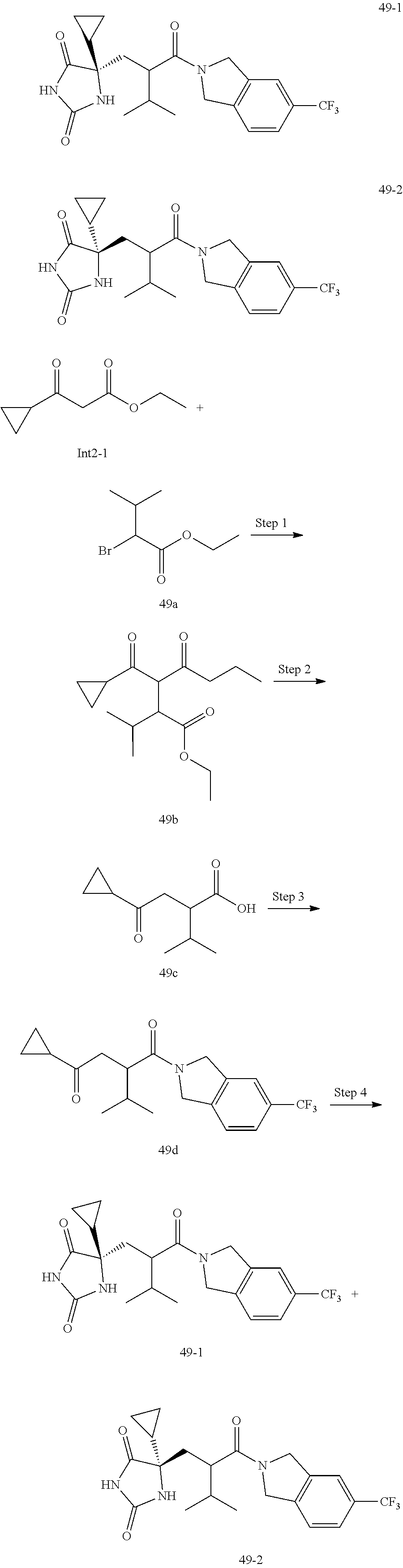

### Step 1

### Diethyl 2-(cyclopropanecarbonyl)-3-isopropyl-butanedioate 49b

To a solution of Int2-1 (3.59 g, 22.99 mmol) and ethyl 2-bromo-3-methylbutanoate 49a (5 g, 23.91 mmol) in methyl ethyl ketone (60 mL) were added K₂CO₃ (6.36 g, 45.99 mmol) and NaI (3.45 g, 22.99 mmol). The resulting mixture was stirred at 110° C. for 48 h. The mixture was poured into water (100 mL), the aqueous phase was extracted with EtOAc (80 mL×3), the combined organic phase was washed with brine (100 mL), dried over Na₂SO₄, filtered and concentrated under reduced pressure. The residue was purified by silica gel column chromatography to afford the tittle compound 49b (4.29 g, 15.09 mmol, 65.61% yield).

### Step 2

### 4-cyclopropyl-2-isopropyl-4-oxo-butanoic acid 49c

To a solution of 49b (2.29 g, 8.05 mmol) in THF (8 mL) and water (8 mL) was added LiGH (482.21 mg, 20.13 mmol). The resulting mixture was stirred at room temperature for 16 h. To the mixture was added HCl (1N, 10 mL), the resulting mixture was stirred at 60° C. for 2 h. Then the mixture was adjusted to pH=13 with the addition of NaOH (aq) and the layers were separated. The combined aq. phase was adjusted to pH-7 with 1 M HCl (10 mL), the mixture was then extracted with DCM (50 mL×3), the organic solution was dried over Na₂SO₄, filtered and concentrated under reduced pressure to afford the crude tittle compound 49c (300 mg, 1.63 mmol, 20.22% yield).

### Step 3

### 4-cyclopropyl-2-isopropyl-1-[5-(trifluoromethyl)-1, 3, 3a, 7a-tetrahydroisoindol-2-yl] butane-1,4-dione 49d

To a solution of 49c (270 mg, 1.52 mmol) in DMF (8 mL) was added TEA (753.42 mg, 7.45 mmol), 5-(trifluoromethyl)isoindoline 1-1 (333 mg, 1.49 mmol) and HATU (679.45 mg, 1.79 mmol). The resulting mixture was stirred at room temperature for 2 h. The mixture was poured into water (80 mL), the aqueous phase was extracted with EtOAc (80 mL×3), the combined organic phase was washed with brine (100 mL), dried over Na₂SO₄, filtered and concentrated under reduced pressure. The residue was purified by silica gel column chromatography (eluting with hexane/EtOAc=3/1) to afford the tittle compound 49d (340 mg, 956.68 umol, 64.25% yield).

LCMS: MS m/z (ESI): 354.1 [M+H]⁺.

### Step 4

### (5R)-5-cyclopropyl-5-(3-methyl-2-(5-(trifluoromethyl)isoindoline-2-carbonyl)butyl)imidazolidine-2,4-dione 49-1

### (5S)-5-cyclopropyl-5-(3-methyl-2-(5-(trifluoromethyl)isoindoline-2-carbonyl)butyl)imidazolidine-2,4-dione 49-2

To a solution of 49d (340 mg, 956.69 umol) in MeOH (5 mL) was added (NH₄)₂CO₃ (735.39 mg, 7.65 mmol), water (5 mL) and NaCN (126.86 mg, 2.39 mmol). The resulting mixture was stirred at 80′° C. for 16 h. The reaction mixture was poured into water (100 mL), the mixture was extracted with EtOAc (80 mL×3), the combined organic phases were washed with brine (100 mL), dried over Na₂SO₄, filtered and concentrated under reduced pressure. The residue was purified with prep-HPLC to afford the tittle compound 49-1 (40 mg, 94.02 umol, 9.83% yield) and 49-2 (250 mg, 587.62 umol, 61.42% yield). 49-1:

¹H NMR (400 MHz, DMSO-d₆): δ 10.46 (s, 1H), 7.77-7.72 (m, 2H), 7.66 (d, 1H), 7.60-7.56 (m, 1H), 4.91-4.88 (m, 2H), 4.72-4.69 (m, 1H), 4.63-4.58 (m, 1H), 2.58-2.50 (m, 1H), 2.42-2.35 (m, 1H), 1.78-1.73 (m, 2H), 1.07-1.03 (m, 1H), 0.92 (dd, 6H), 0.41-0.27 (m, 3H), 0.10-0.07 (m, 1H).

¹⁹F NMR (376.5 MHz, DMSO-d₆): δ-60.57.

HPLC: SunFire C18 5 um 4.6*150 mm, 0.03% TFA CH3CN/H2O, Rt 7.478 min.

LCMS: MS m/z (ESI): 424.1 [M+H]⁺. 49-2:

¹H NMR (400 MHz, DMSO-d₆): δ 10.63 (s, 1H), 7.74 (d, 1H), 7.68-7.65 (m, 1H), 7.60-7.54 (m, 2H), 4.90-4.85 (m, 2H), 4.74-4.71 (m, 2H), 2.33-2.28 (m, 2H), 1.88-1.75 (m, 2H), 1.07-1.03 (m, 1H), 0.92-0.89 (m, 6H), 0.42-0.39 (s, 1H), 0.29-0.25 (m, 2H), 0.01-0.00 (m, 1H).

¹⁹F NMR (376.5 MHz, DMSO-d₆): δ-60.57.

HPLC: SunFire C18 5 um 4.6*150 mm, 0.03% TFA CH3CN/H2O, Rt 8.141 min.

LCMS: MS m/z (ESI): 424.3 [M+H]⁺.

### Examples 49-2a and 49-2b

### (5S)-5-cyclopropyl-5-[(2R)-3-methyl-2-[5-(trifluoromethyl)-1, 3, 3a, 7a-tetrahydroisoindole-2-carbonyl] butyl] imidazolidine-2,4-dione 49-2a

### (5R)-5-cyclopropyl-5-[(2S)-3-methyl-2-[5-(trifluoromethyl)-1, 3, 3a, 7a-tetrahydroisoindole-2-carbonyl] butyl] imidazolidine-2,4-dione 49-2b

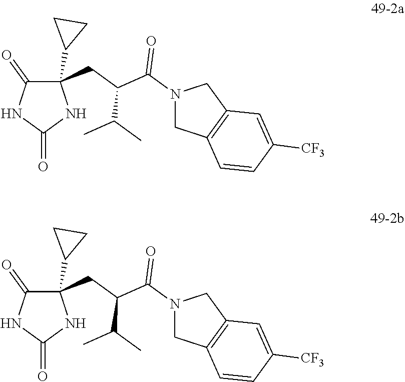 [SMILES: CC(C)[C@H](C[C@@]1(C2CC2)NC(=O)NC1=O)C(=O)N1Cc2ccc(C(F)(F)F)cc2C1 ; CC(C)[C@@H](C[C@@]1(C2CC2)NC(=O)NC1=O)C(=O)N1Cc2ccc(C(F)(F)F)cc2C1]

49-2 (140 mg) was seperated by chiral SFC to afford the title compounds (50 mg and 50 mg).

### Enantiomer (Shorter Retention Time):

¹H NMR (400 MHz, DMSO-d₆): δ 10.63 (s, 1H), 7.74 (d, 1H), 7.68-7.65 (m, 1H), 7.61-7.54 (m, 2H), 4.90-4.86 (m, 2H), 4.74-4.71 (m, 2H), 2.33-2.28 (m, 2H), 1.87-1.75 (m, 2H), 1.05-1.03 (m, 1H), 0.92-0.89 (m, 6H), 0.40-0.38 (m, 1H), 0.28-0.25 (m, 2H), 0.01-0.00 (m, 1H).

¹⁹F NMR (376.5 MHz, DMSO-d₆): δ-60.57.

LCMS: MS m/z (ESI): 424.5 [M+H]⁺.

Chiral HPLC (CO₂/EtOH/DEA 5%-40% 1.5 ml/min IG,3 um,3*100(Daicel)): Rt:3.003 min, ee:100%.

### Enantiomer (Longer Retention Time):

¹H NMR (400 MHz, DMSO-d₆): δ 10.62 (s, 1H), 7.75 (d, 1H), 7.68-7.65 (m, 1H), 7.60-7.54 (m, 2H), 4.90-4.86 (m, 2H), 4.73-4.69 (m, 2H), 2.32-2.28 (m, 2H), 1.88-1.74 (m, 2H), 1.05-1.02 (m, 1H), 0.92-0.86 (m, 6H), 0.42-0.40 (m, 1H), 0.28-0.25 (m, 2H), 0.01-0.00 (m, 1H).

¹⁹F NMR (376.5 MHz, DMSO-d₆): δ −60.57.

LCMS: MS m/z (ESI): 424.2 [M+H]⁺.

Chiral HPLC (CO₂/EtOH/DEA 5%-40% 1.5 ml/min IG,3 um,3*100(Daicel)): Rt:3.494 min, ee:100%.

### Example 50

### (S)-5-(3-(5-chloro-6-nitroisoindolin-2-yl)-3-oxopropyl)-5-cyclopropylimidazolidine-2,4

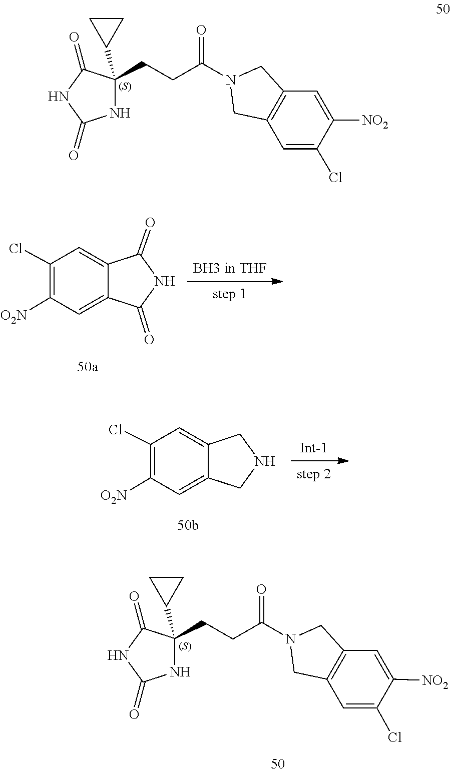

5-chloro-6-nitroisoindoline (50b)

To a solution of 5-chloro-6-nitroisoindoline-1,3-dione 50a (1 g, 4.42 mmol) in THF (15 mL) was added borane-tetrahydrofuran (1M, 35 mL) dropwise under N₂. The resulting mixture was stirred at 60° C. for 24 h. The reaction mixture was cooled to ambient temperature and quenched with MeOH (5 mL) until the bubbling ceased. Then 4N HCl in water (4 mL) was added and the mixture was heated at 80° C. for 3 h. After cooled down to RT, 5N KOH was added to adjust pH to 7. The mixture was concentrated under reduced pressure and the residue was purified by silica-gel column (DCM: MeOH(2% NH₄₀H)=10:1) to afford 5-chloro-6-nitroisoindoline 50b (300 mg, 33% yield).

### Step 2

### (S)-5-(3-(5-chloro-6-nitroisoindolin-2-yl)-3-oxopropyl)-5-cyclopropylimidazolidine-2,4-dione 50

To a mixture of 5-chloro-6-nitroisoindoline 50b (10 mg, 0.05 mol) in DMF (2 mL) was added triethylamine (18 mg, 0.138 mmol), Int-1 (10 mg, 0.047 mmol) and HATU (21 mg, 0.055 mmol). The reaction was stirred at r.t for 18 h. Water (4 mL) was added and the mixture was extracted with EtOAc (20 mL×2). The combined organic layers were washed with brine, dried over Na₂SO₄ and concentrated. The residue was purified with prep-HPLC to give the title compound 50 (4 mg, 0.01 mmol, 21% yield).

¹H NMR (400 MHz, Methanol-d4): 0.94 (d, 1H), 7.67 (d, 1H), 4.95 (d, 2H), 4.81 (d, 2H), 2.57 (m, 1H), 2.45 (m, 1H), 2.33-2.14 (m, 2H), 1.34-1.20 (m, 1H), 0.61 (m, 1H), 0.53-0.30 (m, 3H).

LCMS: MS m/z (ESI): 393 [M+H]⁺.

### Example 51

### (5S)-5-cyclopropyl-5-(3-(5-(1-hydroxyethyl)isoindolin-2-yl)-3-oxopropyl)imidazolidine-2,4-dione 51

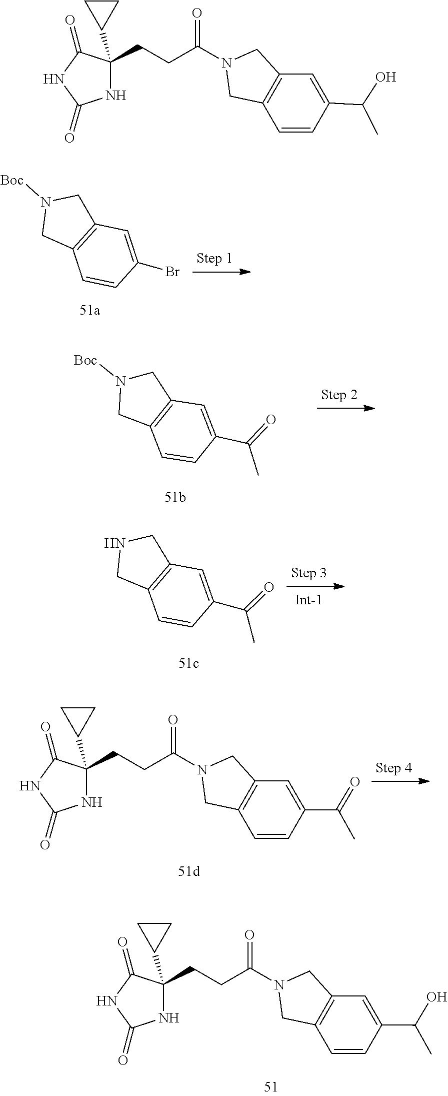 [SMILES: CC(O)c1ccc2c(c1)CN(C(=O)CC[C@@]1(C3CC3)NC(=O)NC1=O)C2 ; CC(C)(C)OC(=O)N1Cc2ccc(Br)cc2C1 ; CC(=O)c1ccc2c(c1)CN(C(=O)OC(C)(C)C)C2 ; CC(=O)c1ccc2c(c1)CNC2 ; CC(=O)c1ccc2c(c1)CN(C(=O)CC[C@@]1(C3CC3)NC(=O)NC1=O)C2 ; CC(O)c1ccc2c(c1)CN(C(=O)CC[C@@]1(C3CC3)NC(=O)NC1=O)C2]

### Step 1

### Tert-butyl 5-acetylisoindoline-2-carboxylate 51b

To a mixture of tert-butyl 5-bromoisoindoline-2-carboxylate 51a (500 mg, 1.68 mmol), tributyl(1-ethoxyvinyl)stannane (726.72 mg, 2.01 mmol) in dioxane (5 mL) was added Pd(PPh₃)₄ (193.78 mg, 167.69 umol). The reaction was stirred under N₂ at 100° C. for 6 h. The reaction was cooled to rt, the mixture was diluted with water (50 mL), the mixture was extracted with ethylacetate (20 mL×3). The organic solution was washed with brine, dried over Na₂SO₄ and concentrated. The residue was purified by prep-TLC (hexane:EtOAc=10:1) to give 51b (105 mg, 401.81 umol, 23.96% yield).

¹H NMR (400 MHz, DMSO-d₆): δ 7.90-7.84 (m, 2H), 7.35-7.32 (m, 1H), 4.73 (br, 2H), 4.70 (br, 2H), 2.61 (s, 3H), 1.45 (s, 9H).

### Step 2

### 1-(isoindolin-5-yl)ethan-1-one 51c

To a mixture of 51b (50 mg, 191.34 umol) in DCM (3 mL) was added dioxane/HCl (1N, 1 mL).

The reaction was stirred at room temperature for 16 h. The mixture was concentrated to give crude 51c, the crude product was used to the next step directly without purification.

### Step 3

### (S)-5-(3-(5-acetylisoindolin-2-yl)-3-oxopropyl)-5-cyclopropylimidazolidine-2,4-dione 51d

To a solution of 51c (56 mg, 347.39 umol) in DMF (10 mL) was added Int-1 (73.72 mg, 347.39 umol) and HATU (198.13 mg, 521.09 umol). The mixture was stirred at room temperature 2 h.

Water (30 mL) was added and the mixture was extracted with EtOAc (20 mL×2). The combined organic layers were washed with water (40 mL) and brine (40 mL×2), dried and concentrated. The crude was purified by prep-HPLC to give 51d (52 mg, 146.32 umol, 42.12% yield).

¹H NMR (400 MHz, CDCl₃): δ 7.94-7.87 (m, 2H), 7.57 (brs, 1H), 7.42-7.34 (m, 1H), 6.09 (d, 1H), 4.83 (brs, 4H), 2.62 (s, 3H), 2.66-2.62 (m, 1H), 2.47-2.27 (m, 3H), 1.26-1.20 (m, 1H), 0.62-0.59 (m, 1H), 0.48-0.36 (m, 3H).

### Step 4

### (5S)-5-cyclopropyl-5-(3-(5-(1-hydroxyethyl)isoindolin-2-yl)-3-oxopropyl)imidazolidine-2,4-dione 51

To a mixture of 51d (22 mg, 61.90 umol) in MeOH (3 mL), NaBH4 (11.70 mg, 309.52 umol) was added. The reaction was stirred at room temperature for 4 h. The reaction was quenched with water (30 mL), and the mixture was extracted with EtOAc (20 mL×3), The organic solution was washed with brine, dried over Na₂SO₄, filtered and concentrated. The crude was purified by prep-HPLC to give 51 (7 mg, 19.59 umol, 31.64% yield).

¹H NMR (400 MHz, DMSO-d₆): δ10.51 (brs, 1H), 7.63 (brs, 1H), 7.21-7.12 (m, 3H), 5.06-5.05 (m, 1H), 4.66-4.59 (m, 3H), 4.48 (d, 2H), 2.33-2.26 (m, 1H), 2.18-2.13 (m, 1H), 1.89 (t, 2H), 1.19 (d, 3H), 1.02-0.98 (m, 1H), 0.35-0.19 (m, 3H), 0.01-0.00 (m, 1H).

LCMS: MS m/z (ESI): 358.2 [M+H]⁺.

### Example 52

### (S)-5-cyclopropyl-5-(3-(5-(difluoromethyl)isoindolin-2-yl)-3-oxopropyl)imidazolidine-2,4-dione 52

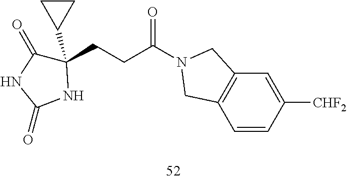 [SMILES: O=C1NC(=O)[C@](CCC(=O)N2Cc3ccc(C(F)F)cc3C2)(C2CC2)N1]

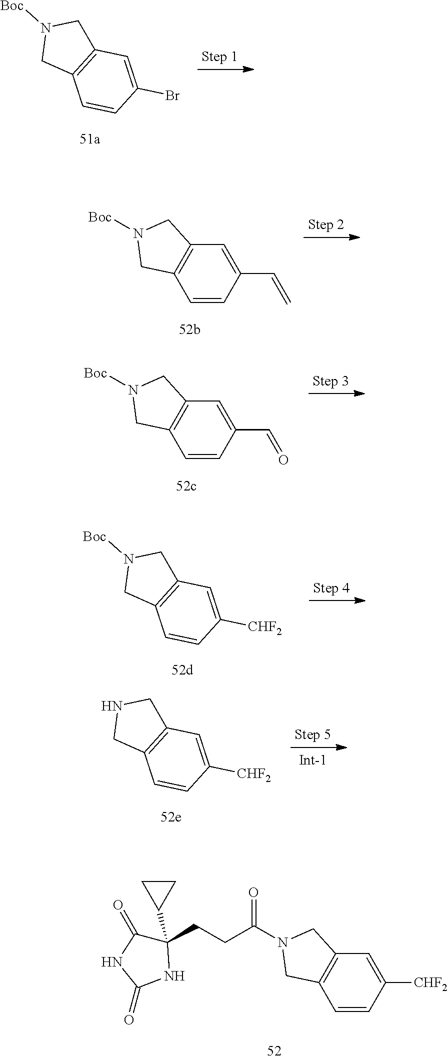 [SMILES: CC(C)(C)OC(=O)N1Cc2ccc(Br)cc2C1 ; C=Cc1ccc2c(c1)CN(C(=O)OC(C)(C)C)C2 ; CC(C)(C)OC(=O)N1Cc2ccc(C=O)cc2C1 ; CC(C)(C)OC(=O)N1Cc2ccc(C(F)F)cc2C1 ; FC(F)c1ccc2c(c1)CNC2 ; O=C1NC(=O)[C@](CCC(=O)N2Cc3ccc(C(F)F)cc3C2)(C2CC2)N1]

### Step 1

### Tert-butyl 5-vinylisoindoline-2-carboxylate 52b

To a mixture of 51a (1 g, 3.35 mmol), K₂CO₃ (925.63 mg, 6.71 mmol) and potassium vinyltrifluoroborate (538.88 mg, 4.02 mmol) in dioxane (10 mL) and water (2 mL) was added Pd(dppf)Cl₂ (197.54 mg, 335.37 umol). The reaction was stirred under N₂ at 80° C. for 3 h. The reaction mixture was cooled to room temperature and diluted with water (50 mL), extracted with EtOAc (30 mL×3). The combined organic layers were washed with brine, dried over Na₂SO₄ and concentrated. The residue was purified by SGC (hexane:EtOAc=10:1) to give 52b (786 mg, 3.20 mmol, 95.54% yield).

¹H NMR (400 MHz, CDCl₃): δ 7.32-7.15 (m, 3H), 6.71 (dd, 1H), 5.73 (dd, 1H), 5.23 (d, 1H), 4.67 (br, 2H), 5.22 (br, 2H), 1.52 (d, 9H).

### Step 2

### Tert-butyl 5-formylisoindoline-2-carboxylate 52c

To a mixture of 52b (400 mg, 1.63 mmol), NaIO₄ (697.88 mg, 3.26 mmol) in dioxane (7 mL) and water (4 mL) was added osmium tetroxide (41.45 mg, 163.05 umol). The reaction was stirred at room temperature for 0.5 h and then diluted with water (40 mL). The mixture was extracted with ethylacetate (20 mL×3), the organic solution was washed with brine, dried over Na₂SO₄ and concentrated. The residue was purified by silica gel chromatography (hexane:EtOAc=5:1) to give 52c (98 mg, 396.30 umol, 24.30% yield).

¹H NMR (400 MHz, CDCl₃): δ 10.01 (s, 1H), 7.82-7.76 (m, 2H), 7.44-7.40 (m, 1H), 4.76-7.73 (m, 411), 1.53 (s, 911).

### Step 3

### Tert-butyl 5-(difluoromethyl)isoindoline-2-carboxylate 52d

To a mixture of 52c (50 mg, 202.19 umol) and EtOH (930.09 ug, 20.22 umol) in DCM (3 mL) was added DAST (162.96 mg, 1.01 mmol). The reaction was stirred at room temperature for 16 h. The reaction was quenched with water (30 mL), and the mixture was extracted with EtOAc (20 mL×3). The organic solution was washed with brine, dried over Na₂SO₄ and concentrated to give crude 52d (52 mg, 193.10 umol, 95.50% yield).

¹H NMR (400 MHz, CDCl₃): δ 7.36-7.19 (m, 3H), 6.57 (t, 1H), 4.65 (br, 2H), 4.61 (br, 2H), 1.45 (s, 9H).

¹⁹F NMR (400 MHz, CDCl₃): δ −109.86.

### Step 4

### 5-(difluoromethyl)isoindoline 52e

To the mixture of 52d (30 mg, 111.41 umol) in DCM (3 mL) was add HCl/dioxane (1N, 1 mL).

The reaction was stirred at room temperature for 16 hours. The mixture was concentrated to give the crude 52e.

### Step 5

### (S)-5-cyclopropyl-5-(3-(5-(difluoromethyl)isoindolin-2-yl)-3-oxopropyl)imidazolidine-2,4-dione 52

To a solution of 52e (35 mg, 206.89 umol) in DMF (3 mL) was added Int-1 (43.90 mg, 206.89 umol), TEA (62.81 mg, 620.67 umol) and HATU (94.40 mg, 248.27 umol). The mixture was stirred at room temperature for 2 h. Water (30 mL) was added and the mixture was extracted with EA (20 mL×2). The combined organic layers were washed with water (30 mL) and brine (30 mL), dried and concentrated. The crude was purified by prep-HTPLC to give 52 (5 mg, 13.76 umol, 6.65% yield).

¹H NMR (400 MHz, DMSO-d₆): δ 10.64 (brs, 0.5H), 7.73 (brs, 0.5H), 7.73-7.70 (m, 1H), 7.58-7.49 (m, 2H), 7.05 (t, 1H), 4.88-4.84 (m, 2H), 4.67-4.65 (m, 2H), 2.50-2.28 (m, 2H), 2.07-1.97 (m, 2H), 1.12-0.85 (m, 1H), 0.46-0.27 (m, 2H), 0.20-0.05 (m, 1H).

¹⁹F NMR (376.5 MHz, DMSO-d₆): δ-108.63.

LCMS: MS m/z (ESI): 364.0 [M+H]⁺.

### Example 53

### (S)-5-(3-(5-chloro-6-iodoisoindolin-2-yl)-3-oxopropyl)-5-cyclopropylimidazolidine-2,4-dione 53

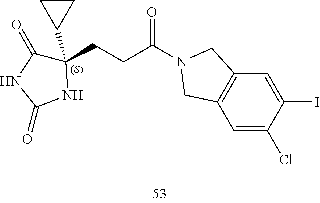

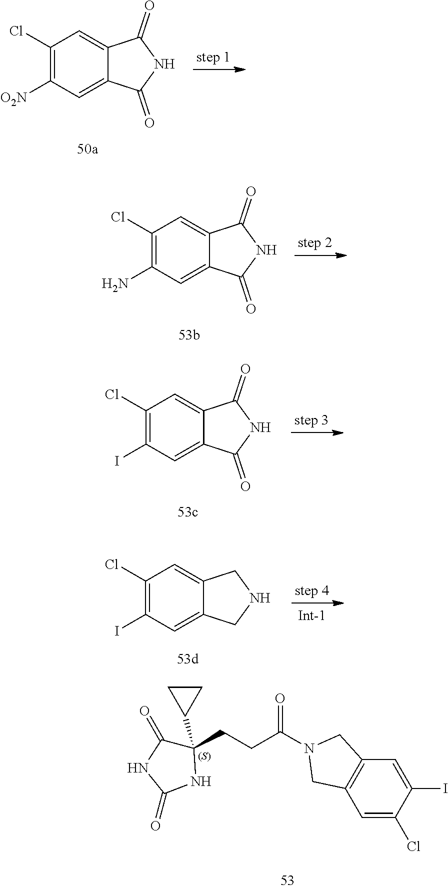

### Step 1

### 5-amino-6-chloroisoindoline-1,3-dione 53b

To a mixture of 50a (1 g, 4.4 mmol) in MeOH (20 mL) was added ammonia solution (3 mL), H₂O (5 ml) and Na₂S204 (7.6 g, 44 mmol). The reaction was stirred at r.t for 24 h. Water (4 mL) was added and the mixture was extracted with EtOAc (30 mL×4). The combined organic layers were washed with brine, dried over Na₂SO₄ and concentrated to afford 53b (0.5 g, 2.5 mmol, yield 56%). The residue was used in next step without further purification.

### Step 2

### 5-chloro-6-iodoisoindoline-1,3-dione 53c

To a stirred suspension of 53b (0.5 g 2.5 mmol) in 15 mL water was added dropwise at 10° C.

A solution of 0.4 mL concentrated sulfuric acid in 5 mL water. After the mixture was cooled to 5° C., a solution of sodium nitrite (276 mg, 4 mmol) in 5 mL water was added dropwise and the stirring continued at 0° C. for 90 mins. A solution of potassium iodide (1.4 g, 8.8 mmol) in 8 mL water was then added dropwise over 40 mins while maintaining the reaction temperature between 0° C. and 5° C. The reaction mixture was then warmed to room temperature and subsequently heated at 35° C. for 45 mins and then 60° C. for 30 min. Then the mixture was cooled down to room temperature and was extracted with EtOAc (30 mL×4). The combined organic layers were dried, filtered and concentrated. The residue was resuspended in 30 mL DCM, stirred for 10 mins at room temperature and the resulting crystals collected by filtration to yield compound 53c (240 mg, 0.78 mmol, 31% yield).

### Step 3

### 5-chloro-6-iodoisoindoline 53d

To a solution of 53c (120 mg, 0.38 mmol) in THF (5 mL) was added borane-tetrahydrofuran (1M, 60 mL) dropwise under N₂. The resulting mixture was stirred at 60° C. for 24 h. The reaction mixture was cooled to ambient temperature and quenched with MeOH (6 mL) until the bubbling ceased. Then 4N HCl in water (2 mL) was added and the mixture was heated at 80° C. for 3 h. Then the mixture was cooled down to room temperature and 5N KOH was added to adjust pH to 7. The mixture was concentrated under reduced pressure and the residue was purified by silica-gel column (DCM: MeOH(2% NH₄₀H)=10:1) to afford 53d (51 mg, 0.18 mmol 47% yield).

### Step 4

### (S)-5-(3-(5-chloro-6-iodoisoindolin-2-yl)-3-oxopropyl)-5-cyclopropylimidazolidine-2,4-dione 53

To a mixture of 53d (10 mg, 0.035 mmol) in DMF (2 mL) was added triethylamine (12 mg, 0.1 mmol), Int-1 (9 mg, 0.042 mmol) and HATU (21 mg, 0.055 mmol). The reaction was stirred at r.t for 18 h. Water (3 mL) was added and the mixture was extracted with EtOAc (20 mL×2). The combined organic layers were washed with brine, dried over Na₂SO₄ and concentrated.

The residue was purified with prep-HPLC to give title compound 53 (4 mg, 0.008 mmol, 22% yield). 15 ¹H NMR (400 MHz, Methanol-d4): δ 7.92 (d, 1H), 7.53 (d, 1H), 4.84 (s, 2H), 4.71 (s, 2H), 2.61-2.33 (m, 2H), 2.30-2.15 (m, 2H), 1.20-1.30 (m, 1H), 0.68-0.32 (m, 4H).

LCMS: m/z (ESI): 474 [M+H]⁺

### Example 54

### (S)-5-(3-(5-amino-6-chloroisoindolin-2-yl)-3-oxopropyl)-5-cyclopropylimidazolidine-2,4-dione 54

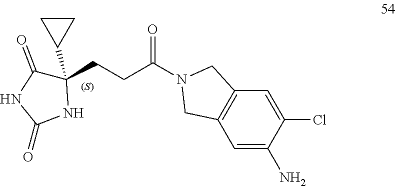

To a mixture of 50 (10 mg, 0.025 mmol) in MeOH (2 mL) was added ammonia solution (0.3 ml), H₂O (lml) and Na₂S204 (65 mg, 0.375 mmol). The reaction was stirred at r.t for 18 h. Water (6 mL) was added and the mixture was extracted with EtOAc (20 mL×2). The combined organic layers were washed with brine, dried over Na₂SO₄ and concentrated. The residue was purified with prep-HPLC to give title compound 54 (6 mg, 0.016 mmol, 64% yield).

¹H NMR (400 MHz, Methanol-d₄): δ 7.64 (d, 1H), 7.32 (d, 1H), 6.98 (d, 2H), 4.79 (d, 2H), 4.67 (s, 2H), 2.55 (m, 1H), 2.42 (m, 1H), 2.31-2.13 (m, 2H), 1.34-1.19 (m, 1H), 0.60 (td, 1H), 0.53-0.30 (m, 3H).

LCMS: MS m/z (ESI): 363 [M+H]⁺.

### Example 55

### (S)-N-(6-chloro-2-(3-(4-cyclopropyl-2,5-dioxoimidazolidin-4-yl)propanoyl)isoindolin-5-yl)acetamide 55

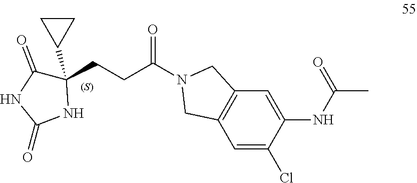

To a mixture of 54 (3 mg, 0.08 mmol) in THF (2 mL) was added acetic anhydride (8 mg 0.075 mmol) and triethylamine (18 mg 0.14 mmol). The reaction was stirred at 30° C. for 18 h. The mixture was concentrated. The residue was purified with prep-HPLC to give title compound 55 (2.1 mg, 0.005 mmol, 62% yield).

¹H NMR (400 MHz, Methanol-d₄): δ 7.74 (s, 1H), 7.47 (d, 1H), 4.84 (s, 2H), 4.74 (s, 2H), 2.68-2.33 (m, 2H), 2.30-2.12 (m, 5H), 1.30-1.21 (m, 1H), 0.68-0.29 (m, 4H).

LCMS: MS m/z (ESI): 405 [M+H]⁺.

### Example 56

### (S)-2-(3-(4-cyclopropyl-2,5-dioxoimidazolidin-4-yl)propanoyl)isoindoline-5-carboxylic acid 56

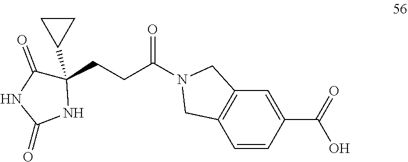 [SMILES: O=C1NC(=O)[C@](CCC(=O)N2Cc3ccc(C(=O)O)cc3C2)(C2CC2)N1]

The title compound was prepared with similar method as Example 1.

LCMS: MS m/z (ESI): 358 [M+H]⁺.

### Example 57

### (S)-5-(3-(5-chloro-6-methylisoindolin-2-yl)-3-oxopropyl)-5-cyclopropylimidazolidine-2,4-dione 57

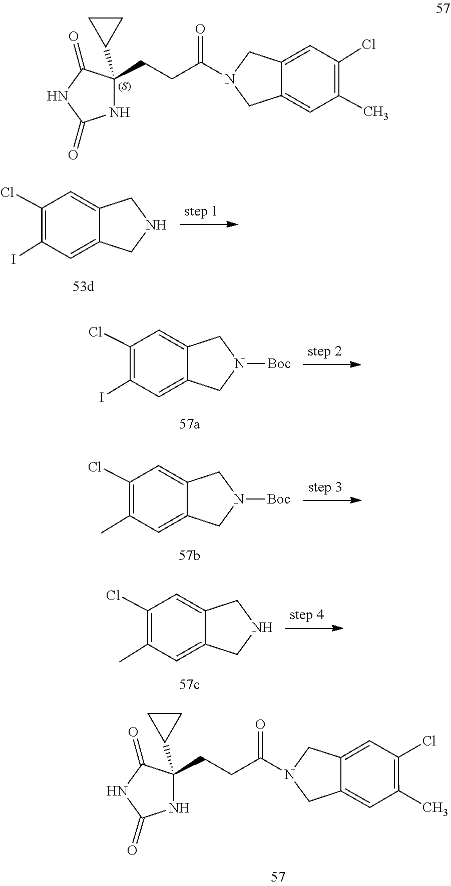

### Step 1

### tert-butyl 5-chloro-6-iodoisoindoline-2-carboxylate 57a

To a mixture of 53d (40 mg, 0.143 mmol) in 5N KOH solution (2 mL) was added di-tert-butyl dicarbonate (100 mg, 0.45 mmol), the reaction was stirred at r.t for 18 h. The mixture was cooled to 0° C., then filtered to give 57a (28 mg, 0.09 mmol, 63% yield).

### Step 2

### tert-butyl 5-chloro-6-methylisoindoline-2-carboxylate 57b

To a solution of 57a (20 mg, 0.052 mmol) in DME (3 mL) was added water (2 mL), CH₃B(OH)₂ (20 mg 0.33 mmol), K₂CO₃ (15 mg 0.1 mmol) and Pd (PPh₃)₂C₁₂ (3 mg, 0.0052 mmol). The reaction mixture was stirred at 80° C. for 18 h under N₂ atmosphere. After the reaction was completed, the reaction mixture was quenched and extracted with EtOAc (20 mL×2). The combined organic layers were washed with brine, dried over Na₂SO₄ and concentrated to afford 57b. The crude product was used without purification for next step.

### Step 3

### 5-chloro-6-methylisoindoline 57c

To a solution of 57b (20 mg, 0.052 mmol) in DCM (3 mL) was added 4N HCl in 1,4 dioxane(2 mL). After the reaction was complete. The mixture was concentrated under reduced pressure and the residue was purified by silica-gel column (DCM: MeOH(2% NH₄₀H)=10:1) to afford 57c (5 mg, 0.029 mmol 60% yield).

### Step 4

### (S)-5-(3-(5-chloro-6-methylisoindolin-2-yl)-3-oxopropyl)-5-cyclopropylimidazolidine-2,4-dione 57

To a mixture of 57c (5 mg, 0.029 mmol) in DMF (2 mL) was added Triethylamine (13 mg, 0.1 mmol), Int-1 (5 mg, 0.022 mmol) and HATU (12 mg, 0.031 mmol). The reaction was stirred at r.t for 18 h. Water (3 mL) added and the mixture was extracted with EtOAc (20 mL×2).

The combined layer was washed with brine, dried over Na₂SO₄ and concentrated. The residue was purified with prep-HPLC to give title compound 57 (3 mg, 0.008 mmol, 36% yield).

¹H NMR (400 MHz, Methanol-d₄): δ 7.36 (d, 1H), 7.28 (d, 1H), 4.83 (s, 2H), 4.71 (d, 2H), 2.63-2.41 (m, 2H), 2.39 (s, 3H), 2.23 (m, 2H), 0.60 (td, 2H), 0.53-0.38 (m, 2H), 0.35 (dt, 1H).

LCMS: m/z (ESI): 362[M+H]⁺.

### Example 58

### (5S)-5-cyclopropyl-5-(3-(5,6-dichloro-1-ethylisoindolin-2-yl)-3-oxopropyl)imidazolidine-2,4-dione 58

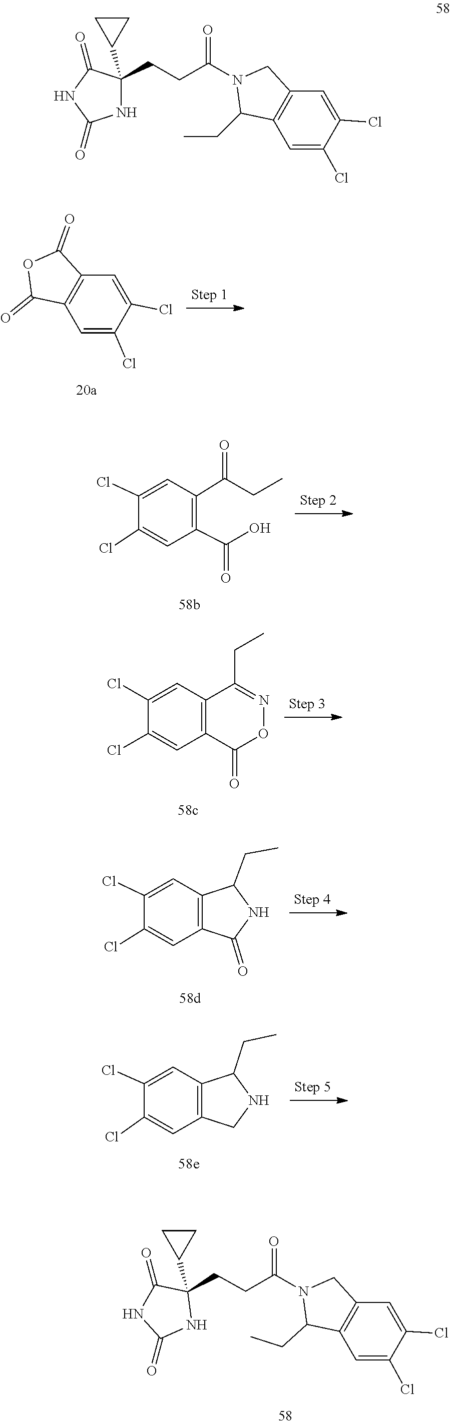

### Step 1

### 4,5-dichloro-2-propionylbenzoic acid 58b

The mixture of 20a (1.0 g, 4.61 mmol), 2-methylpropanedioic acid (761.85 mg, 6.45 mmol) and pyridine (1 mL) was stirred at 75° C. for 3 h. The mixture was diluted with water (2 mL) and concentrated HCl (2 mL), and the mixture was heated to 140° C. and stirred for 2 h. After the reaction mixture was cooled to RT, the mixture was filtered and the cake was dried to give 58b (640 mg, 2.59 mmol, 56.21% yield).

LCMS: MS m/z (ESI): 249.3 [M+H]⁺.

### Step 2

### 6,7-dichloro-4-ethyl-1H-benzo[d][1,2]oxazin-1-one 58c

58b (200 mg, 809.47 umol) was added to the solution of KOH (129.45 mg, 2.31 mmol) in H₂O (1.2 mL), and then hydroxylamine hydrochloride (120.38 mg, 1.73 mmol) was added portion-wise. The reaction was stirred at 30° C. overnight. Then the mixture was filtered and the filter cake was dried to give 58c (80 mg, 327.77 umol, 40.49% yield).

¹HNMR (400 MHz, DMSO-d₆): δ 8.40 (s, 1H), 8.31 (s, 1H), 3.01 (q, 2H), 1.24 (t, 3H).

LCMS: MS m/z (ESI): 244.3 [M+H]⁺.

### Step 3

### 5,6-dichloro-3-ethylisoindolin-1-one 58d

To a solution of 58c (350 mg, 1.43 mmol) in acetic acid (5 mL) was added Zn (937.69 mg, 14.34 mmol). The mixture was heated to 118° C. and stirred overnight. The mixture was filtered and the cake was washed with DCM, the filtrate was concentrated, and the residue was triturated with ether and filtered to give 58d (180 mg, 782.30 umol, 54.55% yield).

¹HNMR (400 MHz, DMSO-d₆): δ 8.96 (brs, 1H), 7.95 (s, 1H), 7.82 (s, 1H), 4.59-4.56 (m, 1H), 2.01-1.90 (m, 1H), 1.65-1.54 (m, 1H), 0.79 (t, 3H).

LCMS: MS m/z (ESI): 230.4 [M+H]⁺.

### Step 4

### 5,6-dichloro-1-ethylisoindoline 58e

To a solution of 58d (1.32 g, 5.74 mmol) in THF (10 mL) was added BH₃/THF (793.68 mg, 57.37 mmol, 55 mL). The mixture was heated to 60° C. and stirred overnight. The reaction was quenched with MeOH (5 mL) and then 6 M HCl (adjusted pH to 1-2). The mixture was heated to 80° C. for 1 h and then cooled to RT. The mixture was adjusted pH to 7-8 with aq. NaOH (6N).

Then the mixture was dried over anhydrous Na₂SO₄ and concentrated in vacuo. The residue was purified by column chromatography (EtOAc/hexane=1:20-1:1) to afford 58e (630 mg, 2.92 mmol, 50.82% yield).

¹HNMR (400 MHz, DMSO-d₆): δ 7.50 (s, 1H), 7.47 (s, 1H), 4.19-4.16 (m, 1H), 4.03 (s, 2H), 1.80-1.71 (m, 1H), 1.47-1.43 (m, 1H), 0.90 (t, 3H).

LCMS: MS m/z (ESI): 216.4 [M+H]⁺.

### Step 5

### (5S)-5-cyclopropyl-5-(3-(5,6-dichloro-1-ethylisoindolin-2-yl)-3-oxopropyl)imidazolidine-2,4-dione 58

To a solution of 58e (50 mg, 231.37 umol) in DMF (2 mL) was added TEA (0.3 mL), Int-1 (53 mg, 249.76 umol) and HATU (100 mg, 263.00 umol). The mixture was stirred at room temperature for 3 h. Water was added, and then the mixture was extracted with EtOAc. The combined organic layers were washed with water and brine, dried over anhydrous Na₂SO₄, filtered and concentrated in vacuo. The residue was purified by prep-HPLC to give 58 (30 mg, 73.12 umol, 31.60% yield).

¹HNMR (400 MHz, DMSO-d₆): δ 10.58 (br, 1H), 7.79-7.63 (m, 3H), 5.27-5.21 (m, 1H), 4.83-4.66 (m, 2H), 2.42-1.72 (m, 6H), 1.16-1.05 (m, 1H), 0.61-0.51 (m, 3H), 0.47-0.29 (m, 3H), 0.16-0.07 (m, 1H).

LCMS: MS m/z (ESI): 410.1 [M+H]⁺.

### Example 59

### (S)-5-(3-(5-bromo-6-(trifluoromethyl)isoindolin-2-yl)-3-oxopropyl)-5-cyclopropylimidazolidine-2,4-dione 59

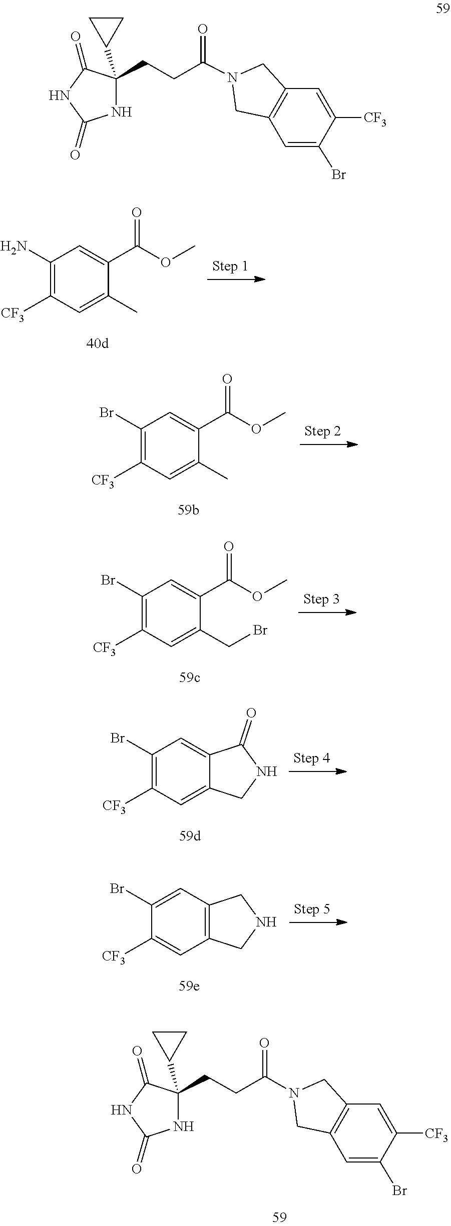

### Step 1

### Methyl 5-bromo-2-methyl-4-(trifluoromethyl)benzoate 59b

To a suspension of 40d (500 mg, 2.14 mmol) in CH₃CN (20 mL) was added isoamyl nitrite (377 mg, 3.22 mmol) and CuBr₂ (960 mg, 4.30 mmol). After the mixture was stirred at 70° C. overnight, the mixture was cooled down to room temperature and poured into ice water (20 mL). Then the mixture was extracted with EtOAc (50 mL). The organic phase was dried over Na₂SO₄(s), filtered. The filtrate was concentrated to afford crude 59b (600 mg, 2.02 mmol, 94.20% yield).

¹H NMR (400 MHz, CDCl₃): δ 8.20 (s, 1H), 7.56 (s, 1H), 3.93 (s, 3H), 2.59 (s, 3H).

### Step 2

### Methyl 5-bromo-2-(bromomethyl)-4-(trifluoromethyl)benzoate 59c

To a solution of 59b (100 mg, 336.62 umol) in CCl₄ (3 mL) was added AIBN (1.66 mg, 10.10 umol) and NBS (71.89 mg, 403.95 umol), and the mixture was stirred at 70° C. overnight. The mixture was cooled to room temperature and filtered, the cake was washed with DCM, the filtrate was concentrated in vacuo to give crude 59c (150 mg, 398.97 umol, 118.52% yield).

### Step 3

### 6-bromo-5-(trifluoromethyl)isoindolin-1-one 59d

To a solution of 59c (150 mg, 398.97 umol) in MeOH (1 mL) was added NH₃/MeOH (4 mL), the mixture was stirred at room temperature overnight. The reaction mixture was concentrated in vacuo, and the residue was purified by silica gel chromatography (EtOAc/hexane=1/5) to give 59d (60 mg, 214.25 umol, 53.70% yield).

¹HNMR (400 MHz, DMSO-d₆): δ 9.03 (brs, 1H), 8.16 (s, 1H), 8.08 (s, 1H), 4.44 (s, 2H).

LCMS: MS m/z (ESI): 280.3 [M+H]⁺.

### Step 4

### 5-bromo-6-(trifluoromethyl)isoindoline 59e

To a solution of 59d (60 mg, 214.25 umol) in THF (2 mL) was added BH₃/THF (29.64 mg, 2.14 mmol, 5 mL), and the mixture was heated to 60° C. and stirred overnight. The reaction was quenched with MeOH (5 mL) and the mixture was adjusted to pH 1-2 with 6M HCl. The mixture was heated to 80° C. and stirred for 1 h. The reaction was cooled to room temperature and adjusted to pH 7-8 with 6 M NaOH. The mixture was extracted with ethylacetate and the organic layer was dried over anhydrous Na₂SO₄ and concentrated in vacuo. The residue was purified by silica gel chromatography (MeOH/DCM=1/20) to afford 59e (20 mg, 75.17 umol, 35.09% yield).

LCMS: MS m/z (ESI): 268.2 [M+H]⁺.

### Step 5

### (S)-5-(3-(5-bromo-6-(trifluoromethyl)isoindolin-2-yl)-3-oxopropyl)-5-cyclopropylimidazolidine-2,4-dione 59

To a solution of 59e (20 mg, 75.17 umol) in DMF (1.5 mL) was added TEA (0.2 mL), Int-1 (16 mg, 75.40 umol) and HATU (30 mg, 78.90 umol). The mixture was stirred at room temperature for 2 h. Water was added and the mixture was extracted with EA. The combined organic layers were washed with water and brine, dried over anhydrous Na₂SO₄, filtered and concentrated in vacuo. The residue was purified by prep-HPLC to give 59 (10 mg, 21.73 umol, 28.90% yield).

¹HNMR (400 MHz, DMSO-d₆): δ 10.62 (brs, 1H), 7.92 (s, 1H), 7.89 (s, 1H), 7.75 (s, 1H), 4.86-4.81 (m, 2H), 4.68-4.63 (m, 2H), 2.41-2.22 (m, 2H), 2.03-1.98 (m, 2H), 1.15-1.07 (m, 1H), 0.50-0.29 (m, 3H), 0.16-0.07 (m, 1H).

¹⁹F NMR (400 MHz, CDCl₃): δ −60.79.

LCMS: MS m/z (ESI): 462.3 [M+H]⁺.

### Example 60

### 5-(3-oxo-3-(5-(trifluoromethyl)isoindolin-2-yl)propyl)-5-(pyridin-2-yl)imidazolidine-2,4-dione 60

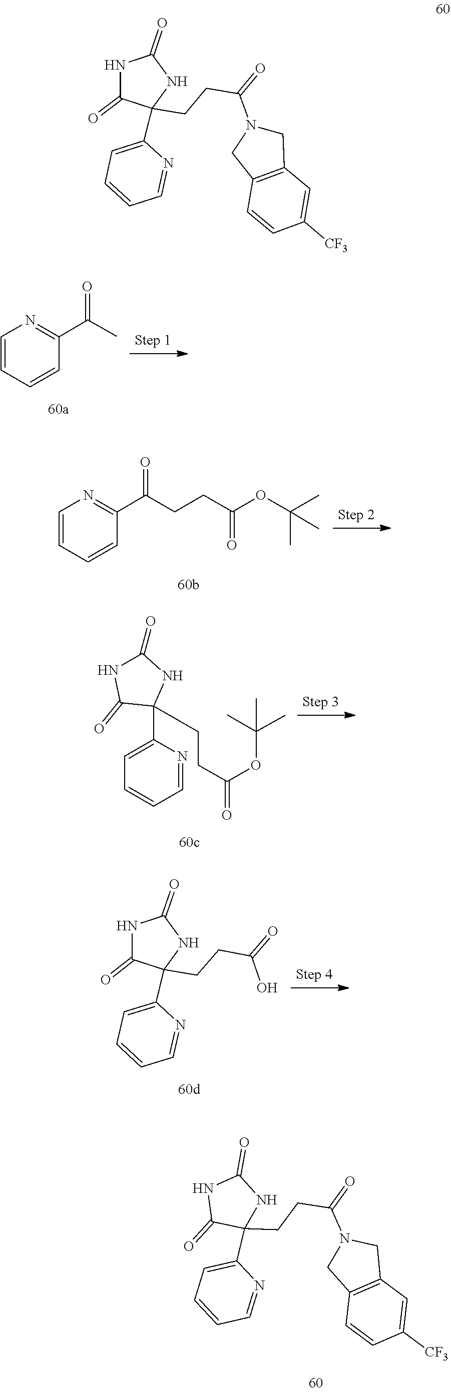

### Step 1

### Tert-butyl 4-oxo-4-(pyridin-2-yl)butanoate 60b

To a solution of 1-(pyridin-2-yl)ethanone 60a (24.2 g, 199 mmol) in THF (300 mL) at −70° C. was added LDA (120 mL, 240 mmol) dropwise. After the addition, the reaction mixture was stirred at this temperature for 30 min before tert-butyl 2-bromoacetate (39 g, 199 mmol) was added dropwise. Then the resulting mixture was stirred atroom temperature for 18 hours. The reaction mixture was diluted with aq. NH₄C₁ (100 mL). The mixture was extracted with EtOAc (400 mL×3). The organic layers were combined, washed with brine (400 mL), dried over Na₂SO₄, and filtered. The filtrate was concentrated under reduced pressure. The residue was purified by column chromatography (EtOAc/hexane=1/10 to 1/2) to afford 60b (11.0 g, 46.7 mmol, yield: 23.4%).

LCMS: MS m/z (ESI): 236.1 [M+H]⁺.

### Step 2

### Tert-butyl 3-(2,5-dioxo-4-(pyridin-2-yl)imidazolidin-4-yl)propanoate 60c

A mixture of 60b (4.7 g, 20.0 mmol), (NH₄)₂CO₃ (16.3 g, 170 mmol), NaCN (2.45 g, 50.00 mmol), EtOH (25 mL) and H₂O (25 mL) was heated in an autoclave at 85° C. for 18 hours. The resulting mixture was diluted with water (60 mL). The mixture was extracted with EtOAc (200 mL×5). The organic layers were combined, washed with brine (200 mL), dried over Na₂SO₄ and filtered. The filtrate was concentrated under reduced pressure. The residue was purified by column chromatography (EtOAc/hexane=1/10 to 1/1) to afford 60c (3.0 g, 9.83 mmol, yield: 49.2%).

LCMS: MS m/z (ESI): 306.1 [M+H]⁺.

### Step 3

### 3-(2,5-dioxo-4-(pyridin-2-yl)imidazolidin-4-yl)propanoic acid 60d

To a solution of 60c (1.0 g, 3.28 mmol) in DCM (10 mL) was added HCl/1,4-dioxane (30 mL, 3.0 M). The resulting mixture was stirred at room temperature for 18 hours. Then the reaction mixture was concentrated under reduced pressure. The residue was washed with Et₂O (10 mL), dried in vacuum to afford 60d (800 mg, 3.21 mmol, 98.01% yield).

LCMS: MS m/z (ESI): 248.2 [M−1]⁻.

### Step 4

### 5-(3-oxo-3-(5-(trifluoromethyl)isoindolin-2-yl)propyl)-5-(pyridin-2-yl)imidazolidine-2,4-dione 60

A mixture of 60d (100 mg, 401.25 umol), 5-(trifluoromethyl)isoindoline 1-1 (89.7 mg, 401 umol), Et₃N (140 mg, 1.39 mmol), HATU (153 mg, 401 umol) and DMF (10 mL) was stirred at room temperature for 18 hours. Then the reaction mixture was purified by prep-HPLC to afford 60 (30 mg, 71.71 umol, 17.9% yield).

¹H NMR (400 MHz, DMSO-d₆): δ 10.92 (brs, 1H), 8.59 (d, 1H), 8.16-8.10 (m, 1H), 7.84 (t, 1H), 7.75 (d, 1H), 7.66 (d, 1H), 7.59-7.51 (m, 2H), 7.37-7.34 (m, 1H), 4.83 (brs, 2H), 4.69 (brs, 2H), 4.43-4.28 (m, 4H).

LCMS: MS m/z (ESI): 419.0 [M+H]⁺.

### Example 61

### 5-(3-(5,6-dichloroisoindolin-2-yl)-3-oxopropyl)-5-(pyridin-2-yl)imidazolidine-2,4-dione 61

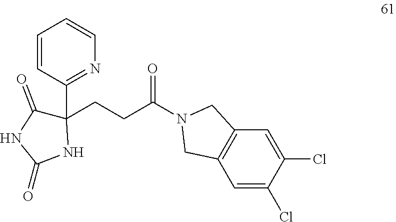 [SMILES: O=C1NC(=O)C(CCC(=O)N2Cc3cc(Cl)c(Cl)cc3C2)(c2ccccn2)N1]

To a solution of 60d (66.87 mg, 268.31 umol) in DMF (6 mL) was added DIEA (173.06 mg, 1.34 mmol), followed by the addition of 20c (60 mg, 268.31 umol), EDCI (128.59 mg, 670.77 umol) and HOBt (47.13 mg, 348.80 umol). The mixture was stirred for 2 hours and then the mixture was purified by prep-HPLC to afford the tittle compound 61 (8.8 mg, 20.93 umol, 7.80% yield).

¹H NMR (400 MHz, DMSO-d₆): δ 10.87 (s, 1H), 8.61 (s, 1H), 8.52 (s, 1H), 7.86 (t, 1H), 7.66 (s, 1H), 7.65 (s, 1H), 7.54 (d, 1H), 7.39-7.37 (m, 1H), 4.74 (brs, 2H), 4.60 (brs, 2H), 2.50-2.39 (m, 2H), 2.33-2.30 (m, 2H).

LCMS: MS m/z (ESI): 419.0 [M+H]⁺.

### Example 62

### 5-(1-methyl-1H-imidazol-2-yl)-5-(3-oxo-3-(5-(trifluoromethyl)isoindolin-2-yl)propyl)imidazolidine-2,4-dione 62

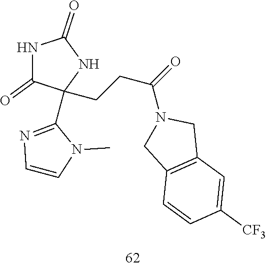 [SMILES: Cn1ccnc1C1(CCC(=O)N2Cc3ccc(C(F)(F)F)cc3C2)NC(=O)NC1=O]

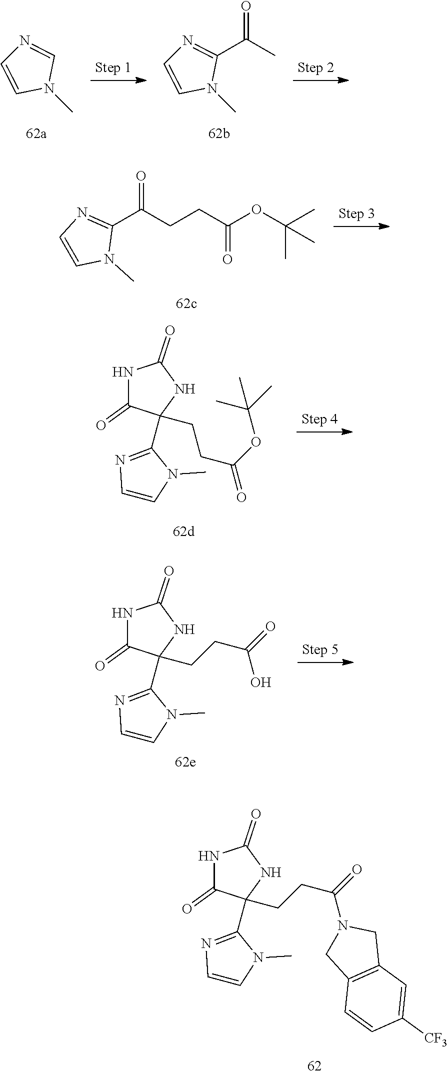

### Step 1

### 1-(1-methyl-1H-imidazol-2-yl)ethan-1-one 62b

To a solution of 1-methyl-1H-imidazole 62a (39 g, 475.01 mmol) in THF (350 mL) at −70° C. was added n-BuLi (356 mL, 1.6N, 570.01 mmol) dropwise. After the addition, the resulting mixture was allowed to warm to 0° C. and stirred at this temperature for 30 min, and then the reaction was re-cooled to −70° C. Ethyl acetate (104.63 g, 1.19 mol) was added dropwise at −70° C. The reaction mixture was stirred at room temperature for 18 hours. The reaction mixture was diluted with aq. NH₄Cl (100 mL). The whole mixture was extracted with EtOAc (400 mL×3). The organic layers were combined, washed with brine (400 mL), dried over Na₂SO₄ and filtered. The filtrate was concentrated under reduced pressure. The residue was purified by column chromatography (EtOAc/hexane=1/10 to 1/2) to afford 62b (26 g, 209.44 mmol, 44.09% yield).

¹H NMR (400 MHz, CDCl₃): δ 7.14 (s, 1H), 7.04 (s, 1H), 4.00 (d, 3H), 2.66 (d, 3H).

### Step 2

### Tert-butyl 4-(1-methyl-1H-imidazol-2-yl)-4-oxobutanoate 62c

A solution of LDA (77.3 mL, 2N, 154.66 mmol) in THF (200 mL) was cooled to −78° C. A solution of 62b (16.0 g, 128.89 mmol) was added dropwise, then the resulting mixture was warmed to 0° C. and stirred for 30 min. The reaction mixture was re-cooled to −78° C. and tert-butyl 2-bromoacetate (25.14 g, 128.89 mmol) was added slowly. The reaction was stirred at room temperature overnight. The reaction was quenched with saturated NH₄C₁ aq. (150 mL), the whole mixture was extracted with EtOAc (150 mL×3). The combined organic layer was washed with brine (100 mL), dried over Na₂SO₄ and concentrated under reduced pressure. The residue was purified by column chromatography (EtOAc/hexane=1/8 to 1/1) to afford 62c (13 g, 54.56 mmol, 42.33% yield).

¹H NMR (400 MHz, CDCl₃): δ 7.15-7.13 (m, 1H), 7.06 (s, 1H), 4.01-3.99 (m, 3H), 3.44-3.38 (m, 2H), 2.67-2.61 (m, 2H), 1.46-1.43 (m, 9H).

### Step 3

### Tert-butyl 3-(4-(1-methyl-1H-imidazol-2-yl)-2,5-dioxoimidazolidin-4-yl)propanoate 62d

A mixture of 62c (4.0 g, 16.79 mmol), (NH₄)₂CO₃ (13.70 g, 142.69 mmol), NaCN (2.23 g, 41.97 mmol), EtOH (25 mL) and H₂O (25 mL) was added to sealed vessel and heated to 85° C. reacted for 18 hours. The reaction mixture was diluted with water (100 mL). The whole mixture was extracted with n-BuOH (100 mL×3). The combined organic layers were washed with brine (50 mL), concentrated under reduced pressure. The residue was purified by column chromatography (DCM/MeOH=100/1 to 10/1) to afford 62d (700 mg, 2.27 mmol, 13.52% yield).

LCMS: m/z (ESI): 309.4 [M+H]⁺.

### Step 4

### 3-(4-(1-methyl-1H-imidazol-2-yl)-2,5-dioxoimidazolidin-4-yl)propanoic acid 62e

To a solution 62d (700 mg, 2.27 mmol) in dioxane (20 mL) was added HCl/dioxane (20 mL, 6N, 120 mmol). The resulting mixture was stirred at room temperature for 18 hours. The reaction mixture was concentrated under reduced pressure to afford crude 62e (700 mg, 2.78 mmol).

LCMS: m/z (ESI): 253.1 [M+H]⁺.

### Step 5

### 5-(1-methyl-1H-imidazol-2-yl)-5-(3-oxo-3-(5-(trifluoromethyl)isoindolin-2-yl)propyl)imidazolidine-2,4-dione 62

The mixture of 62e (100 mg, 396.47 umol) and 5-(trifluoromethyl)isoindoline 1-1 (89 mg, 396.47 umol) in DMF (5 mL) was added HATU (150 mg, 396.47 umol) and Et₃N (160 mg, 1.59 mmol), the reaction was stirred at room temperature for 18 hours. The reaction mixture was purified by prep-HPLC to afford 62 (30 mg, 71.20 umol, 17.96% yield).

¹H NMR (400 MHz, DMSO-d₆): δ 8.59-8.57 (m, 1H), 7.77 (s, 1H), 7.68-7.65 (m, 1H), 7.60-7.58 (m, 1H), 7.19 (s, 1H), 6.85 (s, 1H), 4.86-4.85 (m, 2H), 4.71 (br, 2H), 3.55 (s, 3H), 2.61-2.54 (m, 2H), 2.50-2.38 (m, 2H).

LCMS: MS m/z (ESI): 422.4 [M+H]⁺.

### Examples 62-1 and 62-2

### (S)-5-(1-methyl-1H-imidazol-2-yl)-5-(3-oxo-3-(5-(trifluoromethyl)isoindolin-2-yl)propyl)imidazolidine-2,4-dione 62-1 (R)-5-(1-methyl-1H-imidazol-2-yl)-5-(3-oxo-3-(5-(trifluoromethyl)isoindolin-2-yl)propyl)imidazolidine-2,4-dione 62-2

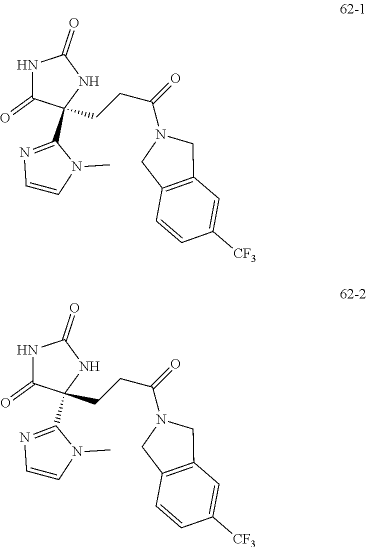 [SMILES: Cn1ccnc1[C@]1(CCC(=O)N2Cc3ccc(C(F)(F)F)cc3C2)NC(=O)NC1=O ; Cn1ccnc1[C@@]1(CCC(=O)N2Cc3ccc(C(F)(F)F)cc3C2)NC(=O)NC1=O]

62 (200 mg) was separated by SFC to give two single enantiomers (62 mg and 63 mg).

### Enantiomer (Shorter Retention Time):

¹H NMR (400 MHz, DMSO-d₆) δ 11.22 (s, 1H), 8.58 (d, 1H), 7.76 (s, 1H), 7.66 (d, 1H), 7.58 (dd, 1H), 7.19 (s, 1H), 6.85 (t, 1H), 4.86-4.84 (m, 2H), 4.71 (brs, 2H), 3.54 (s, 3H), 2.62-2.53 (m, 2H), 2.49-2.39 (m, 2H).

LCMS: m/z (ESI): 422.1 [M+H]⁺.

ChirHPLC (C₀₂/EtOH/DEA 60/40/0.04 2.8 ml/min OZ,5 um,4.6*250(Daicel)): Rt:3.326 min, ee:94.58%.

### Enantiomer (Longer Retention Time):

¹H NMR (400 MHz, DMSO-d₆) δ 11.23 (s, 1H), 8.59 (d, 1H), 7.76 (s, 1H), 7.66 (d, 1H), 7.59 (dd, 1H), 7.20 (s, 1H), 6.86 (s, 1H), 4.86-4.84 (d, 2H), 4.71 (brs, 2H), 3.55 (s, 3H), 2.61-2.53 (m, 2H), 2.49-2.42 (m, 2H).

LCMS: m/z (ESI): 422.1 [M+H]⁺.

ChirHPLC (C₀₂/EtOH/DEA 60/40/0.04 2.8 ml/min OZ,5 um,4.6*250(Daicel)): Rt:4.538 min, ee:100%.

### Example 63

### 5-(methoxymethyl)-5-(3-oxo-3-(5-(trifluoromethyl)isoindolin-2-yl)propyl)imidazolidine-2,4-dione 63

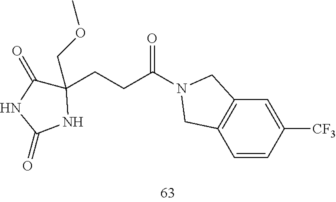 [SMILES: COCC1(CCC(=O)N2Cc3ccc(C(F)(F)F)cc3C2)NC(=O)NC1=O]

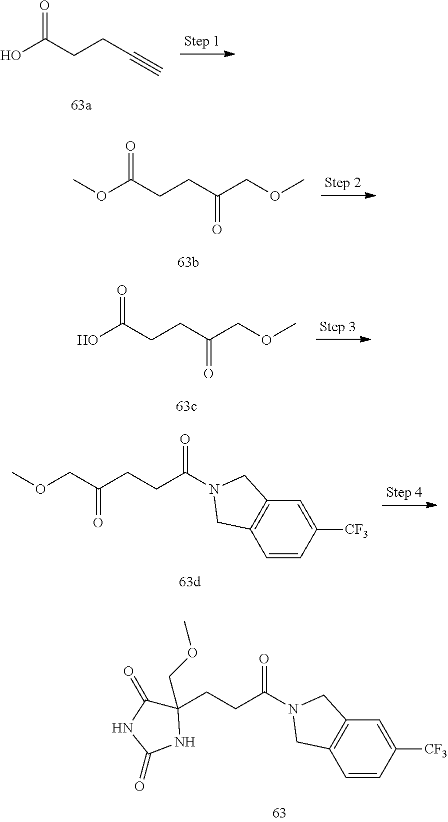

### Step 1

### Methyl 5-methoxy-4-oxopentanoate 63b

To a solution of iodosylbenzene (16.82 g, 76.45 mmol) in DCM (700 mL) at 0° C. was added pent-4-ynoic acid 63a (5 g, 50.97 mmol), boron trifluoride etherate (14.47 g, 101.94 mmol) was added dropwise. The reaction was stirred at r.t for 1 h. The resulting precipitate was separated by filtration and the solid was dried. MeOH (20 mL) was added, the reaction was stirred at r.t for 18 h. The solution was concentrated and the residue was purified by silica gel chromatography (hexane:EtOAc=10:1) to give 63b (3.6 g, 44.10% yield).

¹H NMR (400 MHz, CDCl₃): δ 4.07 (s, 2H), 3.68 (s, 3H), 3.43 (s, 3H), 2.77-2.75 (m, 2H), 2.67-2.64 (m, 2H).

### Step 2

### 5-methoxy-4-oxopentanoic acid 63c

To a solution of 63b (500 mg, 3.12 mmol) in Water (10 mL) and THF (10 mL) was added LiGH (149 mg, 6.24 mmol). The reaction was stirred at r.t for 2 h. Water (20 mL) was added and the mixture was extracted with EtOAc (20 mL×2). The organic solution was washed with brine, dried over Na₂SO₄ and concentrated to give crude 63c (260 mg, 56.99% yield). The crude product was used directly in next step.

### Step 3

### 5-methoxy-1-(5-(trifluoromethyl)isoindolin-2-yl)pentane-1,4-dione 63d

To a solution of 63c (260 mg, 1.78 mmol) and 5-(trifluoromethyl)isoindoline 1-1 (333 mg, 1.49 mmol, HCl) in DMF (20 mL) was added triethylamine (720 mg, 7.12 mmol) and HATU (812 mg, 2.13 mmol). The reaction was stirred at r.t for 2 h. Water (40 mL) was added and the mixture was extracted with EtOAc (30 mL×2), the organic solution was washed with brine, dried over Na₂SO₄ and concentrated. The residue was purified by silica gel chromatography (hexane: EtOAc=2:1) to give 63d (320 mg, 96.10% yield).

LCMS: m/z (ESI): 316.1 [M+H]⁺.

### Step 4

### 5-(methoxymethyl)-5-(3-oxo-3-(5-(trifluoromethyl)isoindolin-2-yl)propyl)imidazolidine-2,4-dione 63

To a solution of 63d (200 mg, 634.34 umol) in EtOH (5 mL) was added to a solution of Ammonium carbonate (488 mg, 5.07 mmol) in water (5 mL). Then Sodium cyanide (78 mg, 1.59 mmol) was added. The reaction was stirred at 80° C. for 18 h in the sealed vessel. The solution was cooled to r.t. Water (50 mL) was added and the mixture was extracted with EtOAc (40 mL×2). The organic solution was washed with brine, dried over Na₂SO₄ and concentrated.

The residue was purified by prep-HPLC to give 63 (140 mg, 57.27% yield).

¹H NMR (400 MHz, DMSO-d₆): δ 10.62 (s, 1H), 7.89 (s, 1H), 7.75 (s, 1H), 7.67 (d, 1H), 7.59 (d, 1H), 4.88-4.85 (m, 2H), 4.68 (br, 2H), 3.51 (d, 1H), 3.35 (d, 1H), 3.25 (s, 3H), 2.43-2.34 (m, 1H), 2.27-2.18 (m, 1H), 1.89-1.75 (m, 2H).

LCMS: m/z (ESI): 386.1 [M+H]⁺.

### Examples 63-1 and 63-2

### (S)-5-(methoxymethyl)-5-(3-oxo-3-(5-(trifluoromethyl)isoindolin-2-yl)propyl)imidazolidine-2,4-dione 63-1 (R)-5-(methoxymethyl)-5-(3-oxo-3-(5-(trifluoromethyl)isoindolin-2-yl)propyl)imidazolidine-2,4-dione 63-2

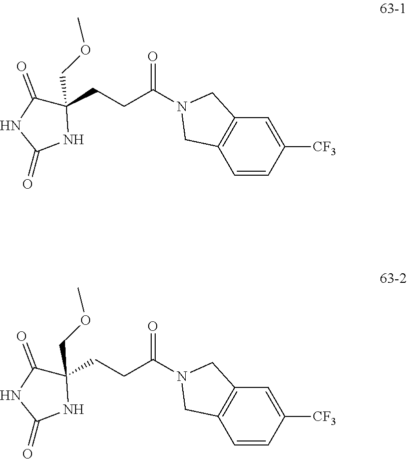 [SMILES: COC[C@]1(CCC(=O)N2Cc3ccc(C(F)(F)F)cc3C2)NC(=O)NC1=O ; COC[C@@]1(CCC(=O)N2Cc3ccc(C(F)(F)F)cc3C2)NC(=O)NC1=O]

63 was seperated by SFC to give the title single enantiomer compounds (42 mg and 55 mg).

### Enantiomer (Shorter Retention Time):

¹H NMR (400 MHz, DMSO-d₆): δ 10.59 (br, 1H), 7.89 (d, 1H), 7.75 (s, 1H), 7.67 (d, 1H), 7.58 (d, 1H), 4.88-4.85 (m, 2H), 4.68 (br, 2H), 3.50 (d, J=9.6 Hz, 1H), 3.35 (d, 1H), 3.25 (s, 3H), 2.42-2.32 (m, 1H), 2.26-2.17 (m, 1H), 1.89-1.76 (m, 2H).

LCMS: m/z (ESI): 386.1 [M+H]⁺.

ChirHPLC (EtOH/DEA 5%-40% 1.5 ml/min IC, 3 um 3.0*100(Daicel)): Rt:4.084 min, ee:100%.

### Enantiomer (Longer Retention Time):

¹H NMR (400 MHz, DMSO-d₆): δ 10.62 (s, 1H), 7.89 (s, 1H), 7.75 (s, 1H), 7.67 (d, 1H), 7.58 (d, 1H), 4.88-4.85 (m, 2H), 4.68 (br, 2H), 3.50 (d, 1H), 3.35 (d, 1H), 3.25 (s, 3H), 2.43-2.32 (m, 1H), 2.27-2.17 (m, 1H), 1.88-1.76 (m, 2H).

LCMS: m/z (ESI): 386.1 [M+H]⁺.

ChirHPLC (EtOH/DEA 5%-40% 1.5 ml/min IC, 3 um 3.0*100(Daicel)): Rt:4.766 min, ee:100%.

### Example 64

### 5-(1-methylcyclopropyl)-5-(3-oxo-3-(5-(trifluoromethyl)isoindolin-2-yl)propyl)imidazolidine-2,4-dione 64

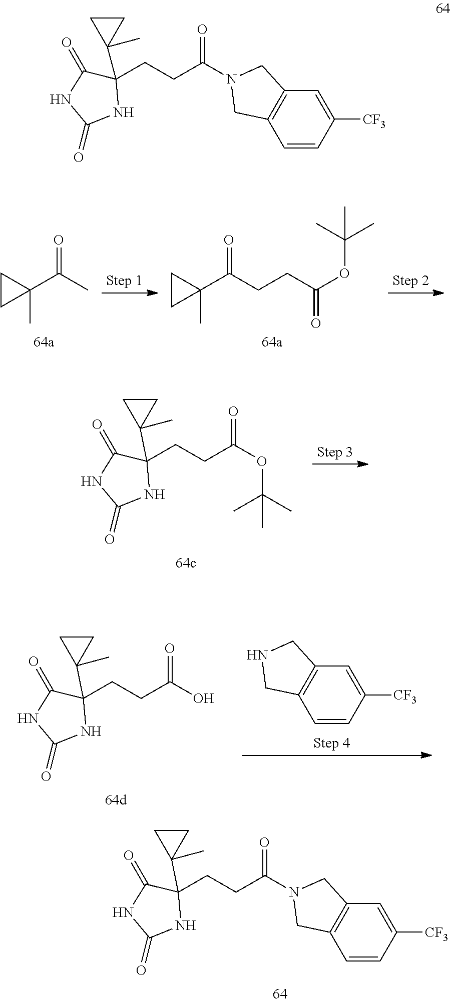

### Step 1

### Tert-butyl 4-(1-methylcyclopropyl)-4-oxobutanoate 64b

The solution of LDA (1.31 g, 12.23 mmol) in THF (5 mL) was colded to −78° C. and stirred for 20 min under N₂ atmosphere. Then the solution of 1-(1-methylcyclopropyl)ethanone 64a (1.0 g, 10.19 mmol) in THF (3 mL) was added dropwise before the reaction mixture was warmed to 20° C. for 30 min. Then the mixture was re-cooded to −78° C. before the solution of tert-butyl 2-bromoacetate (1.99 g, 10.19 mmol) in THF (2 mL) was added dropwise. The mixture was stirred at room temperature overnight. The reaction was quenched by saturated NH₄C₁ and then extracted with ethylacetate. The combined organic layers were washed with water and brine, dried over anhydrous Na₂SO₄, filtered and concentrated. The residue was purified by silica gel chromatography (EtOAc/hexane=1/10) to afford 64b (1.6 g, 7.54 mmol, 73.97% yield).

### Step 2

### Tert-butyl 3-(4-(1-methylcyclopropyl)-2,5-dioxoimidazolidin-4-yl)propanoate 64c

To a solution of 64b (1.7 g, 8.01 mmol) in MeOH (25 mL) was added (NH₄)₂CO₃ (4.16 g, 43.24 mmol), H₂O (25 mL) and NaCN (1.06 g, 20.02 mmol). The reaction mixture was stirred at 80° C. in a seal tube overnight and then cooled to RT. Water was added, the mixture was extracted with ethylacetate. The combined organic layers were washed with water and brine, dried over anhydrous Na₂SO₄, filtered and concentrated in vacuo. The residue was triturated with EtOAc/hexane=1/5, filtered and dried to give 64c (880 mg, 3.28 mmol, 40.96% yield).

¹HNMR (400 MHz, CDCl₃): δ 8.31 (brs, 1H), 6.01 (brs, 1H), 2.32-2.24 (m, 2H), 2.18-2.07 (m, 2H), 1.44 (s, 9H), 1.21 (s, 3H), 0.76-0.70 (m, 1H), 0.61-0.55 (m, 1H), 0.41-0.28 (m, 2H).

### Step 3

### 3-(4-(1-methylcyclopropyl)-2,5-dioxoimidazolidin-4-yl)propanoic acid 64d

To a solution of 64c (880 mg, 3.28 mmol) in MeOH (10 mL) was added HCl/Dioxane (16 mL, 6N), and the mixture was stirred at room temperature for 2 h. The reaction mixture was concentrated in vacuo and the residue was triturated with ether, filtered and dried to give 64d (630 mg, 2.97 mmol, 90.52% yield). The product was directly used for the next reaction.

### Step 4

### 5-(1-methylcyclopropyl)-5-(3-oxo-3-(5-(trifluoromethyl)isoindolin-2-yl)propyl)imidazolidine-2,4-dione 64

To a solution of 5-(trifluoromethyl)isoindoline 1-1 (90 mg, 480.87 umol) in DMF (10 mL) was added TEA (0.3 mL), 64d (100 mg, 471.25 umol) and HATU (197.10 mg, 518.37 umol).

The mixture was stirred at room temperature for 2 h. Water was added, and the mixture was extracted with EtOAc. The combined organic layers were washed with water and brine, dried over anhydrous Na₂SO₄, filtered and concentrated. The residue was purified by prep-HPLC to give 64 (130 mg, 340.90 umol, 72.34% yield).

¹HNMR (400 MHz, DMSO-d₆): δ 10.63 (s, 1H), 7.90 (s, 1H), 7.78 (s, 1H), 7.66 (d, 1H), 7.58 (d, 1H), 4.86 (brs, 2H), 4.70 (brs, 2H), 2.42-2.20 (m, 2H), 2.07-1.94 (m, 2H), 1.15 (s, 3H), 0.65-0.58 (m, 1H), 0.50-0.44 (m, 1H), 0.29-0.18 (m, 2H).

¹⁹FNMR (376.5 MHz, DMSO-d₆): δ-60.55.

LCMS: MS m/z (ESI): 396.4 [M+H]⁺.

### Examples 64-1 and 64-2 (R)-5-(1-methylcyclopropyl)-5-(3-oxo-3-(5-(trifluoromethyl)isoindolin-2-yl)propyl)imidazolidine-2,4-dione 64-1

### (S)-5-(1-methylcyclopropyl)-5-(3-oxo-3-(5-(trifluoromethyl)isoindolin-2-yl)propyl)imidazolidine-2,4-dione 64-2

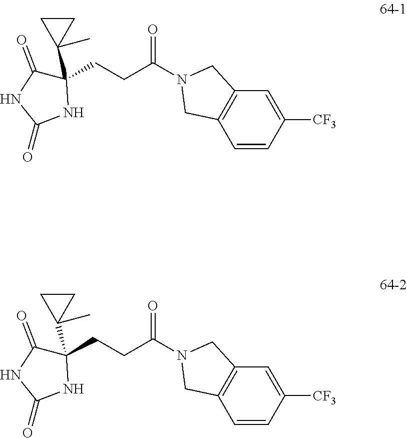

64 (70 mg) was separated by SFC (DAICELCHIRALCEL® IG) to give two enantiomers (22 mg and 18 mg).

### Enantiomer (Shorter Retention Time):

¹HNMR (400 MHz, DMSO-d₆): δ 10.63 (s, 1H), 7.90 (d, 1H), 7.77 (s, 1H), 7.66 (d, 1H), 7.58 (d, 1H), 4.87-4.85 (m, 2H), 4.70 (brs, 2H), 2.40-2.19 (m, 2H), 2.08-1.94 (m, 2H), 1.15 (s, 3H), 0.65-0.58 (m, 1H), 0.50-0.44 (m, 1H), 0.30-0.17 (m, 2H).

¹⁹FNMR (376.5 MHz, DMSO-d₆): δ-60.56.

LCMS: MS m/z (ESI): 396.4 [M+H]⁺.

Chiral HPLC (CO₂/MeOH/DEA 5%-40% 1.5 ml/min IG,3 um,3*100(Daicel)): Rt: 5.027 min, ee: 100%.

### Enantiomer (Longer Retention Time):

¹HNMR (400 MHz, DMSO-d₆): δ 10.63 (s, 1H), 7.90 (s, 1H), 7.77 (s, 1H), 7.66 (d, 1H), 7.58 (d, 1H), 4.86 (brs, 2H), 4.70 (brs, 2H), 2.41-2.20 (m, 2H), 2.04-1.93 (m, 2H), 1.15 (s, 3H), 0.63-0.60 (m, 1H), 0.47 (br, 1H), 0.29-0.17 (m, 2H).

¹⁹FNMR (376.5 MHz, DMSO-d₆): δ-60.56.

LCMS: MS m/z (ESI): 396.4 [M+H]⁺.

Chiral HPLC (CO₂/MeOH/DEA 5%-40% 1.5 ml/min IG,3 um,3*100(Daicel)): Rt: 5.892 min, ee: 100%.

### Example 65

### 5-(2-((benzyloxy)methyl)-3-oxo-3-(5-(trifluoromethyl)isoindolin-2-yl)propyl)-5-cyclopropylimidazolidine-2,4-dione 65

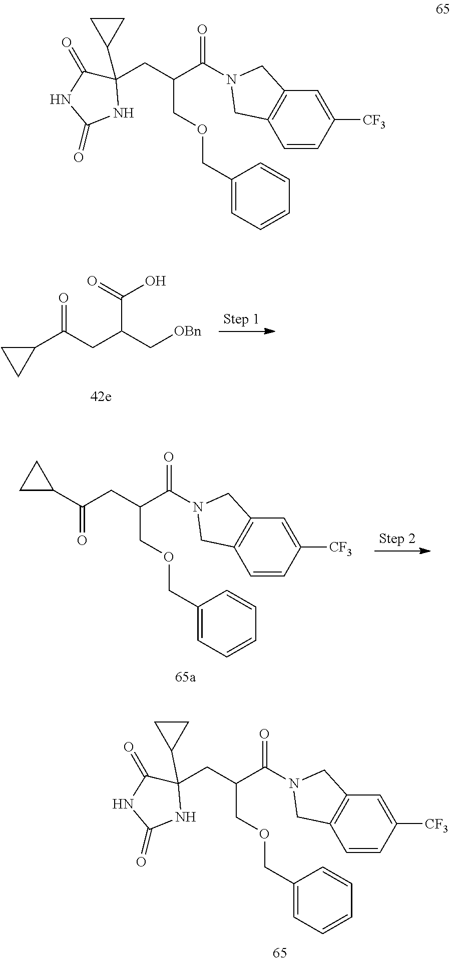

### Step 1

### 2-((benzyloxy)methyl)-4-cyclopropyl-1-(5-(trifluoromethyl)isoindolin-2-yl)butane-1,4-dione 65a

To a solution of 42e (200 mg, 762.48 umol) and 5-(trifluoromethyl)isoindoline 1-1 (120 mg, 638.66 umol) in DMF (10 mL), triethylamine (232 mg, 2.29 mmol) and HATU (347.90 mg, 914.98 umol) were added. The reaction was stirred at room temperature for 2 h. Water (20 mL) was added and the mixture was extracted with EtOAc (20 mL×2), the organic solution was washed with brine, dried over Na₂SO₄ and concentrated. The residue was purified by prep-TLC to give 65a (200 mg, 60.80% yield).

LCMS: m/z (ESI): 432.1 [M+H]⁺.

### Step 2

### 5-(2-((benzyloxy)methyl)-3-oxo-3-(5-(trifluoromethyl)isoindolin-2-yl)propyl)-5-cyclopropylimidazolidine-2,4-dione 65

To a solution of 65a (200 mg, 463.56 umol) in EtOH (2 mL), ammonium carbonate (356.35 mg, 3.71 mmol), NaCN (61.47 mg, 1.16 mmol) and water (2 mL) were added. The reaction was stirred at 80° C. for 18 h. Water (20 mL) was added and the mixture was extracted with EtOAc (20 mL×2), the organic solution was washed with brine, dried over Na₂SO₄ and concentrated. The residue was purified by prep-HPLC to give 65 (85 mg, 36.56% yield).

¹H NMR (400 MHz, DMSO-d₆): δ 10.68 (brs, 1H), 7.78-7.66 (m, 3H), 7.61-7.53 (m, 1H), 7.23 (br, 5H), 5.03-4.64 (m, 4H), 4.45 (s, 2H), 3.55-3.42 (m, 2H), 2.99-2.91 (m, 1H), 2.33-2.23 (m, 1H), 1.86-1.82 (m, 1H), 1.08-0.99 (m, 1H), 0.44-0.24 (m, 3H), 0.04-0.01 (m, 1H).

LCMS: m/z (ESI): 502.2 [M+H]⁺.

### Example 66

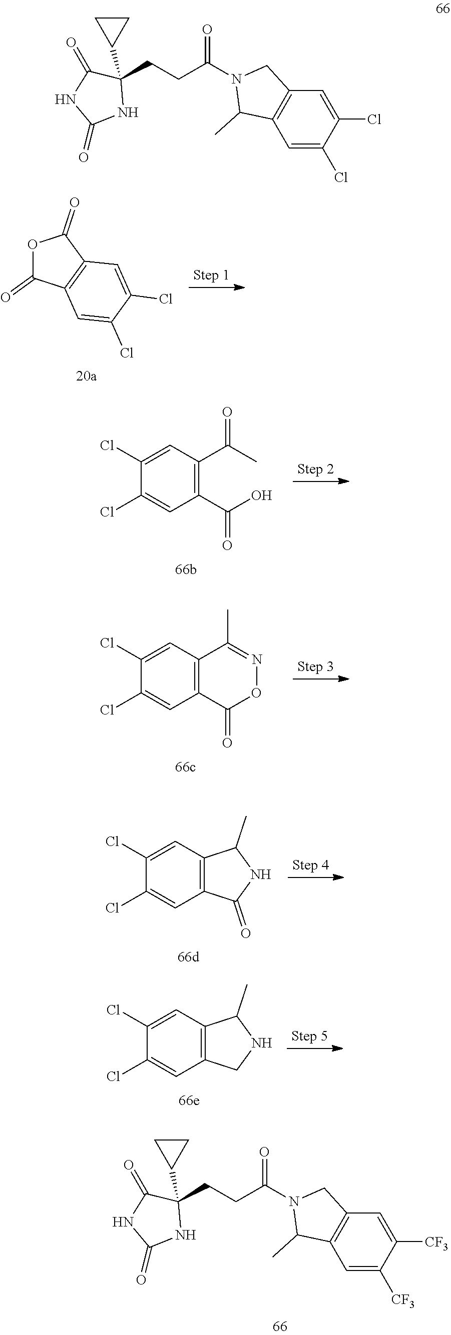

### (5S)-5-cyclopropyl-5-(3-(5,6-dichloro-1-methylisoindolin-2-yl)-3-oxopropyl)imidazolidine-2,4-dione 66

### Step 1

### 2-acetyl-4,5-dichlorobenzoic acid 66b

To a mixture of 5,6-dichloroisobenzofuran-1,3-dione 20a (5 g, 23.04 mmol) and 3,3-dihydroxypropanoic acid (3.67 g, 34.56 mmol) in pyridine (5 mL) was stirred at 75° C. for 2 h.

Water (16 mL) and conc. HCl (16 mL) were added, the reaction was stirred at 130° C. for 30 min.

The mixture was cooled to r.t, then filtered to give 66b (2.1 g, 39.11% yield).

LCMS: MS m/z (ESI): 230.9 [M−H]⁻.

### Step 2

### 6,7-dichloro-4-methyl-1H-benzo[d][1,2]oxazin-1-one 66c

To a solution of 66b (2.1 g, 9.01 mmol) in water (12 mL) was added KOH (1.52 g, 27.03 mmol).

Then hydroxylamine hydrochloride (1.25 g, 18.02 mmol) was slowly added to the solution.

The reaction was stirred at room temperature for 18 h. The solution was cooled to 0° C., the resulting precipitate was filtered. The solid was dried to give 66c (1.2 g, 57.89% yield).

LCMS: MS m/z (ESI): 230.1 [M+H]⁺.

### Step 3

### 5,6-dichloro-3-methylisoindolin-1-one 66d

To a solution of 66c (3 g, 13.04 mmol) in acetic acid (20 mL) was added Zn (10 g, 153.85 mmol). The reaction was stirred at 115° C. for 24 h. The mixture was cooled to room temperature and filtered, the cake was washed with DCM, and the filtrate was concentrated. The residue was purified by trituration (hexane: EtOAc=4:1) to give 66d (2 g, 70.98% yield).

LCMS: MS m/z (ESI): 216.1 [M+H]⁺.

### Step 4

### 5,6-dichloro-1-methylisoindoline 66e

To a solution of 66d (2 g, 9.26 mmol) in THF (10 mL) was added borane-tetrahydrofuran complex (629.44 mg, 37.03 mmol, 20 mL). The reaction was stirred at 60° C. for 18 h. MeOH (2 mL) was added dropwise and HCl (6M, 2 mL) was added, the reaction was stirred at 80° C. for 2 h. Then NaOH (5M) was added to adjust the mixture to pH=7, the solution was dried and concentrated. The residue was purified by was added (DCM:MeOH=20:1) to give 66e (900 mg, 4.45 mmol, 48.11% yield).

¹H NMR (400 MHz, DMSO-d₆): δ 7.62 (s, 2H), 4.63-4.58 (m, 1H), 4.31-4.19 (m, 2H), 1.46 (d, J=6.8 Hz, 3H).

LCMS: m/z (ESI): 202.1 [M+H]⁺.

### Step 5

### (5S)-5-cyclopropyl-5-(3-(5,6-dichloro-1-methylisoindolin-2-yl)-3-oxopropyl)imidazolidine-2,4-dione 66

To a solution of 66e (1 g, 4.95 mmol) and Int-1 (1.16 g, 5.44 mmol) in DMF (50 mL), triethylamine (1.50 g, 14.85 mmol) and HATU (2.26 g, 5.94 mmol) were added. The reaction was stirred at room temperature for 2 h. Water (100 mL) was added and the mixture was extracted with EtOAc (100 mL×2). The combined organic phases were washed with brine, dried over Na₂SO₄ and concentrated. The residue was purified by silica gel chromatagraphy (DCM: MeOH=50:1) to give 66 (900 mg, 46% yield).

LCMS: MS m/z (ESI): 396 [M+H]⁺.

¹H NMR (400 MHz, Methanol-d₄) δ 7.40 (d, 2H), 5.14 (q, 1H), 4.71 (d, 2H), 2.56-2.23 (m, 2H), 2.22-2.01 (m, 2H), 1.39 (t, 3H), 1.13 (m, 1H), 0.48-0.17 (m, 4H).

### Examples 66-1 and 66-2

### (S)-5-cyclopropyl-5-(3-((S)-5,6-dichloro-1-methylisoindolin-2-yl)-3-oxopropyl)imidazolidine-2,4-dione 66-1

### (S)-5-cyclopropyl-5-(3-((R)-5,6-dichloro-1-methylisoindolin-2-yl)-3-oxopropyl)imidazolidine-2,4-dione 66-2

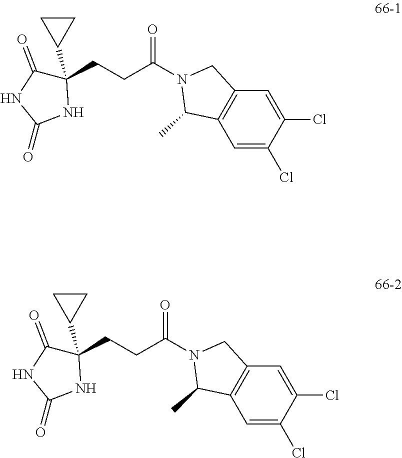 [SMILES: C[C@H]1c2cc(Cl)c(Cl)cc2CN1C(=O)CC[C@@]1(C2CC2)NC(=O)NC1=O ; C[C@@H]1c2cc(Cl)c(Cl)cc2CN1C(=O)CC[C@@]1(C2CC2)NC(=O)NC1=O]

Racemate 66 (900 mg) was seperated by SFC to give to give two isomers (340 mg and 320 mg separately).

Isomer with Longer Retention Time:

¹H NMR (400 MHz, DMSO-d₆): δ10.62 (s, 1H), 7.78-7.65 (m, 3H), 5.20-5.10 (m, 1H), 4.81-4.71 (m, 2H), 2.49-2.18 (m, 2H), 2.03-1.97 (m, 2H), 1.44-1.39 (m, 3H), 1.15-1.07 (m, 1H), 0.49-0.29 (m, 3H), 0.15-0.08 (m, 1H).

LCMS: m/z (ESI): 396.0 [M+H]⁺.

ChirHPLC(CO₂/MeOH/DEA 60/40/0.04 1.8 ml/min IA, 3 um 3*100(Daicel)): Rt:3.001 min, ee 97.10%.

Isomer with Shorter Retention Time:

¹H NMR (400 MHz, DMSO-d₆): δ10.64 (br, 1H), 7.80-7.65 (m, 3H), 5.25-5.10 (m, 1H), 4.81-4.71 (m, 2H), 2.51-2.23 (m, 2H), 2.10-1.93 (m, 2H), 1.43-1.38 (m, 3H), 1.15-1.05 (m, 1H), 0.49-0.29 (m, 3H), 0.14-0.06 (m, 1H).

LCMS: m/z (ESI): 396.0 [M+H]⁺.

ChirHPLC (CO₂/MeOH/DEA 60/40/0.04 1.8 ml/min IA, 3 um 3*100(Daicel)): Rt:1.689 min, ee:100%.

### Examples 67-1, 67-2, 68-1, 68-2

### (S)-5-(3-((R)-8-chloro-9-fluoro-1-methyl-1,2,4,5-tetrahydro-3H-benzo[d]azepin-3-yl)-3-oxopropyl)-5-cyclopropylimidazolidine-2,4-dione 67-1

### (S)-5-(3-((S)-8-chloro-9-fluoro-1-methyl-1,2,4,5-tetrahydro-3H-benzo [d]azepin-3-yl)-3-oxopropyl)-5-cyclopropylimidazolidine-2,4-dione 67-2

### (S)-5-(3-((S)-8-chloro-7-fluoro-1-methyl-1,2,4,5-tetrahydro-3H-benzo [d]azepin-3-yl)-3-oxopropyl)-5-cyclopropylimidazolidine-2,4-dione 68-1

### (S)-5-(3-((R)-8-chloro-7-fluoro-1-methyl-1,2,4,5-tetrahydro-3H-benzo [d]azepin-3-yl)-3-oxopropyl)-5-cyclopropylimidazolidine-2,4-dione 68-2

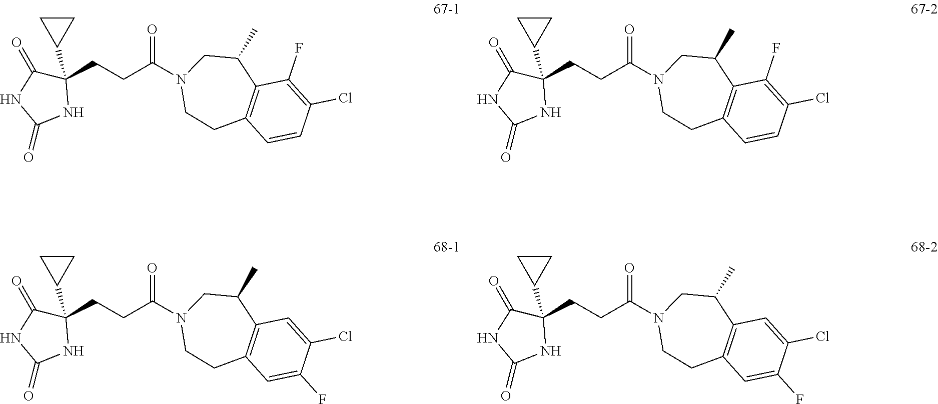 [SMILES: C[C@H]1CN(C(=O)CC[C@@]2(C3CC3)NC(=O)NC2=O)CCc2ccc(Cl)c(F)c21 ; C[C@@H]1CN(C(=O)CC[C@@]2(C3CC3)NC(=O)NC2=O)CCc2ccc(Cl)c(F)c21 ; C[C@@H]1CN(C(=O)CC[C@@]2(C3CC3)NC(=O)NC2=O)CCc2cc(F)c(Cl)cc21 ; C[C@H]1CN(C(=O)CC[C@@]2(C3CC3)NC(=O)NC2=O)CCc2cc(F)c(Cl)cc21]

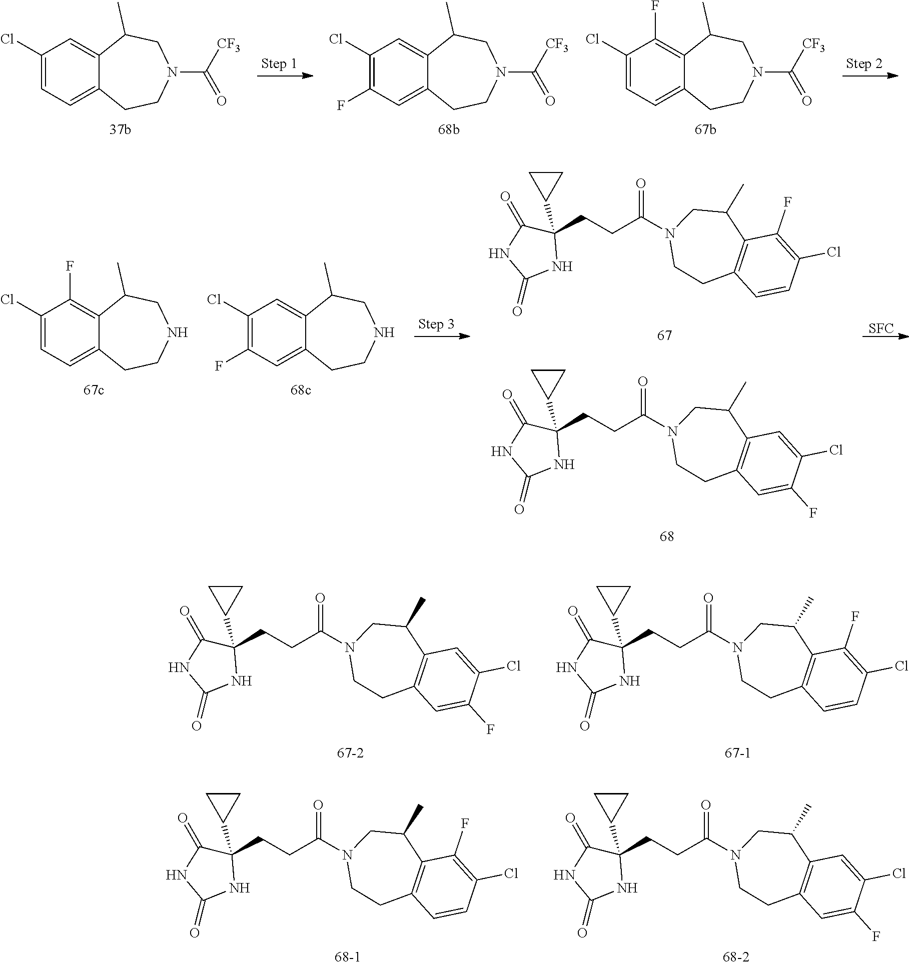

### Step 1

### Mixture of 1-(8-chloro-9-fluoro-1-methyl-1,2,4,5-tetrahydro-3H-benzo[d]azepin-3-yl)-2,2,2-trifluoroethan-1-one 67b & 1-(8-chloro-7-fluoro-1-methyl-1,2,4,5-tetrahydro-3H-benzo[d]azepin-3-yl)-2,2,2-trifluoroethan-1-one 68b

To a solution of 37b (2 g, 6.86 mmol) in 1,2-dichloroethane (12 mL), trifluoromethanesulfonic acid (10.29 g, 68.56 mmol) and Selectfluor (4.86 g, 13.71 mmol) were added. The reaction was stirred at 75° C. for 18 h. Water (50 mL) was added and the mixture was extracted with DCM (20 mL×2). The combined organic phases were washed with brine, dried over Na₂SO₄ and concentrated. The residue was purified by silica gel chromatagraphy (hexane: EtOAc=20:1) to give the mixture of 67b & 68b (800 mg, 37.68% yield).

LCMS: MS m/z (ESI): 310.0 [M+H]⁺.

### Step 2

### Mixture of 8-chloro-9-fluoro-1-methyl-2,3,4,5-tetrahydro-1H-benzo[d]azepine 67c & 1-(8-chloro-9-fluoro-1-methyl-1,2,4,5-tetrahydro-3H-benzo [d]azepin-3-yl)-2,2,2-trifluoroethan-1-one 68c

To a solution of 67b & 68b (800 mg, 2.58 mmol) in MeOH (18 mL) was added a solution of NaOH (207 mg, 5.17 mmol) in water (5 mL). The reaction was stirred at room temperature for 2 h. Water (20 mL) was added and the mixture was extracted with EtOAc (20 mL×3), the combined layers was washed with brine, dried over Na₂SO₄ and concentrated to give the mixture of 67c & 68c (550 mg, 2.57 mmol, 99.64% yield). The crude solid was used to next step directly.

### Step 3

### Mixture of (5S)-5-(3-(8-chloro-9-fluoro-1-methyl-1,2,4,5-tetrahydro-3H-benzo[d]azepin-3-yl)-3-oxopropyl)-5-cyclopropylimidazolidine-2,4-dione 67 & (5S)-5-(3-(8-chloro-7-fluoro-1-methyl-1,2,4,5-tetrahydro-3H-benzo[d]azepin-3-yl)-3-oxopropyl)-5-cyclopropylimidazolidine-2,4-dione 68

To a solution of 67c & 68c (500 mg, 2.34 mmol) and 3-[(4S)-4-cyclopropyl-2,5-dioxo-imidazolidin-4-yl]propanoic acid Int-1 (601 mg, 2.83 mmol) in DMF (20 mL), triethylamine (710.34 mg, 7.02 mmol) and HATU (1.07 g, 2.81 mmol) were added. The reaction was stirred at room temperature for 2 h. The reaction was diluted with water (50 mL) and the mixture was extracted with EtOAc (50 mL×2). The combined organic layers were washed with brine, dried over Na₂SO₄ and concentrated. The residue was purified by prep-HPLC to give the mixture of 67 & 68 (700 mg, 73.35% yield).

### (S)-5-(3-((R)-8-chloro-9-fluoro-1-methyl-1,2,4,5-tetrahydro-3H-benzo [d]azepin-3-yl)-3-oxopropyl)-5-cyclopropylimidazolidine-2,4-dione 67-1 & (S)-5-(3-((S)-8-chloro-9-fluoro-1-methyl-1,2,4,5-tetrahydro-3H-benzo[d]azepin-3-yl)-3-oxopropyl)-5-cyclopropylimidazolidine-2,4-dione 67-2

### (S)-5-(3-((S)-8-chloro-7-fluoro-1-methyl-1,2,4,5-tetrahydro-3H-benzo [d]azepin-3-yl)-3-oxopropyl)-5-cyclopropylimidazolidine-2,4-dione 68-1 & (S)-5-(3-((R)-8-chloro-7-fluoro-1-methyl-1,2,4,5-tetrahydro-3H-benzo[d]azepin-3-yl)-3-oxopropyl)-5-cyclopropylimidazolidine-2,4-dione 68-2

Mixture of 67 & 68 was separated by SFC (MeOH/DEA 5%-40% 1.5 ml/min IA, 3 um 3.0*100(Daicel)) to give four isomers.

Isomer (peak 1, Rt: 4.729 min):

¹H NMR (400 MHz, DMSO-d₆): δ 10.59 (d, 1H), 7.70 (d, 1H), 7.33 (t, 1H), 7.02 (t, 1H), 3.91-3.48 (m, 4H), 3.33-3.30 (m, 1H), 3.28-3.07 (m, 1H), 2.94-2.82 (m, 1H), 2.40-2.15 (m, 2H), 1.97-1.73 (m, 2H), 1.15 (dd, 3H), 1.08-0.99 (m, 1H), 0.43-0.25 (m, 3H), 0.10-0.03 (m, 1H).

LCMS: m/z (ESI): 408.1 [M+H]⁺.

ChirHPLC 1 (MeOH/DEA 5%-40% 1.5 ml/min IA, 3 um 3.0*100(Daicel)): peak 1, Rt: 4.729 min.

ChirHPLC 2 (CO₂/EtOH/DEA 60/40/0.04 2.8 ml/min AD,5 um,4.6*250(Daicel)): Rt:2.736 min, ee:100%.

Isomer (peak 2, Rt: 5.284 min):

¹H NMR (400 MHz, DMSO-d₆): δ 10.59 (d, 1H), 7.71 (d, 1H), 7.33 (t, 1H), 7.02 (t, 1H), 3.89-3.47 (m, 4H), 3.31-3.06 (m, 2H), 2.91-2.82 (m, 1H), 2.36-2.00 (m, 2H), 1.95-1.78 (m, 2H), 1.15 (dd, 3H), 1.08-1.00 (m, 1H), 0.46-0.25 (m, 3H), 0.11-0.04 (m, 1H).

LCMS: m/z (ESI): 408.1 [M+H]⁺.

ChirHPLC 1 (MeOH/DEA 5%-40% 1.5 ml/min IA, 3 um 3.0*100(Daicel)): peak 2, Rt: 5.284 min.

ChirHPLC 2 (CO2/EtOH/DEA 60/40/0.04 2.8 ml/min AD,5 um,4.6*250(Daicel)): Rt:5.410 min, ee:99.42%.

Isomer (peak 3, Rt: 5.970 min):

¹H NMR (400 MHz, DMSO-d₆): δ 10.60 (d, 1H), 7.70 (d, 1H), 7.34 (dd, 1H), 7.23 (t, 1H), 3.65-3.38 (m, 4H), 3.22-2.80 (m, 3H), 2.41-2.21 (m, 2H), 1.93-1.75 (m, 2H), 1.20 (dd, 3H), 1.10-1.01 (m, 1H), 0.46-0.26 (m, 3H), 0.11-0.05 (m, 1H).

LCMS: m/z (ESI): 408.1 [M+H]⁺. Chiral HPLC 1 (MeOH/DEA 5%-40% 1.5 ml/min IA, 3 um 3.0*100(Daicel)): peak 3, Rt: 5.970 min.

Chiral HPLC 2 (CO₂/EtOH/DEA 60/40/0.04 2.8 ml/min AD,5 um,4.6*250(Daicel)): Rt: 3.801 min, ee:100%.

Isomer (peak 4, Rt: 6.442 min):

¹H NMR (400 MHz, DMSO-d₆): δ 10.60 (s, 1H), 7.72 (d, 1H), 7.34 (t, 1H), 7.23 (dd, 1H), 3.66-3.38 (m, 4H), 3.23-2.80 (m, 3H), 2.45-2.16 (m, 2H), 1.93-1.78 (m, 2H), 1.20 (dd, 3H), 1.09-1.00 (m, 1H), 0.48-0.26 (m, 3H), 0.12-0.05 (m, 1H).

LCMS: m/z (ESI): 408.1 [M+H]⁺.

ChirHPLC 1 (MeOH/DEA 5%-40% 1.5 ml/min IA, 3 um 3.0*100(Daicel)): peak 4, Rt: 6.442 min.

ChirHPLC 2 (CO₂/EtOH/DEA 60/40/0.04 2.8 ml/min AD,5 um,4.6*250(Daicel)): Rt: 8.117 min, ee:100%.

### Example 69

### (5S)-5-cyclopropyl-5-(3-(5,6-dichloro-1-methylisoindolin-2-yl-3,3-d₂)-3-oxopropyl)imidazolidine-2,4-dione 69

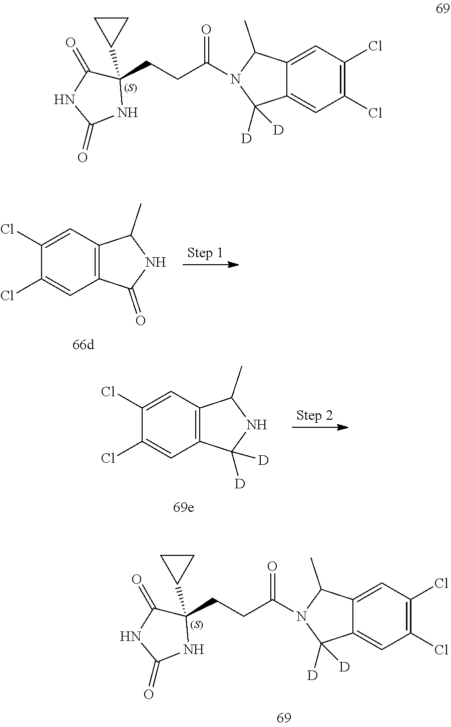

### Step 1

### 5,6-dichloro-1-methylisoindoline-3,3-d2 69e

To a solution of 66d (500 mg, 2.314 mmol) in THF (4 mL), borane-d3-THF complex solution (1M, 15 mL) was added. The reaction was stirred at 60° C. for 18 h. MeOH (2 mL) was added dropwise followed by HCl (6M, 2 mL), the reaction was stirred at 80° C. for 2 h. Then NaOH (5M) was added to adjust the mixture to pH=7, the solution was dried and concentrated. The residue was purified by silica gel chromatagraphy (DCM:MeOH=20:1) to give 69e (320 mg, 1.5 mmol, 65% yield).

LCMS: m/z (ESI): 204 [M+H]⁺.

### Step 2

### (5S)-5-cyclopropyl-5-(3-(5,6-dichloro-1-methylisoindolin-2-yl-3,3-d₂)-3-oxopropyl)imidazolidine-2,4-dione 69

To a mixture of 69e (20 mg, 0.094 mmol) in DMF (2 mL), triethylamine (40 mg, 0.28 mmol), Int-1 (23 mg, 0.108 mmol) and HATU (57 mg, 0.13 mmol) were added. The reaction was stirred at room temperature for 18 h. Water (3 mL) was added and the mixture was extracted with EtOAc (20 mL×3). The combined organic layers were washed with brine, dried over Na₂SO₄ and concentrated. The residue was purified with prep-HPLC to give title compound 69 (22 mg, 0.055 mol, 50% yield).

¹H NMR (400 MHz, Methanol-d₄): 7.39 (s, 2H), 5.14 (q, 1H), 2.58-2.22 (m, 2H), 2.20-2.02 (m, 3H), 0.49 (tt, 1H), 0.41-0.18 (m, 4H).

LCMS: m/z (ESI): 398[M+H]⁺.

### Examples 69-1 and 69-2

### (S)-5-cyclopropyl-5-(3-((S)-5,6-dichloro-1-methylisoindolin-2-yl-3,3-d₂)-3-oxopropyl)imidazolidine-2,4-dione 69-1

### (S)-5-cyclopropyl-5-(3-((R)-5,6-dichloro-1-methylisoindolin-2-yl-3,3-d2)-3-oxopropyl)imidazolidine-2,4-dione 69-2

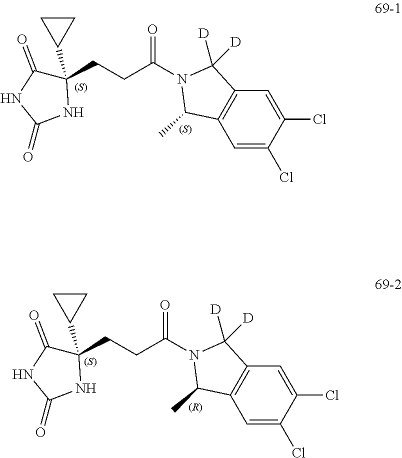

69 (85 mg) was separarted by SFC to give two diastereomers (15 mg, 16 mg).

### Diastereomer (Shorter Retention Time):

¹HNMR (400 MHz, DMSO-d₆): δ10.66-10.63 (m, 1H), 7.80-7.65 (m, 3H), 5.25-5.10 (m, 1H), 2.40-2.22 (m, 2H), 2.09-1.93 (m, 2H), 1.40 (dd, 3H), 1.15-1.06 (m, 1H), 0.49-0.29 (m, 3H), 0.14-0.07 (m, 1H).

LCMS: MS m/z (ESI): 398.0 [M+H]⁺.

Chiral HPLC (CO₂/MeOH/DEA 60/40/0.04 1.8 ml/min IA, 3 um 3*100(Daicel)): Rt: 1.581 min, de: 100%.

### Diastereomer (Longer Retention Time):

¹H NMR (400 MHz, DMSO-d₆): δ 10.63 (s, 1H), 7.79-7.65 (m, 3H), 5.22-5.10 (m, 1H), 2.44-2.36 (m, 1H), 2.26-2.17 (m, 1H), 2.03-1.96 (m, 2H), 1.40 (dd, 3H), 1.15-1.07 (m, 1H), 0.48-0.29 (m, 3H), 0.14-0.08 (m, 1H).

LCMS: m/z (ESI): 398.0 [M+H]⁺.

ChirHPLC (CO₂/MeOH/DEA 60/40/0.04 1.8 ml/min IA, 3 um 3*100(Daicel)): Rt: 3.053 min, de:99.38%.

### Example 70

### (5S)-5-cyclopropyl-5-(3-(5,6-dichloro-1-methylisoindolin-2-yl)-2-methyl-3-oxopropyl)imidazolidine-2,4-dione

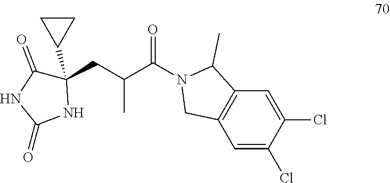 [SMILES: CC(C[C@@]1(C2CC2)NC(=O)NC1=O)C(=O)N1Cc2cc(Cl)c(Cl)cc2C1C]

To a mixture of 66e (10 mg, 0.049 mmol) in DMF (2 mL), triethylamine (20 mg, 0.15 mmol), Int-2A (9 mg, 0.042 mmol) and HATU (22.8 mg, 0.06 mmol) were added. The reaction was stirred at room temperature for 18 h. Water (3 mL) added and the mixture was extracted with EtOAc (20 mL×2). The combined layer was washed with brine, dried over Na₂SO₄ and concentrated. The residue was purified with prep-HPLC to give title compound 70 (6 mg, 0.014 mmol, 33% yield).

¹H NMR (400 MHz, Methanol-d4): δ 7.26 (t, J=3.3 Hz, 2H), 4.99 (q, 1H), 4.52 (d, 2H), 2.21 (m, 1H), 1.76 (m, 2H), 1.29 (dd, 3H), 0.97 (dd, 4H), 0.37-0.05 (m, 4H).

LCMS: m/z (ESI): 411 [M+H]⁺.

### Example 71

### (5S)-5-(3-(5-chloro-1-methyl-6-(trifluoromethyl)isoindolin-2-yl-3,3-d2)-3-oxopropyl)-5-cyclopropylimidazolidine-2,4-dione 71

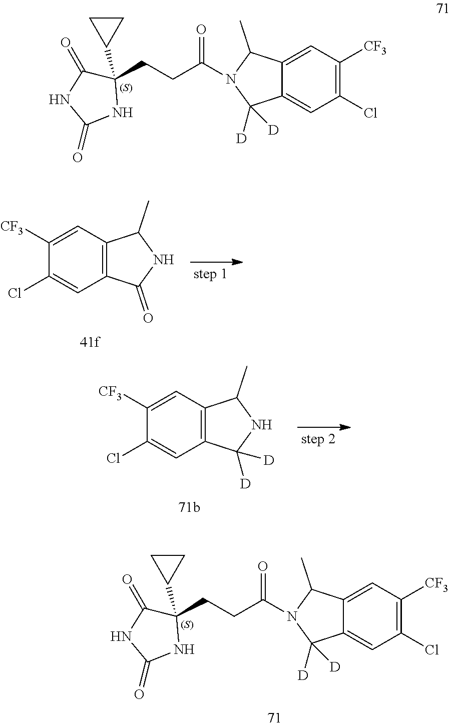

### Step 1

### 5-chloro-1-methyl-6-(trifluoromethyl)isoindoline-3,3-d₂ 71b

### Small Scale

To a solution of 41f (50 mg, 0.2 mmol) in THF (2 mL) was added Borane-d3-THF complex solution (6 mmol, 6 mL). The reaction was stirred at 60° C. for 18 h. MeOH (2 mL) was added dropwise followed by HCl (6M, 2 mL). The reaction was stirred at 80° C. for 2 h. Then NaOH (5M) was added to adjust the mixture to pH=7, the solution was dried and concentrated. The residue was purified by silica gel chromatagraphy (DCM:MeOH=20:1) to give 71b (39 mg, 0.147 mmol, 70% yield).

### Large Scale

To a solution 6-chloro-3-methyl-5-(trifluoromethyl)isoindolin-1-one 41f (800 mg, 3.20 mmol) in THF (10 ml) was added BD₃ (1M in THF, 64 ml, 64 mmol). After addition, the reaction was stirred at 60° C. (in a sealed tube) for 10 hours. It was quenched with MeOH (10 ml), followed by HCl (6 M, 20 ml). It then was stirred at 80° C. for 8 hours. 2 N NaOH was added to adjust pH to 7 and extracted with EtOAc, the combined organic phases were washed with brine (50 mL), dried over Na₂SO₄, filtered and concentrated under reduced pressure. The residue was purified by silica gel column chromatography eluting with 5% MeOH in DCM, to get the desired product 71b which is used for next step.

LCMS: MS m/z (ESI): 238.1 [M+H]⁺.

### Step 2

### (5S)-5-(3-(5-chloro-1-methyl-6-(trifluoromethyl)isoindolin-2-yl-3,3-d₂)-3-oxopropyl)-5-cyclopropylimidazolidine-2,4-dione 71

### Small Scale

To a mixture of 71b (20 mg, 0.084 mmol) in DMF (2 mL) was added triethylamine (40 mg, 0.28 mmol), Int-1 (23 mg, 0.108 mmol) and HATU (57 mg, 0.13 mmol). The reaction was stirred at r.t for 18 h. Water (3 mL) was added and the mixture was extracted with EtOAc (20 mL×3). The combined organic layers were washed with brine, dried over Na₂SO₄ and concentrated. The residue was purified with prep-HPLC to give title compound 71 (24 mg, 0.055 mol, 51% yield).

### Large Scale

To a solution of (S)-3-(4-cyclopropyl-2,5-dioxoimidazolidin-4-yl)propanoic acid Int-1 (680 mg, 3.2 mmol) in DMF (10 ml) was added EDCI (920 mg, 4.8 mmol) and HATU (1.83 g, 4.8 mmol). After stirring for 10 minutes, the isoindoline 71b collected from the previous step was added. The reaction was stirred at ambient temperature for 3 hours. LCMS showed that the reaction was completed. It was directly purified on a reverse phase HPLC to get the desired product 71 (1.10 g, 79.6% yield over two steps).

¹H NMR (400 MHz, CD₃OD): 7.76 (s, 1H), 7.63-7.60 (m, 1H), 5.57-5.53 (m, 1H), 2.59-2.40 (m, 2H), 2.28-2.19 (m, 2H), 1.56-1.50 (m, 3H), 1.28-1.21 (m, 1H), 0.62-0.58 (m, 1H), 0.49-0.41 (m, 3H).

LCMS: MS m/z (ESI): 432 [M+H]⁺.

### Examples 71-1 and 71-2

### (S)-5-(3-((R)-5-chloro-1-methyl-6-(trifluoromethyl)isoindolin-2-yl-3,3-d₂)-3-oxopropyl)-5-cyclopropylimidazolidine-2,4-dione 71-1

### (S)-5-(3-((S)-5-chloro-1-methyl-6-(trifluoromethyl)isoindolin-2-yl-3,3-d₂)-3-oxopropyl)-5-cyclopropylimidazolidine-2,4-dione 71-2

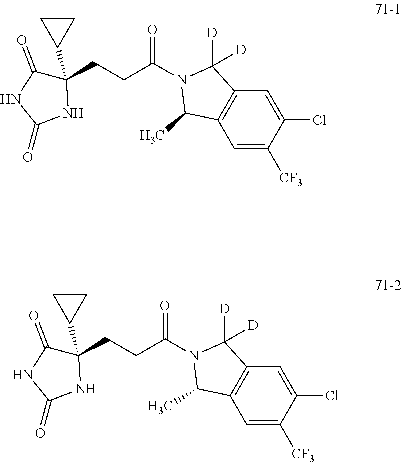 [SMILES: [2H]C1([2H])c2cc(Cl)c(C(F)(F)F)cc2[C@@H](C)N1C(=O)CC[C@@]1(C2CC2)NC(=O)NC1=O ; [2H]C1([2H])c2cc(Cl)c(C(F)(F)F)cc2[C@H](C)N1C(=O)CC[C@@]1(C2CC2)NC(=O)NC1=O]

71 (1.10 g) was separarted by SFC to give two diastereomers (325 mg and 415 mg separately).

### Enantiomers (Shorter Retention Time):

¹H NMR (500 MHz, DMSO-d₆) δ 10.63 (s, 1H), 7.89 (d, 1H), 7.75 (t, 2H), 5.30-5.16 (m, 1H), 4.23 (d, 1H), 2.37-2.27 (m, 2H), 1.99 (dq, 2H), 1.53 (s, 1H), 1.43 (dd, 31H), 1.11 (td, 1H), 0.49-0.30 (m, 3H), 0.11 (dt, 1H).

LCMS: MS m/z (ESI): 432.3 [M+H]⁺.

ChirHPLC (1% DEA in EtOH/hexane 60/40, 1.0 mL/min, 35° C., CHIRALPAK IG, 150*4.6 mm, Sum): Rt: 4.594 min, de:100%.

Enantiomers (longer retention time):

¹H NMR (500 MHz, DMSO-d₆) δ 10.53 (s, 1H), 7.89 (d, 1H), 7.80-7.66 (m, 2H), 5.30-5.12 (m, 1H), 4.23 (d, 1H), 2.44-2.36 (m, 1H), 2.31-2.20 (m, 1H), 2.05-1.95 (m, 2H), 1.44 (dd, 3H), 1.11 (td, 1H), 0.50-0.29 (m, 3H), 0.16-0.08 (m, 1H).

LCMS: MS m/z (ESI): 432.3 [M+H]⁺

ChirHPLC (1% DEA in EtOH/hexane 60/40, 1.0 mL/min, 35° C., CHIRALPAK IG, 150*4.6 mm, Sum): Rt: 10.931 min, de:100%.

### Example 72

### (S)-5-cyclopropyl-5-(3-oxo-3-(5-(2,2,2-trifluoroethyl)isoindolin-2-yl)propyl)imidazolidine-2,4-dione 72

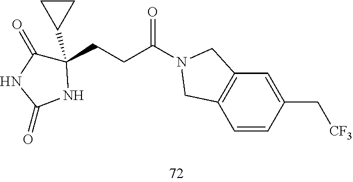 [SMILES: O=C1NC(=O)[C@](CCC(=O)N2Cc3ccc(CC(F)(F)F)cc3C2)(C2CC2)N1]

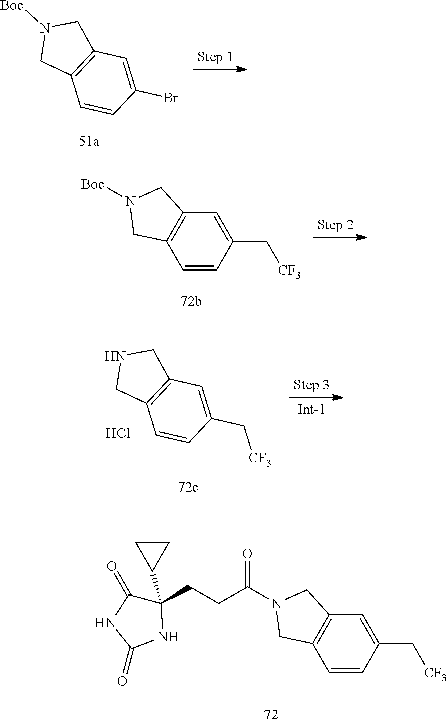 [SMILES: CC(C)(C)OC(=O)N1Cc2ccc(Br)cc2C1 ; CC(C)(C)OC(=O)N1Cc2ccc(CC(F)(F)F)cc2C1 ; Cl ; FC(F)(F)Cc1ccc2c(c1)CNC2 ; O=C1NC(=O)[C@](CCC(=O)N2Cc3ccc(CC(F)(F)F)cc3C2)(C2CC2)N1]

### Step 1

### Tert-butyl 5-(2,2,2-trifluoroethyl)isoindoline-2-carboxylate 72b

To a mixture of 51a (500.00 mg, 1.68 mmol) and 1,1,1-trifluoro-2-iodoethane (1.76 g, 8.38 mmol) in DMSO (3 mL) was added copper (1.07 g, 16.77 mmol). The reaction was stirred at 120° C. for 40 h. The reaction was cooled to rt, and water (100 mL) was added. The mixture was extracted with EtOAc (30 mL×2), and the combined organic layers were washed with brine, dried over Na₂SO₄ and concentrated. The crude was purified by prep-HPLC to give 72b (33 mg, 109.52 umol, 6.53% yield).

¹H NMR (400 MHz, CDCl₃): δ 7.21-7.15 (m, 3H), 4.69-4.65 (m, 4H), 3.37 (q, 2H), 1.52 (s, 9H).

¹⁹FNMR (400 MHz, CDCl₃): δ −66.03.

### Step 2

### 5-(2,2,2-trifluoroethyl)isoindoline hydrochloride 72c

The mixture of 72b (23 mg, 76.41 umol) in DCM (3 mL) was add HCl/dioxane (1N, 1 mL).

The reaction was stirred at room temperature for 16 hours. The mixture was concentrated to give 72c as crude which was used to the next step.

### Step 3

### (S)-5-cyclopropyl-5-(3-(5-(difluoromethyl) isoindolin-2-yl)-3-oxopropyl) imidazolidine-2,4-dione 72

To a solution of 72c (23 mg, 206.89 umol) in DMF (3 mL) was added Int-1 (23 mg, 109.35 umol), TEA (30 mg, 298.23 umol) and HATU (45 mg, 119.29 umol). The mixture was stirred at room temperature 2 h. Water (30 mL) was added and the mixture was extracted with EtOAc (20 mL×2). The combined organic layers were washed with water (30 mL) and brine (30 mL), dried and concentrated. The crude was purified by prep-HPLC to give 72 (13 mg, 32.88 umol, 33.08% yield).

¹H NMR (400 MHz, CDCl₃): δ 10.62 (brs, 1H), 7.74 (s, 1H), 7.37-7.27 (m, 3H), 4.80 (br, 2H), 4.62 (br, 2H), 3.67 (q, 2H), 2.47-2.40 (m, 1H), 2.30-2.26 (m, 1H), 2.01 (t, 2H), 1.13-1.09 (m, 1H), 0.47-0.31 (m, 3H), 0.13-0.10 (m, 1H).

¹⁹F NMR (400 MHz, CDCl₃): δ −64.44.

LCMS: MS m/z (ESI): 396.1 [M+H]⁺.

### Example 73

### 5-(3-(5-chloro-6-(trifluoromethyl)isoindolin-2-yl)-3-oxopropyl)-5-(1-methyl-1H-imidazol-2-yl)imidazolidine-2,4-dione 73

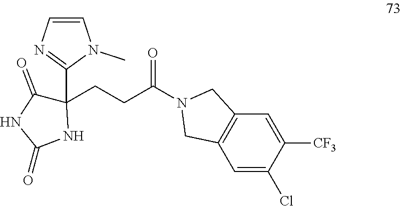 [SMILES: Cn1ccnc1C1(CCC(=O)N2Cc3cc(Cl)c(C(F)(F)F)cc3C2)NC(=O)NC1=O]

The title compound was prepared with similar method as Examples 40 and 62.

¹H NMR (400 MHz, DMSO-d₆): δ 8.57 (s, 1H), 7.91 (s, 1H), 7.77 (s, 1H), 7.19 (d, 1H), 6.84 (d, 1H), 4.85-4.81 (m, 2H), 4.71-4.67 (m, 2H), 3.54 (s, 3H), 2.59-2.54 (m, 2H), 2.45-2.38 (m, 2H).

LCMS: MS m/z (ESI): 455.9 [M+H]⁺.

### Examples 73-1 and 73-2

### (S)-5-(3-(5-chloro-6-(trifluoromethyl)isoindolin-2-yl)-3-oxopropyl)-5-(1-methyl-1H-imidazol-2-yl)imidazolidine-2,4-dione 73-1

### (R)-5-(3-(5-chloro-6-(trifluoromethyl)isoindolin-2-yl)-3-oxopropyl)-5-(1-methyl-1H-imidazol-2-yl)imidazolidine-2,4-dione 73-2

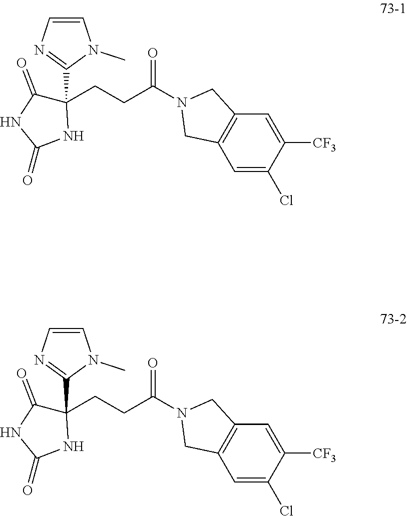 [SMILES: Cn1ccnc1[C@]1(CCC(=O)N2Cc3cc(Cl)c(C(F)(F)F)cc3C2)NC(=O)NC1=O ; Cn1ccnc1[C@@]1(CCC(=O)N2Cc3cc(Cl)c(C(F)(F)F)cc3C2)NC(=O)NC1=O]

73 (24 mg) was chirally separated by SFC to afford two enantiomers (5.0 mg, 5.0 mg).

### Enantiomer (Shorter Retention Time):

¹H NMR (400 MHz, DMSO-d₆): δ 11.16 (br, 1H), 8.57 (s, 1H), 7.91 (s, 1H), 7.77 (s, 1H), 7.19 (s, 1H), 6.84 (s, 1H), 4.85-4.81 (m, 2H), 4.71-4.67 (m, 2H), 3.54 (s, 3H), 2.59-2.54 (m, 2H), 2.45-2.38 (m, 2H).

LCMS: MS m/z (ESI): 456.1 [M+H]⁺.

Chiral HPLC (CO₂/MeOH/DEA 60/40/0.04 2.8 ml/min OD,5 um,4.6*250(Daicel)): Rt: 2.823 min, ee: 100%.

### Enantiomer (Longer Retention Time):

¹H NMR (400 MHz, DMSO-d₆): δ 11.18 (br, 1H), 8.59 (s, 1H), 7.91 (s, 1H), 7.77 (s, 1H), 7.20 (s, 1H), 6.85 (s, 1H), 4.85-4.81 (m, 2H), 4.71-4.67 (m, 2H), 3.54 (s, 3H), 2.59-2.54 (m, 2H), 2.45-2.38 (m, 2H).

LCMS: MS m/z (ESI): 456.0 [M+H]⁺.

Chiral HPLC (CO₂/MeOH/DEA 60/40/0.04 2.8 ml/min OD,5 um,4.6*250(Daicel)): Rt: 3.878 min, ee: 96.48%.

### Example 74

### (S)-5-cyclopropyl-5-(3-(5-(difluoromethyl)-6-fluoroisoindolin-2-yl)-3-oxopropyl)imidazolidine-2,4-dione 74

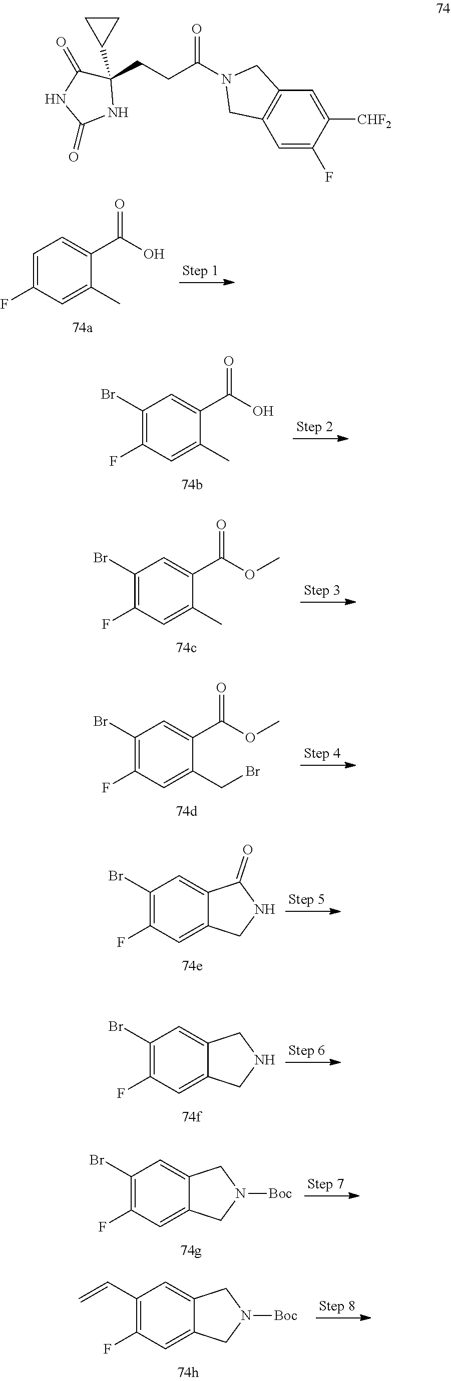

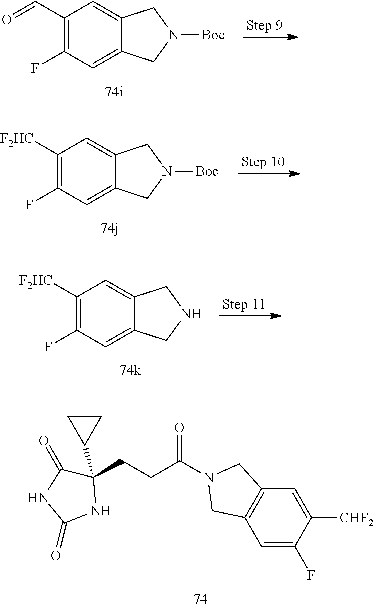 [SMILES: CC(C)(C)OC(=O)N1Cc2cc(F)c(C=O)cc2C1 ; CC(C)(C)OC(=O)N1Cc2cc(F)c(C(F)F)cc2C1 ; Fc1cc2c(cc1C(F)F)CNC2 ; O=C1NC(=O)[C@](CCC(=O)N2Cc3cc(F)c(C(F)F)cc3C2)(C2CC2)N1]

### Step 1

### 5-Bromo-4-fluoro-2-methylbenzoic acid 74b

To a solution of 4-fluoro-2-methylbenzoic acid 74a (10 g, 64.88 mmol) in H₂SO₄ (50 mL) was added NBS (11.6 g, 65.18 mmol) in portions at 0° C. The reaction mixture was stirred at 0-5° C. for 2 hrs. The resulting mixture was poured into ice-water. The solid was collected by filtration and dried in vacuum to give 74b (14 g, 60.08 mmol, 92.60% yield).

¹H NMR (400 MHz, DMSO-d₆): δ 8.07 (d, 1H), 7.39 (d, 1H), 2.51 (s, 3H).

### Step 2

### Methyl 5-bromo-4-fluoro-2-methylbenzoate 74c

To a solution of 74b (15 g, 64.37 mmol) in MeOH (150 mL) at 0° C. was added SOCl₂ (22.97 g, 193.10 mmol, 14 mL) slowly. The reaction mixture was heated to 80° C. for 2 hr. Then the mixture was cooled to room temperature and concentrated in vacuum. The residue was diluted with aq NH₄Cl and extracted with EtOAc. The combined organic layers were washed with brine, dried over Na₂SO₄, filtered and concentrated in vacuum to afford crude 74c (15.2 g, 61.52 mmol, 95.58% yield).

¹H NMR (400 MHz, DMSO-d₆): δ 8.02 (d, 1H), 7.37 (d, 1H), 3.78 (s, 3H), 2.45 (s, 3H).

### Step 3

### Methyl 5-bromo-2-(bromomethyl)-4-fluorobenzoate 74d

To a solution of 74c (15.2 g, 61.52 mmol) in CCl₄ (250 mL) was added NBS (13.14 g, 73.83 mmol) and AIBN (1.01 g, 6.15 mmol). The reaction mixture was stirred at 80° C. overnight.

Then the mixture was cooled down to room temperature and filtered. The cake was washed with CCl₄, the filtrate was concentrated in vacuum. The residue was purified by silica gel chromatagraphy (EtOAc: hexane=1:20) to afford 74d (19 g, 58.29 mmol, 94.74% yield).

¹H NMR (400 MHz, DMSO-d₆): δ 8.16 (d, 1H), 7.71 (d, 1H), 4.98 (s, 2H), 3.88 (s, 3H).

### Step 4

### 6-bromo-5-fluoroisoindolin-1-one 74e

The solution of 74d (4.6 g, 14.11 mmol) in NH₃/MeOH (40 mL, 7N) was stirred at room temperature overnight. The reaction mixture was concentrated in vacuum, the residue was purified by silica gel chromatagraphy (MeOH: DCM=1:50) to give 74e (3.0 g, 13.04 mmol, 92.41% yield).

LCMS: MS m/z (ESI): 230.3 [M+H]⁺.

### Step 5

### 5-bromo-6-fluoroisoindoline 74f

To a solution of 74e (3.0 g, 13.04 mmol) in THF (20 mL) was added BH₃/THF (1N in THF, 90 mmol, 90 mL), and the mixture was heated to 65° C. overnight. The reaction was quenched with methanol (5 mL) and 6M HCl to adjust pH to 2. The mixture was heated to 80° C. for 2 h, and cooled to RT. The mixture was adjusted to pH 7-8 with 6M NaOH, and was extracted with ethyl acetate (3×). The combined organic phases was dried over anhydrous Na₂SO₄, and concentrated in vacuum. The crude mixture was purified by silica gel chromatagraphy (MeOH: DCM=1:20) to afford 74f (350 mg, 1.62 mmol, 12.42% yield).

¹H NMR (400 MHz, DMSO-d₆): δ 7.62 (d, 1H), 7.33 (d, 1H), 4.14-4.10 (m, 4H).

LCMS: MS m/z (ESI): 216.2 [M+H]⁺.

### Step 6

### Tert-butyl 5-bromo-6-fluoroisoindoline-2-carboxylate 74 g

To a solution of 74f (350 mg, 1.62 mmol) in DCM (5 mL) was added TEA (492 mg, 4.86 mmol) and Boc₂O (425 mg, 1.94 mmol), the mixture was stirred at room temperature for 4 h.

The reaction mixture was concentrated in vacuum. The crude mixture was purified by silica gel chromatagraphy (EtOAc:hexane=1:20) to afford 74 g (580 mg, 1.83 mmol, 113.24% yield).

¹H NMR (400 MHz, DMSO-d₆): δ 7.71-7.67 (m, 1H), 7.40-7.35 (m, 1H), 4.55-4.52 (m, 4H), 1.47-1.44 (m, 9H).

LCMS: MS m/z (ESI): 316.2 [M+H]⁺.

### Step 7

### Tert-butyl 5-fluoro-6-vinylisoindoline-2-carboxylate 74 h

To a solution of 74 g (580 mg, 1.83 mmol) in 1,4-dioxane (20 mL) and H₂O (3 mL) was added potassium trifluoro(vinyl)borate (270 mg, 2.02 mmol), Pd(dppf)Cl₂ (150 mg, 183.45 umol) and K₂CO₃ (760 mg, 5.50 mmol), the reaction was replaced with N₂ for three times. The mixture was stirred at 100° C. overnight. Water was added and the reaction mixture was extracted with EtOAc. The combined organic layers were washed with water and brine, dried over anhydrous Na₂SO₄, filtered and concentrated in vacuum. The crude mixture was purified by silica gel chromatagraphy (EtOAc:hexane=1:20) to afford 74 h (400 mg, 1.52 mmol, 82.81% yield).

¹H NMR (400 MHz, DMSO-d₆): δ 7.63-7.58 (m, 1H), 7.22-7.17 (m, 1H), 6.82 (dd, 1H), 5.89 (dd, 1H), 5.41 (d, 1H), 4.58-4.52 (m, 4H), 1.45 (s, 9H).

LCMS: MS m/z (ESI): 208.0 [M-tBu+H]⁺.

### Step 8

### Tert-butyl 5-fluoro-6-formylisoindoline-2-carboxylate 74i

To solution of 74 h (400 mg, 1.52 mmol) in 1,4-dioxane (8 mL) was added NaIO₄ (650 mg, 3.04 mmol) and H₂O (2 mL), and the mixture was stirred at RT. Then OSO₄ (39 mg, 151.91 umol) was added. The reaction mixture was stirred at room temperature for 3 h. Saturated sodium bicarbonate was added, and then the reaction mixture was extracted with EtOAc. The combined organic layers were washed with water and brine, dried over anhydrous Na₂SO₄, filtered and concentrated in vacuum. The crude mixture was purified by silica gel chromatagraphy (EtOAc:hexane=1:20) to give 74i (180 mg, 678.53 umol, 44.67% yield).

¹H NMR (400 MHz, DMSO-d₆): δ 10.20 (s, 1H), 7.81-7.76 (m, 1H), 7.44-7.39 (m, 1H), 4.67-4.58 (m, 4H), 1.45 (s, 9H).

LCMS: MS m/z (ESI): 210.4 [M-tBu+H]⁺.

### Step 9

### Tert-butyl 5-(difluoromethyl)-6-fluoroisoindoline-2-carboxylate 74j

To a solution of 74i (180 mg, 678.53 umol) in DCM (5 mL) was added EtOH (3.1 mg, 67.85 umol), then DAST (547 mg, 3.39 mmol) was added dropwise at RT. The mixture was stirred at room temperature for 3 h. Water was added and the reaction mixture was extracted with DCM. The combined organic layers were washed with water and brine, dried over anhydrous Na₂SO₄, filtered and concentrated in vacuum. The crude mixture was purified by silica gel chromatagraphy (EtOAc:hexane=1:20) to afford 74j (180 mg, 626.57 umol, 92.34% yield).

¹H NMR (400 MHz, DMSO-d₆): δ 7.62-7.58 (m, 1H), 7.40-7.33 (m, 1H), 7.20 (t, 1H), 4.64-4.57 (m, 4H), 1.45 (s, 9H).

LCMS: MS m/z (ESI): 232.4 [M-tBu+H]⁺.

### Step 10

### 5-(difluoromethyl)-6-fluoroisoindoline 74k

To a solution of 74j (180 mg, 626.57 umol) in the flask was added HCl in1,4-dioxane (4N, 10 mL). The mixture was stirred at room temperature for 2 h. The reaction mixture was concentrated in vacuum to give 74k (110 mg, 587.73 umol, 93.80% yield).

LCMS: MS m/z (ESI): 188.1 [M+H]⁺.

### Step 11

### (S)-5-cyclopropyl-5-(3-(5-(difluoromethyl)-6-fluoroisoindolin-2-yl)-3-oxopropyl)imidazolidine-2,4-dione 74

To a solution of 74k (110 mg, 587.73 umol) in DMF (5 mL) was added TEA (0.4 mL), (S)-3-(4-cyclopropyl-2,5-dioxoimidazolidin-4-yl)propanoic acid Int-1 (125 mg, 587.73 umol) and HATU (246 mg, 646.50 umol). The mixture was stirred at room temperature for 2 h. Water was added and reaction mixture was extracted with EtOAc. The combined organic layers were washed with water and brine, dried over anhydrous Na₂SO₄, filtered and concentrated in vacuum. The residue was purified by prep-HPLC to give 74 (24 mg, 62.93 umol, 10.71% yield).

¹H NMR (400 MHz, DMSO-d₆): δ 10.51 (brs, 1H), 7.62 (s, 1H), 7.51 (d, 1H), 7.28 (dd, 1H), 7.09 (t, 1H), 4.73-4.68 (m, 2H), 4.55-4.50 (m, 2H), 2.33-2.25 (m, 1H), 2.20-2.10 (m, 1H), 1.91-1.86 (m, 2H), 1.08-0.96 (m, 1H), 0.38-0.17 (m, 3H), 0.03-0.00 (m, 1H).

¹⁹F NMR (376.5 MHz, DMSO-d₆): δ-112.85, −120.80.

LCMS: MS m/z (ESI): 382.4 [M+H]⁺.

Exampe 75

### 5-(3-(5-chloro-6-(trifluoromethyl)isoindolin-2-yl)-3-oxopropyl)-5-(5-methylisoxazol-3-yl)imidazolidine-2,4-dione 75

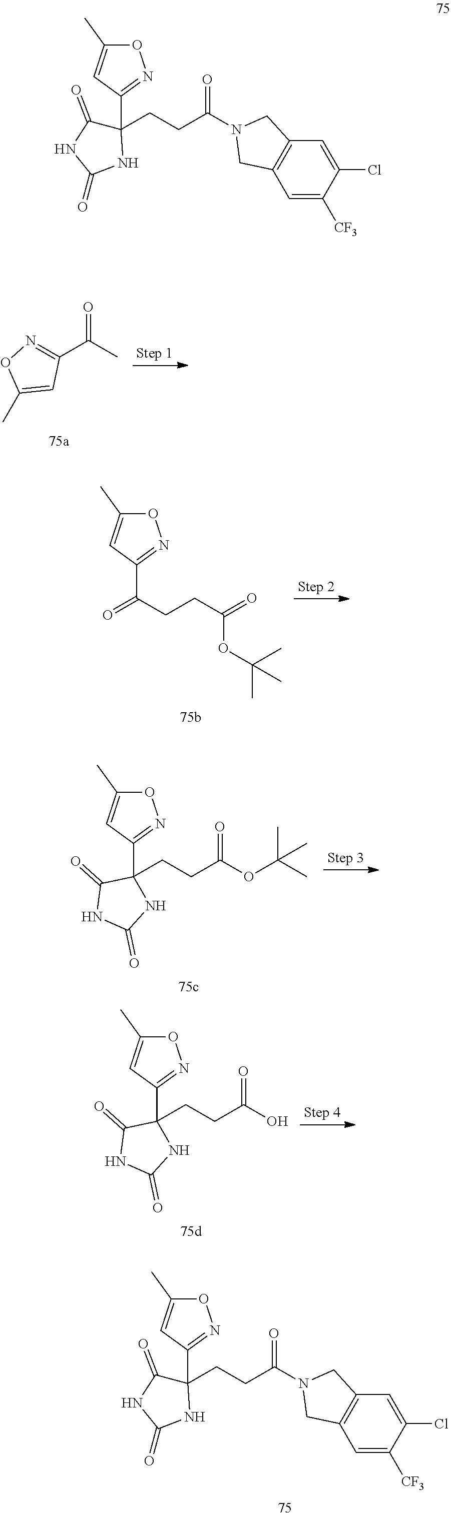

### Step 1

### Tert-butyl 4-(5-methylisoxazol-3-yl)-4-oxobutanoate 75b

To a solution of 1-(5-methylisoxazol-3-yl)ethan-1-one 75a (3.00 g, 23.99 mmol) in THF (10 mL) was added NaHMDS (4.40 g, 23.99 mmol) dropwise at −70° C. The resulting mxiture was stirred at this temperature for 30 min before tert-butyl 2-bromoacetate (4.68 g, 23.99 mmol) was added dropwsie. After addition, the reaction mixture was stirred at −20° C. for 1.0 hour, then warmed to room temperature for 18 hours. The resulting mixture was cooled to 0° C., quenched with aq. NaHCO₃ (20 mL). The whole mixture was extracted with EtOAc (30 mL×4). The organic layers were combined, dried over anhydrous Na₂SO₄ and filtered. The filtrate was concentrated under reduced pressure. The residue was purified by silica gel chromatography (EtOAc/hexane=1/20 to 1/4) to afford 75b (400 mg, 1.67 mmol, 6.97% yield).

LCMS: MS m/z (ESI): 240.5 [M+H]⁺.

### Step 2

### Tert-butyl 3-(4-(5-methylisoxazol-3-yl)-2,5-dioxoimidazolidin-4-yl)propanoate 75c

To the solution of 75b (350 mg, 1.46 mmol) in H₂O (5 mL) and MeOH (5 mL) was added (NH₄)₂CO₃ (1.12 g, 11.70 mmol) and NaCN (176 mg, 3.66 mmol). The reaction was stirred at 85° C. overnight. The mixture was concentrated and the residue was extracted with EtOAc. The organic solution was dried and concentrated. The residue was slurred with Et₂O to give 75c (180 mg, 581.93 umol, 39.78% yield).

LCMS: MS m/z (ESI): 332.1 [M+Na]⁺.

### Step 3

### 3-(4-(5-methylisoxazol-3-yl)-2,5-dioxoimidazolidin-4-yl)propanoic acid 75d

The mixture of 75c (30 mg, 96.99 umol) in HCl/1,4-dixoane (2 mL, 4N) was stirred at room temperature for 1 h. The mixture was oncentrated to give crude 75d (20 mg, 78.99 umol, 81.44% yield).

LCMS: MS m/z (ESI): 254.0 [M+H]⁺.

### Step 4

### 5-(3-(5-chloro-6-(trifluoromethyl)isoindolin-2-yl)-3-oxopropyl)-5-(5-methylisoxazol-3-yl)imidazolidine-2,4-dione 75

To the solution of 5-chloro-6-(trifluoromethyl)isoindoline 40 h (17.50 mg, 78.99 umol) in DMF (2 mL) was added HATU (33 mg, 86.88 umol), Et₃N (24 mg, 236.96 umol) and 75d (20 mg, 78.99 umol). The mixture was stirred at room temperature for 1 h. The reaction mixture was purified by prep-HPLC to give 75 (2.5 mg, 5.47 umol, 6.93% yield).

¹H NMR (400 MHz, CDCl₃): δ 8.45 (br, 1H), 7.62 (br, 1H), 7.48 (br, 1H), 7.00 (br, 1H), 6.20 (brs, 1H), 4.80 (br, 4H), 2.60-2.45 (m, 4H), 2.42 (s, 3H).

LCMS: MS m/z (ESI): 457.0 [M+H]⁺.

### Example 76

### (S)-5-cyclopropyl-5-(3-(5-(methylamino)-6-(trifluoromethyl)isoindolin-2-yl)-3-oxopropyl)imidazolidine-2,4-dione 76

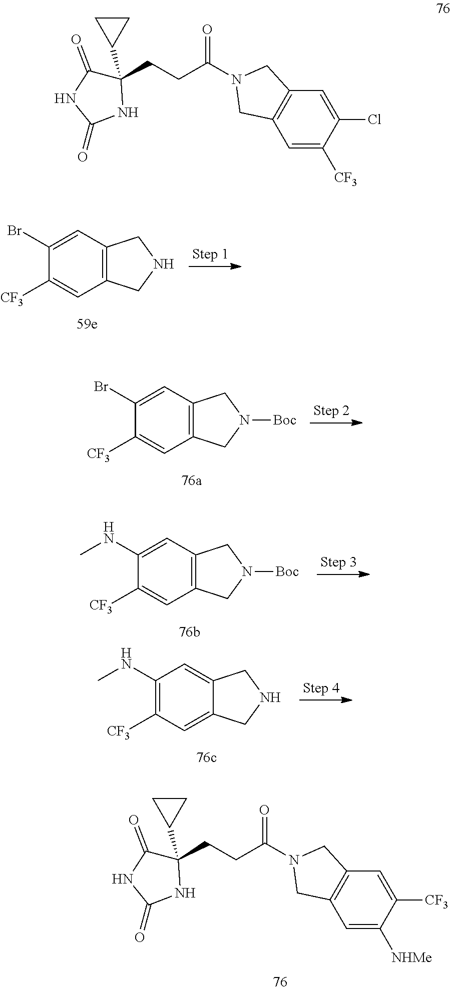 [SMILES: O=C1NC(=O)[C@](CCC(=O)N2Cc3cc(Cl)c(C(F)(F)F)cc3C2)(C2CC2)N1 ; FC(F)(F)c1cc2c(cc1Br)CNC2 ; CC(C)(C)OC(=O)N1Cc2cc(Br)c(C(F)(F)F)cc2C1 ; CNc1cc2c(cc1C(F)(F)F)CN(C(=O)OC(C)(C)C)C2 ; CNc1cc2c(cc1C(F)(F)F)CNC2 ; CNc1cc2c(cc1C(F)(F)F)CN(C(=O)CC[C@@]1(C3CC3)NC(=O)NC1=O)C2]

### Step 1

### Tert-butyl 5-bromo-6-(trifluoromethyl)isoindoline-2-carboxylate 76a

To a solution of 59e (920 mg, 3.46 mmol) in THF (20 mL) was added TEA (1.8 mL) and Boc₂O (906 mg, 4.15 mmol). The mixture was stirred at room temperature for 4 h. The reaction mixture was concentrated in vacuo and the residue was purified by silica gel chromatography (ethyl acetate/hexane=1/20) to afford 76a (810 mg, 2.21 mmol, 63.97% yield).

LCMS: MS m/z (ESI): 311.9 [M+H-tBu]⁺.

### Step 2

### Tert-butyl 5-(methylamino)-6-(trifluoromethyl)isoindoline-2-carboxylate 76b

To a solution of 76a (100 mg, 273.09 umol) in 1,4-dioxane (5 mL) was added MeNH₂/THF (1N in THF, 0.5 mL, 0.5 mmol), Pd₂(dba)₃ (25 mg, 27.31 umol), Cs₂CO₃ (267 mg, 819.28 umol) and XantPhos (32 mg, 54.62 umol). The reaction was stirred at 90° C. in a sealed tube under N₂ overnight. Water was added and the mixture was extracted with EtOAc. The combined organic layers were washed with water and brine, dried over anhydrous Na₂SO₄, filtered and concentrated in vacuo. The residue was purified by prep-HPLC to give 76b (45 mg, 142.26 umol, 52.09% yield).

¹HNMR (400 MHz, DMSO-d₆): δ 7.38 (d, 1H), 6.69 (s, 1H), 5.59 (d, 1H), 4.56-4.52 (m, 2H), 4.48-4.45 (m, 2H), 2.75 (t, 3H), 1.45 (s, 9H).

LCMS: MS m/z (ESI): 358.1 [M+H+CH₃CN]⁺.

### Step 3

### N-methyl-6-(trifluoromethyl)isoindolin-5-amine 76c

The solution of 76b (45 mg, 142.26 umol) of HCl/1,4-dioxane (5 mL, 1N) was stirred at room temperature for 3 h. The reaction mixture was concentrated in vacuo to give 76c (30 mg, 138.76 umol, 97.54% yield).

LCMS: MS m/z (ESI): 217.1 [M+H]⁺.

### Step 4

### (S)-5-cyclopropyl-5-(3-(5-(methylamino)-6-(trifluoromethyl)isoindolin-2-yl)-3-oxopropyl)imidazolidine-2,4-dione 76

To a solution of 76c (30 mg, 138.76 umol) in DMF (2 mL) was successively added TEA (0.1 mL), Int-1 (30 mg, 138.76 umol) and HATU (58 mg, 152.63 umol). The mixture was stirred at room temperature for 2 h. Water was added and the mixture was extracted with EtOAc. The combined organic layers were washed with water and brine, dried over anhydrous Na₂SO₄, filtered and concentrated in vacuo. The crude mixture was purified by prep-HPLC to give 76 (10 mg, 24.37 umol, 17.56% yield).

¹H NMR (400 MHz, DMSO-d₆): δ 10.64 (s, 1H), 7.74 (s, 1H), 7.41 (s, 1H), 6.72 (s, 1H), 5.61 (d, 1H), 4.76 (brs, 1H), 4.67 (brs, 1H), 4.59 (brs, 1H), 4.51 (brs, 1H), 2.77 (d, 3H), 2.44-2.21 (m, 2H), 2.05-1.96 (m, 2H), 1.14-1.09 (m, 1H), 0.50-0.29 (m, 3H), 0.16-0.12 (m, 1H).

¹⁹F NMR (376.5 MHz, DMSO-d₆): δ-61.20.

LCMS: MS m/z (ESI): 411.2 [M+H]⁺.

### Example 77

### 5-(3-(5-chloro-6-(trifluoromethyl)isoindolin-2-yl)-3-oxopropyl)-5-(1-methyl-1H-pyrazol-3-yl)imidazolidine-2,4-dione 77

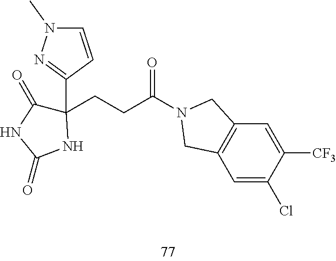 [SMILES: Cn1ccc(C2(CCC(=O)N3Cc4cc(Cl)c(C(F)(F)F)cc4C3)NC(=O)NC2=O)n1]

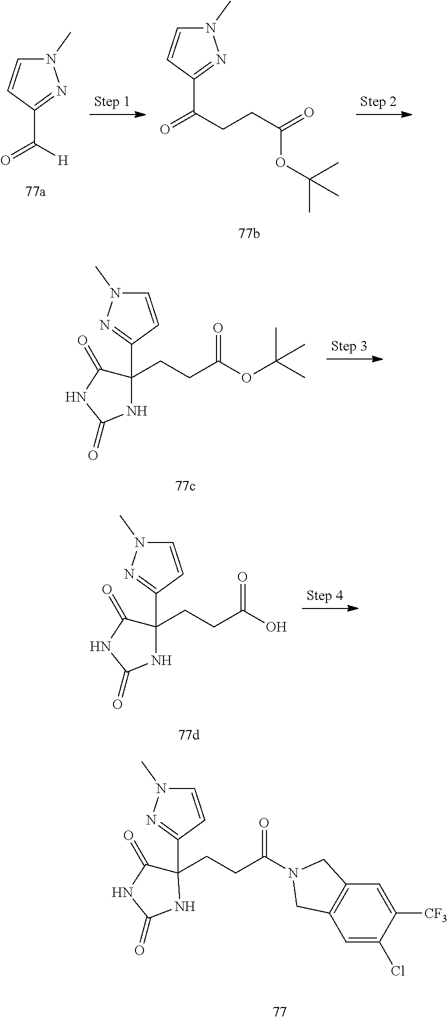

### Step 1

### Tert-butyl 4-(1-methyl-1H-pyrazol-3-yl)-4-oxobutanoate 77b

To the solution of 1-methyl-1H-pyrazole-3-carbaldehyde 77a (3.87 g, 35.11 mmol) in THF (10 mL) was added P(Bu)₃ (5.4 g, 42.13 mmol), and the reaction mixture was heated at 50′° C. for 5 min, tert-butyl prop-2-enoate (4.5 g, 35.11 mmol) was added and the mixture is stirred at 80° C. for 3 h. More tert-butyl prop-2-enoate (4.5 g, 35.11 mmol) was added and this process was repeated until no elution is observerd by TLC. The mixture was purified by silica gel chromatagraphy (hexane/EtOAc=10/1) to give 77b (1.2 g, 5.04 mmol, 14.34% yield).

LCMS: MS m/z (ESI): 183.1 [M+1-tBu]⁺.

### Step 2

### Tert-butyl 3-(4-(1-methyl-1H-pyrazol-3-yl)-2,5-dioxoimidazolidin-4-yl)propanoate 77c

To the solution of 77b (300 mg, 1.26 mmol) in H₂O (2 mL) and MeOH (2 mL) was added NaCN (155 mg, 3.15 mmol) and (NH₄)₂CO₃ (967 mg, 10.07 mmol). The reaction was stirred at 85° C. overnight. LCMS showed the product produced. Water was added and the mixture was extracted with EtOAc (10 mL×2), the combined organic layers were concentrated to give crude 77c (100 mg, 324.33 umol, 25.76% yield).

¹H NMR (400 MHz, DMSO-d₆): δ 10.74 (s, 1H), 8.35 (s, 1H), 7.65 (d, 1H), 6.18 (d, 1H), 3.81 (s, 3H), 2.23-2.19 (m, 4H), 1.39 (s, 9H).

LCMS: MS m/z (ESI): 309.1 [M+H]⁺.

### Step 3

### 3-(4-(1-methyl-1H-pyrazol-3-yl)-2,5-dioxoimidazolidin-4-yl)propanoic acid 77d

The mixture of 77c (110 mg, 356.76 umol) in HCl/1,4-dioxane (2 mL, 2N) was stirred at room temperature for 1 h. LCMS showed that the product formed. The mixture was concentrated to give 77d (100 mg, 396.47 umol, 111.13% yield).

LCMS: MS m/z (ESI): 253.1 [M+H]⁺.

### Step 4

### 5-(3-(5-chloro-6-(trifluoromethyl)isoindolin-2-yl)-3-oxopropyl)-5-(1-methyl-1H-pyrazol-3-yl)imidazolidine-2,4-dione 77

To the solution of 40 h (20 mg, 79.29 umol) in DMF (2 mL) was added HOBT (12.85 mg, 95.15 umol), EDCI (19 mg, 95.15 umol) and 77d (17.57 mg, 79.29 umol). The reaction was stirred atroom temperature overnight. LCMS showed that the product formed.

The mixture was purified by prep-HPLC to give 77 (0.88 mg, 1.93 umol, 2.43% yield).

¹H NMR (400 MHz, CDCl₃): δ 7.63-7.58 (m, 2H), 7.46-7.40 (m, 1H), 7.33 (d, 1H), 6.35-6.31 (m, 2H), 4.80-4.77 (m, 4H), 3.86 (s, 3H), 2.61-2.44 (m, 4H).

¹⁹F NMR (376.5 MHz, CDCl₃): δ −62.45.

LCMS: MS m/z (ESI): 456.4 [M+H]⁺.

### Example 78

### 5-(3-(5-chloro-6-(trifluoromethyl)isoindolin-2-yl)-3-oxopropyl)-5-(5-methyl-1H-pyrazol-3-yl)imidazolidine-2,4-dione 78

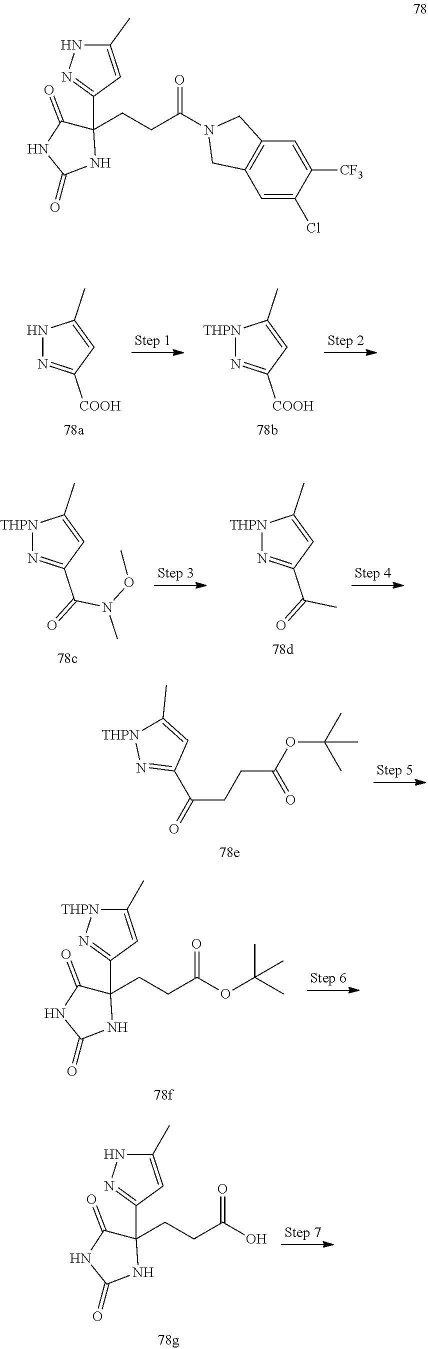

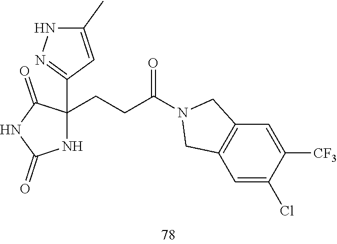 [SMILES: Cc1cc(C2(CCC(=O)N3Cc4cc(Cl)c(C(F)(F)F)cc4C3)NC(=O)NC2=O)n[nH]1]

### Step 1

### 5-methyl-1-(tetrahydro-2H-pyran-2-yl)-1H-pyrazole-3-carboxylic acid 78b

To a solution of 5-methyl-1H-pyrazole-3-carboxylic acid 78a (1 g, 7.94 mmol) and 3,4-dihydro-2H-pyran (1.33 g, 15.88 mmol) in THF (20 mL) was added PTSA (71 mg, 0.4 mmol).

The resulting mixture was stirred at r.t for 18 h. The solution was concentrated, the residue was purified by silica gel column chromatography (eluting with DCM/MeOH=50/1) to afford the tittle compound 78b (907 mg, 4.32 mmol, 54.41% yield).

¹H NMR (400 MHz, CDCl₃): δ 6.63 (s, 1H), 5.37 (d, 1H), 4.04-4.01 (m, 1H), 3.69-3.63 (m, 1H), 2.50-2.41 (m, 1H), 2.38 (s, 3H), 2.14-2.11 (m, 1H), 2.01-1.98 (m, 1H), 1.75-1.55 (m, 4H).

### Step 2

### N-methoxy-N,5-dimethyl-1-(tetrahydro-2H-pyran-2-yl)-1H-pyrazole-3-carboxamide 78c

To a solution of 78b (907 mg, 4.32 mmol) in DCM (15 mL) was added TEA (2.1 mL, 15.12 mmol), the solution was cooled to 0° C. Then HOBt (642 mg, 4.75 mmol) and EDCI (1 g, 5.18 mmol) were added, the reaction mixture was stirred at 0° C. for 30 min. N,O-dimethylhydroxylamine hydrochloride (505 mg, 5.18 mmol) was added. The reaction was stirred at r.t for 18 h. Water (50 mL) was added and the mixture was extracted with DCM (50 mL×2), the organic solution was washed with brine, dried over anhydrous Na₂SO₄ and concentrated. The residue was purified by silica gel chromatography (eluting with hexane/EtOAc=1/1) to afford the tittle compound 78c (810 mg, 3.2 mmol, 74.11% yield).

### Step 3

### 1-(5-methyl-1-(tetrahydro-2H-pyran-2-yl)-1H-pyrazol-3-yl)ethan-1-one 78d

To a solution of 78c (810 mg, 3.2 mmol) in THF (15 mL) was added MeMgCl (3N, 3.2 mL, 9.6 mmmol). The reaction mixture was stirred at 0° C. for 1 h. The reaction was quenched with aqueous NH₄C₁ (20 mL), extracted with EtOAc (20 mL×2), the organic solution was washed with brine, dried over anhydrous Na₂SO₄ and concentrated, the residue was purified by silica gel chromatography (eluting with hexane/EtOAc=4/1) to afford the tittle compound 78d (450 mg, 2.16 mmol, 67.5% yield).

¹H NMR (400 MHz, CDCl₃): δ 6.54 (d, 1H), 5.34 (dd, 1H), 4.04-4.00 (m, 1H), 3.70-3.64 (m, 1H), 2.56 (s, 31H), 2.52-1.45 (m, 1H), 2.36 (s, 3H), 2.18-2.12 (m, 1H), 2.01-1.96 (m, 1H), 1.75-1.61 (m, 3H).

### Step 4

### Tert-butyl 4-(5-methyl-1-(tetrahydro-2H-pyran-2-yl)-1H-pyrazol-3-yl)-4-oxobutanoate 78e

To a solution of 78d (200 mg, 0.96 mmol) in THF (3 mL) was added NaHMDS (2 N, 0.55 mL, 1.1 mmoL) dropwise at −78° C. After 1 h, tert-butyl bromoacetate (0.146 mL, 1 mmoL) was added to the solution dropwise. The reaction was stirred at r.t for 18 h. Water (20 mL) was added and the mixture was extracted with EtOAc (20 mL×2). The combined organic layeres were washed with brine, dried over anhydrous Na₂SO₄ and concentrated. The residue was purified by silica gel column chromatography (eluting with hexane/EtOAc=10/1) to afford the tittle compound 78e (180 mg, 0.87 mmol, 90.58% yield).

¹H NMR (400 MHz, CDCl₃): δ 6.55 (s, 1H), 5.34 (dd, 1H), 4.02-3.99 (m, 1H), 3.69-3.63 (m, 1H), 3.38-3.22 (m, 2H), 2.67-2.56 (m, 2H), 2.51-2.44 (m, 1H), 2.35 (s, 3H), 2.19-2.14 (m, 1H), 1.99-1.95 (m, 1H), 1.73-1.65 (m, 3H), 1.43 (s, 9H).

### Step 5

### Tert-butyl 3-(4-(5-methyl-1-(tetrahydro-2H-pyran-2-yl)-1H-pyrazol-3-yl)-2,5-dioxoimidazolidin-4-yl)propanoate 78f

To a solution of 78e (2 g, 6.21 mmol) in EtOH (30 mL) and water (30 mL) was added (NH₄)₂CO₃ (4.77 g, 49.69 mmol) and NaCN (760.73 mg, 15.53 mmol). The reaction was done in sealed tube and stirred at 90° C. for 18 h. The mixture was poured to water (100 mL) and extracted with EtOAc (50 mL×3), the combined organic layers were washed with brine, dried over anhydrous Na₂SO₄ and concentrated. The residue was triturated with hexane to afford the tittle compound 78f (2.2 g, 5.61 mmol, 90.37% yield).

LCMS: MS m/z (ESI): 393.2 [M+H]⁺.

### Step 6

### 3-(4-(5-methyl-1H-pyrazol-3-yl)-2,5-dioxoimidazolidin-4-yl)propanoic acid 78 g

A solution of 78f (500 mg, 1.28 mmol) in HCl/1,4-dioxane (4N, 10 mL) was stirred at r.t for 18 h. The solution was concentrated to afford the tittle compound 78g (350 g, 1.21 mmol, 94.53% yield).

¹H NMR (400 MHz, DMSO-d₆): δ 10.82 (s, 1H), 8.42 (s, 1H), 6.04 (s, 1H), 2.25-2.18 (m, 7H).

### Step 7

### 5-(3-(5-chloro-6-(trifluoromethyl)isoindolin-2-yl)-3-oxopropyl)-5-(5-methyl-1H-pyrazol-3-yl)imidazolidine-2,4-dione 78

To a solution of 78 g (50 mg, 0.17 mmol) in DMF (5 mL) was added TEA (0.12 mL, 0.85 mmol) and 5-chloro-6-(trifluoromethyl)isoindoline 40 h (44 mg, 0.17 mmol), then HATU (65 mg, 0.17 mmol) was added. The reaction was stirred at r.t for 2 h. Water (20 mL) was added and the mixture was extracted with EtOAc (20 mL×2). The combined organic layers were washed with brine, dried over anhydrous Na₂SO₄ and concentrated. The residue was purified by prep-HPLC to afford the tittle compound 78 (18 mg, 0.04 mmol, 23.53% yield).

¹H NMR (400 MHz, DMSO-d₆): δ12.50 (brs, 1H), 10.71 (brs, 1H), 8.35 (s, 1H), 7.89 (d, 1H), 7.75 (d, 1H), 5.96 (s, 1H), 4.81 (d, 2H), 4.67 (d, 2H), 2.50-2.29 (m, 4H), 2.20 (s, 3H).

LCMS: MS m/z (ESI): 456.1 [M+H]⁺.

### Example 79

### 5-(3-(5-chloro-6-(trifluoromethyl)isoindolin-2-yl)-3-oxopropyl)-5-(pyrimidin-2-yl)imidazolidine-2,4-dione 79

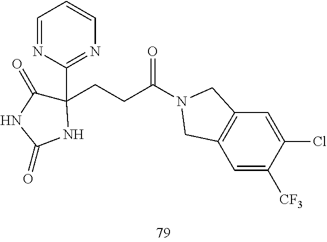 [SMILES: O=C1NC(=O)C(CCC(=O)N2Cc3cc(Cl)c(C(F)(F)F)cc3C2)(c2ncccn2)N1]

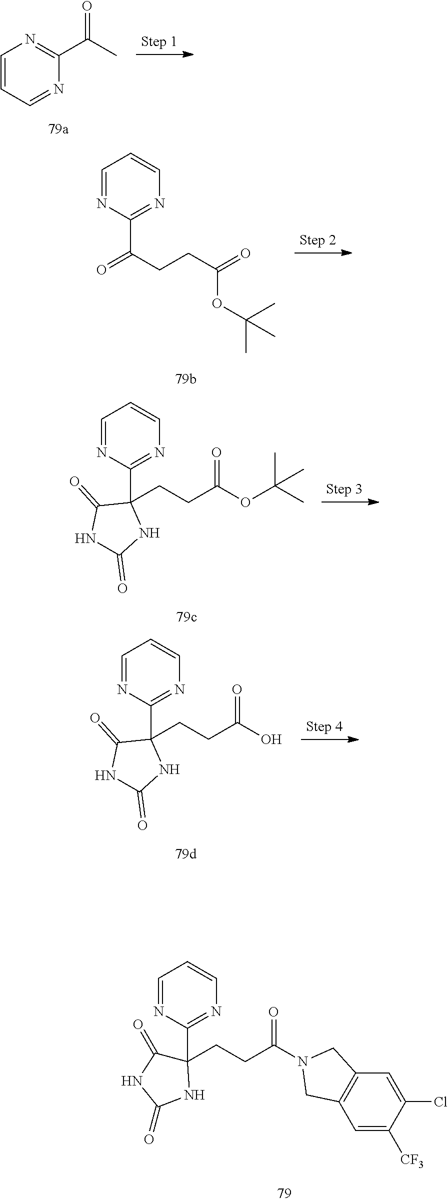

### Step 1

### Tert-butyl 4-oxo-4-(pyrimidin-2-yl)butanoate 79b

To the solution of 1-(pyrimidin-2-yl)ethan-1-one 79a (3 g, 24.57 mmol) in THF (100 mL) was added DMPU (4.15 g, 32.43 mmol) and LiHMDS (5.39 g, 29.48 mmol) at 0° C., the reaction was stirred at this temperature for 30 min. Then tert-butyl 2-bromoacetate (4.79 g, 24.57 mmol) was added at 0° C., and the reaction was stirred for 3 h. The reaction mixture was quenched by water and concentrated. The residue was purified by silica gel chromatography (EtOAc/hexane=1/20) to give 79b (500 mg, 8.61% yield).

LCMS: MS m/z (ESI): 237.1 [M+H]⁺.

### Step 2

### Tert-butyl 3-(2,5-dioxo-4-(pyrimidin-2-yl)imidazolidin-4-yl)propanoate 79c

To the solution of 79b (100 mg, 423.25 umol) in H₂O (1 mL) and MeOH (1 mL) was added NaCN (56.12 mg, 1.06 mmol) and (NH₄)₂CO₃ (325.06 mg, 3.39 mmol). The reaction was stirred at 85° C. overnight. The mixture was concentrated and extracted with EtOAc. The organic solution was dried and concentrated; the residue was slurred with Et₂O to give crude 79c (25 mg, 19.28% yield) for next step as is.

LCMS: MS m/z (ESI): 305.0 [M−H]⁻.

### Step 3

### 3-(2,5-dioxo-4-(pyrimidin-2-yl)imidazolidin-4-yl)propanoic acid 79d

The solution of 79c (25 mg, 81.62 umol) in 4N HCl/dixoane (2 mL) was stirred at room temperature for 1 h. LCMS showed that the product was produced and the mixture was concentrated to give 79d (16 mg, 78.35% yield) for next step as it is.

LCMS: MS m/z (ESI): 251.0 [M+H]⁺.

### Step 4

### 5-(3-(5-chloro-6-(trifluoromethyl)isoindolin-2-yl)-3-oxopropyl)-5-(pyrimidin-2-yl)imidazolidine-2,4-dione 79

To the solution of 79d (16 mg, 0.0634 mmol) in THF (5 mL) was added HATU (231.63 mg, 609.19 umol), DIEA (62.38 mg, 487.35 umol) and 5-chloro-6-(trifluoromethyl)isoindoline 40 h (14 mg, 0.063 mmol). The reaction was stirred at room temperature overnight. The mixture was concentrated and the residue was purified by prep-HPLC to give 79 (2.13 mg, yield 7.34%).

¹H NMR (400 MHz, CDCl₃): δ 8.80 (brs, 1H), 8.79 (brs, 1H), 8.15 (brs, 1H), 7.60 (d, 1H), 7.60 (d, 1H), 7.32 (br, 1H), 6.46 (brs, 1H), 4.84-4.75 (m, 4H), 2.85-2.78 (m, 2H), 2.56 (br, 2H).

LCMS: MS m/z (ESI): 454.1 [M+H]⁺.

### Example 80

### (S)-5-cyclopropyl-5-(3-(5,6-dichloroisoindolin-2-yl-1,1,3,3-d4)-3-oxopropyl)imidazolidine

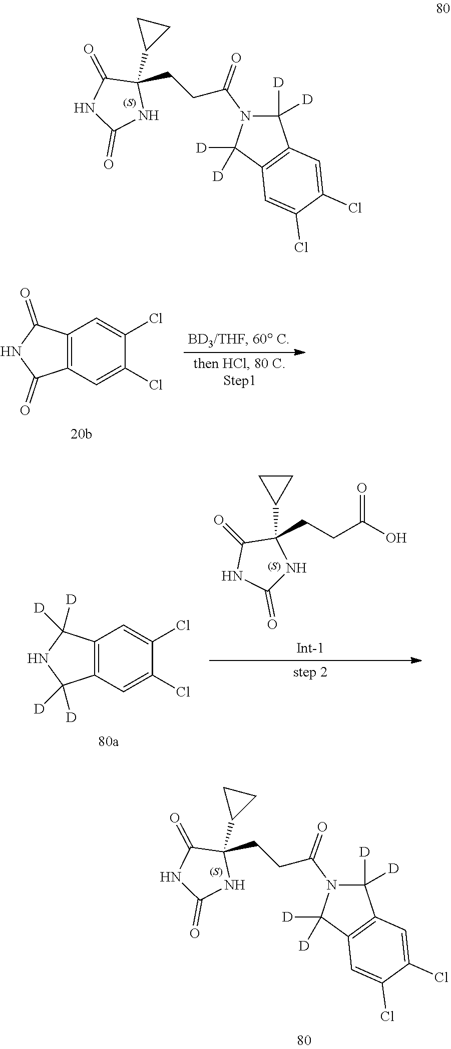

### Step 1

### 5,6-dichloroisoindoline-1,1,3,3-d₄ 80a

To a solution of 4,5-dichlorophthalimide 20b (500 mg, 2.3 mmol) in THF (15 mL) was added BD₃-tetrahydrofuran (1M, 20 mL) dropwise under N₂. The resulting mixture was stirred at 60° C. for 24 h. The reaction mixture was cooled to ambient temperature and quenched with MeOH (6 ml) until the bubbling ceased. Then 4N HCl in water (20 ml) was added and the mixture was heated at 80° C. for 1 h. After cooled down to room temperature and 5N KOH was added to adjust pH to 7. The mixture was concentrated under reduced pressure and the residue was purified by silica-gel column (DCM: MeOH(2% NH₄₀H)=10:1) to afford 5,6-dichloroisoindoline-1,1,3,3-d₄80a (300 mg, 70% yield).

### Step 2

### (S)-5-cyclopropyl-5-(3-(5,6-dichloroisoindolin-2-yl-1,1,3,3-d4)-3-oxopropyl)imidazolidine-2,4-dione 80

To a mixture of 5,6-dichloroisoindoline-1,1,3,3-d₄ 80a (35 mg, 0.19 mmol) in DMF (4 mL) was added triethylamine (72 mg, 0.56 mmol), Int-1 (40 mg, 0.16 mmol) and HATU (98.8 mg, 0.23 mmol). The reaction was stirred at r.t for 18 h. Water (5 mL) was added and the mixture was extracted with EtOAc (40 mL×3). The combined layer was washed with brine, dried over Na₂SO₄ and concentrated. The residue was purified with prep-HPLC to give the title compound (30 mg, 40% yield).

¹H NMR (400 MHz, Methanol-d4): δ 7.54 (d, 2H), 2.56 (ddd, 2H), 2.32-2.13 (m, 2H), 1.34-1.19 (m, 1H), 0.60 (td, 2H), 0.53-0.30 (m, 2H).

LCMS: MS m/z (ESI): 386 [M+H]⁺.

### Example 81

### 5-(3-(5-chloro-6-(trifluoromethyl)isoindolin-2-yl)-3-oxopropyl)-5-(2-methyloxazol-4-yl)imidazolidine-2,4-dione 81

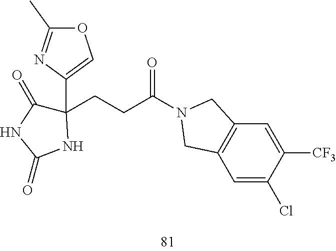 [SMILES: Cc1nc(C2(CCC(=O)N3Cc4cc(Cl)c(C(F)(F)F)cc4C3)NC(=O)NC2=O)co1]

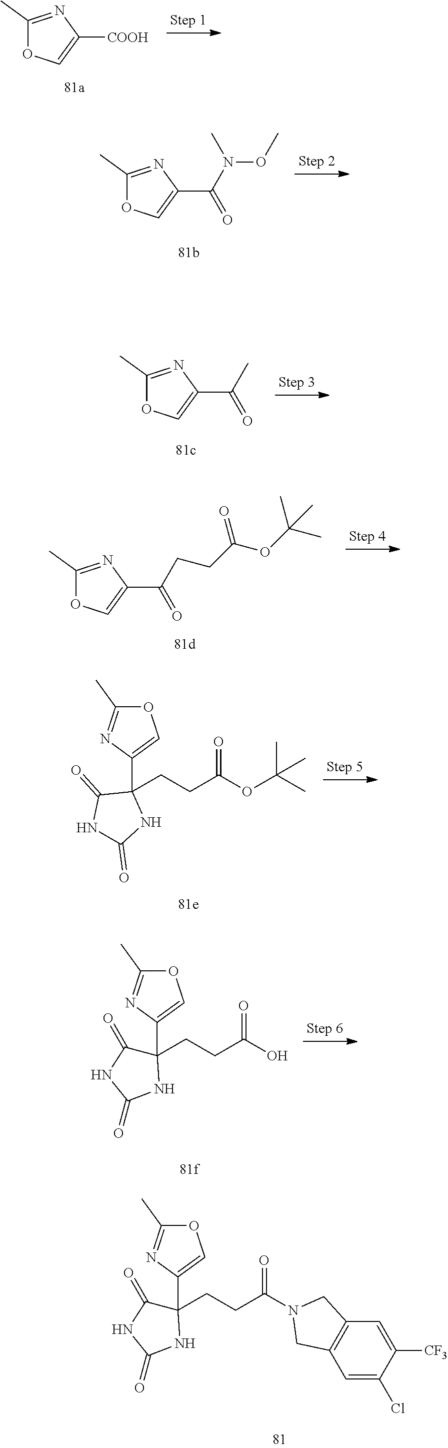

### Step 1

### N-methoxy-N,2-dimethyloxazole-4-carboxamide 81b

To a solution of 2-methyloxazole-4-carboxylic acid 81a (500 mg, 3.94 mmol) in DCM (15 mL) was added TEA (1.9 mL, 13.79 mmol), the solution was cooled to 0° C. Then HOBt (585 mg, 4.33 mmol) and EDCI (906 mg, 4.73 mmol) were added. The reaction mixture was stirred at 0° C. for 30 min. N,O-Dimethylhydroxylamine hydrochloride (460 mg, 4.73 mmol) was added.

The reaction was stirred at r.t for 18 h. Water (50 mL) was added and the mixture was extracted with DCM (50 mL×2). The combined organic layers were washed with brine, dried over anhydrous Na₂SO₄ and concentrated. The residue was purified by silica gel chromatography (eluting with hexane/EtOAc=1/1) to afford the tittle compound 81b (600 mg, 3.53 mmol, 89.58% yield).

¹H NMR (400 MHz, CDCl₃): δ 8.07 (s, 1H), 3.74 (s, 3H), 3.37 (s, 3H), 2.51 (s, 3H).

### Step 2

### 1-(2-methyloxazol-4-yl)ethan-1-one 81c

To a solution of 81b (200 mg, 1.18 mmol) in THF (20 mL) was added MeMgCl (3N, 1.18 mL, 3.52 mmmol). The reaction mixture was stirred at 0° C. for 1 h. The reaction was diluted with aqueue NH₄C₁ (30 mL) and extracted with EA (20 mL×2). The combined organic layers were washed with brine, dried over anhydrous Na₂SO₄ and concentrated. The residue was purified by silica gel chromatography (eluting with hexane/EtOAc=4/1) to afford the tittle compound 81c (100 mg, 0.59 mmol, 49.86% yield).

¹H NMR (400 MHz, CDCl₃): δ 8.10 (s, 1H), 2.51 (s, 6H).

### Step 3

### Tert-butyl 4-(2-methyloxazol-4-yl)-4-oxobutanoate 81d

To a solution of 81c (1.25 g, 10 mmol) in THF (100 mL) was added dropwise NaHMDS (2 N, 5.5 mL,11 mmoL) at −78° C. After 1 h, tert-Butyl bromoacetate (1.46 mL, 10 mmoL) was added to the solution dropwise. The reaction was stirred at r.t for 18 h. Water (200 mL) was added and the mixture was extracted with EtOAc (100 mL×2). The combined organic phases were washed with brine, dried over anhydrous Na₂SO₄ and concentrated. The residue was purified by silica gel chromatography (eluting with hexane/EtOAc=10/1) to afford the tittle compound 81d (680 mg, 2.85 mmol, 28.45% yield).

### Step 4

### Tert-butyl 3-(4-(2-methyloxazol-4-yl)-2,5-dioxoimidazolidin-4-yl)propanoate 81e

To a solution of 81d (700 mg, 2.93 mmol) in EtOH (10 mL) and water (10 mL) was added (NH₄)₂CO₃ (2.25 g, 23.43 mmol) and NaCN (359 mg, 7.33 mmol). The reaction was sealed in a vessel and stirred at 90° C. for 18 h. The mixture was poured to Water (100 mL) and extracted with EtOAc (50 mL×3). The combined organic phases were washed with brine, dried over anhydrous Na₂SO₄ and concentrated. The residue was purified by reversed phase column chromatography to afford the tittle compound 81e (180 mg, 0.58 mmol, 19.88% yield).

LCMS: MS m/z (ESI): 254.5 [M-tBu+H]⁺.

### Step 5

### 3-(4-(2-methyloxazol-4-yl)-2,5-dioxoimidazolidin-4-yl)propanoic acid 81f

A solution of 81e (60 mg, 0.19 mmol) in HCl/1,4-dioxane (4N, 4 mL) was stirred at r.t for 4 h.

The solution was concentrated to afford the tittle compound 81f (55 mg, 0.19 mmol, 100% yield).

### Step 6

### 5-(3-(5-chloro-6-(trifluoromethyl)isoindolin-2-yl)-3-oxopropyl)-5-(2-methyloxazol-4-yl)imidazolidine-2,4-dione 81

To a solution of 81f (55 mg, 0.19 mmol) in DMF (5 mL) was added TEA (0.12 mL, 0.85 mmol) and 5-chloro-6-(trifluoromethyl)isoindoline 40 h (44 mg, 0.17 mmol), then HATU (65 mg, 0.17 mmol) was added. The reaction was stirred at r.t for 2 h. Water (20 mL) was added and the mixture was extracted with EtOAc (20 mL×2). The combined organic phases were washed with brine, dried over anhydrous Na₂SO₄ and concentrated. The residue was purified by prep-HPLC to afford the tittle compound 81 (20 mg, 0.044 mmol, 23.08% yield).

¹H NMR (400 MHz, DMSO-d₆): δ10.88 (s, 1H), 8.35 (s, 1H), 8.06 (s, 1H), 7.90 (d, 3.2 Hz, 1H), 7.76 (d, 1H), 4.84 (d, 2H), 4.68 (d, 2H), 2.39 (s, 3H), 2.36-2.23 (m, 4H).

LCMS: MS m/z (ESI): 457.1 [M+H]⁺.

### Example 82

### 5-(3-(5-chloro-6-(trifluoromethyl)isoindolin-2-yl)-3-oxopropyl)-5-(1-ethyl-1H-imidazol-2-yl)imidazolidine-2,4-dione 82

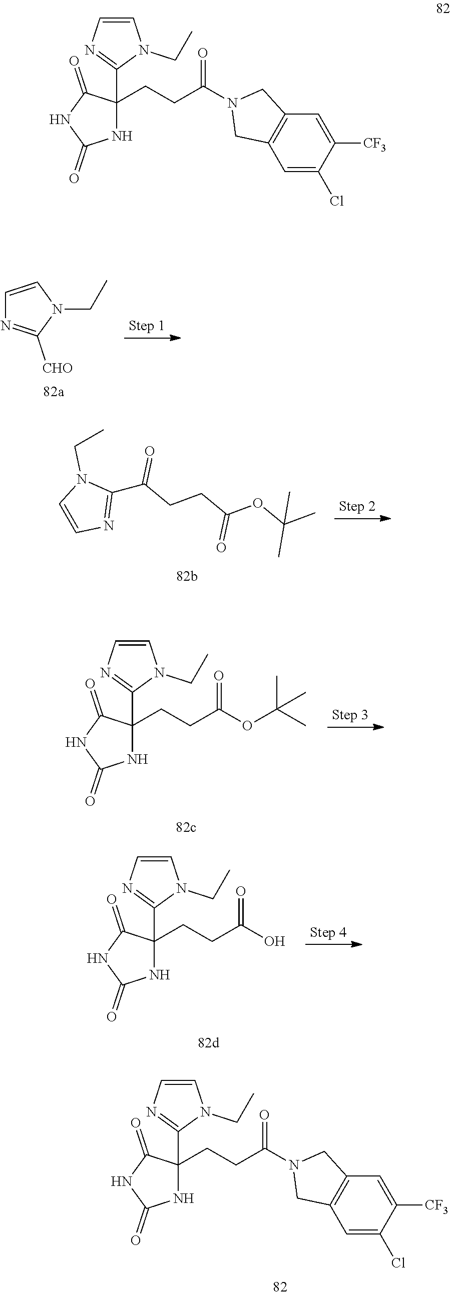

### Step 1

### Tert-butyl 4-(1-ethyl-1H-imidazol-2-yl)-4-oxobutanoate 82b

To a solution of 1-ethyl-1H-imidazole-2-carbaldehyde 82a (500 mg, 4.03 mmol) in THF (6 mL) was added tributylphosphine (0.82 mL, 3.843 mmol), the solution was stirred at 50° C. for 5 min. Then tert-butyl acrylate (468 mg, 3.66 mmol) was added. The reaction was stirred at 80° C. for 18 h. The solution was concentrated. The residue was purified by silica gel chromatography (eluting with hexane/EtOAc=10/1) to afford the tittle compound 82b (84 mg, 0.33 mmol, 8.27% yield).

¹H NMR (400 MHz, CDCl₃): δ 7.14 (d, 1H), 7.08 (d, 1H), 4.43 (q, 2H), 3.41 (t, 2H), 2.63 (t, 2H), 1.43 (s, 9H), 1.41 (t, 3H).

### Step 2

### Tert-butyl 3-(4-(1-ethyl-1H-imidazol-2-yl)-2,5-dioxoimidazolidin-4-yl)propanoate 82c

To a solution of 82b (1.7 g, 6.66 mmol) in EtOH (30 mL) and water (30 mL) was added (NH₄)₂CO₃ (5.12 g, 53.3 mmol) and NaCN (816 mg, 16.66 mmol). The reaction was sealed in a vessel and stirred at 90° C. for 18 h. The mixture was poured to water (100 mL) and extracted with 1-butanol (50 mL×8). The combined organic phases were dried over anhydrous Na₂SO₄ and concentrated. The residue was purified by flash reversed phase column chromatography to afford the tittle compound 82c (220 mg, 0.68 mmol, 10.26% yield).

LCMS: MS m/z (ESI): 323.5 [M+H]⁺.

### Step 3

### 3-(4-(1-ethyl-1H-imidazol-2-yl)-2,5-dioxoimidazolidin-4-yl)propanoic acid 82d

A solution of 82c (50 mg, 0.16 mmol) in HCl/1,4-dioxane (4N, 4 mL) was stirred at r.t for 4 h. The solution was concentrated to afford the tittle compound 82d (45 mg, 0.16 mmol, 100% yield), which was used directly for next step reaction.

### Step 4

### 5-(3-(5-chloro-6-(trifluoromethyl)isoindolin-2-yl)-3-oxopropyl)-5-(1-ethyl-1H-imidazol-2-yl)imidazolidine-2,4-dione 82

To a solution of 82d (45 mg, 0.16 mmol) in DMF (5 mL) was added TEA (0.09 mL, 0.85 mmol) and 5-chloro-6-(trifluoromethyl)isoindoline 40 h (32 mg, 0.12 mmol), then HOBt (22 mg, 0.16 mmol) and EDCI (31 mg, 0.16 mmol) were added. The reaction was stirred at r.t for 18 h. The mixture was purified by prep-HPLC to afford the tittle compound 82 (2.8 mg, 0.006 mmol, 3.75% yield).

¹H NMR (400 MHz, Methanol-d₄): δ 7.78 (d, 1H), 7.67 (s, 1H), 7.62 (d, 1H), 7.46 (s, 1H), 4.93-4.90 (m, 2H), 4.79-4.77 (m, 4H), 4.43 (q, 2H), 2.81-2.60 (m, 4H), 1.53 (t, 7.2 Hz, 3H).

LCMS: MS m/z (ESI): 470.1 [M+H]⁺.

### Examples 83-1 & 83-2

### (S)-5-(3-(5-chloro-6-(trifluoromethyl)isoindolin-2-yl)-3-oxopropyl)-5-(thiazol-2-yl)imidazolidine-2,4-dione 83-1 & (R)-5-(3-(5-chloro-6-(trifluoromethyl)isoindolin-2-yl)-3-oxopropyl)-5-(thiazol-2-yl)imidazolidine-2,4-dione 83-2

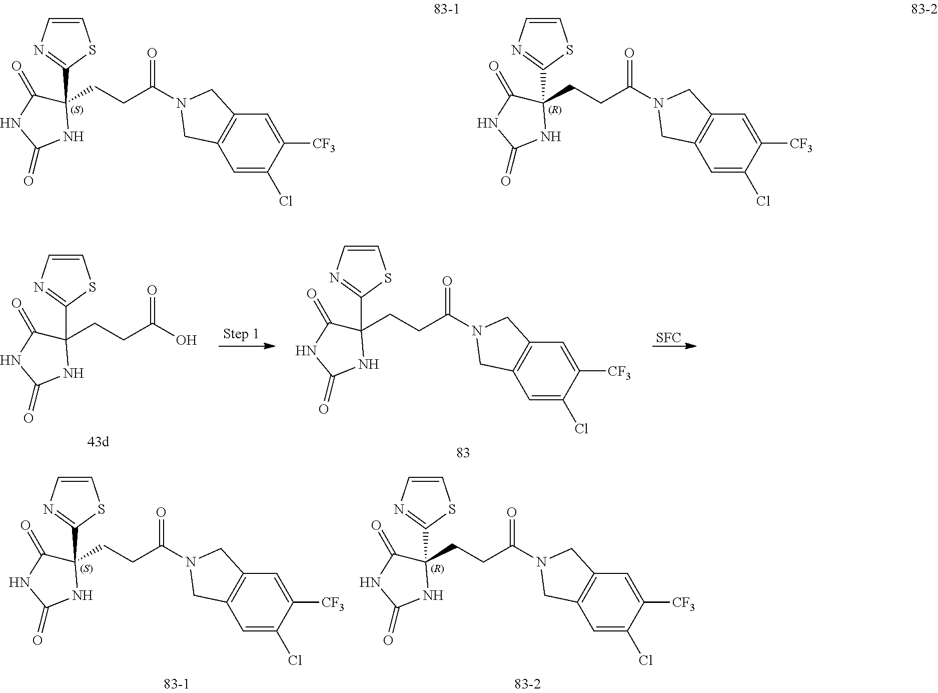

### Step 1

### (S)-5-(3-(5-chloro-6-(trifluoromethyl)isoindolin-2-yl)-3-oxopropyl)-5-(thiazol-2-yl)imidazolidine-2,4-dione 83-1 & (R)-5-(3-(5-chloro-6-(trifluoromethyl)isoindolin-2-yl)-3-oxopropyl)-5-(thiazol-2-yl)imidazolidine-2,4-dione 83-2

A mixture of 43d (102 mg, 400 umol), 40 h (103 mg, 400 umol), HATU (152 mg, 400 umol) and Et₃N (161 mg, 1.60 mmol) in DMF (4 mL) was stirred at room temperature for 18 hours. Then the reaction mixture was purified by prep-HPLC (Waters 2767/2545/2489, Waters Xbridge C18 10 um OBD 19*250 mm, Mobile Phase A: 0.1% NH₄OH in water, Mobile Phase B: CH₃CN, Flow: 20 mL/min, Column temp: RT) to afford racemic mixture 83 (60 mg), which was separated by SFC to afford two enantiomers (18 mg, 39.23 umol, 9.82% yield, and 20 mg, 43.59 umol, 10.9% yield, separately).

Enantiomer with Shorter Retention Time:

¹H NMR (400 MHz, DMSO-d₆): δ11.10 (br, 1H), 8.83 (br, 1H), 7.91-7.85 (m, 2H), 7.81-7.74 (m, 2H), 4.83-4.78 (m, 2H), 4.69-4.66 (m, 2H), 2.44-2.33 (m, 4H).

HPLC: 99.985%® 254 nm, 99.985%® 214 nm.

Chiral HPLC ((C₀₂/EtOH/DEA 60/40/0.04 1.8 ml/min IG,3 um,3*100(Daicel)): Rt: 2.119 min, ee: 100%.

LCMS: MS m/z (ESI): 459.0 [M+H]⁺.

Enantiomer with Longer Retention Time:

¹H NMR (400 MHz, DMSO-d₆): δ11.10 (br, 1H), 8.83 (br, 1H), 7.91-7.85 (m, 2H), 7.81-7.74 (m, 2H), 4.83-4.78 (m, 2H), 4.69-4.66 (m, 2H), 2.44-2.33 (m, 4H).

HPLC: 99.078%® 254 nm, 99.403%® 214 nm.

Chiral HPLC (C₀₂/EtOH/DEA 60/40/0.04 1.8 ml/min IG,3 um,3*100(Daicel)): Rt: 5.782 min; ee: 100%.

LCMS: MS m/z (ESI): 459.0 [M+H]⁺.

### Examples 84-1 and 84-2

### (R)-5-(3-(5-chloro-6-(trifluoromethyl)isoindolin-2-yl)-3-oxopropyl)-5-(pyridin-2-yl)imidazolidine-2,4-dione 84-1

### (S)-5-(3-(5-chloro-6-(trifluoromethyl)isoindolin-2-yl)-3-oxopropyl)-5-(pyridin-2-yl)imidazolidine-2,4-dione 84-2

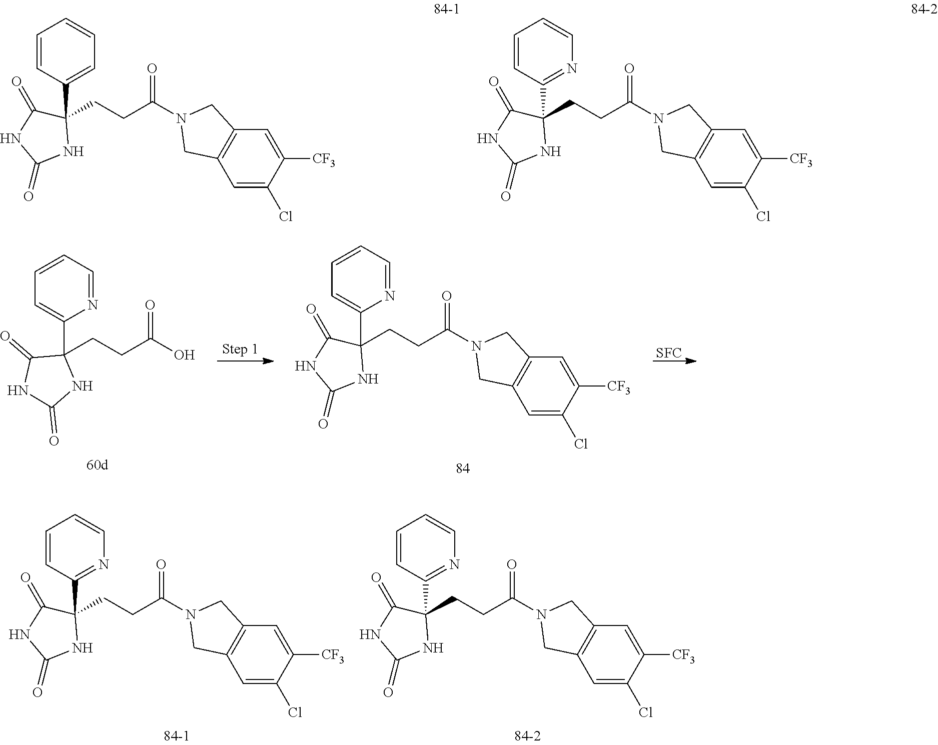

### Step 1

### (R)-5-(3-(5-chloro-6-(trifluoromethyl)isoindolin-2-yl)-3-oxopropyl)-5-(pyridin-2-yl)imidazolidine-2,4-dione 84-1 & (S)-5-(3-(5-chloro-6-(trifluoromethyl)isoindolin-2-yl)-3-oxopropyl)-5-(pyridin-2-yl)imidazolidine-2,4-dione 84-2

A mixture of 60d (114 mg, 399 umol), 40 h (78 mg, 302 umol), HATU (152 mg, 399 umol), TEA (162 mg, 1.60 mmol) and DMF (4.0 mL) was stirred at room temperature for 18 hours.

The reaction mixture was purified by Prep-HPLC (Waters 2767/2545/2489, Waters Xbridge C18 10 um OBD 19*250 mm, Mobile Phase A: 0.1% NH₄₀H in water, Mobile Phase B: CH₃CN, Flow: 20 mL/min, Column temp: RT) to afford racemic mixture 84 (60 mg), which was separated by SFC to afford two enantiomers (15 mg, 33.13 umol, 8.30% yield, and 15 mg, 33.13 umol, 8.30% yield).

Enantiomer with Shorter Retention Time:

¹H NMR (400 MHz, DMSO-d₆): δ 10.87 (brs, 1H), 8.62 (dd, 1H), 8.51 (s, 1H), 7.91-7.84 (m, 2H), 7.76 (d, 1H), 7.54 (d, 1H), 7.41-7.37 (m, 1H), 4.83-4.80 (m, 2H), 4.69-4.66 (m, 2H), 2.51-2.41 (m, 2H), 2.36-2.30 (m, 2H).

LCMS: MS m/z (ESI): 453.0 [M+H]⁺.

Chrial HPLC (C₀₂/EtOH/DEA 60/40/0.04 1.8 ml/min IG,3 um,3*100(Daicel)): RT, 1.852; Purity: 100%.

Enantiomer with Longer Retention Time:

¹H NMR (400 MHz, DMSO-d₆): δ 10.88 (brs, 1H), 8.62 (dd, 1H), 8.53 (s, 1H), 7.91-7.85 (m, 2H), 7.76 (d, 1H), 7.54 (d, 1H), 7.41-7.37 (m, 1H), 4.83-4.80 (m, 2H), 4.69-4.66 (m, 2H), 2.51-2.44 (m, 2H), 2.36-2.30 (m, 2H).

LCMS: MS m/z (ESI): 453.1 [M+H]⁺.

Chrial HPLC (C₀₂/EtOH/DEA 60/40/0.04 1.8 ml/min IG,3 um,3*100(Daicel)): RT, 2.713; Purity: 99.32%.

### Examples 85-1 and 85-2

### (R)-5-(3-(5-chloro-6-(trifluoromethyl)isoindolin-2-yl)-3-oxopropyl)-5-(pyrazin-2-yl)imidazolidine-2,4-dione 85-1

### (S)-5-(3-(5-chloro-6-(trifluoromethyl)isoindolin-2-yl)-3-oxopropyl)-5-(pyrazin-2-yl)imidazolidine-2,4-dione 85-2

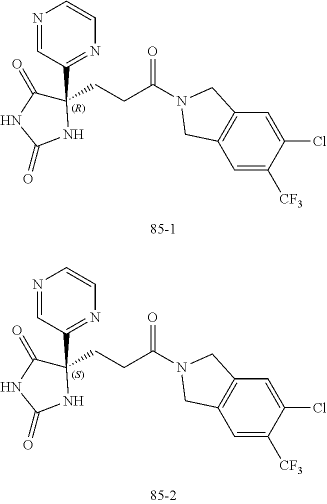

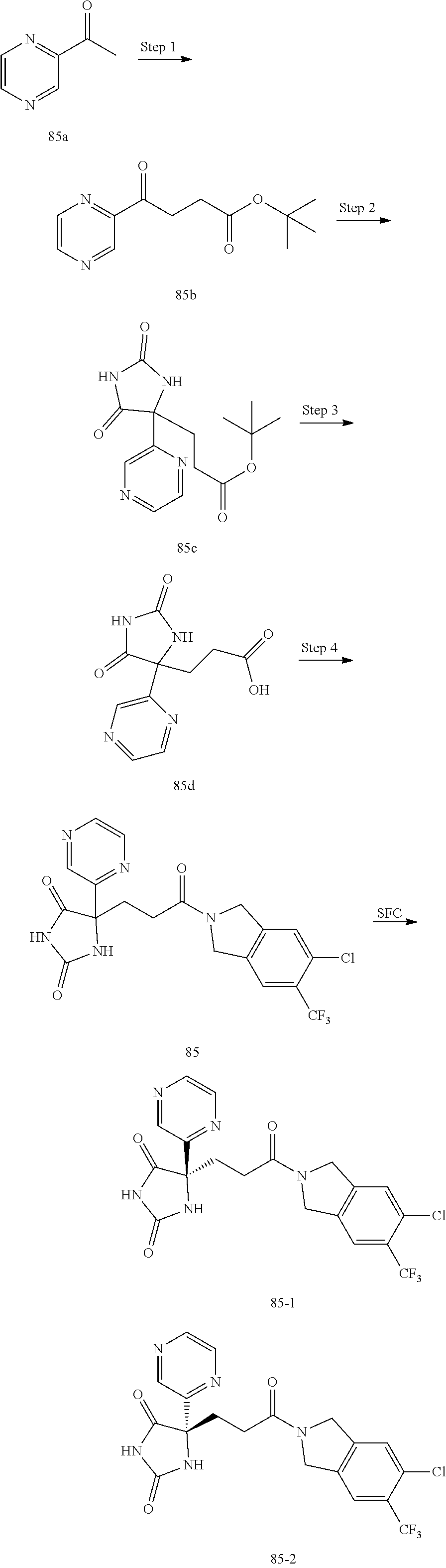

### Step 1

### tert-butyl 4-oxo-4-(thiazol-2-yl)butanoate 85b

To a solution of 1-(pyrazin-2-yl)ethan-1-one 85a (12.0 g, 98.3 mmol) in THF (200 mL) at −70° C. was added NaHMDs (49.2 mL, 98.3 mmol, 2.0 M in THF) dropwise. The resulting mxiture was stirred at this temperature for 30 min before tert-butyl 2-bromoacetate (19.2 g, 98.3 mmol) was added drop-wsie. After addition, the reaction mixture was stirred at −20° C. for 1.0 hour, then warmed to room temperature and stirred for 18 hours. The resulting mixture was cooled to 0° C., then quenched with aq. NaHCO₃ (200 mL). The mixture was extracted with EtOAc (300 mL×4). The organic layers were combined, dried over anhydrous Na₂SO₄ and filtered.

The filtrate was concentrated under reduced pressure. The residue was purified by silica gel chromatography (EtOAc/hexane=1/20 to 1/4) to afford 85b (13.5 g, 57.14 mmol, 58.2% yield).

LCMS: MS m/z (ESI): 237.1 [M+H]⁺.

### Step 2

### tert-butyl 3-(2,5-dioxo-4-(pyrazin-2-yl)imidazolidin-4-yl)propanoate 85c

A mixture of 85b (13.0 g, 55.0 mmol), (NH₄)₂CO₃ (44.9 g, 468 mmol) and NaCN (11.7 g, 220 mmol) in EtOH (60 mL) and H₂O (60 mL) was heated to 120° C. in an autocalve and stirred for 18 hours. The resulting mixture was diluted with water (100 mL). The mixture was extracted with EtOAc (200 mL×3) and n-BuOH (200 mL×3). The combined organic layers were washed with brine (200 mL) and concentrated under reduced pressure. The residue was purified by silica gel chromatography (MeOH/DCM=1/100 to 1/30) to afford 85c (3.0 g, 9.79 mmol, 17.80% yield).

LCMS: MS m/z (ESI): 307.1 [M+H]⁺.

### Step 3

### 3-(2,5-dioxo-4-(pyrazin-2-yl)imidazolidin-4-yl)propanoic acid 85d

To a solution of 85c (900 mg, 2.94 mmol) in DCM (30 mL) was added HCl/1,4-dioxane (30 mL, 4.0 M) drop wise. The reaction mixture was stirred room temperature for 18 hours. The resulting mixture was filtered in vacuum. The filter cake was collected, dried in vacuum to afford 85d (600 mg, 2.40 mmol, 81.62% yield).

LCMS: MS m/z (ESI): 249.0[M−H]⁻.

### Step 4

### (R)-5-(3-(5-chloro-6-(trifluoromethyl)isoindolin-2-yl)-3-oxopropyl)-5-(pyrazin-2-yl)imidazolidine-2,4-dione 85-1

### (S)-5-(3-(5-chloro-6-(trifluoromethyl)isoindolin-2-yl)-3-oxopropyl)-5-(pyrazin-2-yl)imidazolidine-2,4-dione 85-2

A mixture of 85d (130 mg, 519.56 umol), 40 h (134 mg, 519.56 umol), TEA (157.72 mg, 1.56 mmol) and HATU (198 mg, 519.56 umol) in DMF (5 mL) was stirred at room temperature for 18 hours. The resulting mixture was purified by prep-HPLC (Waters 2767/2545/2489, Waters Xbridge C18 10 um OBD 19*250 mm, Mobile Phase A: 0.1% NH₄OH in water, Mobile Phase B: CH₃CN, Flow: 20 mL/min, Column temp: RT) to afford racemic mixture 85 (about 50 mg), which was further separated by SFC to afford two enantiomers (16 mg, 35.26 umol, 6.79% yield, and 15 mg, 33.05 umol, 6.36% yield).

Enantiomer with Shorter Retention Time:

¹H NMR (400 MHz, DMSO-d₆): δ 11.05 (brs, 1H), 8.83 (d, 1H), 8.73-8.71 (m, 1H), 8.67 (d, 1H), 8.65 (br, 1H), 7.90 (d, 1H), 7.75 (d, 1H), 4.84-4.80 (m, 2H), 4.69-4.65 (m, 2H), 2.49-2.46 (m, 2H), 2.37-2.33 (m, 2H).

Chiral HPLC (C₀₂/MeOH/DEA 60/40/0.04 2.8 ml/min AY,5 um,4.6*250(Daicel)): Rt: 2.652 min, ee: 100%.

LCMS: MS m/z (ESI): 454.1 [M+H]⁺.

Enantiomer with Longer Retention Time:

¹H NMR (400 MHz, DMSO-d₆): δ 11.01 (br, 1H), 8.82 (d, 1H), 8.72-8.70 (m, 1H), 8.67 (d, 1H), 8.57 (brs, 1H), 7.90 (d, 1H), 7.75 (d, 1H), 4.84-4.80 (m, 2H), 4.69-4.65 (m, 2H), 2.49-2.46 (m, 2H), 2.37-2.33 (m, 2H).

Chiral HPLC (C₀₂/MeOH/DEA 60/40/0.04 2.8 ml/min AY,5 um,4.6*250(Daicel)): Rt: 4.485; ee: 99.43%.

LCMS: MS m/z (ESI): 454.1 [M+H]⁺.

### Examples 86-1, 86-2, 86-3 and 86-4

### (S)-5-((S)-3-(5-chloro-6-(trifluoromethyl)isoindolin-2-yl)-2-(hydroxymethyl)-3-oxopropyl)

### 5-cyclopropylimidazolidine-2,4-dione 86-1 &

### (S)-5-((R)-3-(5-chloro-6-(trifluoromethyl)isoindolin-2-yl)-2-(hydroxymethyl)-3-oxopropyl)

### 5-cyclopropylimidazolidine-2,4-dione 86-2 &

### (R)-5-((S)-3-(5-chloro-6-(trifluoromethyl)isoindolin-2-yl)-2-(hydroxymethyl)-3-oxopropyl)

### 5-cyclopropylimidazolidine-2,4-dione 86-3 &

### (R)-5-((R)-3-(5-chloro-6-(trifluoromethyl)isoindolin-2-yl)-2-(hydroxymethyl)-3-oxopropyl)

### 5-cyclopropylimidazolidine-2,4-dione 86-4

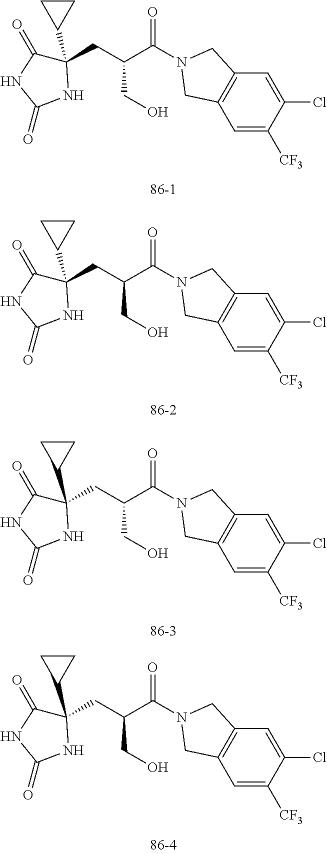 [SMILES: O=C1NC(=O)[C@](C[C@@H](CO)C(=O)N2Cc3cc(Cl)c(C(F)(F)F)cc3C2)(C2CC2)N1 ; O=C1NC(=O)[C@](C[C@H](CO)C(=O)N2Cc3cc(Cl)c(C(F)(F)F)cc3C2)(C2CC2)N1 ; O=C1NC(=O)[C@@](C[C@@H](CO)C(=O)N2Cc3cc(Cl)c(C(F)(F)F)cc3C2)(C2CC2)N1 ; O=C1NC(=O)[C@@](C[C@H](CO)C(=O)N2Cc3cc(Cl)c(C(F)(F)F)cc3C2)(C2CC2)N1]

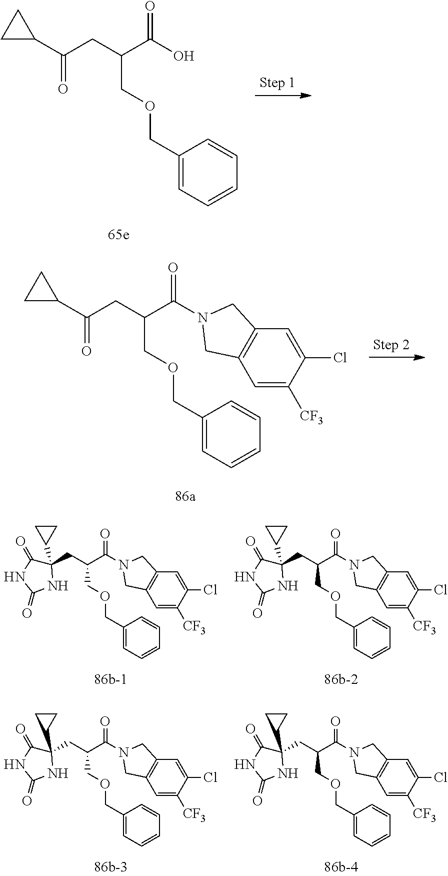 [SMILES: O=C(O)C(COCc1ccccc1)CC(=O)C1CC1 ; O=C(CC(COCc1ccccc1)C(=O)N1Cc2cc(Cl)c(C(F)(F)F)cc2C1)C1CC1 ; O=C1NC(=O)[C@](C[C@@H](COCc2ccccc2)C(=O)N2Cc3cc(Cl)c(C(F)(F)F)cc3C2)(C2CC2)N1 ; O=C1NC(=O)[C@](C[C@H](COCc2ccccc2)C(=O)N2Cc3cc(Cl)c(C(F)(F)F)cc3C2)(C2CC2)N1 ; O=C1NC(=O)[C@@](C[C@@H](COCc2ccccc2)C(=O)N2Cc3cc(Cl)c(C(F)(F)F)cc3C2)(C2CC2)N1 ; O=C1NC(=O)[C@@](C[C@H](COCc2ccccc2)C(=O)N2Cc3cc(Cl)c(C(F)(F)F)cc3C2)(C2CC2)N1]

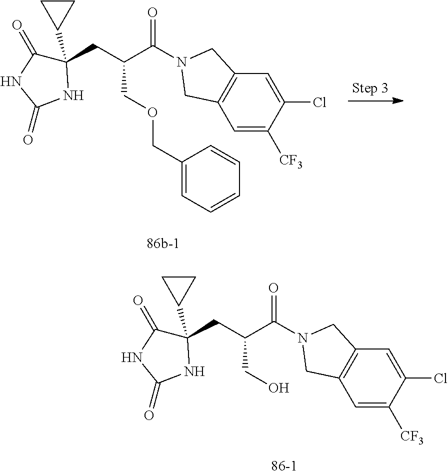 [SMILES: O=C1NC(=O)[C@](C[C@@H](COCc2ccccc2)C(=O)N2Cc3cc(Cl)c(C(F)(F)F)cc3C2)(C2CC2)N1 ; O=C1NC(=O)[C@](C[C@@H](CO)C(=O)N2Cc3cc(Cl)c(C(F)(F)F)cc3C2)(C2CC2)N1]

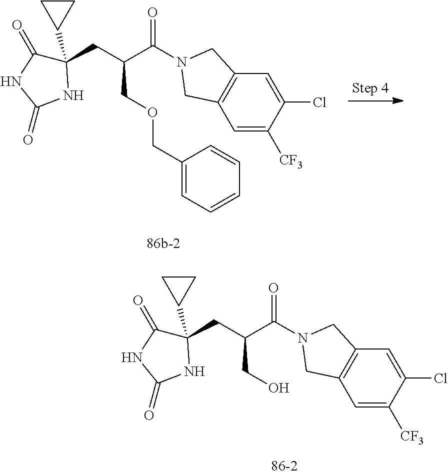 [SMILES: O=C1NC(=O)[C@](C[C@H](COCc2ccccc2)C(=O)N2Cc3cc(Cl)c(C(F)(F)F)cc3C2)(C2CC2)N1 ; O=C1NC(=O)[C@](C[C@H](CO)C(=O)N2Cc3cc(Cl)c(C(F)(F)F)cc3C2)(C2CC2)N1]

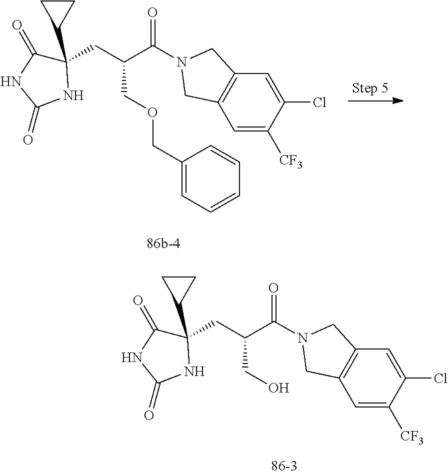 [SMILES: O=C1NC(=O)[C@@](C[C@@H](COCc2ccccc2)C(=O)N2Cc3cc(Cl)c(C(F)(F)F)cc3C2)(C2CC2)N1 ; O=C1NC(=O)[C@@](C[C@@H](CO)C(=O)N2Cc3cc(Cl)c(C(F)(F)F)cc3C2)(C2CC2)N1]

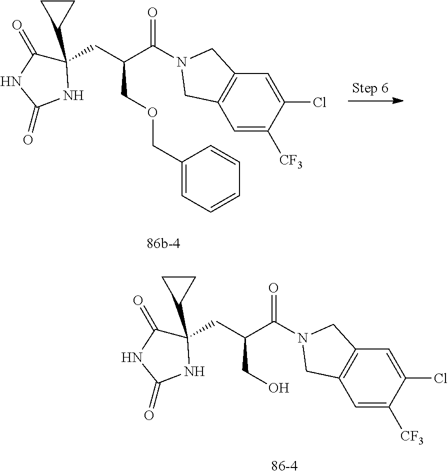 [SMILES: O=C1NC(=O)[C@@](C[C@H](COCc2ccccc2)C(=O)N2Cc3cc(Cl)c(C(F)(F)F)cc3C2)(C2CC2)N1 ; O=C1NC(=O)[C@@](C[C@H](CO)C(=O)N2Cc3cc(Cl)c(C(F)(F)F)cc3C2)(C2CC2)N1]

### Step 1

### 2-((benzyloxy)methyl)-1-(5-chloro-6-(trifluoromethyl)isoindolin-2-yl)-4-cyclopropylbutane-1,4-dione 86a

To a solution of 2-((benzyloxy)methyl)-4-cyclopropyl-4-oxobutanoic acid 65e (350 mg, 1.33 mmol) and 5-chloro-6-(trifluoromethyl)isoindoline 40 h (310 mg, 1.20 mmol) in DMF (10 mL) was added TEA (675 mg, 6.67 mmol) and HATU (609 mg, 1.60 mmol). The reaction was stirred at r.t for 2 h. Water (20 mL) was added and the mixture was extracted with EtOAc (20 mL×2), The combined organic phases were washed with brine, dried over anhydrous Na₂SO₄ and concentrated. The residue was purified by prep-TLC to give 86a (400 mg, 858.57 umol, 64.34% yield).

LCMS: m/z (ESI): 466.1 [M+H]⁺.

### Step 2

### Four isomers of 5-(2-((benzyloxy)methyl)-3-(5-chloro-6-(trifluoromethyl)isoindolin-2-yl)-3-oxopropyl)-5-cyclopropylimidazolidine-2,4-dione 86b

To a solution of 86a (600 mg, 1.29 mmol) in EtOH (20 mL) was added ammonium carbonate (991 mg, 10.32 mmol), NaCN (158 mg, 3.23 mmol) and water (20 mL). The reaction was stirred at 90° C. for 18 h. Water (20 mL) was added and the mixture was extracted with EtOAc (20 mL×3). The combined organic phases were washed with brine, dried over anhydrous Na₂SO₄ and concentrated. The residue was purified by flash reversed phase column chromatography to give 86b (370 mg), which was further separated by SFC to give four isomers (127 mg, 118 mg, 55 mg and 48 mg).

Isomers 1 (1st Peak from SFC):

¹H NMR (400 MHz, DMSO-d₆): δ 10.66 (s, 1H), 7.90 (d, 1H), 7.76 (d, 1H), 7.68 (d, 1H), 7.25-7.22 (m, 51H), 5.01-4.62 (m, 4H), 4.44 (s, 2H), 3.54-3.43 (m, 2H), 2.96-2.90 (m, 1H), 2.28-2.21 (m, 1H), 1.84-1.81 (m, 1H), 1.06-1.00 (m, 1H), 0.42-0.23 (m, 3H), 0.02-0.01 (m, 1H).

¹⁹F NMR (376.5 MHz, DMSO-d₆): δ-60.88.

Chiral HPLC: Rt: 3.12 min, ee: 100%.

LCMS: m/z (ESI): 536.1 [M+H]⁺.

Isomers 2 (2nd Peak from SFC):

¹H NMR (400 MHz, DMSO-d₆): δ 10.66 (s, 1H), 7.91 (d, 1H), 7.77 (d, 1H), 7.71 (d, 1H), 7.25-7.22 (m, 51H), 5.01-4.62 (m, 4H), 4.44 (s, 2H), 3.54-3.43 (m, 2H), 2.96-2.90 (m, 1H), 2.29-2.21 (m, 1H), 1.85-1.81 (m, 1H), 1.06-1.00 (m, 1H), 0.42-0.23 (m, 3H), 0.03-0.01 (m, 1H).

¹⁹F NMR (376.5 MHz, DMSO-d₆): δ-60.87.

Chiral HPLC: Rt: 3.34 min, ee: 100%.

LCMS: m/z (ESI): 536.1 [M+H]⁺.

Isomers 3 (3th Peak from SFC):

¹H NMR (400 MHz, DMSO-d₆): δ 10.52 (s, 1H), 7.91 (s, 1H), 7.77-7.75 (s, 2H), 7.25-7.20 (m, 5H), 5.00-4.54 (m, 4H), 4.44 (s, 2H), 3.55-3.47 (m, 2H), 3.16-3.09 (m, 1H), 2.31-2.25 (m, 1H), 1.73-1.70 (m, 1H), 1.06-1.01 (m, 1H), 0.43-0.26 (m, 3H), 0.13-0.06 (m, 1H).

¹⁹F NMR (376.5 MHz, DMSO-d₆): a −60.85.

Chiral HPLC: Rt: 4.25 min, ee: 100%.

LCMS: m/z (ESI): 536.1 [M+H]⁺.

Isomers 4 (4th Peak from SFC):

¹H NMR (400 MHz, DMSO-d₆): δ 10.52 (s, 1H), 7.91 (s, 1H), 7.77-7.75 (m, 2H), 7.25-7.20 (m, 5H), 5.00-4.54 (m, 4H), 4.44 (s, 2H), 3.55-3.47 (m, 2H), 3.16-3.09 (m, 1H), 2.31-2.25 (m, 1H), 1.73-1.70 (m, 1H), 1.06-1.01 (m, 1H), 0.43-0.26 (m, 3H), 0.13-0.06 (m, 1H).

¹⁹F NMR (376.5 MHz, DMSO-d₆): δ-60.85 nm.

Chiral HPLC: Rt: 4.60 min, ee: 98.76%.

LCMS: m/z (ESI): 536.1 [M+H]⁺.

### Step 3

### (S)-5-((S)-3-(5-chloro-6-(trifluoromethyl)isoindolin-2-yl)-2-(hydroxymethyl)-3-oxopropyl)

### 5-cyclopropylimidazolidine-2,4-dione 86-1

To a solution of isomers 1 from step 2 (120 mg, 0.22 mmol) in EtOAc (60 mL) was added PdCl₂ (60 mg). Then the reaction was stirred at room temperature under H₂ atmosphere for 2 h.

The mixture was filtered, and the filtrate was concentrated. The residue was purified by flash reversed phase column chromatography to give the compound 86-1 (74 mg, 74.75%).

¹H NMR (400 MHz, DMSO-d₆): δ 10.63 (s, 1H), 7.90 (d, 1H), 7.77 (s, 1H), 7.66 (d, 1H), 5.04-4.97 (m, 2H), 4.89-4.83 (m, 1H), 4.77-4.60 (m, 2H), 3.51-3.36 (m, 2H), 2.77-2.70 (m, 1H), 2.20-2.13 (m, 1H), 1.80-1.74 (m, 1H), 1.05-0.98 (m, 1H), 0.42-0.22 (m, 31H), 0.04-0.029 (m, 1H).

¹⁹F NMR (376.5 MHz, DMSO-d₆): δ-60.84 nm.

Chiral HPLC: Rt: 3.29 min, ee: 100%.

LCMS: m/z (ESI): 446.1 [M+H]⁺.

### Step 4

### (S)-5-((R)-3-(5-chloro-6-(trifluoromethyl)isoindolin-2-yl)-2-(hydroxymethyl)-3-oxopropyl)

### 5-cyclopropylimidazolidine-2,4-dione 86-2

To a solution of isomers 2 from step 2 (110 mg, 0.21 mmol) in EtOAc (50 mL) was added PdCl₂ (50 mg). Then the reaction was stirred at room temperature under H₂ atmosphere for 2 h.

The mixture was filtered and the filtrate was concentrated. The residue was purified by flash reversed phase column chromatography to give the compound 86-2 (65 mg, 71.04%).

¹H NMR (400 MHz, DMSO-d₆): δ 10.63 (s, 1H), 7.90 (d, 1H), 7.77 (s, 1H), 7.66 d, 1H), 5.04-4.97 (m, 2H), 4.89-4.82 (m, 1H), 4.77-4.60 (m, 2H), 3.51-3.36 (m, 2H), 2.76-2.70 (m, 1H), 2.20-2.13 (m, 1H), 1.80-1.75 (m, 1H), 1.05-0.98 (m, 1H), 0.43-0.23 (m, 3H), 0.04-0.031 (m, 1H).

¹⁹F NMR (376.5 MHz, DMSO-d₆): δ-60.84 nm.

Chiral HPLC: Rt: 4.66 min, ee: 100%.

LCMS: m/z (ESI): 446.1 [M+H]⁺.

### Step 5

### (R)-5-((S)-3-(5-chloro-6-(trifluoromethyl)isoindolin-2-yl)-2-(hydroxymethyl)-3-oxopropyl)

### 5-cyclopropylimidazolidine-2,4-dione 86-3

To a solution of isomers 3 from step 2 (50 mg, 0.093 mmol) in EtOAc (25 mL) was added PdCl₂ (25 mg). Then the reaction was stirred at room temperature under H₂ atmosphere for 2 h, the mixture was filtered and the filtrate was concentrated. The residue was purified by flash reversed phase column chromatography to give the compound 86-3 (37 mg, 89.16%).

¹H NMR (400 MHz, DMSO-d₆): δ 10.50 (s, 1H), 7.91 (s, 1H), 7.78 (d, 1H), 7.74 (d, 1H), 5.04-4.97 (m, 2H), 4.87-4.80 (m, 1H), 4.74-4.67 (m, 1H), 4.60-4.53 (m, 1H), 3.51-3.34 (m, 2H), 2.96-2.89 (m, 1H), 2.25-2.19 (m, 1H), 1.67-1.63 (m, 1H), 1.07-1.00 (m, 1H), 0.43-0.25 (m, 3H), 0.11-0.05 (m, 1H).

¹⁹F NMR (376.5 MHz, DMSO-d₆): δ-60.84.

Chiral HPLC: Rt: 5.73 min, ee: 97.83%.

LCMS: m/z (ESI): 446.1 [M+H]⁺.

### Step 5

### (R)-5-((R)-3-(5-chloro-6-(trifluoromethyl)isoindolin-2-yl)-2-(hydroxymethyl)-3-oxopropyl)

### 5-cyclopropylimidazolidine-2,4-dione 86-4

To a solution of isomers 4 from step 2 (45 mg, 0.084 mmol) in EtOAc (25 mL) was added PdCl₂ (25 mg). Then the reaction was stirred atroom temperature under H₂ atmosphere for 2 h, the mixture was filtered and the filtrate was concentrated. The residue was purified by flash reversed phase column chromatography to give the compound 86-4 (37 mg, 89.16%).

¹H NMR (400 MHz, DMSO-d₆): δ 10.50 (s, 1H), 7.91 (s, 1H), 7.78 (d, 1H), 7.74 (d, 1H), 5.05-4.97 (m, 2H), 4.87-4.81 (m, 1H), 4.75-4.68 (m, 1H), 4.60-4.53 (m, 1H), 3.51-3.40 (m, 2H), 2.96-2.89 (m, 1H), 2.25-2.20 (m, 1H), 1.67-1.63 (m, 1H), 1.07-1.00 (m, 1H), 0.43-0.24 (m, 3H), 0.11-0.05 (m, 1H).

¹⁹F NMR (376.5 MHz, DMSO-d₆): δ-60.83.

Chiral HPLC: Rt: 7.07 min, ee: 97.16%.

LCMS: m/z (ESI): 446.1 [M+H]⁺.

The following compounds were prepared using the similar methods as Examples 1-86.

| Ex# | Structure and chemical name | ¹H NMR and mass spectra |
|---|---|---|
| 10 | 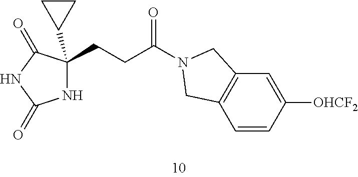 | ¹H NMR (400 MHz, CD₃OD): δ 7.25-7.23 (m, 1H), 7.04-7.01 (m, 1H), 7.02-6.98 (m, 1H), 4.77-4.73 (m, 2H), 4.65-4.62 (m, 2 H), 2.46-2.41 (m, 2H), 2.37-2.33 (m, 1H), 2.14- 2.10 (m, 2H), 1.17-1.15 (m, 1H), 0.50-0.47 (m, 1H), 0.34-0.30 (m, 2H), 0.27-0.22 (m, 1H). LC-MS: MS m/z (ESI): [M + H]⁺. 380.2 |
| | (S)-5-cyclopropyl-5-(3-(5- | |
| | (difluoromethoxy)isoindolin-2- | |
| | yl)-3-oxopropyl)imidazolidine- | |
| | 2,4-dione | |
| 11 | 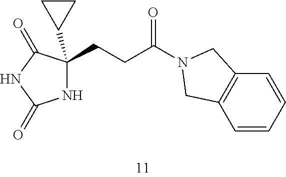 [SMILES: O=C1NC(=O)[C@](CCC(=O)N2Cc3ccccc3C2)(C2CC2)N1] | ¹H NMR (400 MHz, CD₃OD): δ 7.22-1.16 (m, 4H), 4.78-4.73 (m, 2H), 4.67-4.62 (m, 2 H), 2.46-2.44 (m, 1H), 2.37-2.34 (m, 1 H), 2.14-2.10 (m, 2H), 1.16-1.12 (m, 1H), 0.51- 0.47 (m, 1H), 0.34-0.30 (m, 2H), 0.27-0.23 (m, 1H). LC-MS: MS m/z (ESI): [M + H]⁺. 314.2 |
| | (S)-5-cyclopropyl-5-(3- | |
| | (isoindolin-2-yl)-3- | |
| | oxopropyl)imidazolidine-2,4- | |
| | dione | |
| 12 | 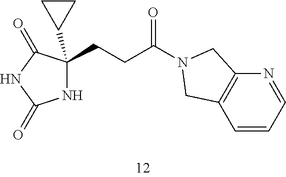 [SMILES: O=C1NC(=O)[C@](CCC(=O)N2Cc3cccnc3C2)(C2CC2)N1] | ¹H NMR (400 MHz, CD₃OD): δ 8.41-8.37 (m, 1H), 7.81-7.78 (m, 1H), 7.34-7.31 (m, 1H), 4.72-4.68 (m, 2H), 3.57-3.55 (m, 2 H), 2.46-2.42 (m, 1H), 2.36-2.34 (m, 1H), 2.14- 2.12 (m, 2H), 1.16-1.14 (m, 1H), 0.50-0.47 (m, 1H), 0.33-0.30 (m, 2H), 0.27-0.23 (m, 1H). LC-MS: MS m/z (ESI): [M + H]⁺. 315.2 |
| | (S)-5-cyclopropyl-5-(3-(5,7- | |
| | dihydro-6H-pyrrolo[3,4- | |
| | b]pyridin-6-yl)-3- | |
| | oxopropyl)imidazolidine-2,4- | |
| | dione | |
| 13 | 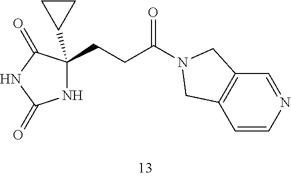 [SMILES: O=C1NC(=O)[C@](CCC(=O)N2Cc3ccncc3C2)(C2CC2)N1] | ¹H NMR (400 MHz, CD₃OD): δ 8.74-8.72 (m, 1H), 8.67-8.63 (m, 1H), 7.90-7.87 (m, 1H), 5.04-5.01 (m, 2H), 4.88-4.85 (m, 2 H), 2.47-2.45 (m, 1H), 2.39-2.35 (m, 1 H), 2.18- 2.11 (m, 2H), 1.15-1.13 (m, 1H), 0.50-0.48 (m, 1H), 0.33-0.30 (m, 2H), 0.27-0.23 (m, 1H). LC-MS: MS m/z (ESI): [M + H]⁺. 315.2 |
| | (S)-5-cyclopropyl-5-(3-(1,3- | |
| | dihydro-2H-pyrrolo[3,4- | |
| | c]pyridin-2-yl)-3- | |
| | oxopropyl)imidazolidine-2,4- | |
| | dione | |
| 14 | 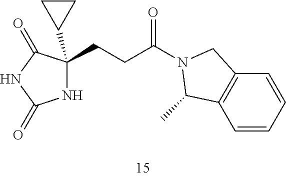 [SMILES: C[C@H]1c2ccccc2CN1C(=O)CC[C@@]1(C2CC2)NC(=O)NC1=O] | ¹H NMR (400 MHz, CD₃OD): δ 7.36-7.28 (m, 4H), 5.28 (q, 1H), 4.89-4.84 (m, 2H), 2.57-2.43 (m, 2H), 2.25-2.22 (m, 2H), 1.50 (d, 3H), 1.40-1.37 (m, 2H), 1.28-1.26 (m, 1H), 0.50-0.48 (m, 1H), 0.34-0.32 (m, 2 H), 0.27-0.23 (m, 1H). LC-MS: MS m/z (ESI): [M + H]⁺. 328.2 |
| | (S)-5-cyclopropyl-5-(3-((R)-1- | |
| | methylisoindolin-2-yl)-3- | |
| | oxopropyl)imidazolidine-2,4- | |
| | dione | |
| 15 | 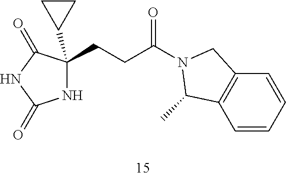 [SMILES: C[C@H]1c2ccccc2CN1C(=O)CC[C@@]1(C2CC2)NC(=O)NC1=O] | ¹H NMR (400 MHz, CD₃OD): δ 7.35-7.28 (m, 4H), 5.28 (q, 1H), 4.89-4.85 (m, 2H), 2.57-2.43 (m, 2H), 2.25-2.21 (m, 2H), 1.50 (d, 3H), 1.39-1.34 (m, 2H), 1.28-1.22 (m, 1H), 0.50-0.44 (m, 1H), 0.34-0.30 (m, 2 H), 0.27-0.23 (m, 1H). LC-MS: MS m/z (ESI): [M + H]⁺. 328.2 |
| | (S)-5-cyclopropyl-5-(3-((S)-1- | |
| | methylisoindolin-2-yl)-3- | |
| | oxopropyl)imidazolidine-2,4- | |
| | dione | |
| 16 | 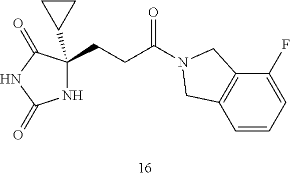 [SMILES: O=C1NC(=O)[C@](CCC(=O)N2Cc3cccc(F)c3C2)(C2CC2)N1] | ¹H NMR (400 MHz, CD₃OD): δ 7.25-7.22 (m, 1H), 7.08-7.06 (m, 1H), 6.96-6.92 (m, 1H), 4.85-4.81 (m, 2H), 4.69-4.66 (m, 2 H), 2.47-2.42 (m, 1H), 2.36-2.34 (m, 1H), 2.12- 2.09 (m, 2H), 1.17-1.14 (m, 1H), 0.50-00.47 (m, 1H), 0.33-0.31 (m, 2H), 0.27-0.23 (m, 1H). LC-MS: MS m/z (ESI): [M + H]⁺. 332.2 |
| | (S)-5-cyclopropyl-5-(3-(4- | |
| | fluoroisoindolin-2-yl)-3- | |
| | oxopropyl)imidazolidine-2,4- | |
| | dione | |
| 17 | 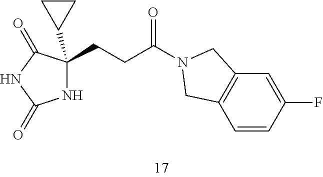 [SMILES: O=C1NC(=O)[C@](CCC(=O)N2Cc3ccc(F)cc3C2)(C2CC2)N1] | ¹H NMR (400 MHz, CD₃OD): δ 7.38-7.27 (m, 1H), 7.16-6.96 (m, 2H), 4.72 (d, 2H), 3.37 (s, 2H), 2.60-2.54 (m, 1H), 2.48-2.42 (m, 1H), 2.33-2.11 (m, 2H), 1.25 (tt, 1H), 0.69-0.53 (m, 1H), 0.53-0.37 (m, 2H), 0.37-0.23 (m, 1H). LC-MS: MS m/z (ESI): [M + H]⁺. 332.2 |
| | (S)-5-cyclopropyl-5-(3-(5- | |
| | fluoroisoindolin-2-yl)-3- | |
| | oxopropyl)imidazolidine-2,4- | |
| | dione | |
| 18 | 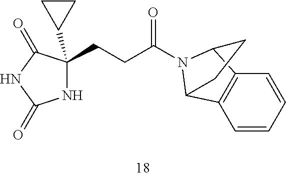 [SMILES: O=C1NC(=O)[C@](CCC(=O)N2C3CCC2c2ccccc23)(C2CC2)N1] | ¹H NMR (400 MHz, CD₃OD): 7.34-7.30 (m, 2H), 7.20-7.17 (m, 2H), 5.50 (d, 1 H), 5.32 (d, 1H), 2.46-2.02 (m, 6H), 1.42-1.30 (m, 2H), 1.19-1.16 (m, 1H), 0.54-0.51 (m, 1H), 0.44-0.27 (m, 3H). LC-MS: MS m/z (ESI): [M + H]⁺. 340.2; |
| | (5S)-5-cyclopropyl-5-(3-oxo-3- | |
| | (1,2,3,4-tetrahydro-1,4- | |
| | epiminonaphthalen-9- | |
| | yl)propyl)imidazolidine-2,4- | |
| | dione | |
| 19 | 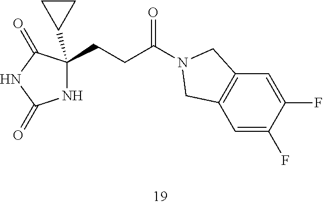 [SMILES: O=C1NC(=O)[C@](CCC(=O)N2Cc3cc(F)c(F)cc3C2)(C2CC2)N1] | ¹H NMR (400 MHz, CD₃OD) δ 7.27 (dt, 2H), 4.85 (s, 2H), 4.72 (s, 2H), 2.58-2.53 (m, 1H), 2.45-2.41 (m, 1H), 2.29-2.18 (m, 2H), 1.32-1.18 (m, 1H), 0.64-0.58 (m, 1H), 0.52-0.23 (m, 3H). LC-MS: MS m/z (ESI): [M + H]⁺. 350.2 |
| | (S)-5-cyclopropyl-5-(3-(5,6- | |
| | difluoroisoindolin-2-yl)-3- | |
| | oxopropyl)imidazolidine-2,4- | |
| | dione | |
| 21 | 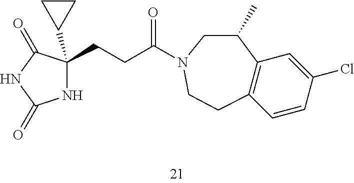 [SMILES: C[C@H]1CN(C(=O)CC[C@@]2(C3CC3)NC(=O)NC2=O)CCc2ccc(Cl)cc21] | ¹H NMR (500 MHz, DMSO-d₆) δ 10.07 (br, 1H), 7.74 (d, 1H), 7.14-7.06 (m, 3H), 3.56- 3.33 (m, 4H), 3.12-3.03 (m, 1H), 2.98-2.70 (m, 2H), 2.41-2.12 (m, 2H), 1.83-1.72 (m, 2H), 1.11 (m, 3H), 0.98-0.96 (m, 1H), 0.40- 0.20 (m, 3H), 0.05--0.06 (m, 1H). LCMS: MS m/z (ESI): 390.1 [M + H]⁺. |
| | (S)-5-(3-((R)-8-chloro-1-methyl- | |
| | 1,2,4,5-tetrahydro-3H- | |
| | benzo[d]azepin-3-yl)-3- | |
| | oxopropyl)-5- | |
| | cyclopropylimidazolidine-2,4- | |
| | dione | |
| 22 | 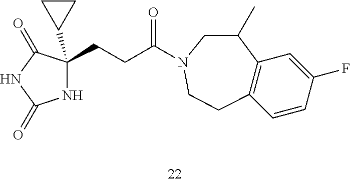 [SMILES: CC1CN(C(=O)CC[C@@]2(C3CC3)NC(=O)NC2=O)CCc2ccc(F)cc21] | Mixture of two diastereomers ¹H NMR (400 MHz, CD₃OD): 7.08-6.98 (m, 1H), 6.90-6.70 (m, 2H), 3.66-3.58 (m, 2H), 3.48-3.35 (m, 2H), 3.18-3.11 (m, 1 H), 3.04- 2.94 (m, 1H), 2.87-2.73 (m, 1H), 2.41-2.18 (m, 2H), 1.98-1.85 (m, 2H), 1.37-1.28 (m, 3H), 1.11-1.02 (m, 1H), 0.48-0.42 (m, 1H), 0.38-0.15 (m, 3H). MS m/z (ESI): [M + H]⁺. 374.2 |
| | Mixture of two diastereomers | |
| | (5S)-5-cyclopropyl-5-(3-(8- | |
| | fluoro-1-methyl-1,2,4,5- | |
| | tetrahydro-3H-benzo[d]azepin-3- | |
| | y1)-3-oxopropyl)imidazolidine- | |
| | 2,4-dione | |
| 23 | 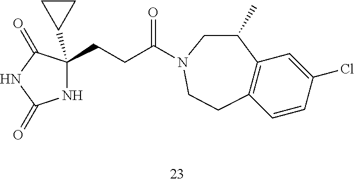 | ¹H NMR (400 MHz, CD₃OD): 7.18-7.16 (m, 1H), 7.01-6.99 (m, 2H), 3.63-3.58 (m, 2H), 3.16-3.14 (m, 1H), 3.10-3.07 (m, 1 H), 2.98- 2.94 (m, 1H), 2.91-2.87 (m, 1H), 2.39-2.36 (m, 1H), 2.29-2.26 (m, 1H), 1.97-1.90 (m, 2H), 1.10-1.06 (m, 2H), 0.46-0.44 (m, 1H), 0.33-0.18 (m, 4H). LC-MS: MS m/z (ESI): [M + H]⁺. 376.2 |
| | (S)-5-(3-(7-chloro-1,2,4,5- | |
| | tetrahydro-3H-benzo[d]azepin-3- | |
| | yl)-3-oxopropyl)-5- | |
| | cyclopropylimidazolidine-2,4- | |
| | dione | |
| 24 | 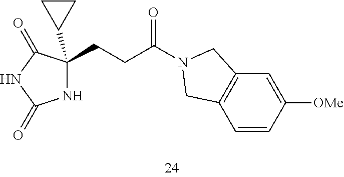 [SMILES: COc1ccc2c(c1)CN(C(=O)CC[C@@]1(C3CC3)NC(=O)NC1=O)C2] | MS m/z (ESI): [M + H]⁺. 344.2 |
| | (S)-5-cyclopropyl-5-(3-(5- | |
| | methoxyisoindolin-2-yl)-3- | |
| | oxopropyl)imidazolidine-2,4- | |
| | dione | |
| 25 | 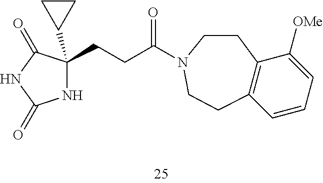 [SMILES: COc1cccc2c1CCN(C(=O)CC[C@@]1(C3CC3)NC(=O)NC1=O)CC2] | ¹H NMR (400 MHz, CD₃OD): δ 7.29-7.25 (m, 1H), 7.0-6.89 (m, 2H), 3.62-3.58 (m, 2H), 3.67 (s, 3H), 3.16-3.14 (m, 1H), 3.10- 3.08 (m, 1H), 2.98-2.96 (m, 1H), 2.90-2.88 (m, 1H), 2.38-2.35 (m, 1H), 2.28-2.25 (m, 1H), 1.95-1.91 (m, 2H), 1.10-1.05 (m, 2H), 0.45-0.42 (m, 1H), 0.33-0.18 (m, 4H). LC-MS: MS m/z (ESI): [M + H]⁺. 372.2 |
| | (S)-5-cyclopropyl-5-(3-(6- | |
| | methoxy-1,2,4,5-tetrahydro-3H- | |
| | benzo[d]azepin-3-yl)-3- | |
| | oxopropyl)imidazolidine-2,4- | |
| | dione | |
| 26 | 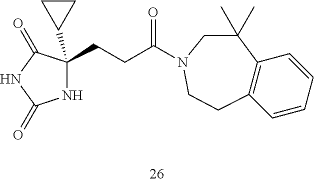 [SMILES: CC1(C)CN(C(=O)CC[C@@]2(C3CC3)NC(=O)NC2=O)CCc2ccccc21] | ¹H NMR (400 MHz, CD₃OD) δ 7.40 (t, 1H), 7.21 (dt, 1H), 7.15-7.02 (m, 2H), 3.81- 3.70 (m, 3H), 3.15 (dt, 2H), 2.60-2.49 (m, 1H), 2.49-2.35 (m, 1H), 2.24-2.03 (m, 3H), 1.44-1.27 (m, 6H), 1.27-1.12 (m, 1H), 0.65-0.57 (m, 1H), 0.49-0.21 (m, 3H). LC-MS: MS m/z (ESI): [M + H]+. 370.2 |
| | (S)-5-cyclopropyl-5-(3-(1,1- | |
| | dimethyl-1,2,4,5-tetrahydro-3H- | |
| | benzo[d]azepin-3-yl)-3- | |
| | oxopropyl)imidazolidine-2,4- | |
| | dione | |
| 27 & 28 | 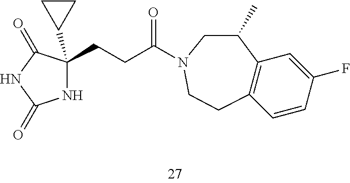 [SMILES: C[C@H]1CN(C(=O)CC[C@@]2(C3CC3)NC(=O)NC2=O)CCc2ccc(F)cc21] | ¹H NMR (500 MHz, DMSO-d₆) δ 10.61 (d, 1H), 7.72 (d, 1H), 7.16 (td, 1H), 6.98-6.92 (m, 2H), 3.68-3.49 (m, 2H), 3.23-2.74 (m, 4H), 2.41-2.36 (m, 1H), 2.27-2.20 (m, 1H), 1.95-1.90 (m, 2H), 1.21 (dd, 4H), 1.09- 1.01 (m, 1H), 0.94-0.80 (m, 1H), 0.47- 0.28 (m, 2H), 0.09 (dd, 1H). Enantiomer 1 with shorter retention time: HPLC: 99.560% @ 214 nm Chiral HPLC: Rt: 13.049 min, ee: 92.3%. LC-MS: MS m/z (ESI): [M + H]⁺. 374.2 |
| | (S)-5-cyclopropyl-5-(3-((R)-8- | |
| | fluoro-1-methyl-1,2,4,5- | |
| | tetrahydro-3H-benzo[d]azepin-3- | |
| | yl)-3-oxopropyl)imidazolidine- | |
| | 2,4-dione | |
| | 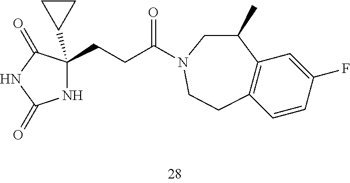 [SMILES: C[C@@H]1CN(C(=O)CC[C@@]2(C3CC3)NC(=O)NC2=O)CCc2ccc(F)cc21] | Enantiomer 2 with longer retention time HPLC: 100.000% @ 214 nm Chiral HPLC: Rt: 13.070 min, ee: 89.4%. LC-MS: MS m/z (ESI): [M + H]⁺. 374.2 |
| | (S)-5-cyclopropyl-5-(3-((S)-8- | |
| | fluoro-1-methyl-1,2,4,5- | |
| | tetrahydro-3H-benzo[d]azepin-3- | |
| | y1)-3-oxopropyl)imidazolidine- | |
| | 2,4-dione | |
| 39 | 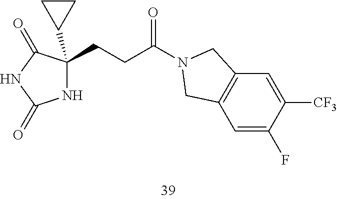 [SMILES: O=C1NC(=O)[C@](CCC(=O)N2Cc3cc(F)c(C(F)(F)F)cc3C2)(C2CC2)N1] | ¹H NMR (400 MHz, CD₃OD) δ 7.78-7.64 (m, 1H), 7.36 (t, 1H), 4.91 (s, 2H), 4.80 (d, 2H), 2.57 (m, 1H), 2.45 (m, 1H), 2.24 (m, 2H), 1.26 (m, 1H), 0.68-0.52 (m, 1H), 0.54-0.28 (m, 3H). ¹⁹F NMR (376.5 MHz, CD₃OD) δ −77.44, −62.82 LCMS: MS m/z (ESI): 400.0 [M + H]⁺ |
| | (S)-5-cyclopropyl-5-(3-(5-fluoro- | |
| | 6-(trifluoromethyl)isoindolin-2- | |
| | yl)-3-oxopropyl)imidazolidine- | |
| | 2,4-dione | |
| 46 | 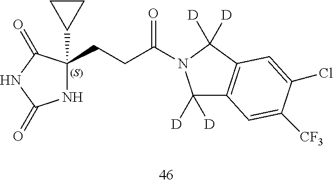 | ¹H NMR (400 MHz, CD₃OD): δ 7.67 (s, 1H), 7.51 (s, 1H), 2.49-2.42 (m, 1H), 2.36- 2.30 (m, 1H), 2.18-2.06 (m, 2H), 1.30-1.10 (m, 1H), 0.52-0.47 (m, 1H), 0.39-0.24 (m, 3H). LC-MS: MS m/z (ESI): [M + H]⁺. 420.2 |
| | (S)-5-(3-(5-chloro-6- | |
| | (trifluoromethyl)isoindolin-2-yl- | |
| | 1,1,3,3-d4)-3-oxopropyl)-5- | |
| | cyclopropylimidazolidine-2,4- | |
| | dione | |
| 47 | 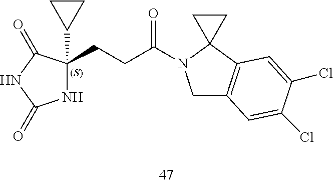 | ¹H NMR (400 MHz, CD₃OD): δ 7.36 (s, 1H), 6.89 (s, 1H), 4.83 (s, 2H), 2.41-2.33 (m, 4H), 2.29-2.21 (m, 1H), 2.09-1.99 (m, 2H), 1.13-1.08 (m, 1H), 0.81-0.78 (m, 2H), 0.50- 0.44 (m, 1H), 0.37-0.31 (m, 3H). LC-MS: MS m/z (ESI): [M + H]⁺ 408.1. |
| | (S)-5-cyclopropyl-5-(3-(5',6'- | |
| | dichlorospiro[cyclopropane-1,1'- | |
| | isoindolin]-2'-yl)-3- | |
| | oxopropyl)imidazolidine-2,4- | |
| | dione | |

The following compounds can be prepared using the similar methods as Examples 1-86.

Structure

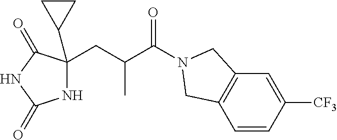 [SMILES: CC(CC1(C2CC2)NC(=O)NC1=O)C(=O)N1Cc2ccc(C(F)(F)F)cc2C1]

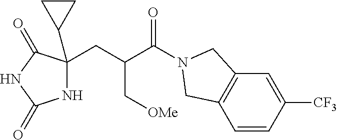 [SMILES: COCC(CC1(C2CC2)NC(=O)NC1=O)C(=O)N1Cc2ccc(C(F)(F)F)cc2C1]

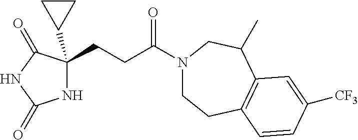 [SMILES: CC1CN(C(=O)CC[C@@]2(C3CC3)NC(=O)NC2=O)CCc2ccc(C(F)(F)F)cc21]

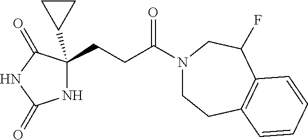 [SMILES: O=C1NC(=O)[C@](CCC(=O)N2CCc3ccccc3C(F)C2)(C2CC2)N1]

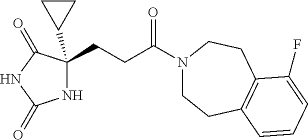 [SMILES: O=C1NC(=O)[C@](CCC(=O)N2CCc3cccc(F)c3CC2)(C2CC2)N1]

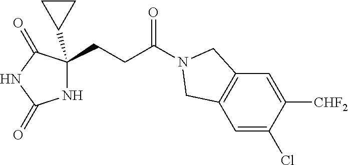 [SMILES: O=C1NC(=O)[C@](CCC(=O)N2Cc3cc(Cl)c(C(F)F)cc3C2)(C2CC2)N1]

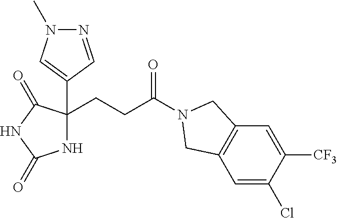 [SMILES: Cn1cc(C2(CCC(=O)N3Cc4cc(Cl)c(C(F)(F)F)cc4C3)NC(=O)NC2=O)cn1]

### Biological Assays

The present disclosure will be further described with reference to the following test examples, but the examples should not be considered as limiting the scope of the disclosure.

### Test Example 1. In Vitro Fluorescence Assay of ADAMTS-4 or ADAMTS-5 Activity

A FRET (fluorescence resonance energy transfer) peptide was cleaved by recombinant ADAMTS-4 or ADAMTS-5 proteins into two separate fragments resulting in an increase of fluorescence signal which was quantified. The peptide was 5-FAM-TEGEARGSVILLK(5-TAMRA)K-NH₂, customized from ANASPEC. ADAMTS-4 recombinant protein (catalog #4307-AD) and ADAMTS-5 recombinant protein (catalog #2198-AD) were purchased from R&D Systems.

An assay buffer containing 50 mM HEPES pH 7.5, 100 mM NaCl, 5 mM CaCl₂, 0.1% CHAPS and 5% Glycerol was prepared. A volume of 2.5 μl of compound in the assay buffer was dispensed to a 384-well plate, and 2.5 μl of ADAMTS-4 or ADAMTS-5 protein (final concentration in the reaction was 10 nM) was added. The compounds and proteins were pre-incubated at room temperature for 15 minutes. Then, 5 μl of substrate was added to each well. The final substrate concentrations for ADAMTS-4 and ADAMTS-5 were 15 μM and 8 μM, respectively. The fluorescence signal in each well was determined, after incubation at 37° C. for 3 hours, on a TECAN plate reader (Excitation, 490 nm; Emission, 520 nm).

### Data Analysis:

The data was inputted into GraphPad Prism, and the IC₅₀ values were calculated using function “log (inhibitor) vs. response—Variable slope (four parameters)”. (See Table 1)

TABLE 1
The IC₅₀ values of the exemplified compounds
in FRET-peptide enzymatic assays.

| | ADAMTS-4 | ADAMTS-5 |
|---|---|---|
| Example No. | (IC₅₀, nM) | (IC₅₀, nM) |
| 1 | 60 | 88 |
| 2 | 91 | 190 |
| 4 | 170 | 480 |
| 5 | 46 | 73 |
| 6 | 56 | 240 |
| 8 | 66 | 53 |
| 9 | 42 | 37 |
| 10 | 41 | 260 |
| 11 | 110 | 410 |
| 12 | 2300 | 1500 |
| 13 | 1400 | 4000 |
| 14 | 290 | 1000 |
| 16 | 1700 | 6200 |
| 17 | 310 | 370 |
| 19 | 260 | 330 |
| 20 | 22 | 37 |
| 21 | 20 | 16 |
| 22 | 51 | 61 |
| 24 | 120 | 360 |
| 25 | 5800 | 3600 |
| 26 | 3800 | 5400 |
| 27 | 170 | 270 |
| 28 | 47 | 48 |
| 29 | 14 | 22 |
| compound with shorter retention time | 8 | 9 |
| in 29-1 and 29-2 | | |
| compound with longer retention time | 12 | 9 |
| in 30-1 and 30-2 | | |
| compound with shorter retention time | 12 | 7.1 |
| in 32 and 33 | | |
| 34-2 | 7 | 9 |
| compound with longer retention time | 3 | 7 |
| in 34-2-A and 34-2-B | | |
| 35 | 230 | 110 |
| 36-1 | 21 | 15 |
| 37 | 5 | 13 |
| compound with longer retention time | 52 | 98 |
| in 37-1 and 37-2 | | |
| compound with shorter retention time | 8 | 12 |
| in 37-1 and 37-2 | | |
| 38 | 62 | 31 |
| compound with longer retention time | 330 | 110 |
| in 38-1 and 38-2 | | |
| compound with shorter retention time | 120 | 42 |
| in 38-1 and 38-2 | | |
| 39 | 110 | 62 |
| 40 | 42 | 25 |
| 41 | 36 | 21 |
| compound with shorter retention time | 50 | 27 |
| in 41-1 and 41-2 | | |
| 42 | 11 | 14 |
| 43 | 150 | 250 |
| 44 | 15 | 37 |
| 45 | 19 | 12 |
| 49-2 | 15 | 20 |
| compound with shorter retention time | 10 | 11 |
| in 49-2a and 49-2b | | |
| 50 | 120 | 220 |
| 52 | 30 | 66 |
| 53 | 240 | 170 |
| 54 | 11 | 110 |
| 57 | 43 | 130 |
| 59 | 82 | 46 |
| 60 | 170 | 220 |
| 61 | 40 | 42 |
| 62 | 100 | 89 |
| compound with longer retention time | 44 | 38 |
| in 62-1 and 62-2 | | |
| compound with longer retention time | 280 | 280 |
| in 64-1 and 64-2 | | |
| 65 | 16 | 21 |
| 66 | 43 | 52 |
| 66-2 | 12 | 10 |
| compound with a retention time of | 21 | 37 |
| 4.729 min in 67-1, 67-2, 68-1 and 68- | | |
| 2 (MeOH/DEA 5%_40% 1.5 ml/min | | |
| IA, 3 um 3.0*100(Daicel) | | |
| compound with a retention time of | 170 | 250 |
| 5.284 min in 67-1, 67-2, 68-1 and 68- | | |
| 2 (MeOH/DEA 5%_40% 1.5 ml/min | | |
| IA, 3 um 3.0*100(Daicel) | | |
| compound with a retention time of | 93 | 104 |
| 6.442 min in 67-1, 67-2, 68-1 and 68- | | |
| 2 (MeOH/DEA 5%_40% 1.5 ml/min | | |
| IA, 3 um 3.0*100(Daicel) | | |
| compound with a retention time of | 7 | 5 |
| 5.970 min in 67-1, 67-2, 68-1 and 68- | | |
| 2 (MeOH/DEA 5%_40% 1.5 ml/min | | |
| IA, 3 um 3.0*100(Daicel) | | |
| 69 | 68 | 40 |
| compound with shorter retention time | 24 | 18 |
| in 69-1 and 69-2 | | |
| 70 | 20 | 26 |
| 71 | 54 | 26 |
| compound with shorter retention time | 18 | 31 |
| in 71-1 and 71-2 | | |
| compound with longer retention time | 4300 | 4900 |
| in 71-1 and 71-2 | | |
| 72 | 43 | 130 |
| 73 | 61 | 27 |
| compound with shorter retention time | 54 | 25 |
| in 73-1 and 73-2 | | |
| 74 | 56 | 100 |
| 75 | 120 | 75 |
| 77 | 400 | 160 |
| 78 | 160 | 57 |
| 79 | 250 | 110 |
| 80 | 28 | 32 |
| 81 | 260 | 77 |
| 82 | 80 | 25 |
| compound with longer retention time | 61 | 34 |
| in 83-1 and 83-2 | | |
| compound with longer retention time | 74 | 32 |
| in 84-1 and 84-2 | | |
| compound with longer retention time | 100 | 39 |
| in 85-1 and 85-2 | | |
| compound with shortest retention time | 16 | 15 |
| in 86-1, 86-2, 86-3 and 86-4 | | |

Conclusion: The compounds of the present disclosure have a significant inhibition effect on the enzymatic activity of ADAMTS-4 and ADAMTS-5.

### Test Example 2. In Vitro ELISA (Enzyme-Linked Immunosorbent Assay) of ADAMTS-5 Activity

In this assay, the enzymatic activity of recombinant ADAMTS-5 protein (catalog #2198-AD, R&D Systems) was assayed with a protein substrate, the aggrecan IGD protein. The aggrecan IGD protein is a polypeptide connecting human aggrecan globular domains 1 and 2 (T331-G458) expressed in E. Coli with a C-terminal His-tag (catalog #30411000, BIOTEZ). The enzymatic product ARGSVIL-peptide was detected using an ELISA kit from BioTEZ (catalog #30510111).

An assay buffer containing 50 mM HEPES pH 7.5, 100 mM NaCl, 5 mM CaCl₂), 0.1% CHAPS and 5% Glycerol was prepared. Recombinant ADAMTS-5 protein was diluted to 0.3 nM in the assay buffer. Ten μl of buffer and 10 μl of compound solution was transferred to each well of a 96-well plate and incubated at room temperature for 15 minutes. Substrate aggrecan-IGD was diluted to 100 nM with the assay buffer and 20 μl was added to each well. The plate was incubated at 37° C. for 45 minutes. After incubation, the newly generated epitope ARGSVIL-peptides were measured using the Aggrecanase Activity ELISA Assay Kit following the manufacturer's instructions. Then, 100 μl of stop solution was added and the absorbance of each well was read at 450 nM, using 620 nM as reference on a TECAN plate reader.

### Data Analysis:

A standard curve of the ELISA assay was generated in GraphPad Prism using Sigmoidal 4PL function and the corresponding peptide concentrations were calculated based on the standard curve. The IC₅₀ values were calculated using function “log (inhibitor) vs. response—Variable slope (four parameters)”. (See Table 2).

TABLE 2
The IC₅₀ values of the exemplified compounds
from the Aggrecan-IGD enzymatic assay.

| Example No. | ADAMTS-5 (IC₅₀, nM) |
|---|---|
| 1 | 140 |
| 2 | 860 |
| 4 | 730 |
| 5 | 240 |
| 6 | 650 |
| 8 | 130 |
| 9 | 56 |
| 11 | 560 |
| 22 | 140 |
| 24 | 840 |
| 29 | 56 |
| compound with shorter retention time | 19 |
| in 29-1 and 29-2 | |
| 31-2 | 33 |
| compound with shorter retention time | 18 |
| in 32 and 33 | |
| 34-2 | 17 |
| compound with longer retention time | 18 |
| in 34-2-A and 34-2-B | |
| 35 | 170 |
| 36-1 | 36 |
| 37 | 16 |
| 38 | 29 |
| compound with longer retention time | 220 |
| in 38-1 and 38-2 | |
| compound with shorter retention time | 52 |
| in 38-1 and 38-2 | |
| 39 | 65 |
| 40 | 92 |
| 41 | 18 |
| compound with shorter retention time | 17 |
| in 41-1 and 41-2 | |
| 42 | 17 |
| 44 | 51 |
| 45 | 25 |
| 49-2 | 55 |
| compound with shorter retention time | 39 |
| in 49-2a and 49-2b | |
| 52 | 48 |
| 54 | 200 |
| 57 | 190 |
| 59 | 46 |
| 61 | 72 |
| 62 | 290 |
| compound with longer retention time | 85 |
| in 62-1 and 62-2 | |
| 65 | 31 |
| 66 | 83 |
| 66-2 | 19 |
| compound with a retention time of | 85 |
| 4.729 min in 67-1, 67-2, 68-1 and 68- | |
| 2 (MeOH/DEA 5%_40% 1.5 ml/min | |
| IA, 3 um 3.0*100(Daicel) | |
| compound with a retention time of | |
| 6.442 min in 67-1, 67-2, 68-1 and 68- | 290 |
| 2 (MeOH/DEA 5%_40% 1.5 ml/min | |
| IA, 3 um 3.0*100(Daicel) | |
| compound with a retention time of | 10 |
| 5.970 min in 67-1, 67-2, 68-1 and 68- | |
| 2 (MeOH/DEA 5%_40% 1.5 ml/min | |
| IA, 3 um 3.0*100(Daicel) | |
| 69 | 47 |
| compound with shorter retention time | 20 |
| in 69-1 and 69-2 | |
| 70 | 83 |
| 71 | 29 |
| compound with shorter retention time | 13 |
| in 71-1 and 71-2 | |
| 73 | 47 |
| compound with shorter retention time | 26 |
| in 73-1 and 73-2 | |
| 75 | 110 |
| 78 | 76 |
| 79 | 210 |
| 81 | 120 |
| compound with longer retention time | 55 |
| in 84-1 and 84-2 | |
| compound with longer retention time | 45 |
| in 85-1 and 85-2 | |

Conclusion: The compounds of the present disclosure have a significant inhibition effect on the enzymatic activity of ADAMTS-5.

### Test Example 3. Mouse Explant Assay

In this assay, fresh mouse femoral head cartilage was treated with IL-1α protein (Sigma-Aldrich, catalog #12778) in culture media, which induces the cartilage catabolism. Then, the GAGs attached to the cleaved aggrecan fragments (released in the media) and the GAGs attached to the intact aggrecan were measured by dimethylmethylene blue dye in the Glycosaminoglycans Assay Kit (Chondrex, catalog #6022).

Femoral head cartilage samples were isolated from mice (25 days old, male, C₅₇BL/6, from Charles River Lab) and put into 2.0 ml tubes filled-up with media (DMEM, 10% FBS, 4 mM Glutamine, penicillin-streptomycin, 20 mM HEPES). Two hundred μl of media without FBS was added to each well of a 48-well plate, and one piece of cartilage was transferred to a well in the plate. Then the media was aspirated, and compounds and IL-1α protein were added to the plate in a total volume of 400 μl of fresh media without FBS. The final concentration of IL-1α was 1 ng/ml. The plate was incubated at 37° C. for 72 hours in a humidified incubator with 5% CO₂ supply.

The supernatant was transferred to a 1.5 ml tube and kept at −20° C. Each cartilage sample was transferred to another 1.5 ml tube containing 400 μl of freshly made papain solution. The papain solution contained 125 μg/ml papain (Sigma-Aldrich, catalog #P3125), 0.1 M sodium acetate (Sigma-Aldrich, catalog #S7899), pH 5.5 and 5 mM EDTA and 5 mM L-cysteine-HCl (Sigma-Aldrich, catalog #C7880). The cartilage samples were kept rocking in a 60° C. water bath for 24 hours. The lysates were vortexed for 10 seconds and spinned at 10,000 rpm for 2 minutes.

Both the supernatant and the lysate samples were diluted with PBS and mixed with 100 μl of dye from the Glycosaminoglycans Assay Kit. The optical density from each well was determined with a TECAN plate reader set to a wavelength of 525 nm.

### Data Analysis:

The concentrations of GAGs in the supernatant and lysates were determined based on the standard curve with a dose range of chondroitin sulfate provided in the kit. The percentage of GAG release was calculated as the following

$$GAG \% = ( \frac{[ GAG ] supernatant}{[ GAG ] supernatant + [ GAG ] lysate} ) \times 100 \% .$$

The test compound effect was expressed as the percent of inhibition using the following formula:

$$Inhibition \% = ( 1 h \frac{GAG \% ( Compound + IL 1 α ) - GAG \% ( Vehicle )}{GAG \% ( Vehicle + IL 1 c ) - GAG \% ( Vehicle )} ) \times 100 \%$$

The inhibition data of selected exemplified compounds at 2 M and 20 μM concentrations were listed in Table 3.

TABLE 3
The IC₅₀ values of the exemplified compounds
from the mouse explant assay.

| | Inhibition % | Inhibition % |
|---|---|---|
| Example No. | at 2 μM | at 20 μM |
| 1 | 27 | 61 |
| 5 | 19 | 51 |
| 8 | 22 | 63 |
| 9 | 22 | 55 |
| 20 | 67 | 100 |
| 21 | 39 | 100 |
| compound with shorter retention | 46 | 99 |
| time in 29-1 and 29-2 | | |
| compound with shorter retention | 65 | 97 |
| time in 32 and 33 | | |
| compound with longer retention | 71 | 100 |
| time in 34-2-A and 34-2-B | | |
| 40 | 2 | 52 |
| 42 | 51 | 92 |
| compound with shorter retention | 50 | 74 |
| time in 41-1 and 41-2 | | |
| 44 | 13 | 70 |
| 45 | 45 | 96 |
| 52 | 10 | 48 |
| 59 | 16 | 66 |
| compound with longer retention | 27 | 60 |
| time in 62-1 and 62-2 | | |
| 66-2 | 23 | 87 |
| compound with shorter retention | 31 | 98 |
| time in 69-1 and 69-2 | | |
| compound with shorter retention | 48 | 89 |
| time in 71-1 and 71-2 | | |
| compound with shorter retention | 31 | 70 |
| time in 73-1 and 73-2 | | |
| 80 | 30 | 73 |

The foregoing embodiments and examples are provided for illustration only and are not intended to limit the scope of the disclosure. Various changes and modifications to the disclosed embodiments will be apparent to those skilled in the art based on the present disclosure, and such changes and modifications may be made without departure from the spirit and scope of the present disclosure. All literature cited are incorporated herein by reference in their entireties without admission of them as prior art.

1. A compound of formula (I), or a tautomer, mesomer, racemate, enantiomer, diastereomer, or mixture thereof, or a pharmaceutically acceptable salt, solvate or prodrug thereof: 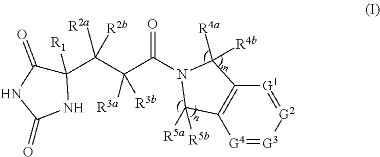
wherein:
G¹, G², G³ and G⁴ are each identical or different, and each is independently N or CR⁶, provided that no more than two of them are N;
R¹ is selected from the group consisting of alkyl, haloalkyl, hydroxyalkyl, cycloalkyl, heterocyclyl and heteroaryl, wherein the alkyl, cycloalkyl, heterocyclyl or heteroaryl is optionally substituted with one or more groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NHC(═O)R^{11a}, NHC(═O)OR^{11a}, cycloalkyl, heterocyclyl, aryl and heteroaryl;
R^{2a}, R^{2b}, R^{3a} and R^{3b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{2a}R^{12b}, NHC(═O)R^{12a} NHC(═O)OR^{12a}, OR^{12a}, cycloalkyl, heterocyclyl, aryl and heteroaryl;
or two of R^{2a}, R^{2b}, R^{3a} and R^{3b} together with the carbon atom to which they are attached form cycloalkyl or heterocyclyl;
R^{4a}, R^{4b}, R^{5a} and R^{5b} are each identical or different, and each is independently selected from the group consisting of hydrogen, deuterium, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups selected from the group consisting of halogen, alkyl, alkoxy, hydroxy, hydroxyalkyl, cyano, amino, nitro, cycloalkyl, heterocyclyl, aryl and heteroaryl;
or two of R^{4a}, R^{4b}, R^{5a} and R^{5b} together with the carbon atom to which they are attached form cycloalkyl or heterocyclyl;
each R⁶ is identical or different, and each is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a}, NHC(═O)OR^{13a}, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
R^{11a}, R^{12a}, R^{13a}, and R^{14a} are each independently selected from the group consisting of hydrogen, alkyl, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, hydroxy, alkoxy, alkyl, aryl and cycloalkyl;
R^{11a}, R^{12b}, R^{13b}, R^{14b} are each independently selected from the group consisting of hydrogen and alkyl, wherein alkyl is optionally substituted with one or more groups independently selected from the group consisting of halogen, hydroxyl and alkoxy;
n is 1 or 2; and
m is 1 or 2.

2. The compound of claim 1, or a tautomer, mesomer, racemate, enantiomer, diastereomer, or mixture thereof, or a pharmaceutically acceptable salt, solvate or prodrug thereof,
wherein:
G¹, G², G³ and G⁴ are each identical or different, and each is independently N or CR⁶, provided that no more than two of them are N;
R¹ is selected from the group consisting of alkyl, haloalkyl, hydroxyalkyl, cycloalkyl, heterocyclyl and heteroaryl, wherein the alkyl, cycloalkyl, heterocyclyl or heteroaryl is optionally substituted with one or more groups independently selected from the group consisting of halogen, hydroxy, cyano, alkyl, alkoxy, hydroxyalkyl, SO₂R^{11a}, NR^{11a}R^{11b}, C(═O)OR^{11a}, C(═O)NR^{11a}R^{11b}, NHC(═O)R^{11a}, NHC(═O)OR^{11a}, cycloalkyl, heterocyclyl, aryl and heteroaryl;
R^{2a}, R^{2b}, R^{3a} and R^{3b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, NR^{12a}R^{12b}, C(═O)OR^{12a}, C(═O)NR^{12a}R^{12b}, NHC(═O)R^{12a}, NHC(═O)OR^{12a}, cycloalkyl, heterocyclyl, aryl and heteroaryl;
or two of R^{2a}, R^{2b}, R^{3a} and R^{3b} together with the carbon atom to which they are attached form cycloalkyl or heterocyclyl;
R^{4a}, R^{4b}, R^{5a} and R^{5b} are each identical or different, and each is independently selected from the group consisting of hydrogen, halogen, alkyl, alkoxy, hydroxy, haloalkyl, haloalkoxy, hydroxyalkyl, cyano, amino, nitro, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, cycloalkyl, heterocyclyl, aryl and heteroaryl;
or two of R^{4a}, R^{4b}, R^{5a} and R^{5b} together with the carbon atom to which they are attached form cycloalkyl or heterocyclyl;
each R⁶ is identical or different, and each is independently selected from the group consisting of hydrogen, halogen, alkyl, alkoxy, haloalkyl, haloalkoxy, hydroxy, hydroxyalkyl, cyano, amino, nitro, SO₂R^{13a}, SO₂NR^{13a}R^{13b}, NR^{13a}R^{13b}, C(═O)OR^{13a}, C(═O)NR^{13a}R^{13b}, NHC(═O)R^{13a} NHC(═O)OR^{13a}, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, alkyl, alkoxy, hydroxy, hydroxyalkyl, cyano, amino, nitro, SO₂R^{14a}, SO₂NR^{14a}R^{14b}, NR^{14a}R^{14b}, C(═O)OR^{14a}, C(═O)NR^{14a}R^{14b}, NHC(═O)R^{14a}, NHC(═O)OR^{14a}, cycloalkyl, heterocyclyl, aryl and heteroaryl;
R^{11a}, R^{12a}, R^{13a}, and R^{14a} are each independently selected from the group consisting of hydrogen, alkyl, cycloalkyl, heterocyclyl, aryl and heteroaryl, wherein the alkyl, cycloalkyl, heterocyclyl, aryl or heteroaryl is optionally substituted with one or more groups independently selected from the group consisting of halogen, hydroxy, alkoxy, alkyl and cycloalkyl;
R^{11a}, R^{12b}, R^{13b}, R^{14b} are each independently selected from the group consisting of hydrogen and alkyl, wherein alkyl is optionally substituted with one or more groups independently selected from the group consisting of halogen, hydroxyl and alkoxy;
n is 1 or 2; and
m is 1 or 2.

3. The compound of claim 1, having a structure of formula (II), or a tautomer, mesomer, racemate, enantiomer, diastereomer, or mixture thereof, or a pharmaceutically acceptable salt, solvate or prodrug thereof: 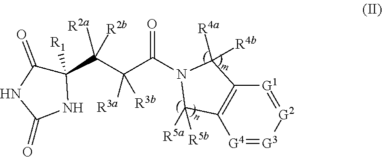
wherein G¹, G², G³, G⁴, R¹, R^{2a} to R^{5a}, R^{2b} to R^{5b}, n and m are each as defined in claim 1.

4. The compound of claim 1, or a tautomer, mesomer, racemate, enantiomer, diastereomer, or mixture thereof, or a pharmaceutically acceptable salt, solvate or prodrug thereof, wherein G¹ and G² are each identical or different, and each is independently N or CR⁶; and G³ and G⁴ are each CR⁶; wherein R⁶ is as defined in claim 1.

5. The compound of claim 1, having a structure of formula (III) or (IIIa), or a tautomer, mesomer, racemate, enantiomer, diastereomer, or mixture thereof, or a pharmaceutically acceptable salt, solvate or prodrug thereof: 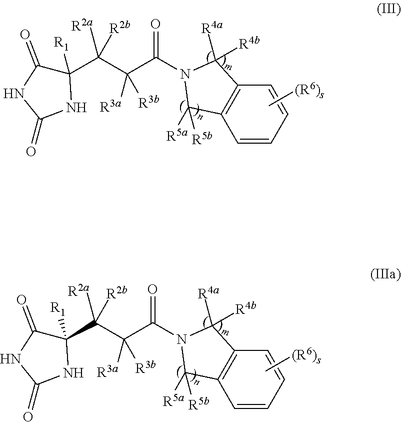
wherein:
s is 0, 1 or 2; and
R¹, R^{2a} to R^{5a}, R^{2b} to R^{5b}, R⁶, n and m are each as defined in claim 1.

6. The compound of claim 1, or a tautomer, mesomer, racemate, enantiomer, diastereomer, or mixture thereof, or a pharmaceutically acceptable salt, solvate or prodrug thereof, wherein R¹ is selected from the group consisting of C₁₋₆ alkyl, 3 to 8-member cycloalkyl and 5 to 10-member heteroaryl, wherein the C₁₋₆ alkyl, 3 to 8-member cycloalkyl and 5 to 10-member heteroaryl are each optionally substituted with one or more groups independently selected from the group consisting of C₁₋₆ alkyl and C₁₋₆ alkoxy.

7. (canceled)

8. The compound of claim 1, or a tautomer, mesomer, racemate, enantiomer, diastereomer, or mixture thereof, or a pharmaceutically acceptable salt, solvate or prodrug thereof, wherein R^{2a} and R^{2b} are both hydrogen.

9. The compound of claim 1, or a tautomer, mesomer, racemate, enantiomer, diastereomer, or mixture thereof, or a pharmaceutically acceptable salt, solvate or prodrug thereof, having a structure of formula (IV) or (IVa), or a tautomer, mesomer, racemate, enantiomer, diastereomer, or mixture thereof, or a pharmaceutically acceptable salt, solvate or prodrug thereof: 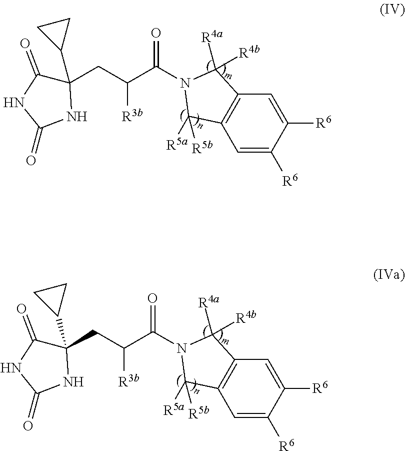
wherein:
R^{4a}, R^{5a}, R^{3b} to R^{5b}, R⁶, n and m are each as defined in claim 1.

10. The compound of claim 1, or a tautomer, mesomer, racemate, enantiomer, diastereomer, or mixture thereof or a pharmaceutically acceptable salt, solvate or prodrug thereof, wherein R^{3a} and R^{3b} are identical or different, and each is selected from the group consisting of hydrogen, halogen, C₁₋₆ alkyl, C₁₋₆ alkoxy and C₁₋₆ hydroxyalkyl, wherein the C₁₋₆ alkyl is optionally substituted with one or more groups independently selected from the group consisting of halogen and C₁₋₆ alkoxy.

11. The compound of claim 1, or a tautomer, mesomer, racemate, enantiomer, diastereomer, or mixture thereof, or a pharmaceutically acceptable salt, solvate or prodrug thereof, wherein R^{4a}, R^{4b}, R^{5a} and R^{5b} are each identical or different, and each is independently selected from the group consisting of hydrogen, deuterium, halogen and C₁₋₆ alkyl; or R^{5a} and R^{5b} together with the carbon atom to which they are attached form 3 to 8-member cycloalkyl, R^{4a} and R^{4b} are each identical or different, and each is independently selected from the group consisting of hydrogen, deuterium, halogen and C₁₋₆ alkyl; or R^{4a} and R^{4b} together with the carbon atom to which they are attached form 3 to 8-member cycloalkyl, R^{5a} and R^{5b} are each identical or different, and each is independently selected from the group consisting of hydrogen, deuterium, halogen and C₁₋₆ alkyl.

12. (canceled)

13. The compound of claim 1, or a tautomer, mesomer, racemate, enantiomer, diastereomer, or mixture thereof, or a pharmaceutically acceptable salt, solvate or prodrug thereof, wherein 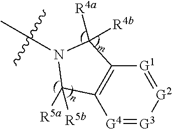
is selected from the group consisting of 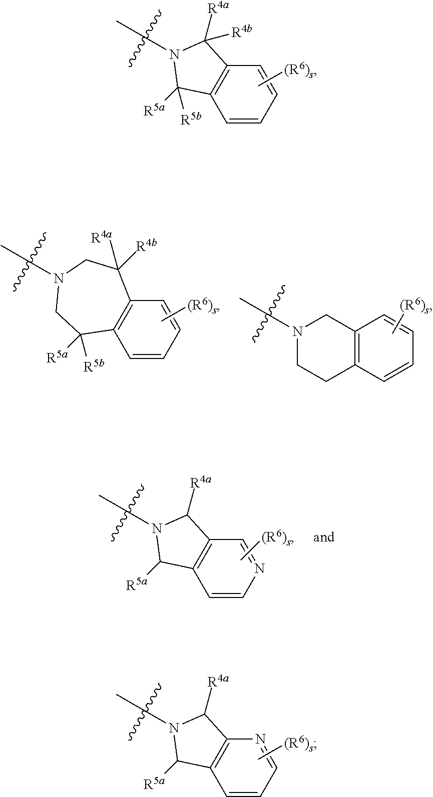
s is 0, 1 or 2; and
R^{4a}, R^{4b}, R^{5a}, R^{5b} and R⁶ are as defined in claim 1.

14. The compound of claim 1, or a tautomer, mesomer, racemate, enantiomer, diastereomer, or mixture thereof, or a pharmaceutically acceptable salt, solvate or prodrug thereof, having a structure of formula (V), or a tautomer, mesomer, racemate, enantiomer, diastereomer, or mixture thereof, or a pharmaceutically acceptable salt, solvate or prodrug thereof: 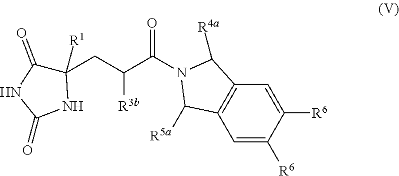
wherein:
R¹ is selected from the group consisting of cycloalkyl and heteroaryl, wherein the cycloalkyl or heteroaryl is optionally substituted with one or more groups independently selected from the group consisting of halogen, hydroxy, cyano, alkyl, alkoxy and hydroxyalkyl; and
R^{3b}, R^{4a}, R^{5a} and R⁶ are each as defined in claim 1.

15. The compound of om claim 1, or a tautomer,
mesomer, racemate, enantiomer, diastereomer, or mixture thereof, or a pharmaceutically acceptable salt, solvate or prodrug thereof, wherein each R⁶ is identical or different, and each is independently selected from the group consisting of hydrogen, halogen, C₁₋₆ alkyl, C₁₋₆ alkoxy, C₁₋₆ haloalkyl, C₁₋₆ haloalkoxy, cyano and C(═O)OR^{13a}; and R^{13a} is hydrogen or C₁₋₆ alkyl.

16. (canceled)

17. A compound selected from the group consisting of: 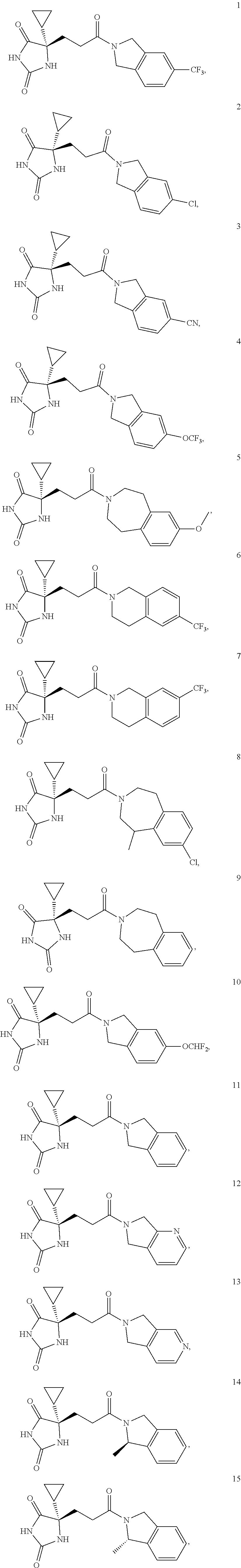 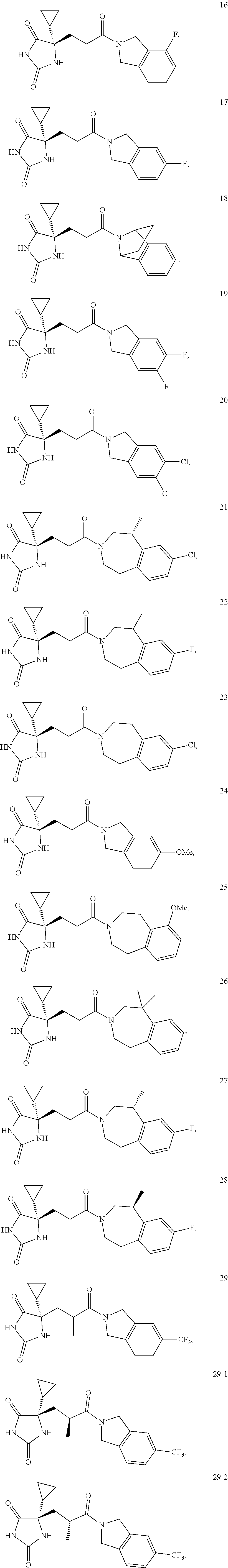 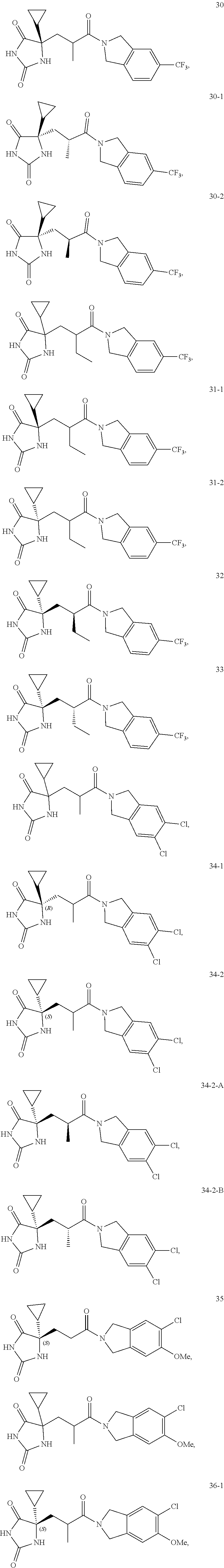 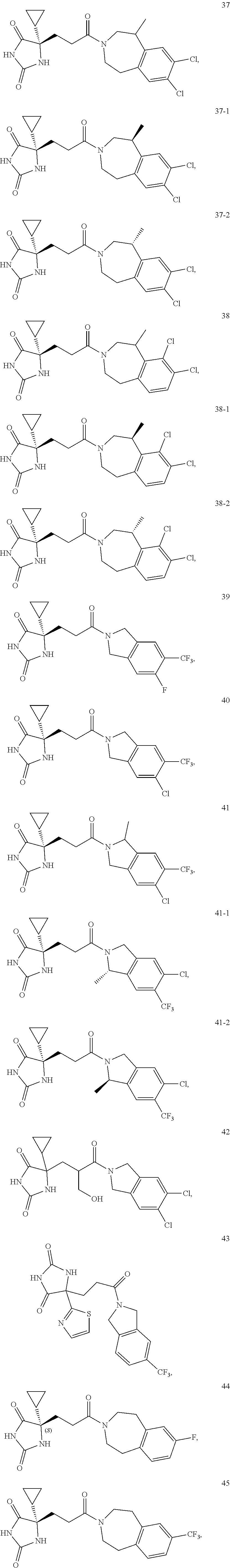 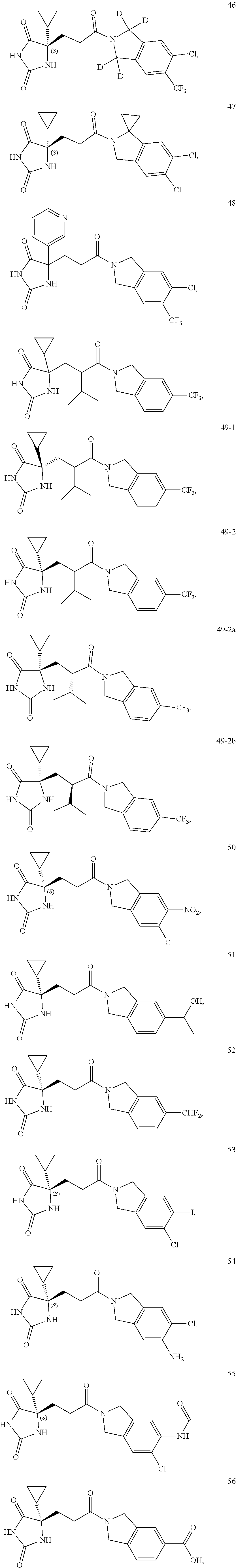 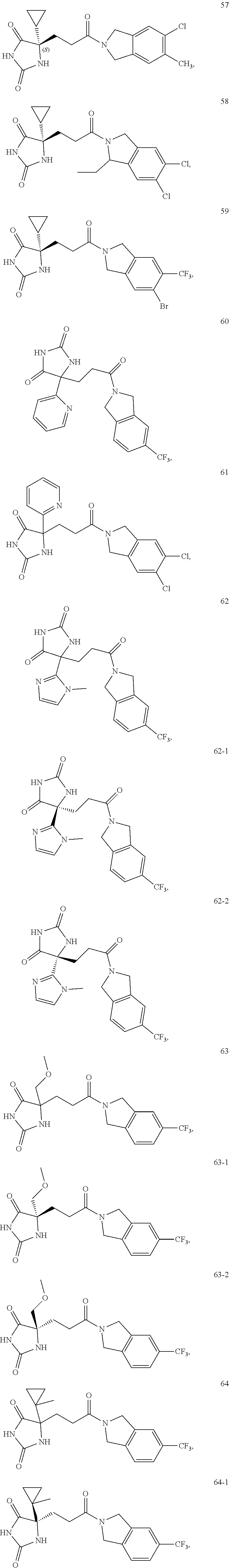 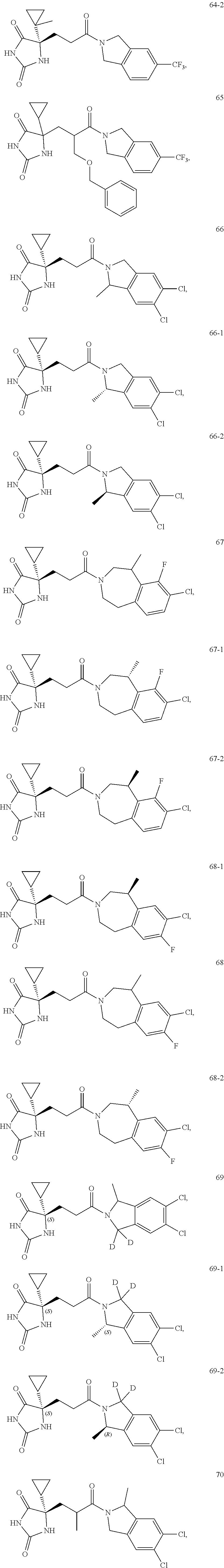 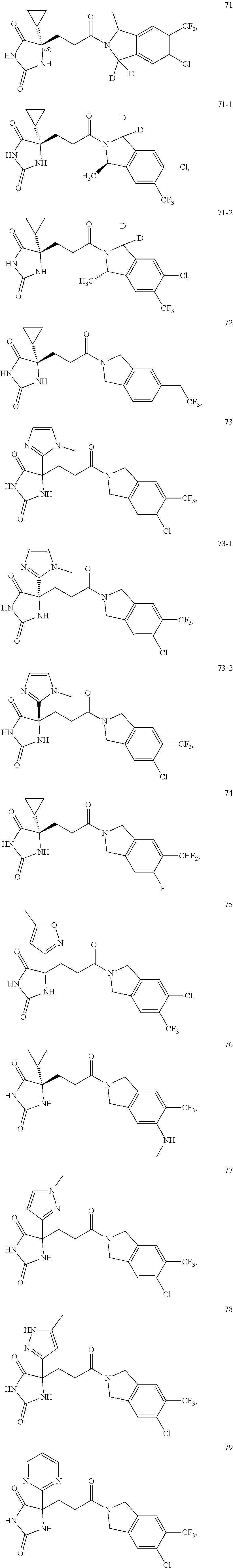 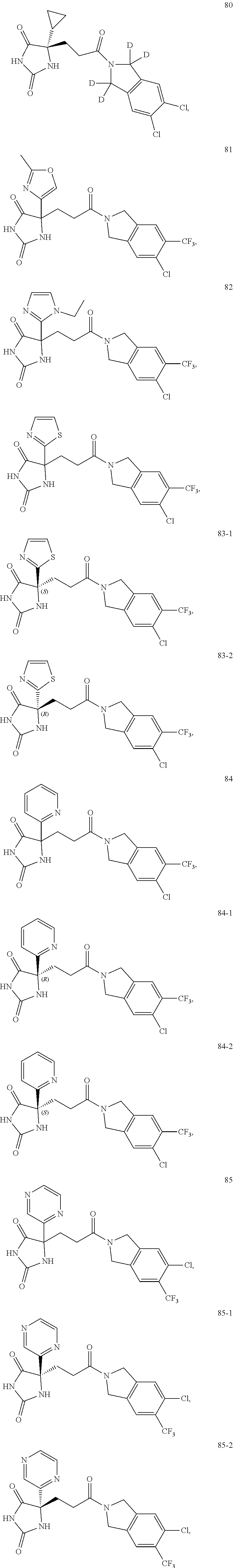 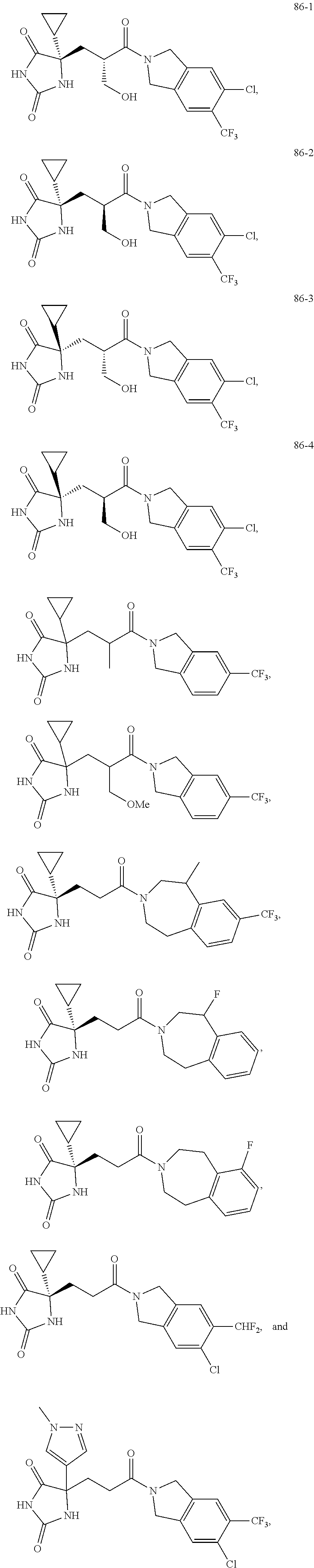
or a tautomer, mesomer, racemate, enantiomer, diastereomer, or mixture thereof, or a pharmaceutically acceptable salt, solvate or prodrug thereof.

18. A process of preparing the compound of formula (I) according to claim 1, or a tautomer, mesomer, racemate, enantiomer, diastereomer, or mixture thereof, or a pharmaceutically acceptable salt, solvate or prodrug thereof, comprising a step of: 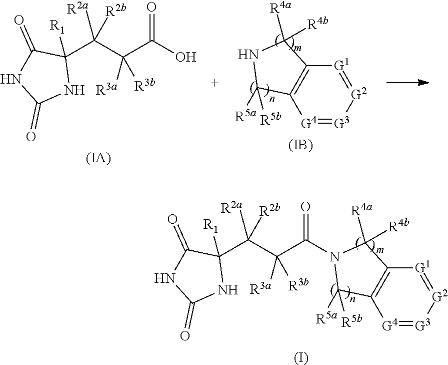
reacting a compound of formula (IA) or a tautomer, mesomer, racemate, enantiomer, diastereomer, or mixture thereof, or a salt thereof with a compound of formula (IB) or a salt thereof to obtain the compound of formula (I) or a tautomer, mesomer, racemate, enantiomer, diastereomer, or mixture thereof, or a pharmaceutically acceptable salt, solvate or prodrug thereof;
wherein:
G¹, G², G³, G⁴, R¹, R^{2a} to R^{5a}, R^{2b} to R^{5b}, n and m are each as defined in claim 1.

19. A pharmaceutical composition comprising a compound of claim 1, or a tautomer, mesomer, racemate, enantiomer, diastereomer, or mixture thereof, or a pharmaceutically acceptable salt, solvate or prodrug thereof, and a pharmaceutically acceptable carrier.

20. A method of inhibiting ADAMTS-5 and/or ADAMTS-4, comprising administering to a subject in need thereof a therapeutically effective amount of the pharmaceutical composition of claim 19.

21. A method of preventing or treating an inflammatory condition or disease involving degradation of cartilage, and/or disruption of cartilage homeostasis, comprising administering to a subject in need thereof a therapeutically effective amount of the pharmaceutical composition of claim 19.

22. A method of preventing or treating arthritis, comprising administering to a subject in need thereof a therapeutically effective amount of the pharmaceutical composition of claim 19.

23. The method of claim 22, wherein the arthritis is selected from the group consisting of rheumatoid arthritis, psoriatic arthritis, osteoarthrosis and hypertropic arthritis.

24. (canceled)

25. (canceled)
